(19) United States
(12) Patent Application Publication
(10) Pub. No.: US 2025/0312287 A1
(43) Pub. Date: Oct. 9, 2025

(54) LIPID NANOPARTICLES COMPRISING AN IMIDAZOLIUM-BASED CATIONIC LIPID

(71) Applicant: POLYPLUS TRANSFECTION, ILLKIRCH-GRAFFENSTADEN (FR)

(72) Inventors: Malik HELLAL, ILLKIRCH (FR); Claire GUEGUEN, PLOBSHEIM (FR)

(21) Appl. No.: 18/865,240

(22) PCT Filed: Jul. 7, 2023
(86) PCT No.: PCT/EP2023/068940

§ 371 Date: Nov. 12, 2024

(30) Foreign Application Priority Data
Jul. 8, 2022 (EP) 22306027.8

(51) Int. Cl. A61K 9/51; A61K 31/7105

(52) U.S. Cl. CPC A61K 9/5123; A61K 31/7105

(57) ABSTRACT

The present invention relates to compositions for in vitro and in vivo delivery of nucleic acids, in particular messenger RNAs (mRNAs), into a target cell and their applications. The present invention is directed to a composition comprising (A) at least one nucleic acid and (B) at least one lipid nanoparticle (LNP) comprising (i) at least one ionizable lipid; (ii) at least one phospholipid; (iii) at least one sterol, especially neutral sterol; (iv) at least one poly(ethyleneglycol)-lipid (PEG-lipid); and (v) an imidazolium-based cationic lipid of formula (I), wherein R₁, R₂, R₃, R₄, R₅, R₆, R₇, R₈ and R₉, Y and A⁻ are as defined in the description. The present invention also relates to a method for in vitro or in vivo transfection of live cells and uses of said composition.

The present invention relates to compositions based on the use of lipid nanoparticles for in vitro or in vivo delivery of nucleic acids, in particular messenger RNAs (mRNAs), into a target cell and their applications. The present invention is directed to a composition comprising (A) at least one nucleic acid and (B) at least one lipid nanoparticle (LNP) comprising (i) at least one ionizable lipid; (ii) at least one phospholipid; (iii) at least one sterol, especially a neutral sterol; (iv) at least one poly(ethyleneglycol)-lipid (PEG-lipid); and (v) an imidazolium-based cationic lipid of formula (I), wherein R₁, R₂, R₃, R₄, R₅, R₆, R₇, R₈ and R₉, Y and A⁻ are as defined in the description. The present invention also relates to a method for in vitro or in vivo transfection of live cells and uses of said composition.

Common methods for therapeutic nucleic acid delivery include viral- and non-viral-mediated approaches. Although viral-mediated delivery of nucleic acids for gene therapy holds tremendous promise, non-viral-mediated delivery may be a more suitable alternative. Nucleic acid delivery via viral vectors indeed presents the risk of ectopic vector integration, which may lead to persistent transgene expression and deleterious consequences for some therapies including gene editing. Alternatively, non-viral nucleic acid, in particular mRNA delivery approaches, can enable transient nucleic acid expression without the risk of genome integration of the nucleic acid.

The European patent EP2004156 discloses a composition of transfection comprising an oligonucleotide active for gene silencing and an amphiphilic cationic molecule. This document discloses preparation of a liposome.

The European patent application EP3646854 discloses a composition for transfecting a messenger RNA (mRNA) into a cell. The composition comprises a neutral lipid and a cationic lipid.

The document discloses formulation of a liposome.

None of this document disclose a complex composition comprising LNP and enabling encapsulation of a nucleic acid.

The international patent applications WO2020/051220 and WO2020/051223 disclose the use of lipid nanoparticle compositions comprising a selective organ targeting compound. The selective organ targeting compound may be a lipid such as a permanently cationic lipid or a permanently anionic lipid. WO2020/051220 particularly describes the use of DOTAP (1,2-Dioleoyl-3-trimethylammonium-propane chloride) as permanently cationic lipid. DOTAP mDLNP formulations (lipid nanoparticles comprising DOTAP) are described with different DOTAP molar percentages, to deliver mRNA. The higher transfection efficiency is shown with DOTAP10 (10% of DOTAP, molar percentage) [cf. FIG. 6A and paragraph 72, page 28]. Moreover, with concentrations of DOTAP lower than 25%, encapsulation efficiency is low, but increases to at least 80% with a molar percentage of DOTAP above 25% [cf. Paragraph 5, p. 90 and FIG. 5C]. The encapsulation efficiency of DOTAP10 is around 40%. Organ biodistributions of specific DOTAP formulations was studied [FIG. 7], DOTAP10 is expressed mostly in liver. Another experiment [FIG. 1B] shows that with an increasing molar percentage of DOTAP, luciferase expression moves from liver to spleen, then to lung, demonstrating organ specific delivery. Despite some showing of organ targeting may be shown in this patent application, the transfection efficiency of the DOTAP mDLNP remains very low and the other LNP characteristics (zeta/size/encapsulation efficiency) are not acceptable.

The international patent application WO2021/178396 discloses imidazole-based synthetic lipidoid LNPs for in vivo mRNA delivery into immune cells. mRNA-LNPs (such as COVID-19 vaccines BNT162b2 and mRNA-1273) containing an ionizable lipid exhibit a neutral charge, which is mainly responsible of the targeting into the liver (Schoenmaker et al., Int. J. Pharm., 2021, 601, 120586).

It has been reported that the replacement of helper lipids with charged alternatives in LNPs facilitates targeted mRNA delivery to the spleen and lungs but significantly reduces the transfection efficiency (Journal of Controlled Release, 2022, 345, 819-831).

Despite high transfection efficiency of cationic lipid-based lipoplexes, the clearance of cationic liposomes through the draining lymphatics after intramuscular administration is much attenuated and a depot is formed at the injection site. Moreover, cationic lipid-based lipoplexes are less efficient in endosomal escape than ionizable lipid-based ones. Another challenge for cationic lipid-based lipoplexes is toxicity. Cationic liposomes, when administered in vivo, can induce liver damage and increase the total number of leukocytes significantly (Nanoplatforms for mRNA Therapeutics. Chaoyang Meng, et al. Adv. Therap. 2020, 2000099).

There remains a need for improved LNPs comprising cationic lipids for the delivery of nucleic acids both in vitro and in vivo. Preferably these LNPs would provide in vitro and/or in vivo transfection efficiency associated with organ targeting capability that may include in vivo flexibility of delivery or biodistribution toward various organs. These LNPs should provide high transfection efficiency and/or high encapsulation efficiency, and/or exhibit other suitable LNPs characteristics such as zeta, size.

Thus, it is an object of the present invention to provide a composition comprising LNPs comprising an imidazolium-based cationic lipid in order to modify usual LNPs biodistribution through systemic administration toward lungs, spleen and liver without affecting transfection efficiency. In the composition, the LNPs may be of the same type (i.e., of the same composition, account being taken or not of the nucleic acid contents) or may be provided as an admixture of distinct types of LNPs wherein the LNPs are selected from the herein disclosed LNPs.

It is another object of the present invention to provide a method for transfecting nucleic acids, in particular mRNAs, using said composition.

The present invention relates to a composition comprising:
(A) at least one nucleic acid; and
(B) at least one lipid nanoparticle (LNP) comprising:
   (i) at least one ionizable lipid;
   (ii) at least one phospholipid;
   (iii) at least one sterol, especially a neutral sterol;
   (iv) at least one poly(ethyleneglycol)-lipid (PEG-lipid); and
   (v) an imidazolium-based cationic lipid of formula (I):

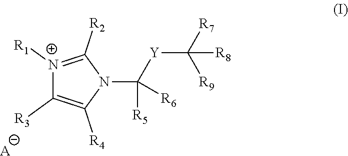

wherein
   R₁ represents a C₁-C₁₀ linear or branched hydrocarbon chain or a C₁-C₁₀ hydroxylated chain, preferably R₁ represents a C₁-C₁₀ linear or branched hydrocarbon chain;
   R₂, R₃ and R₄, which may be identical or different, represent H or a C₁-C₁₀ linear or branched hydrocarbon chain or a C₁-C₁₀ hydroxylated chain, preferably R₂, R₃ and R₄ represent H;
   R₅, R₆, R₇, R₈ and R₉, which may be identical or different, represent H; a saturated or unsaturated, linear or branched hydrocarbon chain selected among a C₆-C₃₃ chain, a NH—C₆-C₃₃, a —COO—C₆-C₃₃, a —O—C₆-C₃₃, a —S—C₆-C₃₃; or a saturated or unsaturated C₆ cycle, preferably R₅, R₆, R₇, R₈ and R₉, which may be identical or different, represent H, a saturated or unsaturated, linear or branched C₆-C₃₃ chain or a NH—C₆-C₃₃;
   Y represents —(CR₁₀R₁₁)ₘ—SO₂—; —(CR₁₀R₁₁)ₘ—COO—; —(CR₁₀R₁₁)ₘ—(CH₂)ₙ—; —(CR₁₀R₁₁)ₘ—CO—NH₂; —(CR₁₀R₁₁)ₘ—CH₂—[O—(CH₂)₂O]ₚ—; —(CR₁₀R₁₁)ₘ—(CH₂)ₙ—S—S—; —(CR₁₀R₁₁)ₘ—(CH₂)ₙ—S—; —(CR₁₀R₁₁)ₘ—(CH₂)ₙ—O—; —(CR₁₀R₁₁)ₘ—(CH₂)ₙ—NH—; —CH₂—; —COO—; or —CO—NH—, preferably —(CR₁₀R₁₁)ₘ—(CH₂)ₙ—; with:
      n representing an integer between 1 and 10 inclusive, preferably n=1 or 4;
      R₁₀ and R₁₁ representing H; a C₂-C₃₃ saturated or unsaturated, linear or branched hydrocarbon chain; or a saturated or unsaturated C₆ cycle, preferably R₁₀ and R₁₁ represent H;
      m representing an integer between 1 and 4 inclusive, preferably m=1 or 3; and
      p representing an integer between 1 and 4 inclusive, preferably p=1.
A⁻ represents a biocompatible anion, preferably A⁻ is selected from the group consisting of Cl⁻, Br⁻, MsO⁻, I⁻, NaCO₃⁻, HCO₃⁻, H₂CO₃²⁻, CO₃²⁻, H₂PO⁴⁻, HPO₄²⁻, HSO₄²⁻, SO₃²⁻, SO₄²⁻, PO₄³⁻, NO₃⁻, citrate, fumarate, succinate, maleate, and tartrate, preferably A⁻ is Cl⁻ or Br⁻;
wherein the at least one nucleic acid is encapsulated in the at least one LNP.

As defined herein, “A-” is a biocompatible anion naturally present in biological systems and is thus compatible with transfection.

As defined herein, the term “lipid nanoparticle” refers to a particle having at least one dimension on the order of nanometers. It refers to nanoparticles with an outer shell of lipids molecules (LNP or LNPs), that have the ability to encapsulate and transport complex active ingredients, in particular nucleic acids (such as mRNA, RNA, siRNA, or DNA-based active pharmaceutical ingredients (APIs)) to target cells in the human body to enable their delivery to the target cells.

As defined herein, the term “encapsulated” refers full encapsulation or partial encapsulation of active ingredients such as nucleic acid into the LNP. Partial encapsulation means that a proportion, preferably a minor proportion, of nucleic acid provided for encapsulation remains free in the encapsulation medium.

In a particular embodiment of the invention, the at least one nucleic acid is partially or fully encapsulated in the at least one LNP, preferably is fully encapsulated in the at least one LNP.

As defined herein, the term “chargedlipid” refers to any lipid that exists in either a positively charged or negatively charged form independently of the pH of the composition.

The composition of the invention is a cationic composition, in particular a cationic composition which is able to interact with negatively charged nucleic acid and cell membranes.

In a particular embodiment of the invention, the at least one nucleic acid, i.e., nucleic acid cargo, is either single-, or double-stranded, or combined single and double-stranded on distinct regions of the nucleic acid strand; and is selected from the group consisting of deoxyribonucleic acid (DNA), ribonucleic acid (RNA), small interfering RNA (siRNA), asymmetrical interfering RNA (aiRNA), dicer-substrate short interfering RNA (dsiRNA), small hairpin RNA (shRNA), RNA transcripts, microRNA (miRNA), messenger RNA (mRNA), circular RNA (circRNA), guide RNA (gRNA), small activating RNA (saRNA), small regulatory RNA (srRNA), long non-coding (lncRNA) and antisense oligonucleotide.

In an embodiment of the invention, the at least one nucleic acid is a RNA, in particular a mRNA, preferably a eukaryotic mRNA, in particular a mRNA encoding a protein of a mammal, especially a human protein.

In another embodiment of the invention, the at least one nucleic acid is a small interfering RNA (siRNA) leading to RNA interference (RNAi).

In another embodiment of the invention, the at least one nucleic acid is a DNA, in particular a plasmid DNA.

As defined herein, the term “ionizable lipid” refers to a lipid that is positively charged at acidic pH to condense the nucleic acid into the LNP but is neutral at physiological pH to minimize toxicity. The at least one ionizable lipid of the invention is involved in the intracellular LNPs' disassembly and release of nucleic acid into the cytoplasm or its integration into membrane of acidic intracellular vesicles. The ionizable lipid may be an ionizable cationic lipid or an ionizable neutral lipid, preferably is an ionizable cationic lipid. Ionizable lipids are well known in the art.

As defined herein, “a cationic lipid” refers to any lipid carrying a positive charge independently of pH.

As defined herein, “a neutral lipid” refers to any lipid that exists either in an uncharged or neutral zwitterionic form at a selected pH, especially from physiological pH (7.4) to pH 4 such as in the lysosomes.

In a particular embodiment of the invention, the at least one ionizable lipid is selected from the group consisting of 2-dioleyloxy-N,N-dimethyl-3-aminopropane (DODMA), dilinoleylmethyl-4-dimethylaminobutyrate (DLin-MC3-DMA), Heptadecan-9-yl 8-{(2-hydroxyethyl)[6-oxo-6-(undecyloxy)hexyl]amino}octanoate (SM-102), [(4-Hydroxybutyl)azanediyl]di(hexane-6,1-diyl) bis(2-hexyldecanoate) (ALC-0315) and 3β-[N—(N′,N′-dimethylaminoethane)-carbamoyl]cholesterol hydrochloride (DC-cholesterol), preferably is selected from the group consisting of 2-dioleyloxy-N,N-dimethyl-3-aminopropane (DODMA), dilinoleylmethyl-4-dimethylaminobutyrate (DLin-MC3-DMA), [(4-Hydroxybutyl)azanediyl]di(hexane-6,1-diyl) bis(2-hexyldecanoate) (ALC-0315) and 3β-[N—(N′,N′-dimethylaminoethane)-carbamoyl]cholesterol hydrochloride (DC-cholesterol), more preferably is DODMA.

In a particular embodiment of the invention, the at least one ionizable lipid, preferably selected from the above-mentioned list, is in the composition with the imidazolium-based cationic lipid of formula (I) selected from the group consisting of compounds W12.7, W16.7, W19.7, W20.7, W21.7 and W22.7, preferably W21.7.

The composition of the invention may comprise at least one (in particular one) ionizable lipid as defined herein. Preferably the at least one ionizable lipid is DODMA.

Unless otherwise specified, in the present disclosure “mole % of” a determined compound refers to a mole percent of total lipids, especially of total lipids of the LNP.

In a particular embodiment of the invention, the composition comprises from 1 mole % to 50 mole % of the at least one ionizable lipid, preferably from 10 mole % to 40 mole %, preferably 30 mole %.

The terms “phospholipid” and “neutral lipid” can be used interchangeably. As defined herein, the term “phospholipid” refers to a lipid that exists either in an uncharged or a neutral zwitterionic form at a selected pH. Phospholipids are a class of lipids whose molecule has a hydrophilic “head” containing a phosphate group and two hydrophobic “tails” derived from fatty acids, joined by an alcohol residue (usually a glycerol molecule). Phospholipids are well known in the art and may be synthetic or naturally derived. The at least one phospholipid of the invention may be responsive of the nucleic acids endosomal escape.

In a particular embodiment of the invention, the at least one (in particular the one) phospholipid includes any triglycerides which consist of three fatty acids attached to a glycerol molecule. Preferably, the at least one phospholipid is selected from the group consisting of phosphatidylserine (PS), phosphatidylcholine (PC), phosphatidylinositol (PI), 1,2-Dioleoyl-sn-glycero-3-phosphoethanolamine (DOPE), 1,2-diphenytanoyl-sn-glycero-3-phosphoethanolamine (DPyPE), 1,2-distearoyl-sn-glycero-3-phosphocholine (DSPC), palmitoyl linoleoyl phosphatidylethanolamine (PaLiPE), dilinoleoyl phosphatidylethanolamine (DiLiPE) and phosphatidylethanolamine (PE), preferably is DPyPE, DOPE or DSPC, more preferably DPyPE.

In a particular embodiment of the invention, the at least one phospholipid, preferably selected from the above-mentioned list, is in the composition with the imidazolium-based cationic lipid of formula (I) selected from the group consisting of compounds W12.7, W16.7, W19.7, W20.7, W21.7 and W22.7, preferably W21.7.

The composition of the invention may comprise at least one phospholipid, as defined herein, more preferably selected from the group consisting of DPyPE, DOPE or DSPC. Preferably the at least one phospholipid is DPyPE.

In a particular embodiment of the invention, the composition comprises from 1 mole % to 50 mole % of the at least one phospholipid, preferably from 10 mole % to 40 mole %, preferably 10 mole %.

As defined herein, the term “sterol” refers to a compound comprising the following carbon skeleton

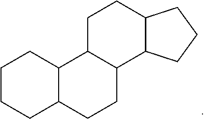 [SMILES: C1CCC2C(C1)CCC1C3CCCC3CCC21]

Sterols, also named steroids, are well-known in the art.

In a particular embodiment of the invention, the at least one sterol is a neutral sterol. In an embodiment the sterol is selected from the group consisting of cholesterol, stigmasterol, beta-sitosterol, ergosterol, campesterol, oxysterol, antrosterol, desmosterol and nicasterol, preferably is selected from the group consisting of cholesterol, stigmasterol, and beta-sitosterol, more preferably is cholesterol. The at least one sterol of the invention is involved in the particles stabilization and in the fusion with cytoplasmic membrane. Accordingly, unless otherwise stated, when reference is made to a sterol in the disclosure, it is in particular directed to a neutral sterol.

In a particular embodiment of the invention, the at least one sterol, preferably selected from the above-mentioned list, is in the composition with the imidazolium-based cationic lipid of formula (I) selected from the group consisting of compounds W12.7, W16.7, W19.7, W20.7, W21.7 and W22.7, preferably W21.7.

The composition of the invention may comprise at least one sterol (in particular one sterol) as defined herein, preferably selected from the group consisting of cholesterol, stigmasterol, and beta-sitosterol, more preferably cholesterol and beta-sitosterol. Even more preferably the at least one sterol is cholesterol.

In a particular embodiment of the invention, the composition comprises from 1 mole % to 50 mole % of the at least one sterol, especially neutral sterol, preferably from 10 mole % to 40 mole %, preferably 10 mole %.

As defined herein, the term “PEG-lipid” refers a compound comprising both a lipid portion and a PEG portion (polyethylene glycol portion). PEG-lipids are well known in the art. The at least one PEG-lipid of the invention is present on the external surface of LNPs and is responsive of its stealthiness. In an embodiment, the average molecular weight of the at least one PEG-lipid is from 0.2k to 10k, preferably from 0.6k to 5k, preferably 0.6k-3.5k, more preferably 2k wherein xk features the average molecular weight (g/mol) of the PEG molecules in the PEG-lipid.

In a particular embodiment of the invention, the at least one PEG-lipid is selected from the group consisting of 1,2-Dimyristoyl-sn-glycero-3-methoxypolyethylene glycol (DMG-PEG), 1,2-Distearoyl-rac-glycero-3-methylpolyoxyethylene (DSG-PEG), diacylglycerol-polyethylene glycol (DAG-PEG), 1,2-dipalmitoyl-sn-glycero-3-phosphoethanolamine-N-[azido(polyethylene glycol) (DPPE-PEG) and 3-N-[(w-methoxypoly(ethylene glycol)2000)carbamoyl]-1,2-dimyristyloxy-propylamine (PEG-c-DMA), preferably is DMG-PEG or DSG-PEG, more preferably DSG-PEG.

In a particular embodiment of the invention, the at least one PEG-lipid, preferably selected from the above-mentioned list, is in the composition with the imidazolium-based cationic lipid of formula (I) selected from the group consisting of compounds W12.7, W16.7, W19.7, W20.7, W21.7 and W22.7, preferably W21.7.

The composition of the invention may comprise at least one (in particular one) PEG-lipid, as defined herein. Preferably the at least one PEG-lipid is DMG-PEG or DSG-PEG, more preferably DSG-PEG.

In a particular embodiment of the invention, the at least one ionizable lipid, the at least one phospholipid, the at least one sterol, especially a neutral sterol and the at least one PEG-lipid are preferably selected from the above-mentioned respective lists and the imidazolium-based cationic lipid of formula (I) is selected from the group consisting of compounds W12.7, W16.7, W19.7, W20.7, W21.7 and W22.7, preferably W21.7. Such embodiment is especially provided for a composition of the invention wherein the nucleic acid is DNA or RNA, especially mRNA as defined herein.

In a particular embodiment of the invention, the composition comprises from 0.1 mole % to 10 mole % of the at least one PEG-lipid, preferably from 0.3 mole % to 5 mole %, preferably 1.5 mole %.

The imidazolium-based cationic lipid of formula (I) (FIG. 16) has been identified by the inventors following a structure activity screening.

The imidazolium-based cationic lipid of formula (I) may be prepared according to various methods well known in the art, for example as disclosed in the patent EP2004156 and in patent application EP3646854.

As defined herein, the term “imidazolium” refers to an organic compound containing the cationic form by protonation of imidazole in which two of the five atoms that make up the ring are nitrogen atoms.

The imidazolium-based cationic lipid of formula (I) is a permanently positively charged lipid.

The imidazolium-based cationic lipid of formula (I) is capable of interacting with nucleic acids. The imidazolium-based cationic lipid of formula (I) may be responsible of the external positive charge of the LNP.

In a particular embodiment of the invention, R₁, R₂, R₃ and R₄ are different. In a particular embodiment, at least 3 or at least 2 compounds selected from the group consisting of R₁, R₂, R₃ and R₄ are different. Preferably, R₁ and R₂ are different, R₁ and R₃ are different, R₁ and R₄ are different, R₂ and R₃ are different, R₂ and R₄ are different, R₃ and R₄ are different.

In another particular embodiment of the invention, R₂, R₃ and R₄ are identical. In a particular embodiment, at least 2 compounds selected from the group consisting of R₂, R₃ and R₄ are identical. Preferably, R₂ and R₃ are identical, R₂ and R₄ are identical, R₃ and R₄ are identical.

In a particular embodiment of the invention, R₅, R₆, R₇, R₈ and R₉ are different. In a particular embodiment, at least 4, at least 3 or at least 2 compounds selected from the group consisting of R₅, R₆, R₇, R₈ and R₉ are different. Preferably, R₅ and R₆ are different, R₅ and R₇ are different, R₅ and R₈ are different, R₅ and R₉ are different, R₆ and R₇ are different, R₆ and R₈ are different, R₆ and R₉ are different, R₇ and R₈ are different, R₇ and R₉ are different, R₈ and R₉ are different.

In another particular embodiment of the invention, R₅, R₆, R₇, R₈ and R₉ are identical. In a particular embodiment, at least 4, at least 3 or at least 2 compounds selected from the group consisting of R₅, R₆, R₇, R₈ and R₉ are identical. Preferably, R₅ and R₆ are identical, R₅ and R₇ are identical, R₅ and R₈ are identical, R₅ and R₉ are identical, R₆ and R₇ are identical, R₆ and R₈ are identical, R₆ and R₉ are identical, R₇ and R₈ are identical, R₇ and R₉ are identical, R₈ and R₉ are identical.

As defined herein, the term “a C₁-C₁₀ linear or branched hydrocarbon chain” represents a hydrocarbon chain comprising from 1 to 10 carbon atoms, preferably from 1 to 6 carbon atoms, more preferably more 1 to 4 carbon atoms. Preferably the C₁-C₁₀ linear or branched hydrocarbon chain is a linear or branched, saturated or unsaturated C₁-C₁₀ alkyl, preferably a linear or branched, saturated or unsaturated C₁-C₆ alkyl, more preferably a linear or branched, saturated or unsaturated C₁-C₄ alkyl.

As defined herein, the term “C₁-C₁₀ alkyl” represents any alkyl group having 1 to 10 carbon atoms. The term “C₁-C₆ alkyl” represents an alkyl group having 1 to 6 carbon atoms. The term “C₁-C₄ alkyl” represents an alkyl group having 1 to 4 carbon atoms. Examples of suitable C₁-C₁₀ alkyl groups include, but are not limited to, C₁-C₄ alkyl groups such as methyl, ethyl, n-propyl, i-propyl, n-butyl, i-butyl, s-butyl or t-butyl, C₆—C alkyl groups such as n-hexyl, n-heptyl or n-octyl, as well as n-pentyl, 2-ethylhexyl, 3,5,5-trimethylhexyl, n-nonyl, n-decyl, n-undecyl, n-dodecyl or n-octadecyl.

As defined herein, the term “a C₆-C₃₃ saturated or unsaturated, linear or branched hydrocarbon chain” represents a saturated or unsaturated, linear or branched hydrocarbon chain comprising from 6 to 33 carbon atoms, preferably from 6 to 24 carbon atoms, preferably from 12 to 18 carbon atoms.

As defined herein, the term “C₂-C₃₃ saturated or unsaturated, linear or branched hydrocarbon chain” represents a saturated or unsaturated, linear or branched hydrocarbon chain comprising from 2 to 33 carbon atoms, preferably from 2 to 24 carbon atoms, preferably from 12 to 18 carbon atoms.

As defined herein, the term “C₁-C₁₀ hydroxylated chain” represents a hydroxylated chain comprising from 1 to 10 carbon atoms, preferably from 1 to 6 carbon atoms, more preferably more 1 to 4 carbon atoms.

As defined herein, the term “a saturated or unsaturated C₃-C₈ cycle” represents an aromatic hydrocarbon comprising 3 to 8 carbon atoms, preferably “a saturated or unsaturated C₄-C₆ cycle” comprising 4 to 6 carbon atoms, preferably “a saturated or unsaturated C₆ cycle” comprising 6 carbon atoms Examples of suitable saturated or unsaturated C₆ cycle include, but are not limited to, substituted or unsubstituted phenyl, for example a phenyl substituted by a halogen. As defined herein, the term “halogen” represents an atom of F, Cl, Br or I.

In a particular embodiment of the invention, the imidazolium-based cationic lipid of formula (I) is selected from the group consisting of the following compounds:

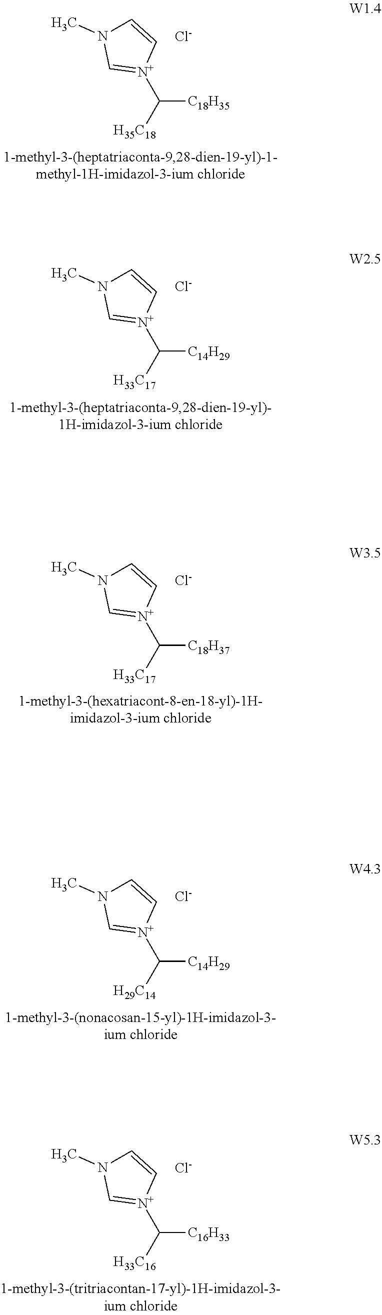

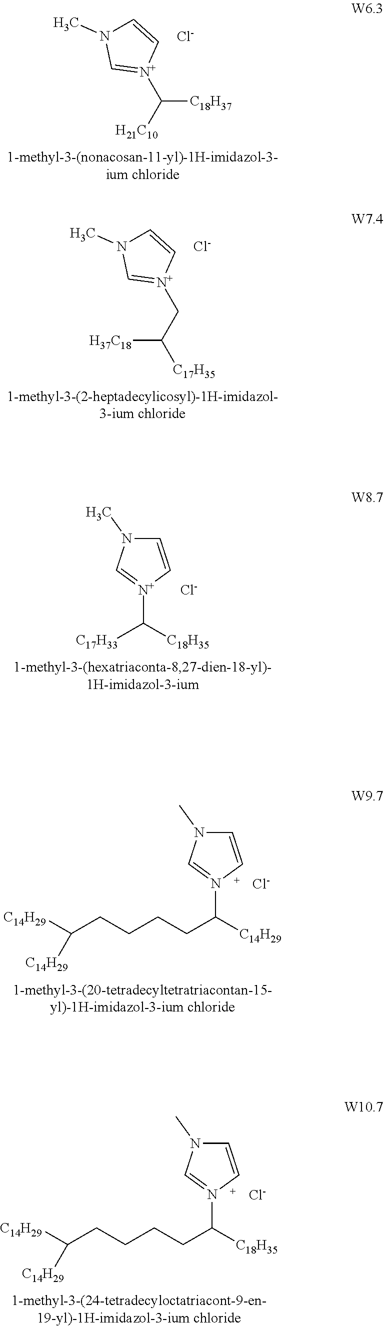

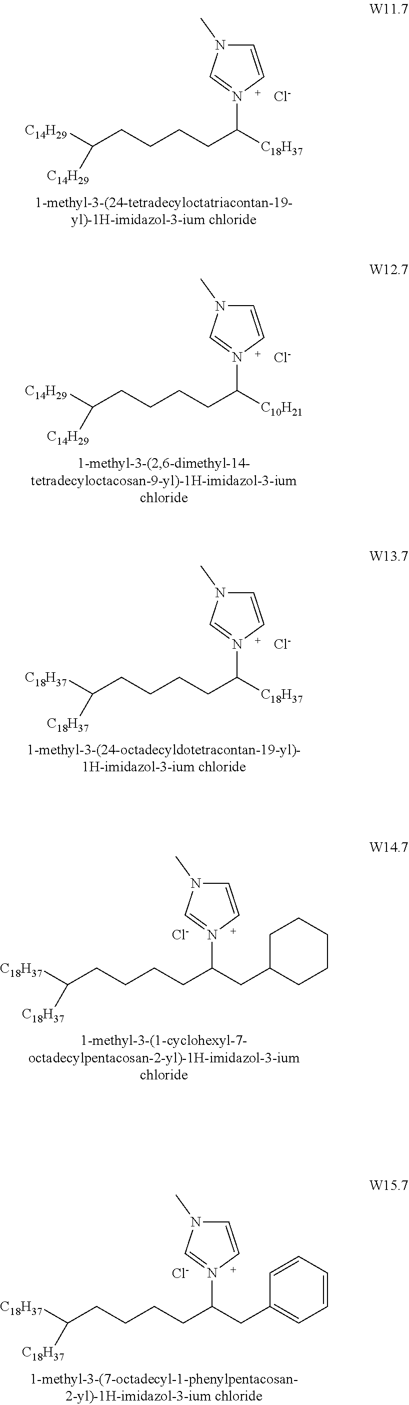

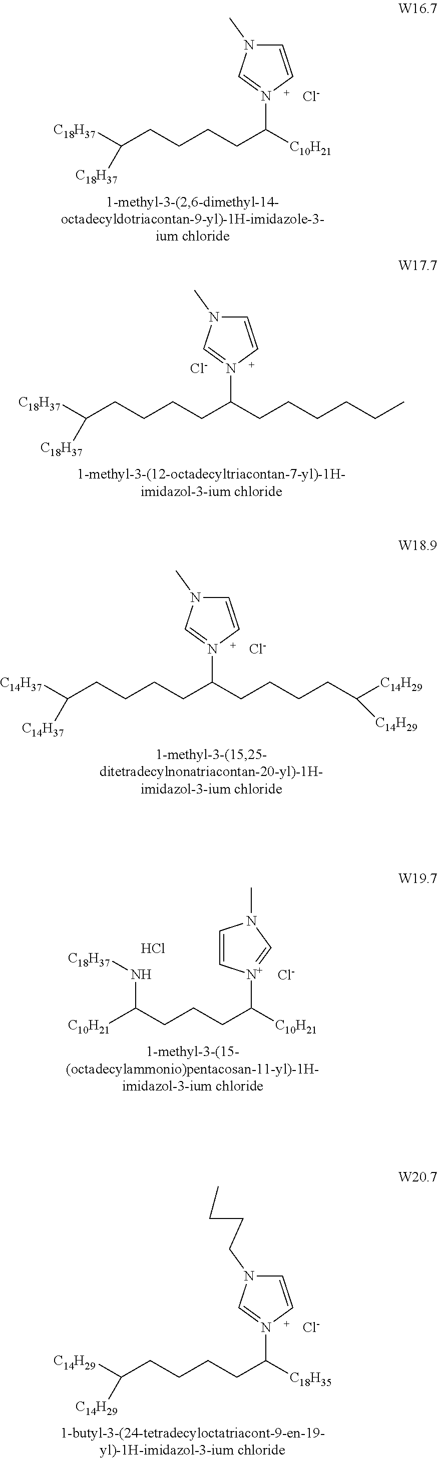

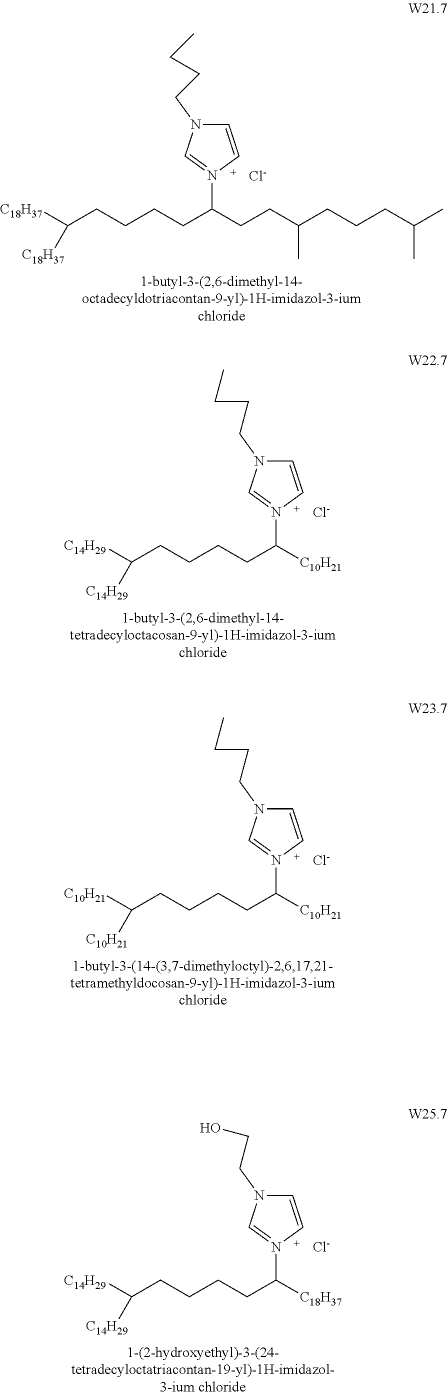

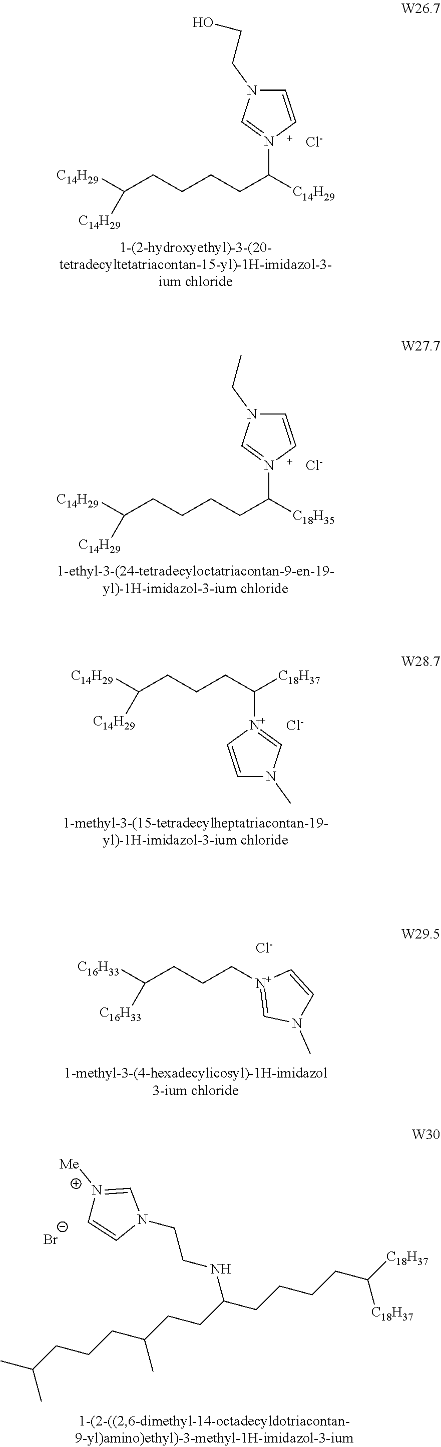

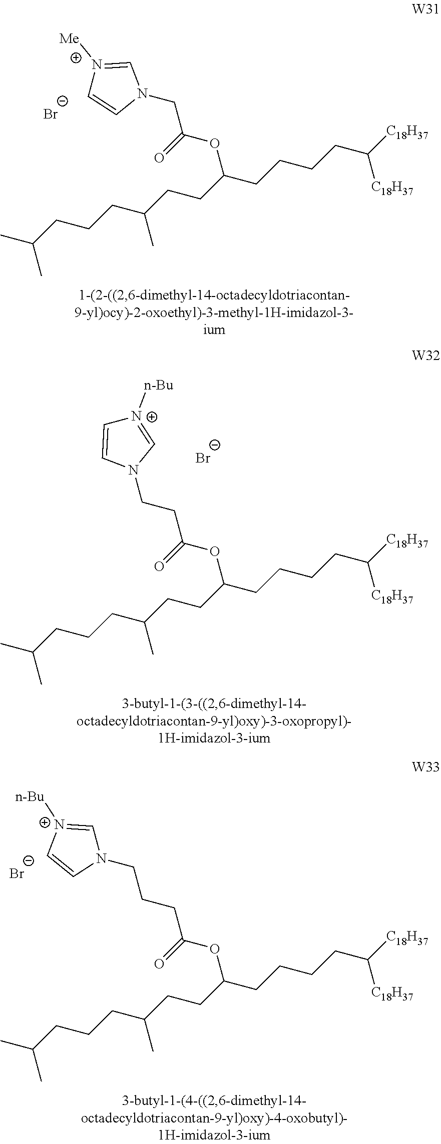

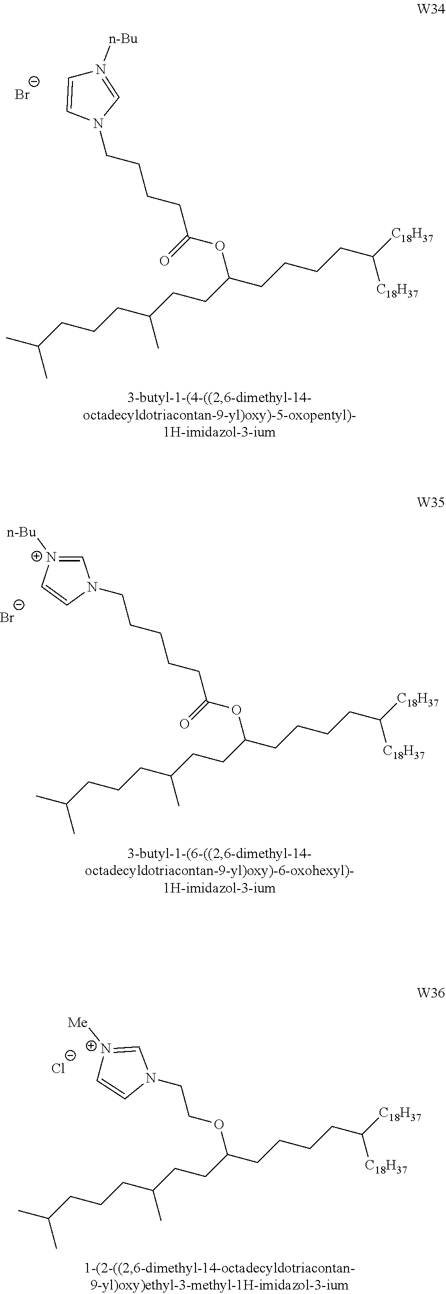

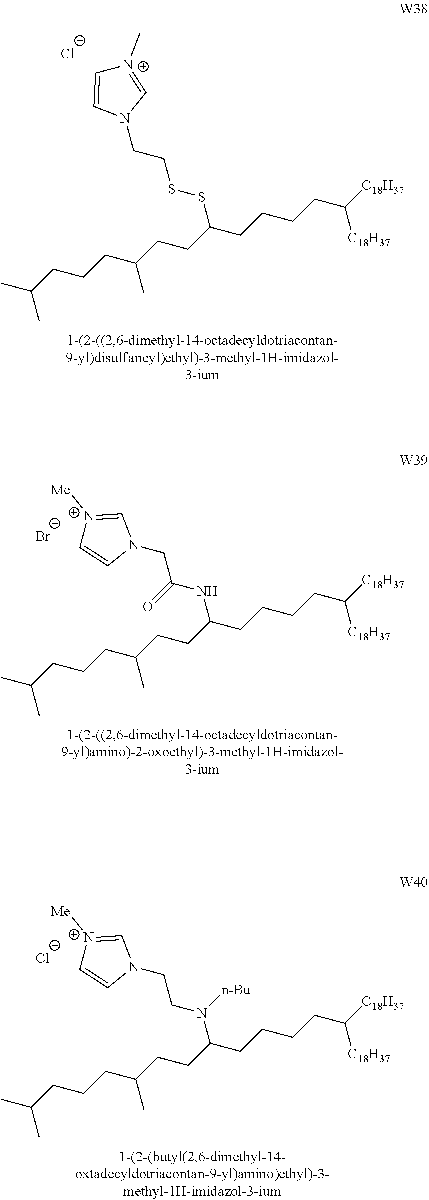

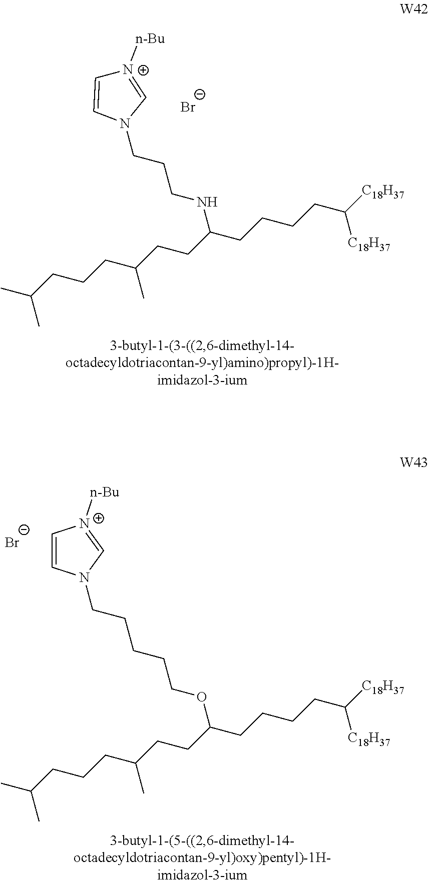

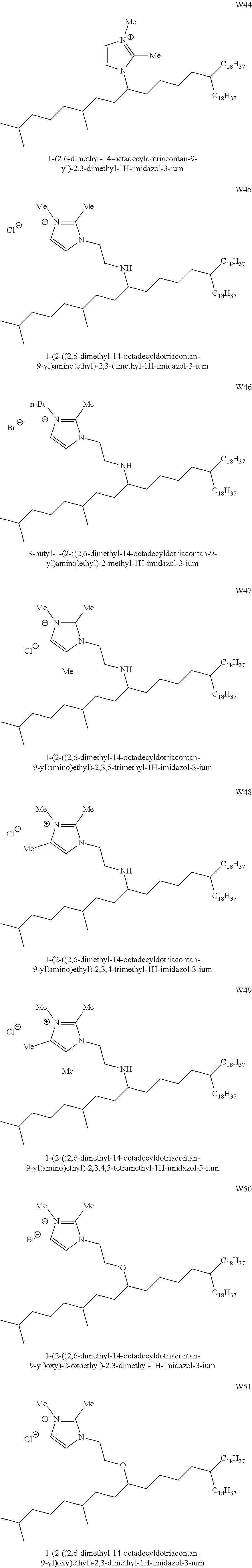

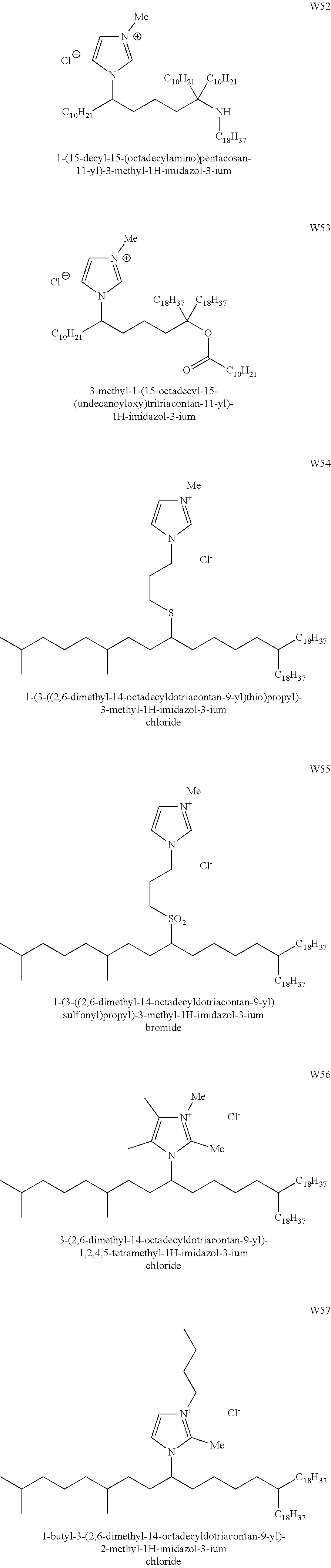

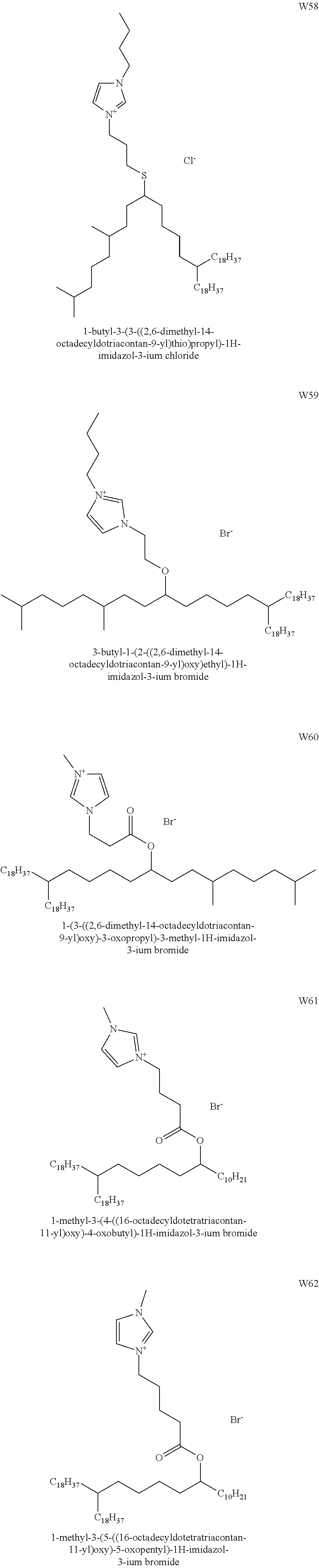

In an embodiment of the invention, the imidazolium-based cationic lipid of formula (I) is selected from the group consisting of compounds W12.7, W16.7, W19.7, W20.7, W21.7 and W22.7, preferably W21.7.

In an embodiment of the invention, the imidazolium-based cationic lipid of formula (I) is associated in the composition with at least one ionizable lipid, preferably DODMA; at least one phospholipid, preferably DPyPE, DOPE or DSPC, more preferably DPyPE; at least one sterol, especially neutral sterol, preferably cholesterol; and at least one PEG-lipid, preferably DMG-PEG or DSG-PEG, more preferably DSG-PEG.

In an embodiment, the imidazolium-based cationic lipid of formula (I) is selected from the group consisting of compounds W12.7, W16.7, W19.7, W20.7, W21.7 and W22.7, preferably W21.7 and is associated in the composition with DODMA and/or with DPyPE, DOPE or DSPC, more preferably DPyPE and/with cholesterol and/or with DMG-PEG or DSG-PEG, more preferably DSG-PEG.

In a more particular embodiment of the invention, in the at least one LNP, the imidazolium-based cationic lipid of formula (I) is W21.7, the at least one ionizable lipid is DODMA, the at least one phospholipid is DPyPE, the at least one sterol is cholesterol, and the at least one PEG-lipid is DSG-PEG.

In another more particular embodiment of the invention, in the composition, the at least one nucleic acid is RNA, in particular mRNA, the imidazolium-based cationic lipid of formula (I) is W21.7, the at least one ionizable lipid is DODMA, the at least one phospholipid is DPyPE, the at least one sterol is cholesterol, and the at least one PEG-lipid is DSG-PEG.

In a particular embodiment of the invention, the composition comprises from 1 mole % to 50 mole % of the imidazolium-based cationic lipid, preferably from 5 mole % to 40 mole %, preferably 40 mole %.

According to an embodiment of the invention, the ionizable lipid, the phospholipid, the sterol, especially neutral sterol, the PEG-lipid and imidazolium-based cationic lipid are provided in relative mole % to achieve together a total mole % of 100% in the formed LNP.

In a particular embodiment of the invention, the percentage of encapsulation of the at least one nucleic acid in the at least one LNP is at least 80%, preferably at least 85%, more preferably at least 90%.

In a particular embodiment of the invention, the at least one nucleic acid encodes a protein, in particular a peptide, such as an antigenic peptide suitable for vaccination purpose or an enzyme, in particular a nuclease such as an endonuclease or an exonuclease, and optionally the protein is a therapeutic protein, more preferably a therapeutic protein to correct genetic disorders, a therapeutic protein against cancer such as a cytokine, an anti-oncogene, an antibody such as a blocking antibody, an anti-tumor suppressor or a toxin, a therapeutic protein with antiviral activity or a therapeutic protein for immunotherapy.

In a particular embodiment of the invention, the at least one nucleic acid encodes a nucleic acid, such as shRNA.

In a particular embodiment of the invention, the at least one nucleic is a non-coding nucleic acid.

In a particular embodiment of the invention, the molar ratio of the imidazolium-based cationic lipid of formula (I) to the at least one ionizable lipid ranges from 6:1 to 2:5, preferably is 4:3.

In a particular embodiment of the invention, the at least one LNP comprises from 1 mole % to 50 mole % of the at least one ionizable lipid as defined above, preferably DODMA and from 1 mole % to 50 mole % of the at least one phospholipid as defined above, preferably DPyPE, provided the cumulative mole % of ionizable lipid and phospholipid does not amount to 100%.

In another particular embodiment of the invention, the at least one LNP comprises from 1 mole % to 50 mole % of the at least one ionizable lipid as defined above, preferably DODMA and from 1 mole % to 50 mole % of the at least one sterol, especially neutral sterol, as defined above, preferably cholesterol, provided the cumulative mole % of ionizable lipid and sterol does not amount to 100%.

In another particular embodiment of the invention, the at least one LNP comprises from 1 mole % to 50 mole % of the at least one ionizable lipid as defined above, preferably DODMA and from 0.1 mole % to 10 mole % of the at least one PEG-lipid as defined above, preferably DSG-PEG.

In a preferred embodiment of the invention, the composition comprises:
(A) at least one nucleic acid; and
(B) at least one LNP comprising:
   (i) from 1 mole % to 50 mole % of the at least one ionizable lipid as defined above, preferably DODMA;
   (ii) from 1 mole % to 50 mole % of the at least one phospholipid as defined above, preferably DPyPE;
   (iii) from 1 mole % to 50 mole % of the at least one sterol, especially neutral sterol, as defined above, preferably cholesterol;
   (iv) from 0.1 mole % to 10 mole % of the at least one PEG-lipid as defined above, preferably DSG-PEG; and
   (v) from 5 mole % to 40 mole % of the imidazolium-based cationic lipid of formula (I) as defined above, preferably compound W21.7,
   wherein compounds (i), (ii), (iii), (iv) and (v) are provided to achieve together a total mole % of 100% in the formed LNP.

In a preferred embodiment of the invention, the composition comprises:
(A) at least one mRNA; and
(B) at least one LNP comprising:
   (i) the at least one ionizable lipid as defined above, preferably DODMA;
   (ii) the at least one phospholipid as defined above, preferably DPyPE;
   (iii) the at least one sterol, especially neutral sterol, as defined above, preferably cholesterol;
   (iv) the at least one PEG-lipid as defined above, preferably DSG-PEG; and
   (v) the imidazolium-based cationic lipid of formula (I) as defined above, preferably compound W21.7
   wherein compounds (i), (ii), (iii), (iv) and (v) are provided to achieve together a total mole % of 100% in the formed LNP.

In a more preferred embodiment of the invention, the composition comprises:
(A) at least one mRNA; and
(B) at least one LNP comprising:
   (i) from 1 mole % to 50 mole % of the at least one ionizable lipid as defined above, preferably DODMA;
   (ii) from 1 mole % to 50 mole % of the at least one phospholipid as defined above, preferably DPyPE;
   (iii) from 1 mole % to 50 mole % of the at least one sterol, especially neutral sterol, as defined above, preferably cholesterol;
   (iv) from 0.1 mole % to 10 mole % of the at least one PEG-lipid as defined above, preferably DSG-PEG; and
   (v) from 5 mole % to 40 mole % of the imidazolium-based cationic lipid of formula (I) as defined above, preferably compound W21.7
   wherein compounds (i), (ii), (iii), (iv) and (v) are provided to achieve together a total mole % of 100% in the formed LNP.

In a particular embodiment of the invention, the at least one nucleic acid is a therapeutic ingredient for use as a therapeutic agent or a prophylactic vaccine against viral infections, or a therapeutic vaccine against cancers. Preferably, the composition is for use in in vitro or in vivo delivering the at least one nucleic acid to a target cell.

Generally, in this aspect, the vaccine is delivered through direct administration such as systemic, intramuscular, intradermal, intraperitoneal, intratumoral, oral, topical, or sub-cutaneous administration, and, in said vaccine, the composition is in association with a pharmaceutically acceptable vehicle. In other words, the vaccine can be injected directly into the body, in particular in a human individual, for inducing a cellular and/or a humoral response.

According to a particular embodiment of the invention, the composition hence comprises a pharmaceutically acceptable vehicle.

As defined herein, “a pharmaceutically acceptable vehicle” refers to any substance or combination of substances physiologically acceptable i.e., appropriate for its use in a composition in contact with a host, especially a human, and thus non-toxic. It can refer to a solid, semi-solid or liquid filler, diluent, encapsulating material or formulation auxiliary of any conventional type.

In a particular embodiment of the invention, the composition is for use to deliver the nucleic acid to a target organ selected from the group consisting of lungs, heart, brain, spleen, the nodes, bone marrow, bones, skeletal muscles, stomach, small intestine, large intestine, kidneys, bladder, breast, liver, testes, ovaries, uterus, spleen, thymus, brainstem, cerebellum, spinal cord, eye, ear, tongue and skin, preferably lungs and spleen.

The present invention is also directed to the composition according to the invention for use in in vivo applications for nucleic acid-based therapy, in particular mRNA-based therapy.

In another particular embodiment of the invention, the composition is for use according to the invention for in vivo delivering the at least one nucleic acid to a target cell.

The present invention also relates to the use of the composition for in vitro delivering the at least one nucleic acid to a target cell.

The present invention also concerns a method for in vivo or in vitro transfection of live cells comprising introducing in the cells the composition according to the invention.

The present invention also relates to the use of the composition according to the invention to in vitro transfect a nucleic acid into a cell, preferably a mammalian cell, an insect cell, a cell line, a primary cell, an adherent cell, a suspension cell, a cancer or a tumor cell, more preferably a cell selected from the group consisting of CaCo2 cells, HeLa cells, HepG2 cells, A549 cells, Jurkat cells, Antigen Presenting cells, Dendritic cells, human primary T cells and human CD34+ cells. Some of these cells are known to be difficult to transfect (“hard-to-transfect cells”), e.g., CaCo2 cells, Jurkat cells.

As defined herein, the term “an adherent cell” refers to a cell that needs solid support for growth, and thus is anchorage-dependent. Examples of adherent cells include MRC-5 cells, HeLa cells, Vero cells, NIH-3T3 cells, L293 cells, CHO cells, BHK-21 cells, MCF-7 cells, A549 cells, COS cells, HEK 293 cells, Hep G2 cells, SNN-BE(2) cells, BAE-1 cells and SH-SY5Y cells.

As defined herein, the term “a suspension cell” refers to a cell that does not need solid support for growth, and thus is anchorage-independent. Examples of suspension cells include NSO cells, U937 cells, Namalawa cells, HL60 cells, WEH1231 cells, Yac 1 cells, Jurkat cells, THP-1 cells, K562 cells and U266B1 cells.

The present invention also relates to the use of the composition according to the invention for in vitro or ex vivo cell reprogramming, in particular for the in vitro or ex vivo reprogramming of differentiated cells into induced pluripotent stem cells (iPCs), for in vitro or ex vivo differentiating cells, for in vitro or ex vivo gene-editing or genome engineering.

Such use may be carried out in a culture of cells in vitro or ex vivo for the production of biologics, for the preparation of cells for therapy purpose, or for the study of cell functions or behaviour in particular with a step of expansion of cells after their transfection or may be carried out in vivo for a therapeutic purpose in a host in need thereof.

The present invention also relates to the use of the composition according to the invention in the production of in vitro or ex vivo biologics encoding a recombinant protein or antibody, or in the production of recombinant virus.

The present invention also relates to a LNP, in particular a positively charged LNP, as defined above, in particular a LNP comprising:
(i) the at least one ionizable lipid as defined above;
(ii) the at least one phospholipid as defined above;
(iii) the at least one sterol, especially neutral sterol, as defined above;
(iv) the at least one PEG-lipid as defined above; and
(v) the imidazolium-based cationic lipid of formula (I) as defined above.

In a particular embodiment of the invention, the LNP comprises a diameter ranging from 10 nm to 200 nm, preferably from 10 nm to 110 nm.

In a particular embodiment of the invention, the LNP comprises a zeta potential ranging from −15 mV to +30 mV, preferably from 9 mV to 23 mV.

In a particular embodiment of the invention, the LNP is stable at a temperature, of 4° C., for several weeks, e.g. at least 13 weeks.

The present invention also relates to a method for delivering a nucleic acid, preferably a mRNA, to a cell comprising contacting the cell with the composition according to the invention.

The present invention also relates to a method of producing a LNP formulation, preferably a positively charged LNP composition, the method comprising mixing an organic phase with an aqueous phase wherein the organic phase is composed of lipids and ethanol and the aqueous phase is composed of nucleic acid and acetate buffer.

The LNP composition may be performed on a microfluidic device, preferably a NanoAssemblr device, with a volume ratio of aqueous phase:organic phase from 1:1 to 5:1, preferably 3:1 and a flow rate of 5 mL/min to 50 mL/min, preferably 10 mL/min.

Other features and advantages of the invention will be apparent from the examples which follow and will also be illustrated in the figures.

### BRIEF DESCRIPTION OF THE FIGURES

FIG. 1A. Caco2 cells were transfected with in vivo-jetRNA®/Fluc mRNA complexes or LNP 1, LNP 2, LNP 3, LNP 4, LNP 5, LNP 6, LNP 7, LNP 8, LNP 9, or LNP 10.

FIG. 1B. Caco2 cells were transfected with in vivo-jetRNA®/Fluc mRNA complexes or (top panel) LNP 11C, or LNP 16; (middle panel) LNP 11L, LNP 14, or LNP 15; (bottom panel) LNP 11N, LNP 12, LNP13, or LNP 17.

FIG. 1C. Caco2 cells were transfected with in vivo-jetRNA®/Fluc mRNA complexes or (top panel) LNP 11L, LNP 18, LNP 19, LNP 20, LNP 21, LNP 22, LNP 23, LNP 24, LNP 25, or LNP 26; (bottom panel) LNP 11M, LNP 27, LNP 28, LNP 29, LNP 30, LNP 31, or LNP 32.

FIG. 1D. Caco2 cells were transfected with in jetMESSENGER®/Fluc mRNA complexes or LNP 110, LNP 33B, LNP 34B, LNP 36B, LNP 37B, LNP 38B, LNP 39B, LNP 42, LNP 40, or LNP 35.

FIG. 2. HepG2 cells were transfected with jetMESSENGER®/Fluc mRNA complexes or LNP 110, LNP 33B, LNP 34B, LNP 36B, LNP 37B, LNP 38B, LNP 39B, LNP 42, LNP 40, or LNP 35.

FIG. 3. HeLa cells were transfected with jetMESSENGER®/Fluc mRNA complexes or LNP 110, LNP 33B, LNP 34B, LNP 36B, LNP 37B, LNP 38B, LNP 39B, LNP 42, LNP 40, or LNP 35.

FIG. 4. A459 cells were transfected with jetMESSENGER®/Fluc mRNA complexes or LNP 110, LNP 33B, LNP 34B, LNP 36B, LNP 37B, LNP 38B, LNP 39B, LNP 42, LNP 40, or LNP 35.

FIG. 5. Jurkat cells were transfected with jetMESSENGER®/Fluc mRNA complexes or LNP 110, LNP 33B, LNP 34B, LNP 36B, or LNP 35.

FIG. 6. Human primary T cells were transfected with jetMESSENGER®/Fluc mRNA complexes or LNP 110, LNP 33B, LNP 34B, LNP 36B, or LNP 35.

FIG. 7. Human primary T cells were transfected with jetMESSENGER®/GFP mRNA complexes or LNP 43B, or LNP 44.

FIG. 8. Human primary CD34+ cells were transfected with jetMESSENGER®/GFP mRNA complexes or LNP 43B.

FIG. 9. HeLa cells were transfected with jetPRIME®/GFP mRNA complexes or LNP 45.

FIG. 10. A549_luc cells were transfected with INTERFERin®/siRNA GL3_Luc or GL2_Mm complexes or LNP 46 or LNP 47.

FIG. 11. Retro-orbital (RO) injection of 10 ug of mRNA with in vivo-jetRNA®/Fluc mRNA complexes, LNP 48B or LNP 49. Luciferase expression was assessed in three organs (lung, spleen and liver) in RLU/organ (top panel) and RLU/mg of proteins (bottom panel).

FIG. 12. Retro-orbital (RO) injection of 10 ug of mRNA with LNP 48B. Luciferase expression was assessed in six organs (lung, spleen, liver, heart, pancreas and kidneys) in RLU/organ (top panel) and RLU/mg of proteins (bottom panel).

FIG. 13. Intramuscular (IM) injection of 5 ug of mRNA with in vivo-jetRNA®/Fluc mRNA complexes or LNP 48B. Luciferase expression was assessed in the muscle in RLU/organ (top panel) and RLU/mg of proteins (bottom panel).

FIG. 14. Retro-orbital (RO) injection of 7.5 μg of mRNA with LNP 48D, LNP 50B, LNP 51C or LNP 52, LNP 53, LNP 54, or LNP 55. Luciferase expression was assessed in three organs (lung, spleen and liver) in RLU/organ (top panel) and RLU/mg of proteins (bottom panel).

FIG. 15. Stability of LNPs at 4° C.: Caco2 cells were transfected with in vivo-jetRNA®/Fluc mRNA complexes or LNP 11D (stored 13 weeks at 4° C.), LNP 11K (stored 10 weeks at 4° C.) and LNP 11L (stored 5 weeks at 4° C.).

FIG. 16. Chemical structure of an imidazolium-based cationic lipid of formula (I).

FIG. 17A. Caco-2 cells were transfected with in vivo-jetRNA®+/Fluc mRNA complexes or LNP 99H, LNP 217, LNP 218, LNP 219, LNP 220 or LNP 221.

FIG. 17B. HEK-293(a) cells were transfected with in vivo-jetRNA®+/Fluc mRNA complexes or LNP 99H, LNP 217, LNP 218, LNP 219, LNP 220 or LNP 221.

FIG. 18A.Caco-2 cells were transfected with in vivo-jetRNA®+/Fluc mRNA complexes or LNP 99H, LNP 227, LNP 228, LNP 229, LNP 230 or LNP 231.

FIG. 18B. Hek-293(a) cells were transfected with in vivo-jetRNA®+/Fluc mRNA complexes or LNP 99H, LNP 227, LNP 228, LNP 229, LNP 230 or LNP 231.

FIG. 19A. Caco-2 cells were transfected with in vivo-jetRNA®+/Fluc mRNA complexes or LNP 86AC, LNP 243, LNP 244, LNP 245, LNP 246 or LNP 247.

FIG. 19B. HEK-293(a) cells were transfected with in vivo-jetRNA®+/Fluc mRNA complexes or LNP 86AC, LNP 243, LNP 244, LNP 245, LNP 246 or LNP 247.

FIG. 20A. Caco-2 cells were transfected with in vivo-jetRNA®+/Fluc mRNA complexes or LNP 86AD, LNP 248, LNP 249, LNP 250, LNP 251, LNP 252 and LNP 253.

FIG. 20B. HEK-293(a) cells were transfected with in vivo-jetRNA®+/Fluc mRNA complexes or LNP 86AD, LNP 248, LNP 249, LNP 250, LNP 251, LNP 252 and LNP 253.

FIG. 21A. Caco-2 cells were transfected with in vivo-jetRNA®+/Fluc mRNA complexes or LNP 86AD, LNP 255, LNP 256, LNP 257, LNP 258, LNP 259, LNP 260, LNP 261 or LNP 262.

FIG. 21B. HEK-293(a) cells were transfected with in vivo-jetRNA®+/Fluc mRNA complexes or LNP 86AD, LNP 255, LNP 256, LNP 257, LNP 258, LNP 259, LNP 260, LNP 261 or LNP 262.

FIG. 22A. Caco-2 cells were transfected with in vivo-jetRNA®+/Fluc mRNA complexes or LNP 99J, LNP 285, LNP 286, LNP 287, LNP 288, LNP 290, LNP 291, LNP 292, LNP 293, LNP 294 or LNP 295.

FIG. 22B. HEK-293(a) were transfected with in vivo-jetRNA®+/Fluc mRNA complexes or LNP 99J, LNP 285, LNP 286, LNP 287, LNP 288, LNP 290, LNP 291, LNP 292, LNP 293, LNP 294 or LNP 295.

FIG. 23A. Intra-peritoneal (IP) injection of 20 μg of mRNA with in vivo-jetRNA®+/Fluc mRNA complexes or LNP 99F. Luciferase expression was assessed in organs in RLU/organ.

FIG. 23B. Intra-peritoneal (IP) injection of 20 μg of mRNA with in vivo-jetRNA®+/Fluc mRNA complexes or LNP 99F. Luciferase expression was assessed in organs in RLU/mg of proteins.

FIG. 24. Humoral immune response of OVA mRNA encapsulated in LNP 161 with compound W21.7 (data shown are mean plus SEM (n=6)).

FIG. 25. GFP expression in lung cells following IV injection of LNP190B and LNP222. Statistical significance was analyzed by a one-way Anova analysis with Tukey's multiple comparisons test (**p<0.01 and ****p<0.0001).

FIG. 26. GFP expression in spleen cells following IV injection of LNP190C and LNP222B.

FIG. 27. HEK-293 cells were transfected with jetPRIME®/Fluc DNA complexes or LNP 109C and LNP A32.

FIG. 28A. Retro-orbital (RO) injection of 20 μg of mRNA with in vivo-jetRNA®+/Fluc mRNA complexes or LNP 99G or 10 μg or 20 μg of DNA with LNP 191. Luciferase expression was assessed in organs in RLU/organ (28A).

FIG. 28B. Retro-orbital (RO) injection of 20 μg of mRNA with in vivo-jetRNA®+/Fluc mRNA complexes or LNP 99G or 10 μg or 20 μg of DNA with LNP 191. Luciferase expression was assessed in organs in RLU/mg of proteins.

FIG. 29. HEK-293 cells were transfected with in vivo-jetRNA®+/nLUC saNA complexes or LNP 284.

FIG. 30. MoDCs were transfected with jetMESSENGER®/eGFP mRNA complexes or LNP123E or LNP 141E.

FIG. 31A. Caco-2 cells were transfected with in vivo-jetRNA®+/Fluc mRNA complexes or LNP 86AA, LNP 199, LNP 200, LNP 201, LNP 202 or LNP 203.

FIG. 31B. HEK293 cells were transfected with in vivo-jetRNA®+/Fluc mRNA complexes or LNP 86AA, LNP 199, LNP 200, LNP 201, LNP 202 or LNP 203.

FIG. 32A. Caco-2 cells were transfected with in vivo-jetRNA®+/Fluc mRNA complexes or LNP 86Y, LNP 199B, LNP 204, LNP 205, LNP 206, LNP 207 or LNP 208.

FIG. 32B. HEK293 cells were transfected with in vivo-jetRNA®+/Fluc mRNA complexes or LNP 86Y, LNP 199B, LNP 204, LNP 205, LNP 206, LNP 207 or LNP 208.

FIG. 33A. Retro-orbital (RO) injection of 10 μg of mRNA with in vivo-jetRNA®+/Fluc mRNA complexes or LNP 991, LNP 281, LNP 282 or LNP 283. Luciferase expression was assessed in organs in RLU/organ.

FIG. 33B. Retro-orbital (RO) injection of 10 μg of mRNA with in vivo-jetRNA®+/Fluc mRNA complexes or LNP 991, LNP 281, LNP 282 or LNP 283. Luciferase expression was assessed in organs in RLU/mg of proteins.

FIG. 34A. Caco-2 cells were transfected with in vivo-jetRNA®+/Fluc mRNA complexes or LNP 86Y, LNP 199B, LNP 209, LNP 210, LNP 211, LNP 212, LNP 213 or LNP 214.

FIG. 34B. HEK-293 cells were transfected with in vivo-jetRNA®+/Fluc mRNA complexes or LNP 86Y, LNP 199B, LNP 209, LNP 210, LNP 211, LNP 212, LNP 213 or LNP 214.

FIG. 35. Caco-2 cells were transfected with in vivo-jetRNA®+/Fluc mRNA complexes or LNP 86AB, LNP 232, LNP 233, or LNP 234.

FIG. 36. Caco-2 cells were transfected with in vivo-jetRNA®+/Fluc mRNA complexes or LNP 86AC, LNP 237, LNP 238, LNP 239, LNP 240, or LNP 241.

FIG. 37. Caco-2 cells were transfected with in vivo-jetRNA®+/Fluc mRNA complexes or LNP 86AB, LNP 235 or LNP 236.

FIG. 38. Caco-2 cells were transfected with in vivo-jetRNA®+/Fluc mRNA complexes or LNP 86AD, LNP 263, LNP 264, LNP 265, or LNP 266.

### EXAMPLES

### Example 1. Syntheses of Imidazolium-Based Cationic Lipid of Formula (I) of the Invention

### Synthesis of W21.7: 1-butyl-3-(2,6-dimethyl-14-octadecyldotriacontan-9-yl)-1H-imidazol-3-ium chloride

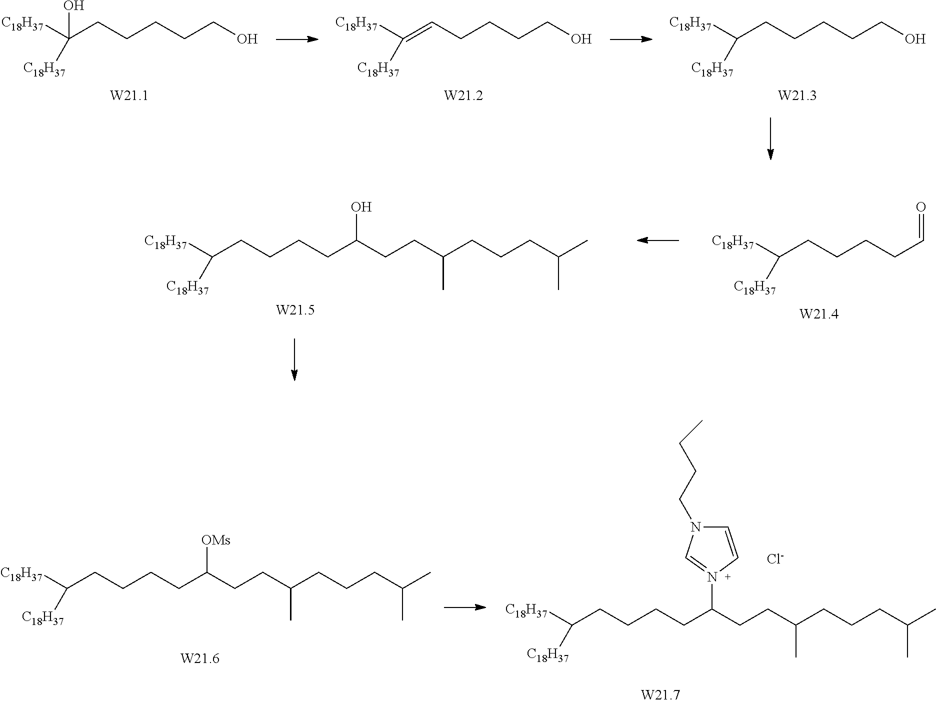

### a) Synthesis of Diol W21.1:

To a solution of 100 mL of octadecylmagnesium chloride at 0.5 M in THE (50 mmoles) was added drop-wise 2.51 mL of ε-Caprolactone (2.58 g; 22.65 mmoles; MW=114.14) dissolved in 20 mL diethyl ether. The obtained reaction mixture was stirred under an argon atmosphere at room temperature for 24 hours. Then, the reaction mixture was poured onto 600 mL split ice, acidified with concentrated hydrochloric acid for 1 hour. The solid residue in suspension was filtered off and washed with water. The solid thus obtained was recrystallized from acetone and dried to afford 12.2 g of pure diol W21.1 (19.58 mmoles; MW=623.13, 86% yield).

### Analysis of Diol W21.1:

TLC: Rf=0.25; solvent: ethyl acetate-heptane 3:7 (V:V); detection with vanillin/sulfuric acid (Merck TLC plates silica gel 60 F₂₅₄).

¹H-NMR (400 MHz, CDCl₃): δ=3.63 (t, J=6.6 Hz, 2H), 1.57 (quint, J=6.7 Hz, 2H), 1.45-1.05 (m, 74H), 0.86 (t, J=6.9 Hz, 6H).

### B) Synthesis of Alcenol W21.2

Diol W21.1 (12.2 g; 19.58 mmoles; MW=623.13) and p-toluenesulfonic acid monohydrate (750 mg; 3.9 mmoles; MW=190.22) were dissolved in 300 mL toluene. The mixture was refluxed for 3 hours, water was removed with a Dean-Stark trap. The solvents were removed under reduced pressure to give a crude that was chromatographed on silica gel (CH₂Cl₂-Heptane 1:1) to afford 10.80 g of pure alcenol W21.2 (mixture of isomers) (17.85 mmoles; MW=605.12, 91% yield).

### Analysis of Alcenol W21.2:

TLC: Rf=0.35; solvent: CH₂Cl₂-Heptane 7:3; detection with vanillin/sulfuric acid (Merck TLC plates silica gel 60 F₂₅₄).

¹H-NMR (400 MHz, CDCl₃): δ=5.11-5.03 (m, 1H), 3.62 (td, J=6.6 Hz, 1.9 Hz, 2H), 2.03-1.89 (m, 6H), 1.60-1.51 (m, 2H), 1.49-0.99 (m, 66H), 0.86 (t, J=6.8 Hz, 6H).

### c) Synthesis of Alcohol W21.3:

Mixture of alcenol isomers W21.2 (10.80 g, 17.85 mmoles, MW=605.12) was dissolved in 300 mL ethyl acetate and catalytic hydrogenation with Palladium on charcoal (Pd/C 10%, 2 g) for 24 hours at 1 atmosphere pressure of hydrogen was applied. After replacement of hydrogen by argon, the mixture was filtered through Celite® 545. The filter cake was washed with 2×250 mL of hot CH₂Cl₂. Combined solvents were removed under reduced pressure to afford 10.10 g of pure alcohol W21.3 (16.63 mmoles, MW=607.13, 93% yield).

### Analysis of Alcohol W21.3:

TLC: Rf=0.35; solvent: CH₂Cl₂-Heptane 7:3; detection with vanillin/sulfuric acid (Merck TLC plates silica gel 60 F₂₅₄).

¹H-NMR (400 MHz, CDCl₃): δ=3.62 (t, J=6.7 Hz, 2H), 1.55 (quint, J=6.9 Hz, 2H), 1.44-1.00 (m, 75H), 0.86 (t, J=6.7 Hz, 6H).

### d) Formation of Aldehyde W21.4:

Alcohol W21.3 (10.10 g, 16.63 mmoles, MW=607.13) was dissolved in 300 mL CH₂Cl₂, Pyridinium chlorochromate (7 g, 32.47 mmoles, MW=215.56) was added and the reaction stirred at room temperature for 3 hours under an argon atmosphere. The mixture was filtered through Celite® 545 and the filter cake was washed with CH₂Cl₂. Combined solvents were removed under reduced pressure and the obtained crude was chromatographed on silica gel (CH₂Cl₂-Heptane 1:4) to afford 8.08 g of pure aldehyde W21.4 (13.35 mmoles, MW=605.12, 80% yield).

### Analysis of Aldehyde W21.4:

TLC: Rf=0.35; solvent: CH₂Cl₂-Heptane 3:7; detection with vanillin/sulfuric acid (Merck TLC plates silica gel 60 F₂₅₄).

¹H-NMR (400 MHz, CDCl₃): δ=9.74 (t, J=2.0 Hz, 1H), 2.40 (td, J=7.3 Hz, 2.0 Hz, 2H), 1.59 (quint, J=7.3 Hz, 2H), 1.36-1.12 (m, 73H), 0.86 (t, J=6.9 Hz, 6H).

### e) Synthesis of W21.5:

Aldehyde W21.4 (8.08 g, 13.35 mmoles, MW=605.12) was dissolved in 100 mL THE and then introduced drop-wise on 50 mL of a stirred solution of 3,7-Dimethyloctylmagnesium bromide at 1 M in diethyl ether (50 mmoles). The obtained reaction mixture was stirred under an argon atmosphere at room temperature for 24 hours. Then, the reaction mixture was poured onto 600 mL split ice, acidified with concentrated chlorhydric acid for 1 hour. This solution was then extracted with 3×200 mL of CH₂Cl₂, dried over anhydrous sodium sulfate and solvents were removed under reduced pressure. The residue was then resuspended in 200 mL of Acetone and cooled in an ice-water bath for 1 hour. Desired product precipitated as a white solid that was recovered by filtration. After drying, 9.80 g of alcohol W21.5 were obtained (13.11 mmoles, MW=747.40, 98% yield).

### Analysis of Alcohol W21.5:

TLC: Rf=0.40; solvent: CH₂Cl₂-Heptane 3:7; detection with vanillin/sulfuric acid (Merck TLC plates silica gel 60 F₂₅₄).

¹H-NMR (400 MHz, CDCl₃): δ=3.75-3.50 (m, 1H), 1.63-0.96 (m, 89H), 0.92-0.75 (m, 15H).

### f) Synthesis of W21.6

Alcohol W21.5 (9.80 g; 13.11 mmoles; MW=747.40) was dissolved in 250 mL of dry CH₂Cl₂ and 18 mL of triethylamine (13.07 g; 129.16 mmoles; MW=101.19) were added followed by 8 mL of methanesulfonyl chloride (11.84 g; 103.36 mmoles; MW=114.55) introduced drop-wise. The mixture was stirred overnight at room temperature. After removal of the solvents under reduced pressure, the residue was dissolved in 200 mL of methanol and cooled in an ice-water bath for 1 hour. Desired product precipitated as a white solid collected by filtration on a filter paper. After solubilization in CH₂Cl₂ and evaporation, 9.60 g of compound W21.6 were obtained (11.63 mmoles, MW=825.49, 88% yield).

### Analysis of Mesylate W21.6:

¹H-NMR (400 MHz, CDCl₃): δ=4.66 (quint, J=5.9 Hz, 1H), 2.97 (s, 3H), 1.76-1.59 (m, 4H), 1.57-0.98 (m, 85H), 0.91-0.78 (m, 15H).

### g) Synthesis of W21.7

Mesylate W21.6 (9.60 g; 11.63 mmoles; MW=825.49) was resuspended in 40 mL of 1-butylimidazole and was stirred at 80° C. for 5 days under an argon atmosphere. The mixture was diluted with 400 mL of methanol and insoluble matter was removed by filtration. Filtrate was cooled in an ice-water bath, slowly acidified with 100 mL of hydrochloric acid 37% and evaporated to dryness. The residue was resuspended in 400 mL of ultra-pure water and cooled in an ice-water bath for 1 hour. Desired product, insoluble in water, was collected by filtration on a filter paper. The obtained residue was further chromatographed on silica gel (CH₂Cl₂-methanol 98:2) to afford 6.78 g of compound W21.7 as a white solid (7.62 mmoles, MW=890.03, 74% yield).

### Analysis of Compound W21.7:

TLC: Rf=0.45; solvent: CH₂Cl₂-methanol 9:1; detection with iodine (Merck TLC plates silica gel 60 F₂₅₄).

¹H-NMR (400 MHz, CDCl₃): δ=11.30 (s, 1H), 7.23-7.20 (m, 1H), 7.13-7.07 (m, 1H), 4.54-4.45 (m, 1H), 4.41 (t, J=7.3 Hz, 2H), 1.98-1.64 (m, 8H), 1.54-0.98 (m, 88H), 0.88-0.77 (m, 15H).

### Synthesis of W20.7: 1-butyl-3-(24-tetradecyloctatriacont-9-en-19-yl)-1H-imidazol-3-ium chloride

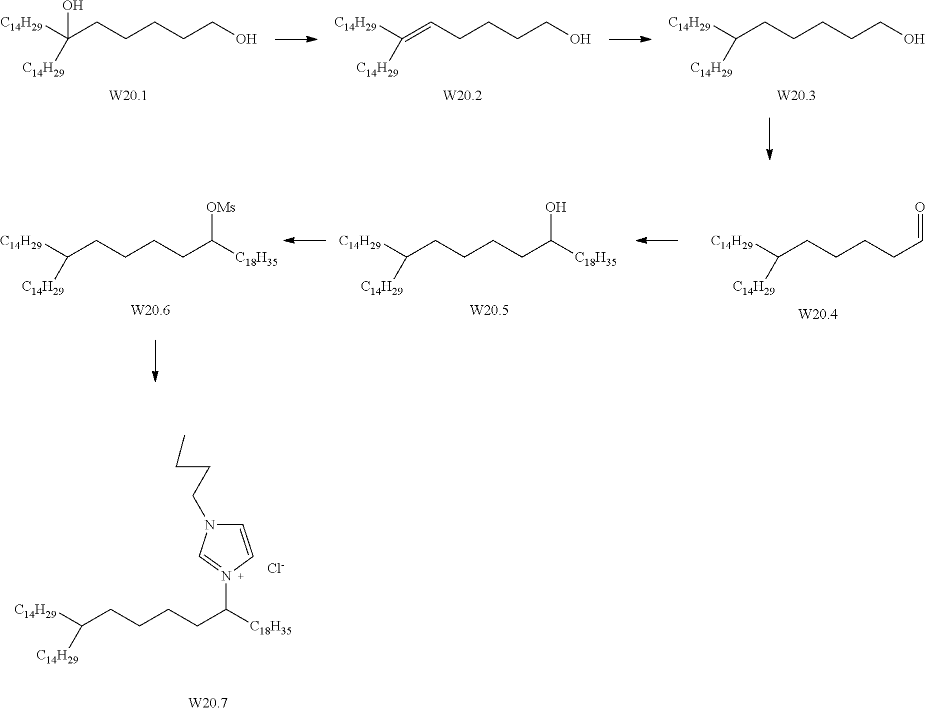

Compounds W20.1 to W20.7 were obtained following procedures similar to the ones described above (W21.1 to W21.7).

### Analysis of Compound W20.7:

TLC: Rf=0.5; solvent: CH₂Cl₂-methanol 9:1; detection with iodine (Merck TLC plates silica gel 60 F₂₅₄).

¹H-NMR (400 MHz, CDCl₃): δ=11.31 (s, 1H), 7.22-7.18 (m, 1H), 7.12-7.08 (m, 1H), 5.37-5.25 (m, 2H), 4.56-4.46 (m, 1H), 4.40 (t, J=7.3 Hz, 2H), 2.03-1.70 (m, 10H), 1.42-0.90 (m, 88H), 0.85 (t, J=6.8 Hz, 9H).

### Synthesis of W22.7: 1-butyl-3-(2,6-dimethyl-14-tetradecyloctacosan-9-yl)-1H-imidazol-3-ium chloride

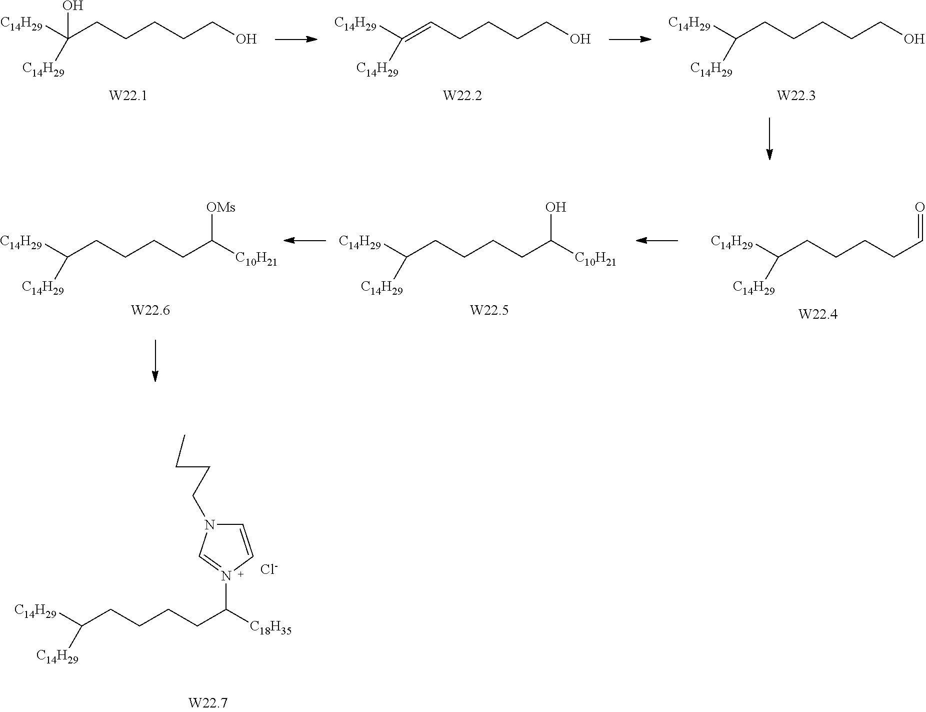

Compounds W22.1 to W22.7 were obtained following procedures similar to the ones described above (W21.1 to W21.7).

### Analysis of Compound W22.7:

TLC: Rf=0.45; solvent: CH₂Cl₂-methanol 9:1; detection with iodine (Merck TLC plates silica gel 60 F₂₅₄).

¹H-NMR (400 MHz, CDCl₃): δ=11.30 (s, 1H), 7.25-7.22 (m, 1H), 7.13-7.09 (m, 1H), 4.55-4.45 (m, 1H), 4.42 (t, J=7.3 Hz, 2H), 1.96-1.67 (m, 6H), 1.55-0.99 (m, 71H), 0.95 (t, J=7.3 Hz, 3H), 0.89-0.77 (m, 15H).

### Synthesis of W23.7: 1-butyl-3-(14-(3,7-dimethyloctyl)-2,6,17,21-tetramethyldecan-9-yl)-1H-imidazol-3-ium chloride

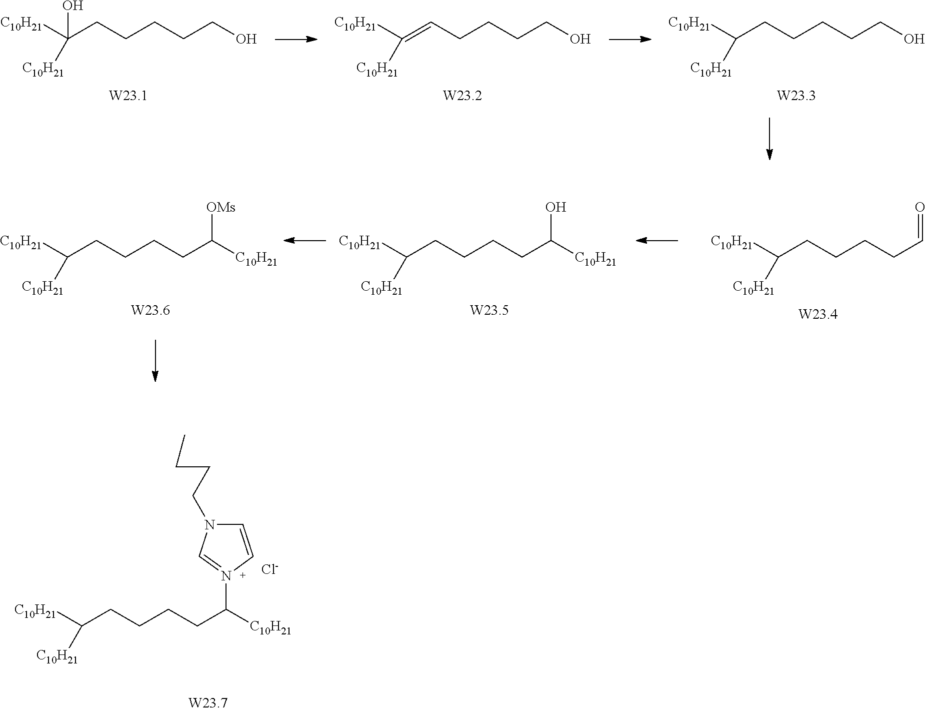

Compounds W23.1 to W23.7 were obtained following procedures similar to the ones described above (W21.1 to W21.7).

### Analysis of Compound W23.7:

TLC: Rf=0.60; solvent: CH₂Cl₂-methanol 9:1; detection with iodine (Merck TLC plates silica gel 60 F₂₅₄).

¹H-NMR (400 MHz, CDCl₃): δ=11.12 (s, 1H), 7.42-7.36 (m, 1H), 7.18-7.12 (m, 1H), 4.52-4.42 (m, 1H), 4.39 (t, J=7.5 Hz, 2H), 1.97-1.64 (m, 6H), 1.55-0.88 (m, 46H), 0.87-0.68 (m, 27H).

### Synthesis of W9.7: 1-methyl-3-(20-tetradecyltetratriacontan-15-yl)-1H-imidazol-3-ium chloride

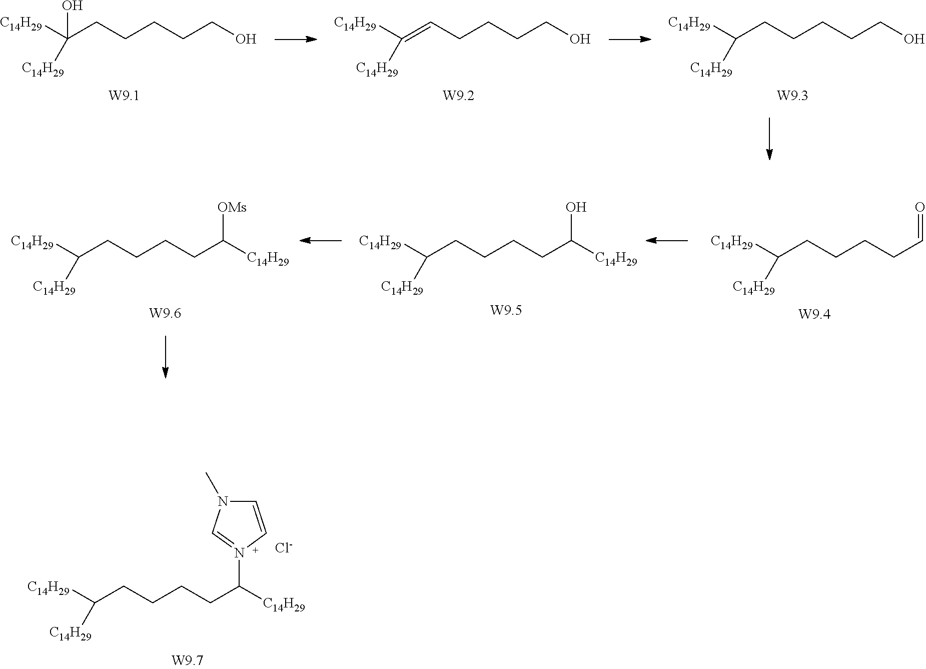

Compounds W9.1 to W9.7 were obtained following procedures similar to the ones described above (W21.1 to W21.7).

### Analysis of Compound W9.7:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=11.10 (s, 1H), 7.28-7.25 (m, 1H), 7.12-7.08 (m, 1H), 4.46-4.36 (m, 1H), 4.14 (s, 3H), 1.92-1.71 (m, 4H), 1.35-0.95 (m, 83H), 0.85 (t, J=6.8 Hz, 9H).

### Synthesis of W10.7: 1-methyl-3-(24-tetradecyloctatriacont-9-en-19-yl)-1H-imidazol-3-ium chloride

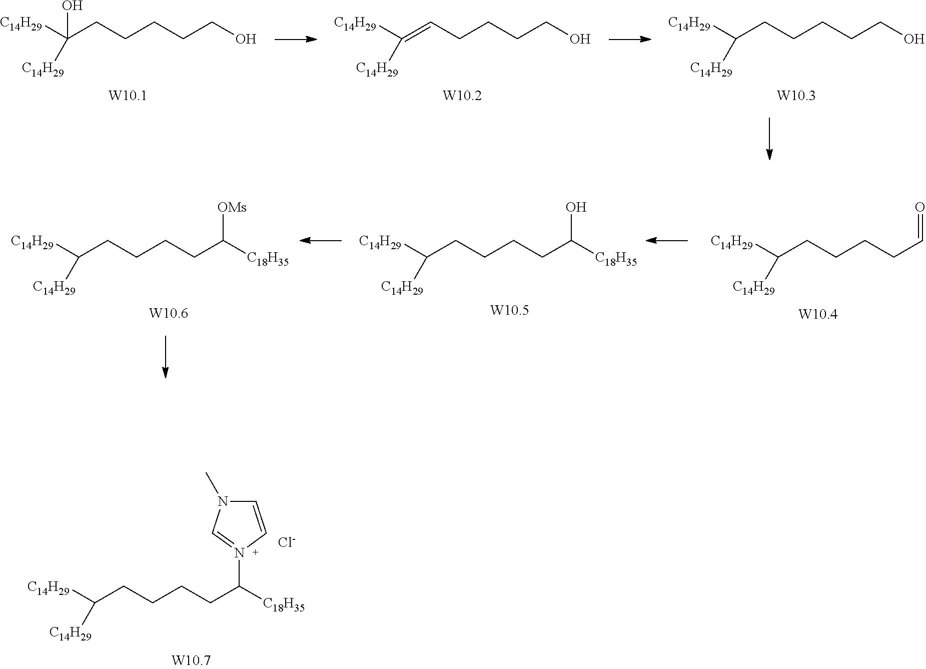

Compounds W10.1 to W10.7 were obtained following procedures similar to the ones described above (W21.1 to W21.7).

### Analysis of Compound W10.7:

TLC: Rf=0.40; solvent: CH₂Cl₂-methanol 9:1; detection with iodine (Merck TLC plates silica gel 60 F₂₅₄).

¹H-NMR (400 MHz, CDCl₃): δ=11.12 (s, 1H), 7.28-7.25 (m, 1H), 7.12-7.08 (m, 1H), 5.37-5.27 (m, 2H), 4.46-4.36 (m, 1H), 4.13 (s, 3H), 2.03-1.70 (m, 8H), 1.35-0.95 (m, 83H), 0.85 (t, J=6.9 Hz, 9H).

### Synthesis of W11.7: 1-methyl-3-(24-tetradecyloctatriacontan-19-yl)-1H-imidazol-3-ium chloride

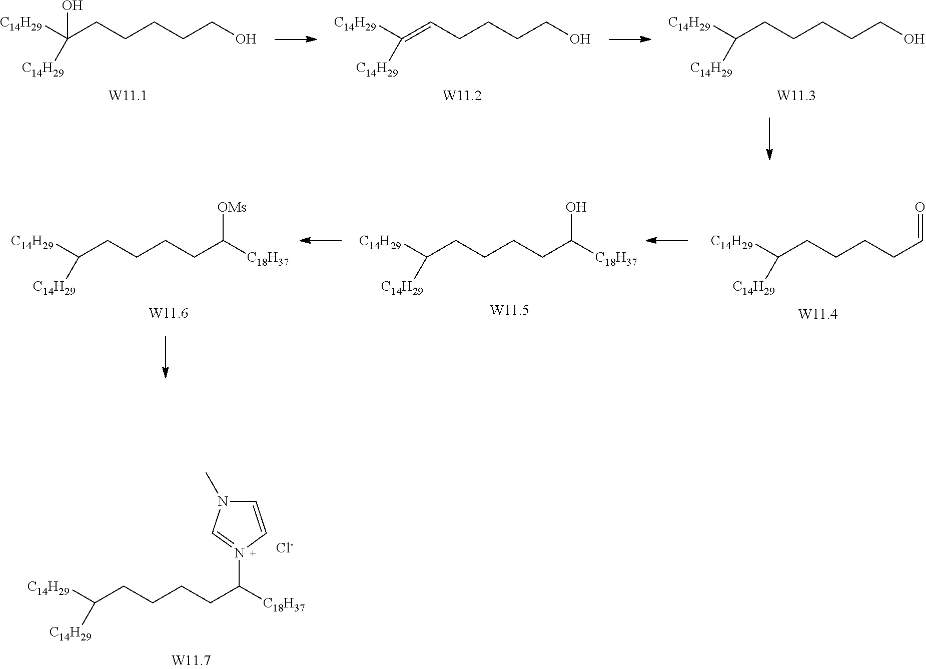

Compounds W11.1 to W11.7 were obtained following procedures similar to the ones described above (W21.1 to W21.7).

### Analysis of Compound W11.7:

TLC: Rf=0.50; solvent: CH₂Cl₂-methanol 9:1; detection with iodine (Merck TLC plates silica gel 60 F₂₅₄).

¹H-NMR (400 MHz, CDCl₃): δ=10.98 (s, 1H), 7.31-7.27 (m, 1H), 7.13-7.09 (m, 1H), 4.47-4.37 (m, 1H), 4.13 (s, 3H), 1.92-1.71 (m, 4H), 1.47-0.96 (m, 91H), 0.85 (t, J=6.8 Hz, 9H).

### Synthesis of W12.7: 1-methyl-3-(2,6-dimethyl-14-tetradecyloctacosan-9-yl)-1H-imidazol-3-ium chloride

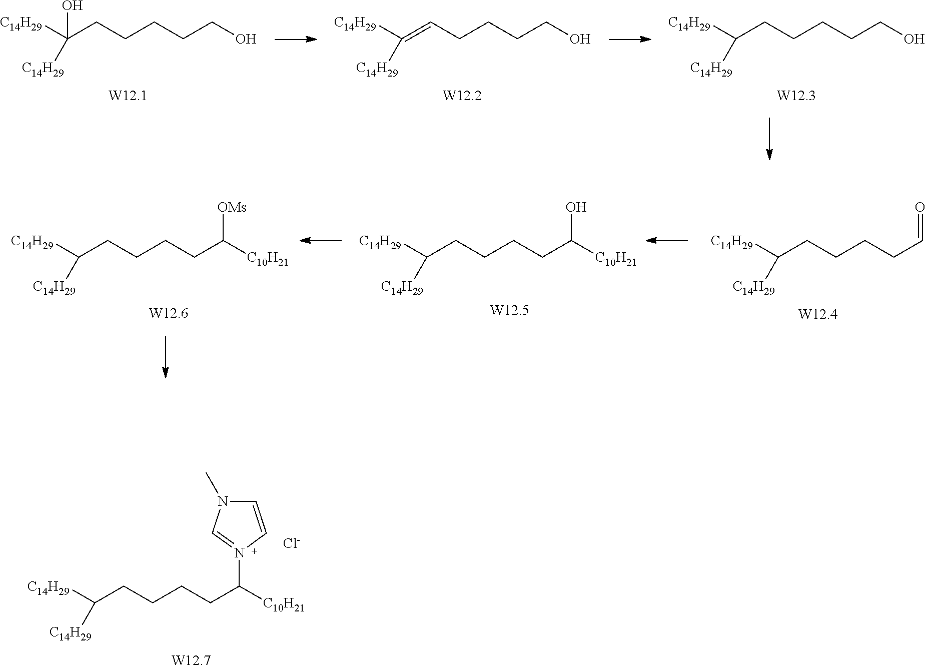

Compounds W12.1 to W12.7 were obtained following procedures similar to the ones described above (W21.1 to W21.7).

### Analysis of Compound W12.7:

TLC: Rf=0.40; solvent: CH₂Cl₂-methanol 9:1; detection with iodine (Merck TLC plates silica gel 60 F₂₅₄).

¹H-NMR (400 MHz, CDCl₃): δ=11.08 (s, 1H), 7.27 (t, J=1.7 Hz, 1H), 7.11 (t, J=1.7 Hz, 1H), 4.47-4.36 (m, 1H), 4.14 (s, 3H), 1.91-1.72 (m, 4H), 1.35-0.96 (m, 75H), 0.89-0.81 (m, 9H).

### Synthesis of W13.7: 1-methyl-3-(24-octadecyldotetracontan-19-yl)-1H-imidazol-3-ium chloride

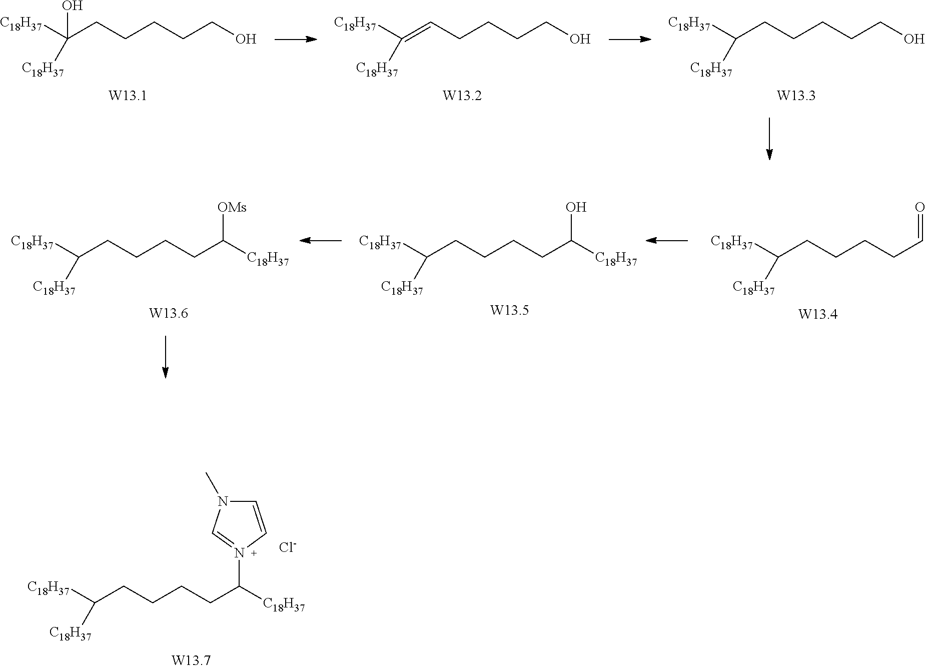

Compounds W13.1 to W13.7 were obtained following procedures similar to the ones described above (W21.1 to W21.7).

### Analysis of Compound W13.7:

TLC: Rf=0.35; solvent: CH₂Cl₂-methanol 9:1; detection with iodine (Merck TLC plates silica gel 60 F₂₅₄).

¹H-NMR (400 MHz, CDCl₃): δ=11.11 (s, 1H), 7.29-7.26 (m, 1H), 7.12-7.08 (m, 1H), 4.46-4.36 (m, 1H), 4.13 (s, 3H), 1.92-1.70 (m, 4H), 1.43-0.94 (m, 107H), 0.85 (t, J=6.8 Hz, 9H).

### Synthesis of W14.7: 1-methyl-3-(1-cyclohexyl-7-octadecylpentacosan-2-yl)-1H-imidazol-3-ium chloride

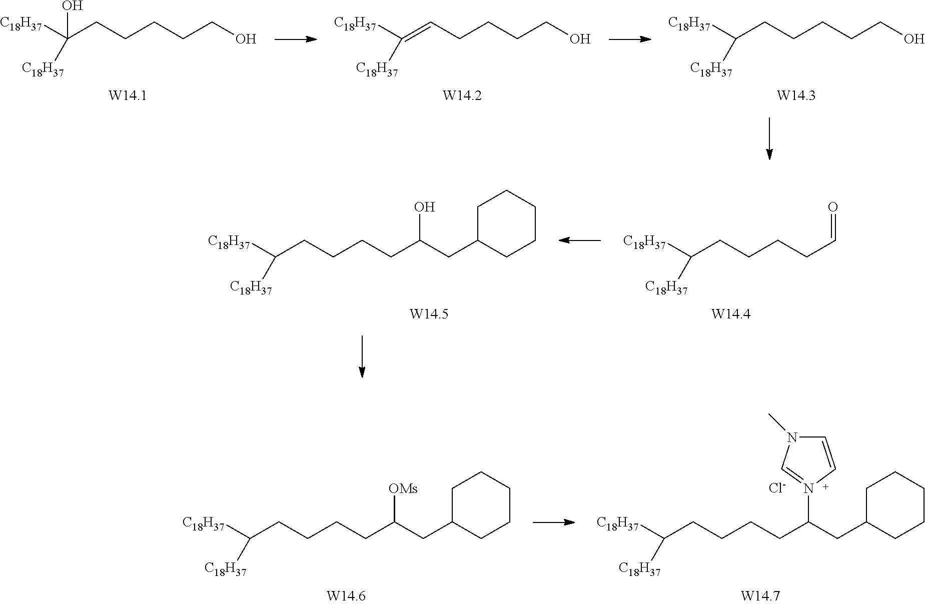

Compounds W14.1 to W14.7 were obtained following procedures similar to the ones described above (W21.1 to W21.7).

### Analysis of Compound W14.7:

TLC: Rf=0.40; solvent: CH₂Cl₂-methanol 9:1; detection with iodine (Merck TLC plates silica gel 60 F₂₅₄).

¹H-NMR (400 MHz, CDCl₃): δ=11.04 (s, 1H), 7.27-7.23 (m, 1H), 7.12-7.07 (m, 1H), 4.52-4.42 (m, 1H), 4.15 (s, 3H), 1.89-1.52 (m, 8H), 1.35-0.89 (m, 82H), 0.85 (t, J=6.8 Hz, 6H).

### Synthesis of W15.7: 1-methyl-3-(7-octadecyl-1-phenylpentacosan-2-yl)-1H-imidazol-3-ium chloride

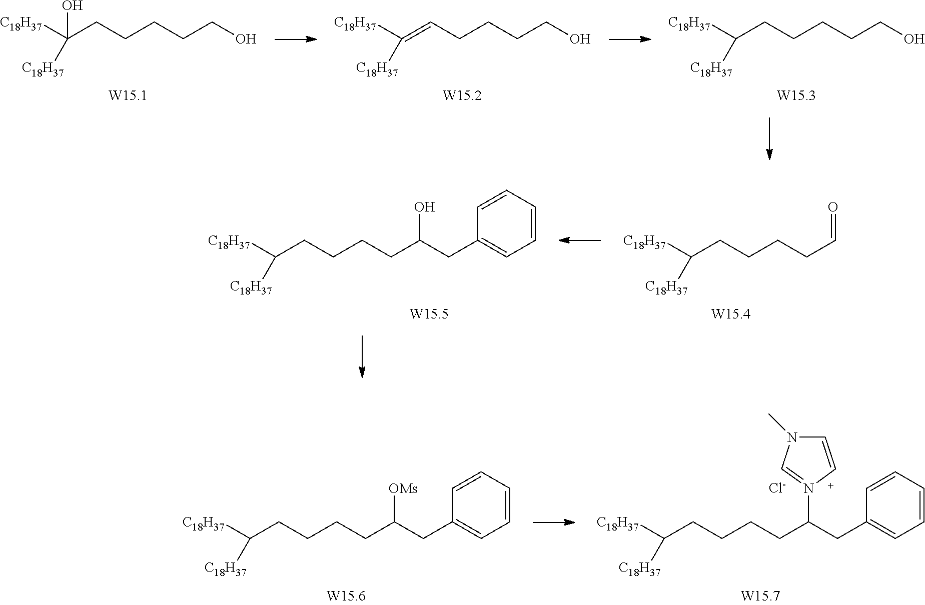

Compounds W15.1 to W15.7 were obtained following procedures similar to the ones described above (W21.1 to W21.7).

### Analysis of Compound W15.7:

TLC: Rf=0.35; solvent: CH₂Cl₂-methanol 9:1; detection with iodine (Merck TLC plates silica gel 60 F₂₅₄).

¹H-NMR (400 MHz, CDCl₃): δ=10.94 (s, 1H), 7.24-7.17 (m, 3H), 7.17-7.13 (m, 1H), 7.13-7.07 (m, 2H), 6.92-6.88 (m, 1H), 4.72-4.62 (m, 1H), 4.05 (s, 3H), 3.26-3.11 (m, 2H), 2.01-1.91 (m, 2H), 1.42-0.97 (m, 75H), 0.85 (t, J=6.8 Hz, 6H).

### Synthesis of W16.7: 1-methyl-3-(2,6-dimethyl-14-octadecyldotriacontan-9-yl)-1H-imidazole-3-ium chloride

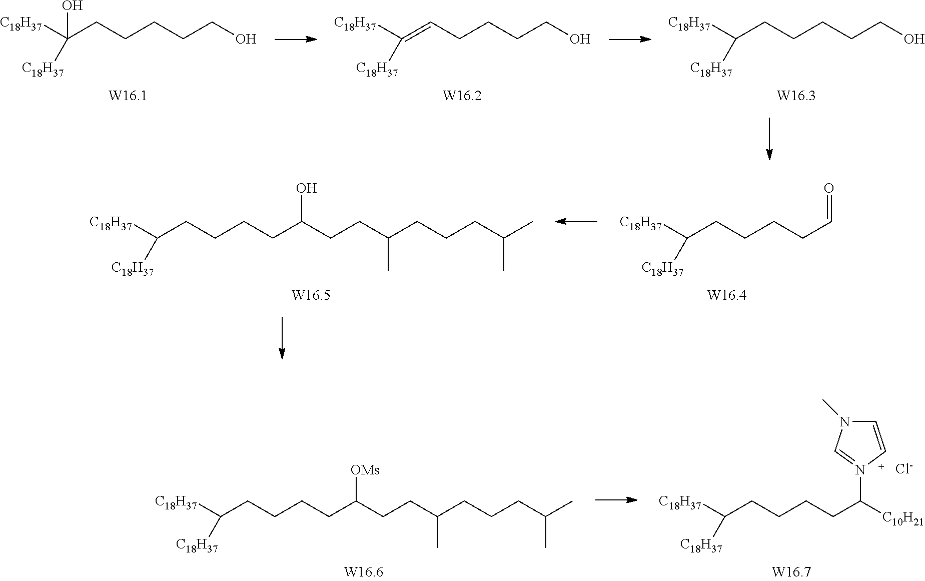

Compounds W16.1 to W16.7 were obtained following procedures similar to the ones described above (W21.1 to W21.7).

### Analysis of Compound W16.7:

TLC: Rf=0.35; solvent: CH₂Cl₂-methanol 9:1; detection with iodine (Merck TLC plates silica gel 60 F₂₅₄).

¹H-NMR (400 MHz, CDCl₃): d=δ=11.03 (s, 1H), 7.31-7.25 (m, 1H), 7.15-7.06 (m, 1H), 4.48-4.33 (m, 1H), 4.15 (s, 3H), 1.96-1.65 (m, 4H), 1.55-0.93 (m, 85H), 0.91-0.75 (m, 15H).

### Synthesis of W17.7: 1-methyl-3-(12-octadecyltriacontan-7-yl)-1H-imidazol-3-ium chloride

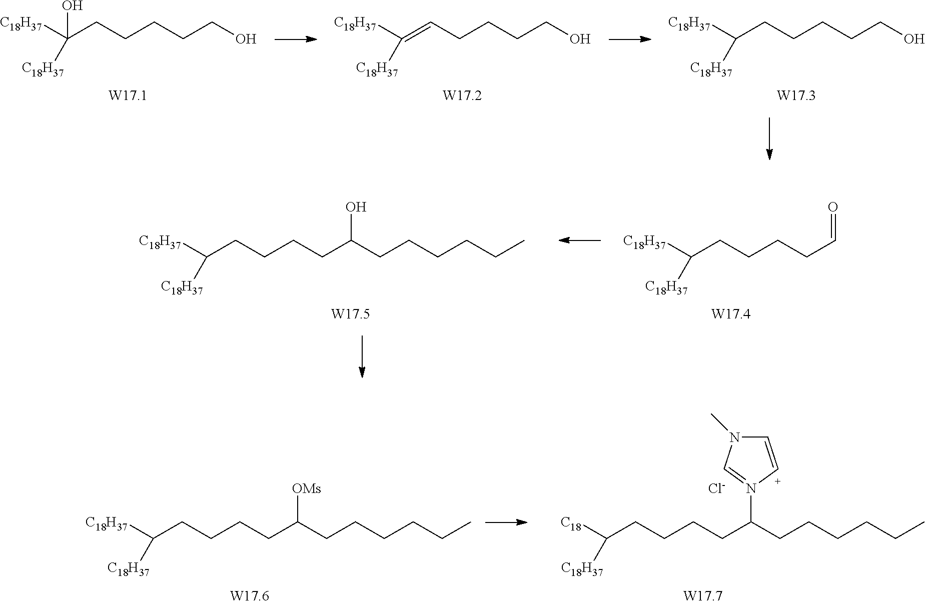

Compounds W17.1 to W17.7 were obtained following procedures similar to the ones described above (W21.1 to W21.7).

### Analysis of Compound W17.7:

TLC: Rf=0.30; solvent: CH₂Cl₂-methanol 9:1; detection with iodine (Merck TLC plates silica gel 60 F₂₅₄).

¹H-NMR (400 MHz, CDCl₃): δ=11.05 (s, 1H), 7.27 (t, J=1.8 Hz, 1H), 7.11 (t, J=1.8 Hz, 1H), 4.47-4.37 (m, 1H), 4.13 (s, 3H), 1.93-1.70 (m, 4H), 1.48-0.95 (m, 83H), 0.91-0.77 (m, 9H).

### Synthesis of W18.9: 1-methyl-3-(15,25-ditetradecylnonatriacontan-20-yl)-1H-imidazol-3-ium chloride

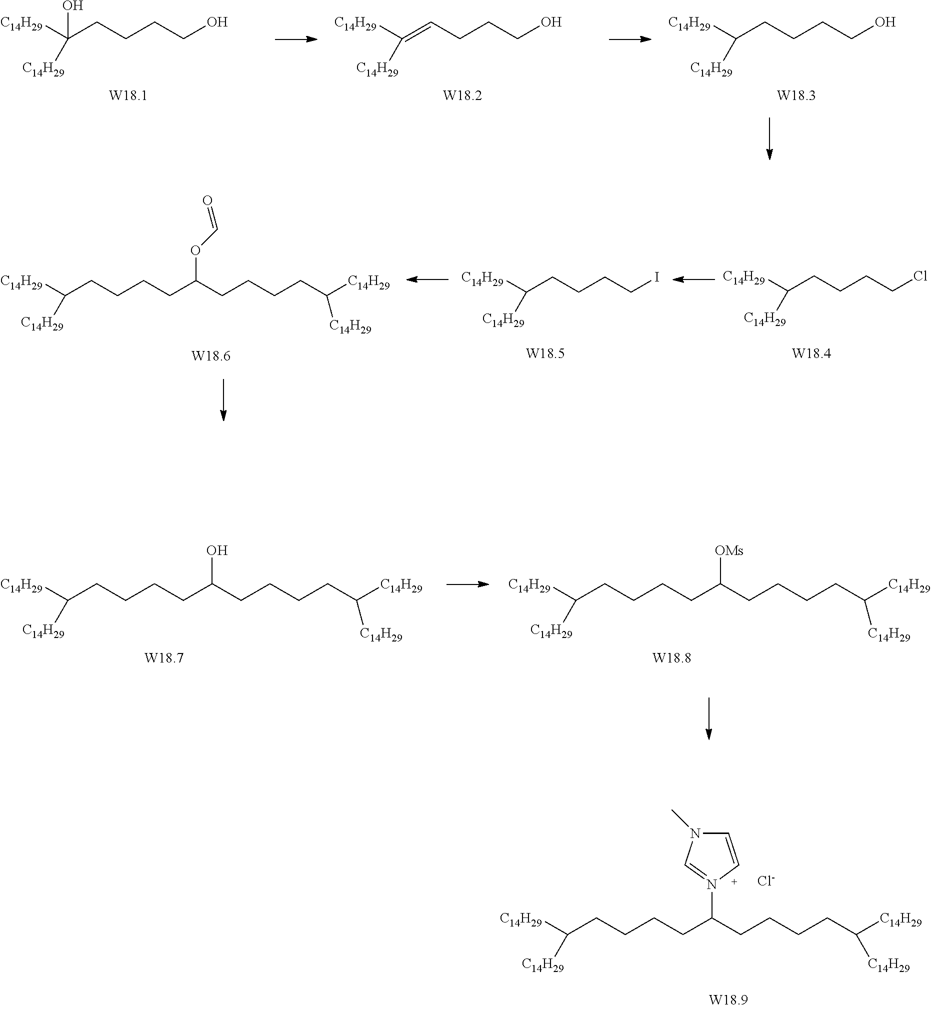

Compounds W18.1 to W18.3, W18.8 and W18.9 were obtained following procedures similar to the ones described above (W21.1 to W21.3, W21.8 and W21.9).

### a) Synthesis of W18.4

To 1.50 g of cyanuric chloride (8.13 mmoles; MW=184.41) in 20 mL of N,N-dimethylformamide was added alcohol W18.3 (2.00 g; 4.16 mmoles; MW=480.89) resuspended in 100 mL of CH₂Cl₂. The reaction mixture was stirred under an argon atmosphere at room temperature for 24 hours. Insoluble matter was removed by filtration, the organic layer was washed with acidified water, dried over anhydrous sodium sulfate and evaporated to dryness. The obtained residue was further chromatographed on silica gel (heptane) to afford 1.65 g of compound W18.4 (3.30 mmoles, MW=499.34, 79% yield).

### Analysis of Compound W18.4:

TLC: Rf=0.90; solvent: heptane; detection with vanillin-sulfuric acid reagent (Merck TLC plates silica gel 60 F₂₅₄).

¹H-NMR (400 MHz, CDCl₃): δ=3.52 (t, J=6.7 Hz, 2H), 1.73 (quint, J=7.2 Hz, 2H), 1.45-1.05 (m, 57H), 0.86 (t, J=6.7 Hz, 6H).

### b) Synthesis of W18.5

To 1.65 g of compound W18.4 (3.30 mmoles; MW=499.34) in 100 mL of acetone was added 1 g of sodium iodide (6.67 mmoles; MW=149.89). The mixture was refluxed under an argon atmosphere for 6 days. Solvent was removed under reduced pressure. The residue was dissolved in 100 mL of CH₂Cl₂, the solution was washed with acidified water, dried over anhydrous sodium sulfate and evaporated to dryness. The obtained residue was further chromatographed on silica gel (heptane) to afford 1.84 g of compound W18.5 (3.11 mmoles, MW=590.79, 94% yield).

### Analysis of Compound W18.5:

TLC: Rf=0.90; solvent: heptane; detection with vanillin-sulfuric acid reagent and UV (Merck TLC plates silica gel 60 F₂₅₄).

¹H-NMR (400 MHz, CDCl₃): δ=3.17 (t, J=7.0 Hz, 2H), 1.78 (quint, J=7.1 Hz, 2H), 1.45-1.00 (m, 57H), 0.86 (t, J=6.7 Hz, 6H).

### c) Synthesis of W18.6

A solution of 1.84 g of iodide W18.5 (3.11 mmoles; MW=590.79) in 10 mL of diethyl ether was added drop-wise to 0.15 g of magnesium turnings (6.17 mmoles; MW=24.31) in 5 mL of diethyl ether while heating. After 3 hours of reflux, the mixture was cooled to room temperature and 0.1 mL of ethyl formate (1.24 mmoles; MW=74.08) were added drop-wise. After 4 hours, the mixture was poured onto 100 mL of split ice, acidified with concentrated hydrochloric acid for 1 hour. The solid residue in suspension was filtered off and washed with water and was further chromatographed on silica gel (CH₂Cl₂-heptane 1:9) to afford 0.37 g of compound W18.6 (0.38 mmoles, MW=965.80, 30% yield).

### d) Analysis of Compound W18.6

TLC: Rf=0.30; solvent: heptane; detection with vanillin-sulfuric acid reagent (Merck TLC plates silica gel 60 F₂₅₄).

¹H-NMR (400 MHz, CDCl₃): δ=8.07 (s, 1H), 4.96 (quint, J=6.1 Hz, 1H), 1.61-1.47 (m, 8H), 1.42-1.05 (m, 113H), 0.86 (t, J=6.9 Hz, 12H).

Analysis of compound W18.9:

TLC: Rf=0.45; solvent: CH₂Cl₂-ethanol 9:1; detection with iodine (Merck TLC plates silica gel 60 F₂₅₄).

¹H-NMR (400 MHz, CDCl₃): δ=10.96 (s, 1H), 7.23-7.19 (m, 1H), 7.13-7.06 (m, 1H), 4.47-4.36 (m, 1H), 4.13 (s, 3H), 1.93-1.69 (m, 4H), 1.43-0.95 (m, 118H), 0.85 (t, J=6.7 Hz, 12H).

### Synthesis of W19.7: 1-methyl-3-(15-(octadecylammonio)pentacosan-11-yl)-1H-imidazol-3-ium chloride

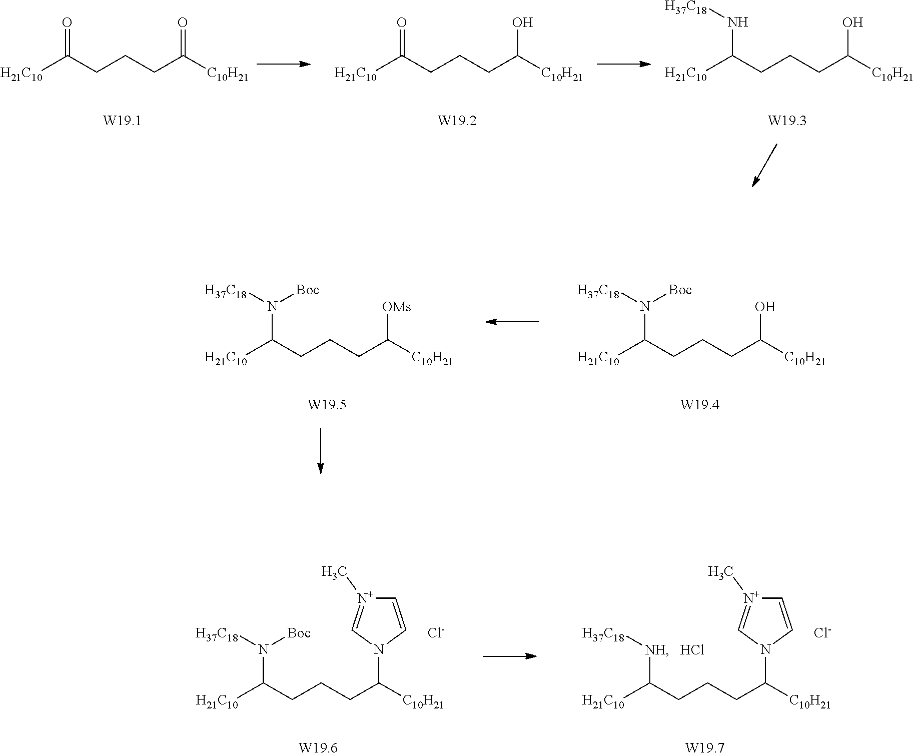

### a) Synthesis of W19.1

To 5 mL of glutaryl chloride (39.17 mmoles; MW=169.01) in 100 mL of CH₂Cl₂ was added N,O-dimethylhydroxylamine hydrochloride (8.40 g; 86.11 mmoles; MW=97.54). The mixture was cooled in an ice-water bath and 19 mL of pyridine (234.92 mmoles; MW=79.10) were added drop-wise. After 2 hours at room temperature, insoluble matter was removed by filtration and the filtrate was evaporated to dryness. The residue was resuspended in 100 mL of tetrahydrofuran, the mixture was cooled in an ice-water bath and 94 mL of decylmagnesium bromide solution 1M in Diethyl ether (94.00 mmoles) were added drop-wise. After 2 hours, the mixture was poured onto 400 mL of split ice, acidified with concentrated hydrochloric acid for 1 hour. The solid residue in suspension was filtered off and washed with water and with ethyl acetate to afford 10.10 g of compound W19.1 (26.53 mmoles, MW=380.65, 68% yield).

### Analysis of Compound W19.1:

TLC: Rf=0.45; solvent: CH₂Cl₂-heptane 1:1; detection with vanillin-sulfuric acid reagent (Merck TLC plates silica gel 60 F₂₅₄).

¹H-NMR (400 MHz, CDCl₃): δ=2.40 (t, J=7.2 Hz, 4H), 2.35 (t, J=7.5 Hz, 4H), 1.81 (quint, J=7.2 Hz, 2H), 1.56-1.46 (m, 4H), 1.34-1.14 (m, 28H), 0.85 (t, J=6.8 Hz, 6H).

### b) Synthesis of W19.2

To 6.00 g of compound W19.1 (15.76 mmoles; MW=380.65) in 250 mL of tetrahydrofuran was added 2.40 g of sodium borohydride (63.42 mmoles; MW=37.83). After 3 hours at room temperature, solvent was removed under reduced pressure. The residue was resuspended in ethyl acetate and insoluble matter was removed by filtration. The organic layer was washed with water, dried over anhydrous sodium sulfate and evaporated to dryness. The obtained residue was further chromatographed on silica gel (CH₂Cl₂-methanol 98:2) to afford 0.88 g of compound W19.2 (2.30 mmoles, MW=382.66, 14% yield).

### Analysis of Compound W19.2:

TLC: Rf=0.60; solvent: CH₂Cl₂-methanol 98:2; detection with vanillin-sulfuric acid reagent (Merck TLC plates silica gel 60 F₂₅₄).

¹H-NMR (400 MHz, CDCl₃): δ=3.58-3.48 (m, 1H), 2.45-2.32 (m, 4H), 1.48-1.15 (m, 38H), 0.86 (t, J=6.9 Hz, 6H).

### c) Synthesis of W19.3

To 0.88 g of compound W19.2 (2.30 mmoles; MW=382.66) in 50 mL of tetrahydrofuran was added 0.68 g of octadecylamine (2.52 mmoles; MW=269.51) and 0.132 mL of acetic acid (2.30 mmoles, MW=60.05). After 1 hour at room temperature, 0.087 g of sodium borohydride (2.30 mmoles; MW=37.83) in 5 mL of water was added. The mixture was evaporated to dryness and the residue was further chromatographed on silica gel (CH₂Cl₂-methanol 9:1) to afford 0.48 g of compound W19.3 (0.75 mmole, MW=636.17, 32% yield).

Analysis of compound W19.3:

TLC: Rf=0.50; solvent: CH₂Cl₂-methanol 9:1; detection with vanillin-sulfuric acid reagent or ninhydrin (Merck TLC plates silica gel 60 F₂₅₄).

¹H-NMR (400 MHz, CDCl₃): δ=3.62-3.52 (m, 1H), 3.03-2.88 (m, 1H), 2.88-2.72 (m, 2H), 1.76-1.10 (m, 74H), 0.86 (t, J=7.0 Hz, 9H).

### d) Synthesis of W19.4

To 0.48 g of compound W19.3 (0.75 mmole; MW=636.17) in 30 mL of CH₂Cl₂ was added 1 mL of triethylamine (7.17 mmoles; MW=101.19) and 0.30 g of Di-tert-butyl dicarbonate (1.37 mmoles, MW=218.25). After 3 hours at room temperature, the organic layer was washed with water acidified with HCl, dried over anhydrous sodium sulfate and evaporated to dryness. 0.56 g of compound W19.4 were obtained without further purification (0.75 mmole, MW=736.29, quantitative yield).

### Analysis of Compound W19.4:

TLC: Rf=0.25; solvent: CH₂Cl₂; detection with vanillin-sulfuric acid reagent (Merck TLC plates silica gel 60 F₂₅₄).

¹H-NMR (400 MHz, CDCl₃): δ=4.05-3.92 (m, 1H), 3.60-3.47 (m, 1H), 3.00-2.81 (m, 2H), 1.50-1.15 (m, 83H), 0.86 (t, J=6.8 Hz, 9H).

Compounds W19.5 and W19.6 were obtained following procedures similar to the ones described above (W21.6 and W21.7).

### e) Synthesis of W19.7

0.10 g of compound W19.6 (0.12 mmole; MW=836.84) was treated with 1 mL of trifluoroacetic acid (13.06 mmoles; MW=114.02). After 1 hour at room temperature, the mixture was evaporated to dryness. The residue was dissolved in 2 mL of methanol and 0.5 mL of 37% hydrochloric acid and the mixture was evaporated to dryness. The residue was further chromatographed on silica gel (CH₂Cl₂-methanol 9:1) to afford 0.08 g of compound W19.7 (0.10 mmole, MW=773.18, 87% yield).

### Analysis of Compound W19.7:

TLC: Rf=0.35; solvent: CH₂Cl₂-methanol 9:1; detection with vanillin-sulfuric acid reagent or ninhydrin (Merck TLC plates silica gel 60 F₂₅₄).

¹H-NMR (400 MHz, MeOD): d=9.09 (s, 1H), 7.77-7.72 (m, 1H), 7.66-7.61 (m, 1H), 4.44-4.33 (m, 1H), 3.96 (s, 3H), 3.16-3.07 (m, 1H), 2.99-2.92 (m, 2H), 1.99-1.82 (m, 4H), 1.78-1.58 (m, 6H), 1.54-1.04 (m, 64H), 0.95-0.85 (m, 9H).

### Synthesis of W25.7: 1-(2-hydroxyethyl)-3-(24-tetradecyloctatriacontan-19-yl)-1H-imidazol-3-ium chloride

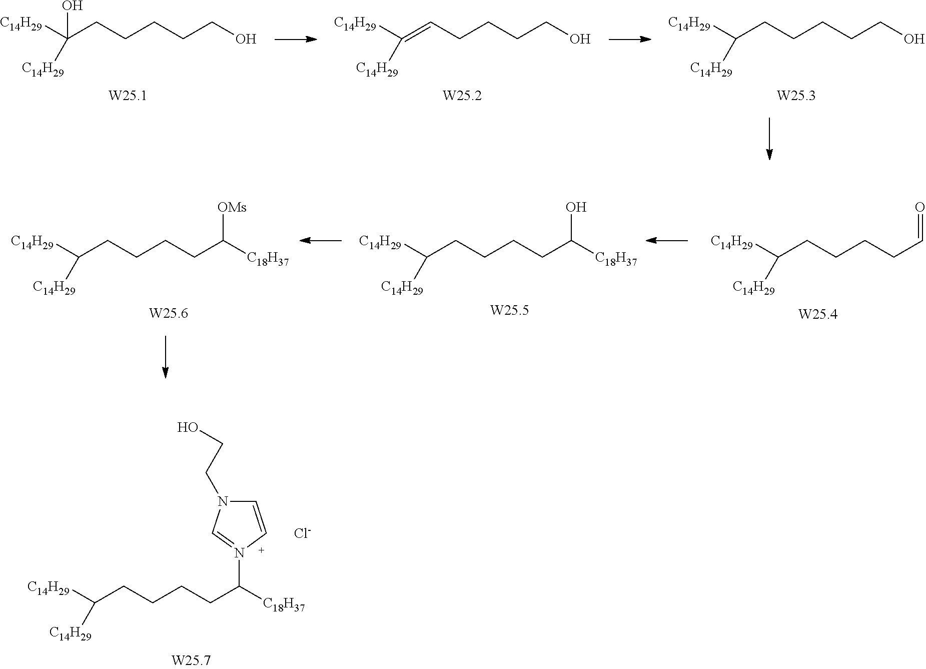

Compounds W25.1 to W25.7 were obtained following procedures similar to the ones described above (W21.1 to W21.7).

### Analysis of Compound W25.7:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=10.09 (s, 1H), 7.47 (t, J=1.8 Hz, 1H), 7.11 (t, J=1.8 Hz, 1H), 4.57 (t, J=4.4 Hz, 2H), 4.33-4.22 (m, 1H), 3.98 (t, J=4.4 Hz, 2H), 1.94-1.70 (m, 4H), 1.51-0.98 (m, 91H), 0.85 (t, J=6.8 Hz, 9H).

### Synthesis of W26.7: 1-(2-hydroxyethyl)-3-(20-tetradecyltetratriacontan-15-yl)-1H-imidazol-3-ium chloride

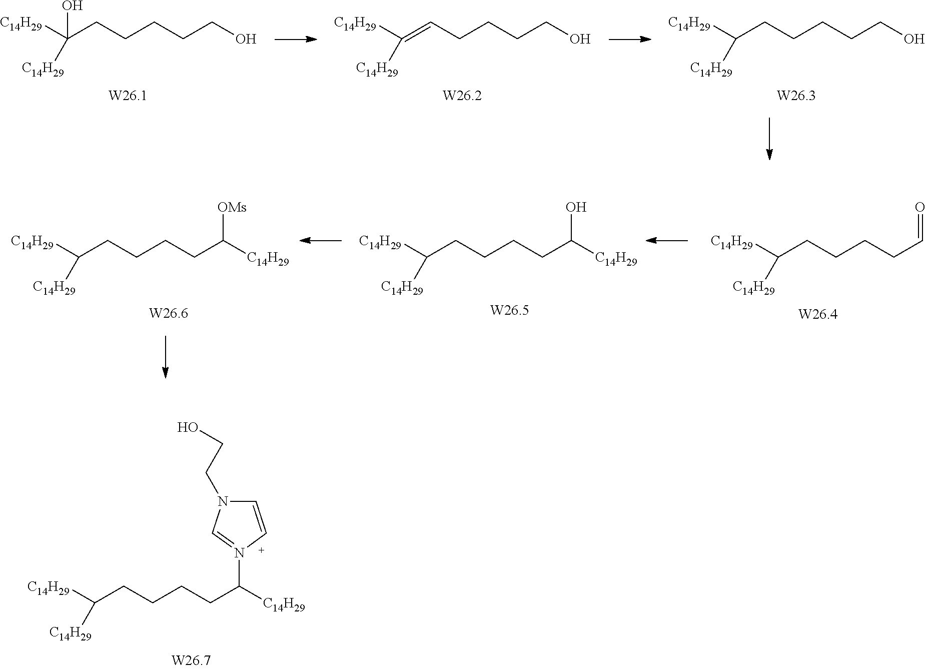

Compounds W26.1 to W26.7 were obtained following procedures similar to the ones described above (W21.1 to W21.7).

### Analysis of Compound W26.7:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=11.05 (s, 1H), 7.27 (t, J=1.8 Hz, 1H), 7.11 (t, J=1.8 Hz, 1H), 4.47-4.37 (m, 1H), 4.13 (s, 3H), 1.93-1.70 (m, 4H), 1.48-0.95 (m, 83H), 0.91-0.77 (m, 9H).

### Synthesis of W27.7: 1-ethyl-3-(24-tetradecyloctatriacont-9-en-19-yl)-1H-imidazol-3-ium chloride

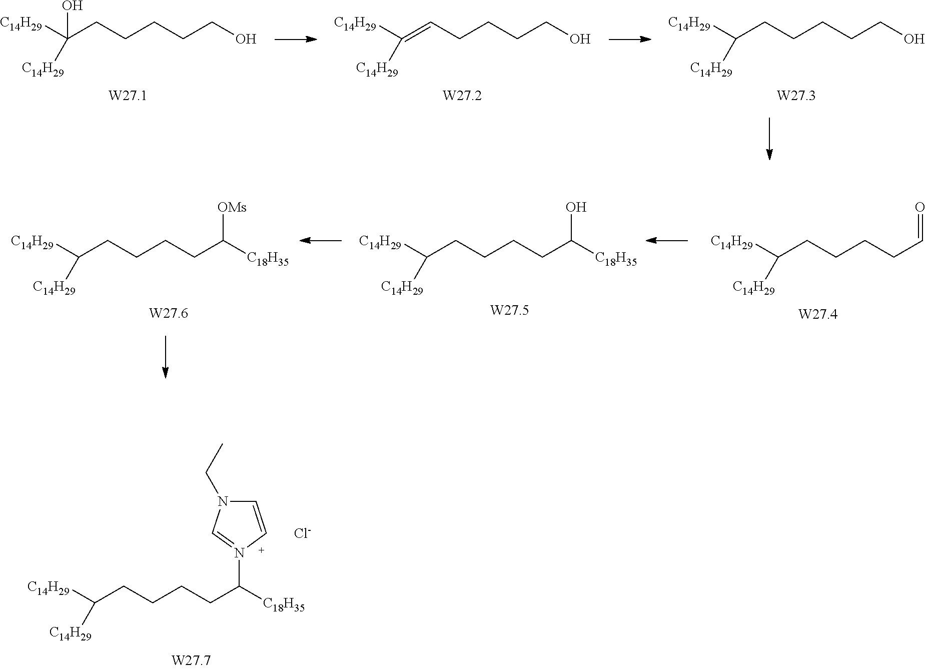

Compounds W27.1 to W27.7 were obtained following procedures similar to the ones described above (W21.1 to W21.7).

### Analysis of Compound W27.7:

TLC: Rf=0.4; solvent: CH₂Cl₂-methanol 9:1; detection with iodine (Merck TLC plates silica gel 60 F₂₅₄).

¹H-NMR (400 MHz, CDCl₃): δ=11.25 (s, 1H), 7.28 (t, J=1.6 Hz, 1H), 7.11 (t, J=1.6 Hz, 1H), 5.37-5.26 (m, 2H), 4.48 (q, J=7.3 Hz, 2H), 2.01-1.91 (m, 4H), 1.89-1.83 (m, 4H), 1.59 (t, J=7.3 Hz, 3H), 1.42-0.95 (m, 84H), 0.85 (t, J=6.8 Hz, 9H).

### Synthesis of W28.7: 1-methyl-3-(15-tetradecylheptatriacontan-19-yl)-1H-imidazol-3-ium chloride

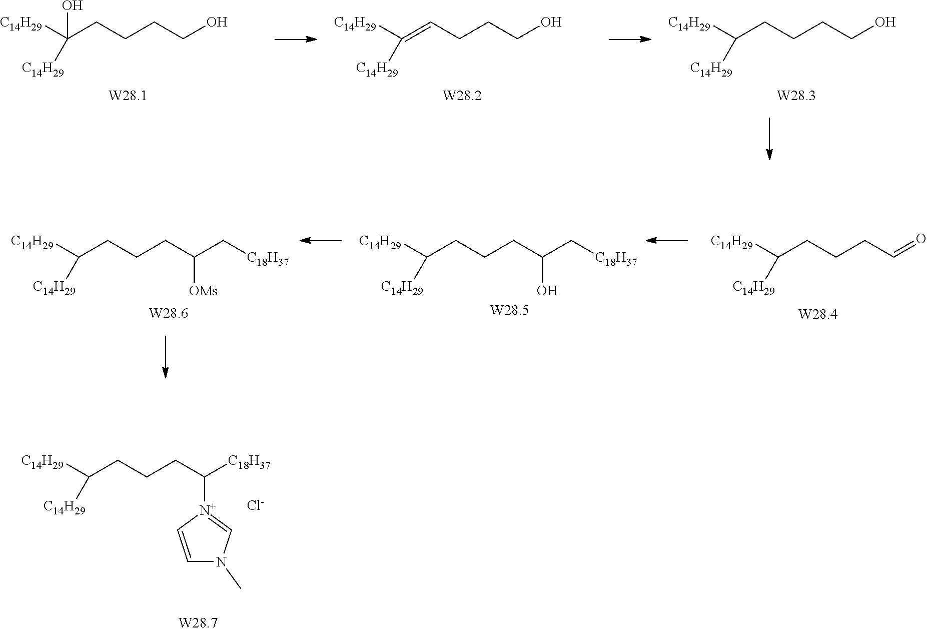

Compounds W28.1 to W28.7 were obtained following procedures similar to the ones described above (W21.1 to W21.7).

### Analysis of Compound W28.7:

TLC: Rf=0.4; solvent: CH₂Cl₂-methanol 9:1; detection with iodine (Merck TLC plates silica gel 60 F₂₅₄).

¹H-NMR (400 MHz, CDCl₃): δ=11.15 (s, 1H), 7.24 (t, J=1.5 Hz, 1H), 7.10 (t, J=1.5 Hz, 1H), 4.45-4.36 (m, 1H), 4.14 (s, 3H), 1.91-1.79 (m, 4H), 1.43-0.99 (m, 89H), 0.85 (t, J=6.8 Hz, 9H).

### Synthesis of W29.5: 1-methyl-3-(4-hexadecylicosyl)-1H-imidazol-3-ium chloride

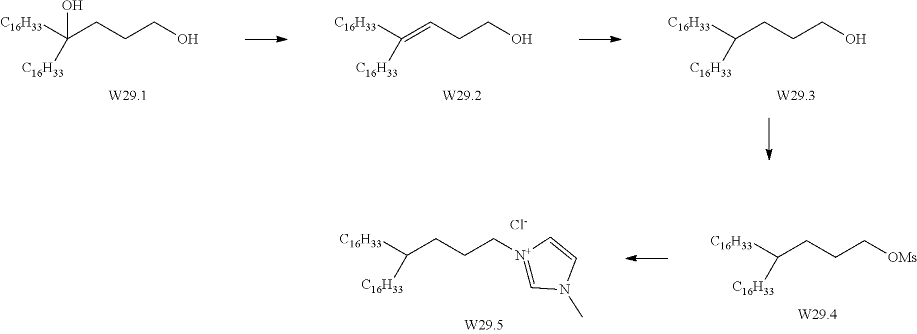

Compounds W29.1 to W29.5 were obtained following procedures similar to the ones described above (W21.1 to W21.3 and W21.5 to W21.7).

### Analysis of Compound W29.5:

TLC: Rf=0.43; solvent: CH₂Cl₂-methanol 9:1; detection with vanillin-sulfuric acid reagent (Merck TLC plates silica gel 60 F₂₅₄).

¹H-NMR (400 MHz, CDCl₃): δ=10.65 (s, 1H), 7.49 (s, 1H), 7.28 (s, 1H), 4.27 (t, J=7.5 Hz, 2H), 4.12 (s, 3H), 1.85 (q, J=6.1 Hz, 2H), 1.24 (s, 1H), 1.19-1.24 (m, 66H).
Synthesis of W8.7: 1-methyl-3-(hexatriaconta-8,27-dien-18-yl)-1H-imidazol-3-ium

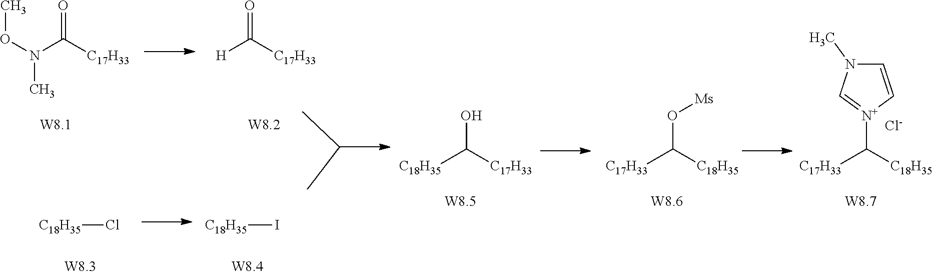

### a) Synthesis of Amide W8.1:

To a solution of 3 mL Pyridine (2.93 g; 37.09 mmoles; MW=79.10) and 1.80 g of N,O-dimethylhydroxylamine hydrochloride (19.48 mmoles; MW=97.54) in 50 mL of CH₂Cl₂ cooled in an ice-water bath was added drop-wise 5.5 mL of oleoyl chloride (5.00 g; 16.63 mmoles; MW=300.91). The mixture was allowed to stay at room temperature for 1 hour. The solvents were removed under reduced pressure to give a crude that was purified with liquid/liquid partition (ethyl acetate/water) to give 5.46 g of pure amide W8.1 (16.77 mmoles; MW=325.53; quantitative yield).

### Analysis of Compound W8.1:

TLC: Rf=0.3; solvent: CH₂Cl₂; detection with iodine (Merck TLC plates silica gel 60 F₂₅₄).

¹H-NMR (400 MHz, CDCl₃): δ=5.35-5.29 (m, 2H), 3.66 (s, 3H), 3.15 (s, 3H), 2.38 (t, J=7.5 Hz, 2H), 1.98 (q, J=6.1 Hz, 4H), 1.60 (quint, J=7.5 Hz, 2H), 1.37-1.18 (m, 20H), 0.86 (t, J=6.9 Hz, 3H).

### b) Synthesis of Aldehyde W8.2:

To a solution of 1 g of amide W8.1 (3.07 mmoles; MW=325.53) in 20 mL of CH₂Cl₂ cooled in a dry ice bath was added drop-wise 6.7 mL of diisobutylaluminum hydride solution at 1M in cyclohexane (6.70 mmoles). 5 hours later, 10 mL of methanol were added and the mixture was allowed to stay at room temperature overnight. The solvents were removed under reduced pressure to give a crude that was purified with liquid/liquid partition (diethyl ether/water) followed by chromatography on silica gel (CH₂Cl₂-cyclohexane 2:8) to afford 0.25 g of aldehyde W8.2 (0.93 mmole; MW=266.46; 31% yield).

### Analysis of Compound W8.2:

TLC: Rf=0.3; solvent: CH₂Cl₂-cyclohexane 3:7; detection with iodine (Merck TLC plates silica gel 60 F₂₅₄).

¹H-NMR (400 MHz, CDCl₃): δ=9.74 (t, J=1.9 Hz, 1H), 5.39-5.27 (m, 2H), 2.40 (td, J=7.4 Hz, 1.9 Hz, 2H), 1.99 (q, J=5.8 Hz, 4H), 1.61 (quint, J=7.4 Hz, 2H), 1.38-1.18 (m, 20H), 0.86 (t, J=6.8 Hz, 3H).

### c) Synthesis of Chloride W8.3:

To a solution of 3.78 g of cyanuric chloride (20.50 mmoles; MW=184.41) in 10 mL of dimethylformamide was added a solution of 5 g of oleyl alcohol (18.62 mmoles; MW=268.48) in 50 mL of CH₂Cl₂. The mixture was allowed to stay overnight at room temperature. The mixture was poured onto 100 mL of split ice. After liquid/liquid partition and removal of solvents, the crude was chromatographed on silica gel (CH₂Cl₂-cyclohexane 1:9) to afford 3.00 g of chloride W8.3 (10.46 mmoles; MW=286.92; 56% yield).

### Analysis of Chloride W8.3:

¹H-NMR (400 MHz, CDCl₃): δ=5.40-5.26 (m, 2H), 3.51 (t, J=6.8 Hz, 2H), 2.08-1.90 (m, 4H), 1.75 (quint, J=7.2 Hz, 2H), 1.40 (quint, J=6.8 Hz, 2H), 1.40-1.18 (m, 20H), 0.86 (t, J=6.8 Hz, 3H).

### d) Synthesis of Iodide W8.4:

To a solution of 3.00 g of chloride W8.3 (10.46 mmoles; MW=286.92) in 100 mL of acetone was added 3.14 g of sodium iodide (20.95 mmoles; MW=149.89). The mixture was refluxed during 4 days. After removal of insoluble matter by filtration, solvents were removed and the crude was chromatographed on silica gel (cyclohexane) to afford 3.76 g of iodide W8.4 (9.94 mmoles; MW=378.37; 95% yield).

### Analysis of Iodide W8.4:

¹H-NMR (400 MHz, CDCl₃): δ=5.42-5.26 (m, 2H), 3.17 (t, J=7.1 Hz, 2H), 2.06-1.90 (m, 4H), 1.80 (quint, J=7.1 Hz, 2H), 1.46-1.16 (m, 22H), 0.86 (t, J=6.8 Hz, 3H). Traces of chloride W8.3 (10%) are remaining.

### e) Synthesis of Alcohol W8.5:

A solution of 1.10 g of iodide W8.4 (2.91 mmoles; MW=378.37) in 5 mL of diethyl ether was added drop-wise to 0.10 g of magnesium turnings (4.11 mmoles; MW=24.31) in 5 mL of diethyl ether while heating. After 2 hours of reflux, the mixture was cooled to room temperature and 0.50 g of aldehyde W8.2 (1.88 mmoles; MW=266.46) in 10 mL of diethyl ether were added drop-wise. After 4 hours, the mixture was poured onto 100 mL split, ice acidified with concentrated hydrochloric acid for 1 hour. After extraction with ethyl acetate and removal of solvents, the crude was chromatographed on silica gel (CH₂Cl₂-cyclohexane 3:7) to afford 0.27 g of alcohol W8.5 (0.52 mmole; MW=518.94; 27% yield).

### Analysis of Alcohol W8.5:

TLC: Rf=0.15; solvent: CH₂Cl₂-cyclohexane 3:7; detection with iodine (Merck TLC plates silica gel 60 F₂₅₄).

¹H-NMR (400 MHz, CDCl₃): δ=5.42-5.27 (m, 4H), 3.60-3.51 (m, 1H), 2.17-1.82 (m, 8H), 1.50-1.06 (m, 50H), 0.86 (t, J=6.8 Hz, 6H).

### f) Synthesis of Mesylate W8.6:

Alcohol W8.5 (0.27 g; 0.52 mmole; MW=518.94) was dissolved in 15 mL of dry CH₂Cl₂ and 0.75 mL of triethylamine (0.54 g; 5.38 mmoles; MW=101.19) were added, followed by 0.33 mL of methanesulfonyl chloride (0.49 g; 4.26 mmoles; MW=114.55) introduced drop-wise. The mixture was stirred overnight at room temperature. After removal of the solvents under reduced pressure, the residue was resuspended in 20 mL of methanol. After decantation of the solvents, 0.26 g of mesylate W8.6 were obtained (0.44 mmole, MW=597.03, 84% yield).

### Analysis of Mesylate W8.6:

¹H-NMR (400 MHz, CDCl₃): δ=5.41-5.26 (m, 4H), 4.68 (quint, J=6.1 Hz, 1H), 2.97 (s, 3H), 2.09-1.90 (m, 8H), 1.75-1.58 (m, 4H), 1.47-1.07 (m, 46H), 0.86 (t, J=6.8 Hz, 6H).

### g) Synthesis of Compound W8.7

Mesylate W8.6 (0.26 g; 0.44 mmole; MW=597.03) was dissolved in 10 mL of 1-methylimidazole and was stirred at 80° C. for 5 days under an argon atmosphere. The mixture was evaporated to dryness under high vacuum, the residue was then solubilized in 20 mL of methanol and 10 mL of a 3 M hydrochloric acid was added. After removal of solvents, the crude was chromatographed on silica gel (CH₂Cl₂-methanol 9:1) to afford 0.13 g of compound W8.7 (0.21 mmole, MW=619.49, 48% yield).

### Analysis of Compound W8.7:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=10.96 (s, 1H), 7.29 (t, J=1.6 Hz, 1H), 7.11 (t, J=1.6 Hz, 1H), 5.40-5.24 (m, 4H), 4.46-4.35 (m, 1H), 4.12 (s, 3H), 2.03-1.89 (m, 8H), 1.88-1.71 (m, 4H), 1.36-0.96 (m, 46H), 0.85 (t, J=6.8 Hz, 6H).

### Synthesis of W2.5: 1-methyl-3-(heptatriaconta-9,28-dien-19-yl)-1H-imidazol-3-ium chloride

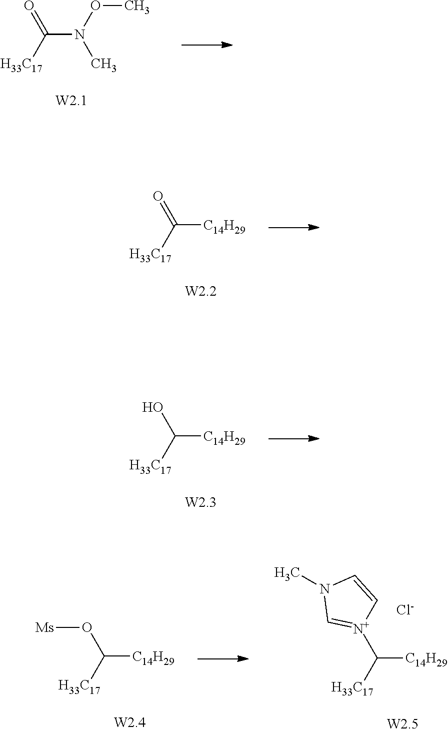

Compounds W2.1 to W2.5 were obtained following procedures similar to the ones described above (W8.1, W8.5, W19.2, W21.6 and W21.7 respectively).

### Analysis of Compound W2.5:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=11.02 (s, 1H), 7.38 (t, J=1.6 Hz, 1H), 7.13 (t, J=1.6 Hz, 1H), 5.36-5.24 (m, 2H), 4.45-4.35 (m, 1H), 4.12 (s, 3H), 2.05-1.89 (m, 4H), 1.89-1.70 (m, 4H), 1.35-0.95 (m, 48H), 0.84 (t, J=6.7 Hz, 6H).

### Synthesis of W3.5: 1-methyl-3-(hexatriacont-8-en-18-yl)-1H-imidazol-3-ium chloride

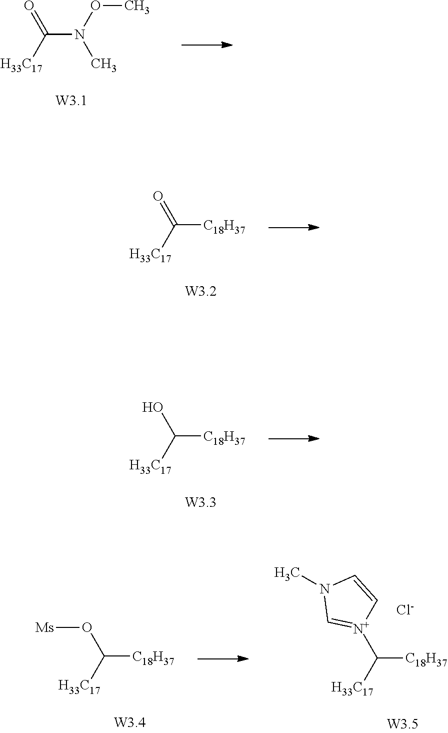

Compounds W3.1 to W3.5 were obtained following procedures similar to the ones described above (W8.1, W8.5, W19.2, W21.6 and W21.7 respectively).

### Analysis of Compound W3.5:

TLC: Rf=0.25; solvent: CH₂Cl₂-methanol 9:1; detection with iodine (Merck TLC plates silica gel 60 F₂₅₄).

¹H-NMR (400 MHz, CDCl₃): δ=11.12 (s, 1H), 7.29 (t, J=1.6 Hz, 1H), 7.11 (t, J=1.6 Hz, 1H), 5.36-5.24 (m, 2H), 4.46-4.36 (m, 1H), 4.13 (s, 3H), 2.05-1.90 (m, 4H), 1.90-1.70 (m, 4H), 1.40-0.95 (m, 56H), 0.85 (t, J=6.8 Hz, 6H).

### Synthesis of W4.3: 1-methyl-3-(nonacosan-15-yl)-1H-imidazol-3-ium chloride

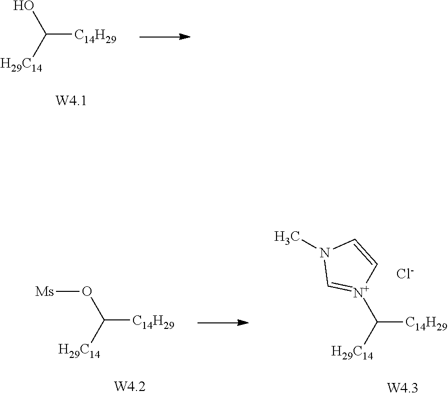

Compounds W4.1 to W4.3 were obtained following procedures similar to the ones described above (W21.1, W21.6 and W21.7 respectively).

### Analysis of Compound W4.3:

¹H-NMR (400 MHz, CDCl₃): δ=11.15 (s, 1H), 7.37 (t, J=1.6 Hz, 1H), 7.17 (t, J=1.6 Hz, 1H), 4.50-4.40 (m, 1H), 4.18 (s, 3H), 2.05-1.90 (m, 4H), 1.40-0.95 (m, 48H), 0.85 (t, J=6.8 Hz, 6H).

### Synthesis of W5.3: 1-methyl-3-(tritriacontan-17-yl)-1H-imidazol-3-ium chloride

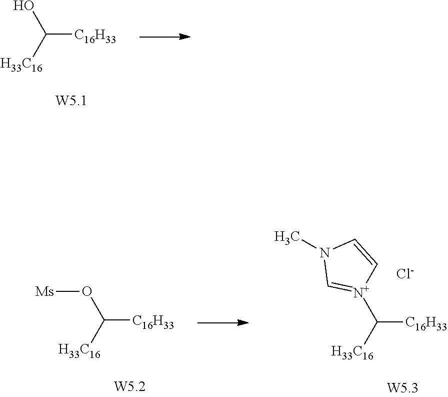

Compounds W5.1 to W5.3 were obtained following procedures similar to the ones described above (W21.1, W21.6 and W21.7 respectively).

### Analysis of Compound W5.3:

¹H-NMR (400 MHz, CDCl₃): δ=11.15 (s, 1H), 7.32 (t, J=1.6 Hz, 1H), 7.16 (t, J=1.6 Hz, 1H), 4.50-4.40 (m, 1H), 4.18 (s, 3H), 2.00-1.85 (m, 4H), 1.40-0.95 (m, 56H), 0.85 (t, J=6.8 Hz, 6H).

### Synthesis of W7.4: 1-methyl-3-(2-heptadecylicosyl)-1H-imidazol-3-ium chloride

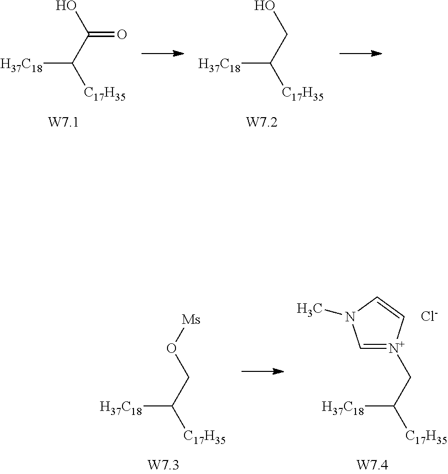

### a) Synthesis of Compound W7.1

To a solution of lithium diisopropylamide (3.59 g; 33.51 mmoles; MW=107.12) in 100 mL of tetrahydrofuran cooled in an ice-water bath was added drop-wise a solution of 10 g of nonadecanoic acid (33.50 mmoles; MW=298.50) in 100 mL of tetrahydrofuran. 4.8 mL of 1,3-dimethyl-3,4,5,6-tetrahydro-2(1H-pyrimidinone (39.70 mmoles; MW=128.17) were then added. The reaction mixture was allowed to stay 30 minutes at room temperature. The mixture was then cooled at −10° C. and 12.74 g of 1-Iodooctadecane (33.50 mmoles; MW=380.39) in 80 mL of tetrahydrofuran were added drop-wise. After 24 hours at room temperature, the mixture was poured onto 400 mL split ice acidified with 150 mL of concentrated hydrochloric acid for 1 hour. After extraction with ethyl acetate and removal of solvents, the crude was chromatographed on silica gel (CH₂Cl₂-heptane 1:1). The residue was further recrystallized from acetone to afford 4.66 g of compound W7.1 (8.46 mmoles, MW=550.98, 25% yield).

### Analysis of Compound W7.1:

TLC: Rf=0.20; solvent: CH₂Cl₂-heptane 1:1; detection with vanillin-sulfuric acid reagent (Merck TLC plates silica gel 60 F₂₅₄).

¹H-NMR (400 MHz, CDCl₃): δ=2.42-2.32 (m, 1H), 1.70-1.52 (m, 2H), 1.52-1.38 (m, 2H), 1.35-1.00 (m, 62H), 0.85 (t, J=6.8 Hz, 6H).

### b) Synthesis of Compound W7.2

To a solution of 4.50 g of compound W7.1 (8.17 mmoles; MW=550.98) in 100 mL of tetrahydrofuran cooled in an ice-water bath was added drop-wise 64 mL of a 1M Borane tetrahydrofuran complex solution in Tetrahydrofuran (64 mmoles; MW=85.94). After 24 hours at room temperature, the mixture was poured onto 400 mL of methanol and the solvents were removed under reduced pressure. The residue was chromatographed on silica gel (CH₂Cl₂-heptane 3:7) to afford 3.58 g of compound W7.2 (6.67 mmoles, MW=537.00, 81% yield).

### Analysis of Compound W7.2:

TLC: Rf=0.50; solvent: CH₂Cl₂-heptane 1:1; detection with vanillin-sulfuric acid reagent (Merck TLC plates silica gel 60 F₂₅₄).

¹H-NMR (400 MHz, CDCl₃): δ=3.56 (m, 2H), 1.40-1.00 (m, 67H), 0.85 (t, J=6.8 Hz, 6H).

### C) Syntheses of Compounds W7.3 and W7.4

Compounds W7.3 and W7.4 were obtained following procedures similar to the ones described above (W21.6 and W21.7).

### Analysis of Compound W7.4:

¹H-NMR (400 MHz, CDCl₃): δ=10.78 (s, 1H), 7.43 (t, J=1.6 Hz, 1H), 7.17 (t, J=1.6 Hz, 1H), 4.30-4.00 (m, 5H), 1.40-0.95 (m, 67H), 0.88 (t, J=6.8 Hz, 6H).

### Synthesis of W1.4: 1-methyl-3-(heptatriaconta-9,28-dien-19-yl)-1-methyl-1H-imidazol-3-ium chloride

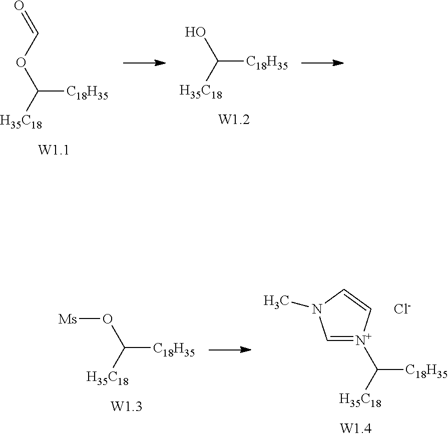

Compounds W1.1 to W1.4 were obtained following procedures similar to the ones described above (W18.6, W18.7, W21.6 and W21.7 respectively).

### Analysis of Compound W1.4:

TLC: Rf=0.50; solvent: CH₂Cl₂-methanol 9:1; detection with iodine (Merck TLC plates silica gel 60 F₂₅₄).

¹H-NMR (400 MHz, CDCl₃): δ=10.90 (s, 1H), 7.30 (t, J=1.6 Hz, 1H), 7.17 (t, J=1.6 Hz, 1H), 5.40-5.20 (m, 4H), 4.40-4.20 (m, 1H), 4.10 (s, 3H), 2.00-1.60 (m, 12H), 1.40-0.95 (m, 48H), 0.85 (t, J=6.8 Hz, 6H).

### Synthesis of W6.3: 1-methyl-3-(nonacosan-11-yl)-1H-imidazol-3-ium chloride

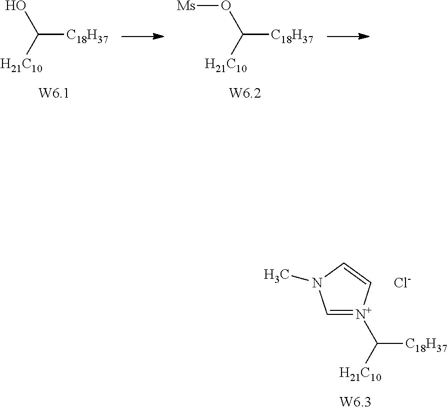

Compounds W6.1 to W6.3 were obtained following procedures similar to the ones described above (W21.1, W21.6 and W21.7 respectively).

### Analysis of Compound W6.3:

¹H-NMR (400 MHz, CDCl₃): δ=10.90 (s, 1H), 7.50 (t, J=1.6 Hz, 1H), 7.20 (t, J=1.6 Hz, 1H), 4.45-4.35 (m, 1H), 4.20 (s, 3H), 2.00-1.60 (m, 4H), 1.40-0.95 (m, 48H), 0.85 (t, J=6.8 Hz, 6H).

### Example 2. Formulation Using Microfluidics

mRNA-LNPs have been prepared with a specific equipment (NanoAssemblr Ignite from Precision Nanosystem).

The mRNA-LNPs' formulation involved the study of several parameters:
Choice of lipids as described above in Table 2;
Quantity of each lipid as described above in Table 2;
Total concentration of lipids in the final formulation: from 5 mM to 10 mM;
Quantity of mRNA: final concentration from 10 to 200 ng/μl;
Selection of buffer used during the mixing (pH, type and concentration of salts, volume, etc.), in particular wherein the buffer is PBS buffer;
Ratio EtOH/buffer during the mixing; 3:1 aqueous phase (mRNA and acetate buffer): organic phase (lipids and ethanol);
Flow rate of mixing: 10 mL/min.

### Example 3. Physical Properties: Size & Zeta

Particle characterizations and physical behaviors have been studied by Dynamic Light Scattering (DLS) measurements using a Zetasizer from Malvern Panalytical.

DLS gives access to the particle size (in nm) and charge (in mV).

### Equipment and Consumables:

Zetasizer Nano ZS (Malvern Panalytical)
Zeta cell (Malvern Panalytical, DTS1070)
Cuvette 40 μL (Malvern Panalytical, ZEN0040)
Eppendorf tube 1.5 mL (Dutscher, 033508)
PBS 1× (Sigma-Aldrich, D8537)
Nuclease Free Water (Thermofisher, AM9930)

### Protocol

### Size:

A solution of LNP was diluted by 40-fold in 1× (7.5 μL LNP in 292.5 μL of PBS 1×) in a 1 mL Eppendorf tube, then transferred in the cuvette 40 μL.
Particle size analysis
   5 measures per sample
   10 runs per measure
   10 seconds per run
   Results expressed as size number distribution of particles

### Zêta Potential:

A solution of LNP was diluted by 20-fold in 1× (40 μL LNP in 760 μL of Nuclease Free Water) in a 1 mL Eppendorf tube, then transferred in the Zeta cell.
Particle charge analysis:
   3 measures per sample
   30 seconds per run
   10 runs per measure
   Results expressed as mV

### Example 4. Encapsulation Efficiency

Encapsulation efficiency is a crucial property in LNP development. The measure determines the rate of mRNA inside the LNP relative to the initial amount used in the formulation. Usually, the encapsulation is higher to 90% to be considered as effective or as optimal.

### Equipment/Consumables:

FLUOstar® Omega (BMG Labtech)
Quant-it™ RiboGreen RNA Assay Kit (Thermofisher, R11490)
96 well plate Cellstar (Dutscher, 020034)
PBS 1× (Sigma-Aldrich, D8537)
Nuclease Free Water (Thermofisher, AM9930)
Triton X-100 (Sigma-Aldrich, T8787)

### Protocol

### Buffers Preparation:

TE 1× buffer: Dilute 10 mL of tampon TE 20× buffer (in Quant-it™ kit) in 190 mL of Nuclease Free Water
Triton Buffer: Dilute 2 mL of triton X-100 in 98 mL of TE 1×

### Analysis:

Samples preparation:
   Add 50 μL de TE 1× per well in line n°1 (2 wells par LNP+2 blanks)
   Add 50 μL of Triton Buffer per well in line n°2 (2 wells per LNP+2 blanks)
   Dilute 15 μL de LNP in 235 μL of TE 1×
   Add 50 μL of diluted LNP dans lines n° 1 (TE 1×) and n°2 (Triton Buffer)
Standard solutions preparation:
   Dilute Nucleic acid at 20 μg/mL in TE 1× (a 150 μL volume is required by plate)
   Prepare a calibration curve in the same plate following Table 1 below (2 wells per concentration)

TABLE 1
Calibration curve

| | [RNA] | Triton | TE | RNA at |
|---|---|---|---|---|
| Standard | (μg/mL) | buffer (μL) | 1X (μL) | 20 μg/mM (μL) |
| S0 | 0 | 50 | 50 | 0 |
| S1 | 0.1 | | 49 | 1 |
| S2 | 0.25 | | 47.5 | 2.5 |
| S3 | 0.5 | | 45 | 5 |
| S4 | 1 | | 40 | 10 |
| S5 | 2 | | 30 | 20 |
| S6 | 2.5 | | 25 | 25 |

Incubate the plate for 30 min at 37° C.
Dilute Quant-it™ RNA RiboGreen Reagent by 100 in TE 1× (100 μL required per well). Store the solution in the dark.
When incubation is done, add 100 μL of diluted Quant-it™ RNA RiboGreen Reagent per well
Total dilution factor: 66.67

### Fluorescence Spectroscopy:

Put the plate in FLUOstar
Load Ribogreen protocol et indicate the plate mapping by selecting the type of well (Sample/Blank/Standard)
Check parameters of Ribogreen protocol
   Fluorescence Intensity—End Point
   Top Optic
   Microplate: Greiner 96 F-Bottom
   Filter Settings No. Of multichromatics: 1
   Excitation Filter: 485-12
   Emission Filter: Em520
   Gain 500

### Calculation

Standard S0 is used as blank, the 2 wells mean is removed for each well before the final mean calculation.

The blank diluted in TE 1× is used only for line TE 1×, 2 wells mean is removed for each well before the final mean calculation. This measure gives the amount of free RNA per.

The blank diluted in Triton Buffer is used only for line Triton Buffer, 2 wells mean is removed for each well before the final mean calculation. This measure gives the total amount of RNA per sample.

### Encapsulation Efficiency

$$EE ( \% ) = \frac{total RNA - free RNA}{total RNA} \times 100$$

$$EE ( \% ) = [ ( Total RNA - Free RNA ) / Total RNA ] \times 100$$

### Example 5. Transfection Efficiency

Both in vitro and in vivo transfection efficiency have been evaluated. A first formulation's screening was performed in vitro to select the best composition. Then, only promising formulation was tested in vivo.

### Cell Lines

### Caco2 Cells

Human epithelial cells from colorectal adenocarcinoma (Caco2) were grown on cell flask coated with fibronectin (0.05 mg/ml) and cultured in DMEM glucose 4.5 g/L supplemented with Fetal bovine serum (FBS, 20%), Na Pyruvate (1%), L-Glutamine (1%), non-essential amino acids (AANE, 1%) and Penicillin-Streptomycin (1%).

### HeLa Cells

Immortalized cell line derived from human epithelial cells of cervix adenocarcinoma (HeLa) were cultured in MEM Eagle+AANE (1%)+FBS (10%)+L-Glutamine (1%)+Penicillin-Streptomycin (2%).

### HepG2 Cells

Human cells from a liver hepatocellular carcinoma (HepG2) were cultured in MEM Eagle+AANE (1%)+FBS (10%)+L-Glutamine (1%)+Na Pyruvate (1%)+Penicillin-Streptomycin (2%).

### A549 Cells

Adenocarcinomic human alveolar basal epithelial cells (A549) cells were cultured in RPMI FBS (10%)+L-Glutamine (1%)+Penicillin-Streptomycin (2%).

### Jurkat Cells

Jurkat, Clone E6-1 is a clone of the Jurkat-FHCRC cell line, a derivative of the Jurkat cell line, which was established from the peripheral blood of a 14-year-old, male, acute T-cell leukemia patient. Jurkat cells were purchased from ATCC (TIB-152) and cultured in RPMI+FBS (10%)+L-Glutamine (1%)+Penicillin-Streptomycin (2%).

### Human Primary T Cells

Human primary T cells from healthy donors were isolated from peripheral blood by magnetic activated cell sorting and were frozen for later use. Thawed T cells were cultured in complete medium: IMDM (Gibco, 21980032)+FBS (10%)+L-Glutamine (1%)+Penicillin-Streptomycin (1%) or X-VIVO 15 (Lonza, BE02-060F) supplemented with recombinant human IL-21 (10 ng/ml, Peprotech, 200-21) at a density of 1.5×10⁶ cells/ml in a flask and activated with T Cell TransAct™, human (10 μl/1·10⁶ cells, Miltenyi Biotec, 130-111-160) for 48 hours in a 37° C. and 5% C₀₂ humidified incubator.

### Human CD34+ Cells

Human primary CD34+ cells from healthy donors were isolated from placental blood (Lymphobank, SC-159-02) and were frozen for later use. Thawed CD34+ cells were expanded for 7 days and cultivated in StemMACS™ HSC Expansion Media XF (Miltenyi Biotec, 130-100-463) supplemented with StemMACS™ HSC Expansion Cocktail (Miltenyi Biotec, 130-100-843) and UM729 (STEMCELL technologies, 72332).

### In Vitro Transfection

Fluc mRNA Transfection of Adherent Cell Lines

For transfection experiments, 4×10⁴ Caco2 cells, 5×10⁴ HeLa cells, 1×10⁵ HepG2 cells or 6×10⁴ A549 cells were seeded per well of 24-well plates in complete medium 1 day before transfection. On the day of transfection, jetMESSENGER® or in vivo-jetRNA®/Fluc mRNA complexes were prepared according to the manufacturers' recommendations. Briefly, transfection with jetMESSENGER® or in vivo-jetRNA® was performed as described: 500 ng (Caco2, HepG2 and A549) or 250 ng (HeLa) of Fluc-encoding mRNA (per well of 24-well plate) were first diluted in the provided mRNA Buffer, followed by the mixing-in of 0.5-1 μl jetMESSENGER® or in vivo-jetRNA®. Following an incubation of 15 minutes at room temperature, jetMESSENGER® or in vivo-jetRNA® complexes or 500 ng (Caco2, HepG2 and A549) or 250 ng (HeLa) of LNP X (a list of the different LNP may be found in Table 2) were simply added dropwise to cells in their complete growth medium. Transfection efficiency was assessed 24 hours post-transfection by luminescence reading.

Fluc mRNA Transfection of Jurkat Cells

On the day of transfection, jetMESSENGER®/Fluc mRNA complexes were prepared according to the manufacturers' recommendations. Briefly, transfection with jetMESSENGER® was performed as described: 400 ng of Fluc-encoding mRNA (per well of 24-well plate) were first diluted in the provided mRNA Buffer, followed by the mixing-in of 0.8 μl jetMESSENGER®. Following an incubation of 15 minutes at room temperature, jetMESSENGER® complexes or 400 ng of LNP X (a list of the different LNP may be found in Table 2) were simply added to the plate and 8×10⁴ cells per well of 24-well plates of Jurkat were added on top of the complexes in their growth medium. 4 hours after 700 ul of complete growth medium were added. Transfection efficiency was assessed 24-hours post-transfection by luminescence reading.

Fluc mRNA Transfection of T Cells

On the day of transfection, jetMESSENGER®/Fluc mRNA complexes were prepared according to the manufacturers' recommendations. Briefly, transfection with jetMESSENGER® was performed as described: 750 ng of Fluc-encoding mRNA (per well of 24-well plate) were first diluted in the provided mRNA Buffer, followed by the mixing-in of 2.25 μl jetMESSENGER®. Following an incubation of 15 minutes at room temperature, jetMESSENGER® complexes or 750 ng of LNP X (a list of the different LNP may be found in Table 2) were simply added to the plate and 375×10³ cells per well of 48-well plates of T cells were added on top of the complexes in their growth medium (without IL-21 and T cell transAct). 4 hours after 350 μl of complete growth medium were added. Transfection efficiency was assessed 48-hours post-transfection by luminescence reading.

Gfp mRNA Transfection of CD34+ and T Cells

On the day of transfection, jetMESSENGER®/GFP mRNA complexes were prepared according to the manufacturers' recommendations. Briefly, transfection with jetMESSENGER® was performed as described: 500 ng (CD34+ cells) or 125 ng (T cells) of Fluc-encoding mRNA (per well of 96-well plate) were first diluted in the provided mRNA Buffer, followed by the mixing-in of 0.5 or 0.375 μl jetMESSENGER®. Following an incubation of 15 minutes at room temperature, jetMESSENGER® complexes or 125 ng (CD34+ cells) or 75 ng (T cells) of LNP X were simply added to the plate and 187 500 cells of CD34+ cells or T cells were added per well of 96-well plates on top of the complexes in StemMACS™ HSC Expansion Media XF supplemented with StemMACS™ HSC Expansion Cocktail. 4 hours after 175 μl of complete StemMACS™ medium were added (with IL-21 and T cell transAct for T cells). Transfection efficiency was assessed 24- or 48-hours post-transfection by flow cytometry respectively for CD34+ or T cells.

### Gfp DNA Transfection of HeLa Cells

For transfection experiments, HeLa cells were seeded at 12 500 cells per well of 96-well plates in complete medium 1 day before transfection. On the day of transfection, jetPRIME®/GFP DNA complexes were prepared according to the manufacturers' recommendations. Briefly, transfection with jetPRIME® was performed as described: 150 ng of GFP-encoding DNA (per well of 96-well plate) were first diluted in the provided jetPRIME® Buffer, followed by the mixing-in of 0.3 μl jetPRIME®. Following an incubation of 10 minutes at room temperature, jetPRIME® complexes or 150 ng of LNP X were simply added dropwise to cells in their complete growth medium. Transfection efficiency was assessed 24 hours post-transfection by flow cytometry.

### Sirna Transfection of A549 Luc

For transfection experiments, A549_Luc cells were seeded at 25×10³ cells per well of 24-well plates in complete medium 1 day before transfection. On the day of transfection, INTERFERin®/siRNA GL3_Luc complexes were prepared according to the manufacturers' recommendations. Briefly, transfection with INTERFERin® was performed as described: 10 mM of siRNA GL3_Luc or GL2_Mn (per well of 24-well plate) were first diluted in OPTI-MEM, followed by the mixing-in of 2 μl INTERFERin®. Following an incubation of 10 minutes at room temperature, INTERFERin® complexes or 10 mM of LNP X were simply added dropwise to cells in their complete growth medium. Transfection efficiency was assessed 48 hours post-transfection by luminescence reading.

### Example 6. In Vitro Activity

### Part a: Formulation-Activity Relationship

Several mRNA-lipid nanoparticles were formulated to define optimized compositions following several parameters (size, charge, encapsulation efficiency, polydispersity, transfection efficiency, stability).

To our knowledge, only few examples of LNPs using permanently cationic lipids (such as DOTAP or DOTMA) have been described. The results are depicted in Table 2.

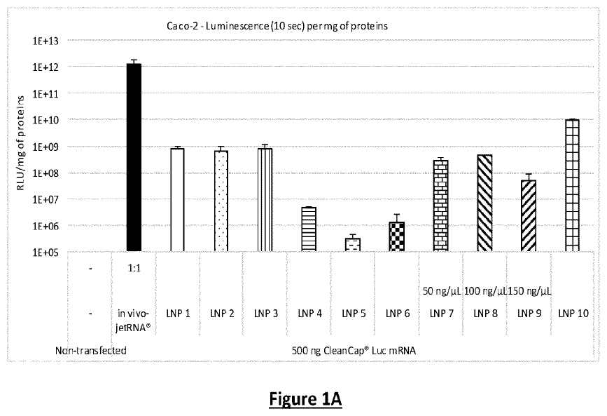

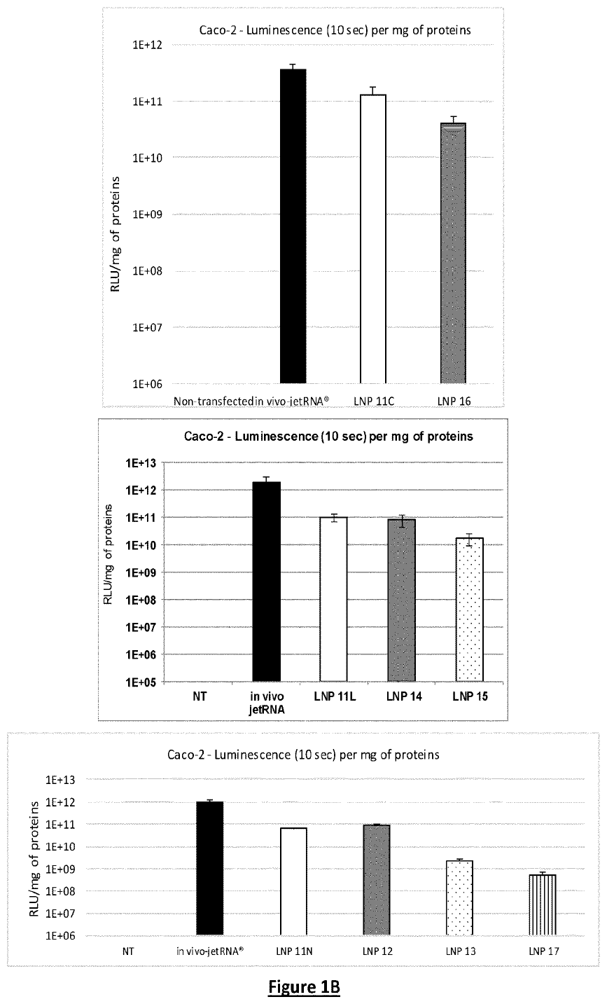

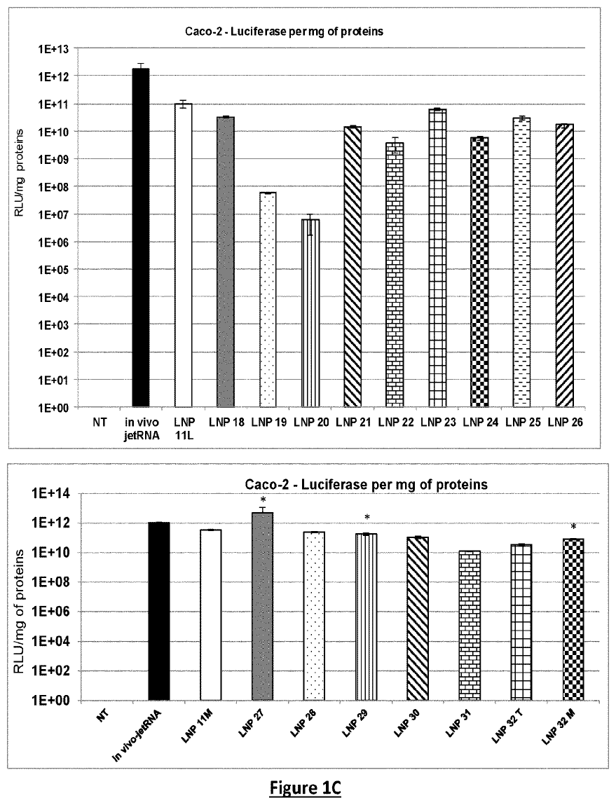

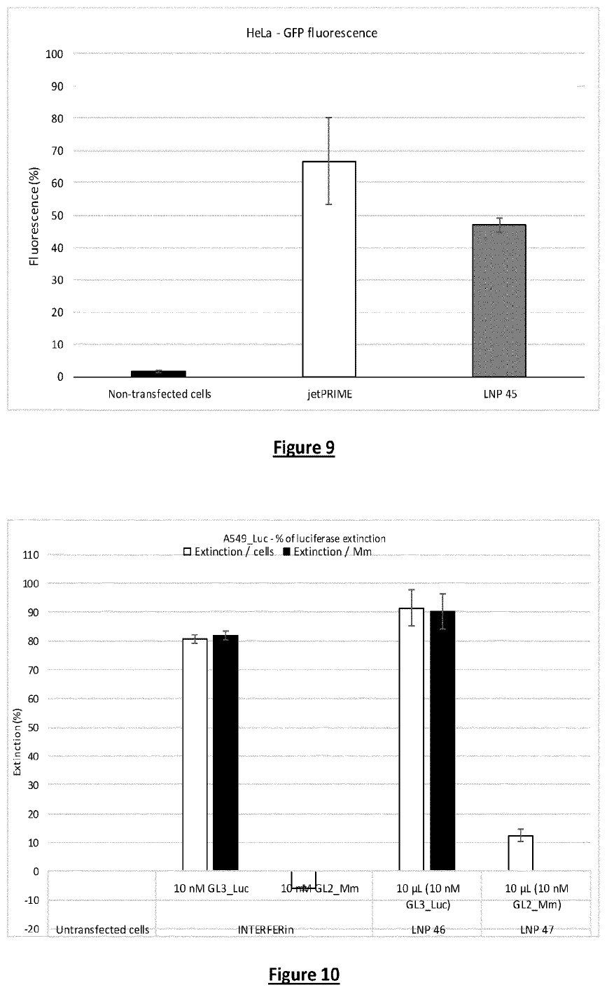

TABLE 2
Composition of mRNA-LNP

| | | Imidazolium-based | | | | | |
|---|---|---|---|---|---|---|---|
| | | cationic lipid of | | | | | |
| | | formula (I) (mM) | | | | | |
| | LNP | and ionizable | | Phospholipid | | | |
| FIGS. | No | lipid (mM) | | (mM) | | Sterol (mM) | |
| FIG. 1A | 1 | W21.7 | 4 | DPyPE | 1 | Cholesterol | 3.85 |
| | | DODMA | 1 | | | | |
| | 2 | W21.7 | 5 | DPyPE | 1 | Cholesterol | 2.85 |
| | | DODMA | 1 | | | | |
| | 3 | W21.7 | 4 | DSPC | 1 | Cholesterol | 1.85 |
| | | DODMA | 3 | | | | |
| | 4 | W21.7 | 5 | DOPE | 1 | Cholesterol | 2.85 |
| | | DODMA | 1 | | | | |
| | 5 | W21.7 | 4 | DPyPE | 1 | Stigmasterol | 3.85 |
| | | DODMA | 1 | | | | |
| | 6 | W21.7 | 4 | DPyPE | 1 | Beta- | 3.85 |
| | | DODMA | 1 | | | sitosterol | |
| | 7 | W21.7 | 4 | DPyPE | 1 | Cholesterol | 3.85 |
| | | DODMA | 1 | | | | |
| | 8 | W21.7 | 4 | DPyPE | 1 | Cholesterol | 3.85 |
| | | DODMA | 1 | | | | |
| | 9 | W21.7 | 4 | DPyPE | 1 | Cholesterol | 3.85 |
| | | DODMA | 1 | | | | |
| | 10 | W21.7 | 4 | DPyPE | 1 | Cholesterol | 1.85 |
| | | DODMA | 3 | | | | |
| FIG. 1B | 11C, | W21.7 | 4 | DPyPE | 1 | Cholesterol | 1.85 |
| | D, K, | DODMA | 3 | | | | |
| | L, M, | | | | | | |
| | N, O | | | | | | |
| | 12 | Imidazolium | 4 | DPyPE | 1 | Cholesterol | 3.85 |
| | | DODMA | 3 | | | | |
| | 13 | Imidazolium | 4 | DPyPE | 1 | DC-Cholesterol | 1.85 |
| | | DODMA | 3 | | | | |
| | 14 | W21.7 | 4 | DPyPE | 1 | Cholesterol | 0.00 |
| | | DODMA | 3 | | | | |
| | 15 | W21.7 | 4 | DPyPE | 0.00 | Cholesterol | 1.85 |
| | | DODMA | 3 | | | | |
| | 16 | W21.7 | 4 | DPyPE | 1 | Cholesterol | 1.85 |
| | | DLin-MC3-DMA | 3 | | | | |
| | 17 | Imidazolium | 4 | DPyPE | 1 | Cholesterol | 1.85 |
| | | DC-Cholesterol | 3 | | | | |
| FIG. 1C | 18 | W21.7 | 4 | DPyPE | 1 | Cholesterol | 1.85 |
| | | DODMA | 0.00 | | | | |
| | 19 | W21.7 | 0.00 | DPyPE | 1 | Cholesterol | 1.85 |
| | | DODMA | 3 | | | | |
| | 20 | W21.7 | 4 | DPyPE | 1 | Cholesterol | 1.85 |
| | | DODMA | 3 | | | | |
| | 21 | W21.7 | 4 | DPyPE | 1 | Cholesterol | 1.70 |
| | | DODMA | 3 | | | | |
| | 22 | W21.7 | 4 | DPyPE | 1 | Cholesterol | 1.50 |
| | | DODMA | 3 | | | | |
| | 23 | W21.7 | 4 | DPyPE | 1 | Cholesterol | 1.85 |
| | | DODMA | 3 | | | | |
| | 24 | W21.7 | 3 | DPyPE | 1 | Cholesterol | 2.85 |
| | | DODMA | 3 | | | | |
| | 25 | W21.7 | 2 | DPyPE | 1 | Cholesterol | 2.85 |
| | | DODMA | 3 | | | | |
| | 26 | W21.7 | 1 | DPyPE | 1 | Cholesterol | 2.85 |
| | | DODMA | 3 | | | | |
| | 27 | W21.7 | 5 | DPyPE | 1 | Cholesterol | 1.85 |
| | | DODMA | 2 | | | | |
| | 28 | W21.7 | 4 | DPyPE | 1 | Cholesterol | 1.85 |
| | | DODMA | 2 | | | | |
| | 29 | W21.7 | 4 | DPyPE | 1 | Cholesterol | 1.85 |
| | | DODMA | 1 | | | | |
| | 30 | W21.7 | 3 | DPyPE | 1 | Cholesterol | 1.85 |
| | | DODMA | 4 | | | | |
| | 31 | W21.7 | 2 | DPyPE | 1 | Cholesterol | 1.85 |
| | | DODMA | 5 | | | | |
| | 32 | W21.7 | 6 | DPyPE | 1 | Cholesterol | 1.85 |
| | | DODMA | 1 | | | | |
| FIGS. | 33B | W22.7 | 4 | DPyPE | 1 | Cholesterol | 1.85 |
| 1D, 2, 3, 4, | | DODMA | 3 | | | | |
| 5 and 6 | 34B | W12.7 | 4 | DPyPE | 1 | Cholesterol | 1.85 |
| | | DODMA | 3 | | | | |
| | 35 | W20.7 | 4 | DPyPE | 1 | Cholesterol | 1.85 |
| | | DODMA | 3 | | | | |
| | 36B | W16.7 | 4 | DPyPE | 1 | Cholesterol | 1.85 |
| | | DODMA | 3 | | | | |
| | 37B | SM-102 | 5 | DSPC | 1 | Cholesterol | 3.85 |
| | 38B | ALC-0315 | 4.63 | DSPC | 0.94 | Cholesterol | 4.27 |
| | 39B | DOTAP | 4 | DPyPE | 1 | Cholesterol | 1.85 |
| | | DODMA | 3 | | | | |
| | 40 | DOTMA | 4 | DPyPE | 1 | Cholesterol | 1.85 |
| | | DODMA | 3 | | | | |
| | 41 | W21.7 | 4 | DPyPE | 1 | DC-Cholesterol | 4.85 |
| | 42 | DOTMA | 4 | DOPE | 1.4 | Cholesterol | 2.9 |
| | | DODMA | 1.4 | | | | |
| FIG. 7 | 43B | W21.7 | 4 | DPyPE | 1 | Cholesterol | 1.85 |
| and 8 GFP | | DODMA | 3 | | | | |
| | 44 | W21.7 | 4 | DPyPE | 1 | Cholesterol | 1.70 |
| | | DODMA | 3 | | | | |
| FIG. 9 DNA | 45 | W21.7 | 4 | DPyPE | 1 | Cholesterol | 1.85 |
| | | DODMA | 3 | | | | |
| FIG. 10 siRNA | 46 | W16.7 | 4 | DPyPE | 1 | Cholesterol | 1.85 |
| Fluc | | DODMA | 3 | | | | |
| FIG. 10 siRNA | 47 | W16.7 | 4 | DPyPE | 1 | Cholesterol | 1.85 |
| mismatched | | DODMA | 3 | | | | |
| FIGS. 11, 12, 13 | 48B, | W21.7 | 4 | DPyPE | 1 | Cholesterol | 1.85 |
| and 14 | and D | DODMA | 3 | | | | |
| In vivo | | | | | | | |
| FIG. 11 | 49 | DLin-MC3- | 2.75 | DSPC | 0.55 | Cholesterol | 2.1175 |
| In vivo | | DMA | | | | | |
| FIG. 14 | 50B | Imidazolium | 4 | DPyPE | 1 | Cholesterol | 1.85 |
| In vivo | | DODMA | 3 | | | | |
| | 51C | SM-102 | 5 | DSPC | 1 | Cholesterol | 3.85 |
| | 52 | W21.7 | 4 | DPyPE | 1 | Cholesterol | 1.70 |
| | | DODMA | 3 | | | | |
| | 53 | W21.7 | 4 | DPyPE | 1 | Cholesterol | 1.50 |
| | | DODMA | 3 | | | | |
| | 54 | W21.7 | 4 | DPyPE | 1 | Cholesterol | 1.85 |
| | | DODMA | 0 | | | | |
| | 55 | DOTAP | 4 | DPyPE | 1 | Cholesterol | 1.85 |
| | | DODMA | 3 | | | | |

| | LNP | PEG-lipid | | mRNA | | | | |
|---|---|---|---|---|---|---|---|---|
| FIGS. | No | (mM) | | (ng/μL) | Size | PDI | Zeta | EE % |
| FIG. 1A | 1 | DSG- | 0.15 | 10 | 77 ± 1 | 0.062 | +13 | ND |
| | | PEG₂ₖ | | | | | | |
| | 2 | DSG- | 0.15 | 10 | 100 ± 7 | 0.094 | +19 | ND |
| | | PEG₂ₖ | | | | | | |
| | 3 | DSG- | 0.15 | 10 | 68 ± 13 | 0.179 | +26 | ND |
| | | PEG₂ₖ | | | | | | |
| | 4 | DSG- | 0.15 | 10 | 108 ± 3 | 0.079 | +6 | ND |
| | | PEG₂ₖ | | | | | | |
| | 5 | DSG- | 0.15 | 10 | 43 ± 15 | 0.385 | NA | ND |
| | | PEG₂ₖ | | | | | | |
| | 6 | DSG- | 0.15 | 10 | 16 ± 19 | 0.426 | NA | ND |
| | | PEG₂ₖ | | | | | | |
| | 7 | DSG- | 0.15 | 50 | 81 ± 3 | 0.079 | +15 | ND |
| | | PEG₂ₖ | | | | | | |
| | 8 | DSG- | 0.15 | 100 | 74 ± 2 | 0.08 | +15 | ND |
| | | PEG₂ₖ | | | | | | |
| | 9 | DSG- | 0.15 | 150 | 79 ± 1 | 0.064 | +16 | ND |
| | | PEG₂ₖ | | | | | | |
| | 10 | DSG- | 0.15 | 10 | 80 ± 2 | 0.062 | +15 | ND |
| | | PEG₂ₖ | | | | | | |
| FIG. 1B | 11C, | DSG- | 0.15 | 50 | 64 ± 2 | 0.068 | +12 | 98% |
| | D, K, | PEG₂ₖ | | | | | | |
| | L, M, | | | | | | | |
| | N, O | | | | | | | |
| | 12 | DSG- | 0.15 | 50 | ND | ND | ND | ND |
| | | PEG 2k | | | | | | |
| | 13 | DSG- | 0.15 | 50 | ND | ND | ND | ND |
| | | PEG 2k | | | | | | |
| | 14 | DSG- | 0.15 | 50 | 71 ± 2 | 0.07 | +12 | 98% |
| | | PEG₂ₖ | | | | | | |
| | 15 | DSG- | 0.15 | 50 | 47 ± 1 | 0.126 | +13 | 98% |
| | | PEG₂ₖ | | | | | | |
| | 16 | DSG- | 0.15 | 50 | 80 ± 3 | 0.077 | +12 | ND |
| | | PEG 2k | | | | | | |
| | 17 | DSG- | 0.15 | 50 | ND | ND | ND | ND |
| | | PEG 2k | | | | | | |
| FIG. 1C | 18 | DSG- | 0.15 | 50 | 71 ± 3 | 0.152 | +17 | 98% |
| | | PEG₂ₖ | | | | | | |
| | 19 | DSG- | 0.15 | 50 | 32 ± 3 | 0.156 | 0 | 93% |
| | | PEG₂ₖ | | | | | | |
| | 20 | DSG- | 0.00 | 50 | 45 ± 10 | 0.291 | −15 | ND |
| | | PEG₂ₖ | | | | | | |
| | 21 | DSG- | 0.3 | 50 | 32 ± 1 | 0.221 | +17 | 99% |
| | | PEG₂ₖ | | | | | | |
| | 22 | DSG- | 0.5 | 50 | 22 ± 2 | 0.292 | +12 | 98% |
| | | PEG₂ₖ | | | | | | |
| | 23 | DSG- | 0.15 | 50 | 49 ± 5 | 0.336 | +22 | 98% |
| | | PEG₂ₖ | | | | | | |
| | 24 | DSG- | 0.15 | 50 | 43 ± 1 | 0.085 | +9 | 98% |
| | | PEG₂ₖ | | | | | | |
| | 25 | DSG- | 0.15 | 50 | 72 ± 3 | 0.042 | +12 | 97% |
| | | PEG₂ₖ | | | | | | |
| | 26 | DSG- | 0.15 | 50 | 26 ± 1 | 0.144 | +9 | 98% |
| | | PEG₂ₖ | | | | | | |
| | 27 | DSG- | 0.15 | 50 | 87 ± 4 | 0.137 | +22 | 96% |
| | | PEG₂ₖ | | | | | | |
| | 28 | DSG- | 0.15 | 50 | 61 ± 3 | 0.11 | +12 | 97% |
| | | PEG₂ₖ | | | | | | |
| | 29 | DSG- | 0.15 | 50 | 61 ± 2 | 0.12 | +11 | 97% |
| | | PEG₂ₖ | | | | | | |
| | 30 | DSG- | 0.15 | 50 | 59 ± 4 | 0.107 | +11 | 98% |
| | | PEG₂ₖ | | | | | | |
| | 31 | DSG- | 0.15 | 50 | 51 ± 1 | 0.131 | +11 | 99% |
| | | PEG₂ₖ | | | | | | |
| | 32 | DSG- | 0.15 | 50 | 110± | 0.035 | +15 | 84% |
| | | PEG₂ₖ | | | | | | |
| FIGS. | 33B | DSG- | 0.15 | 50 | 65 ± 2 | 0.098 | +16 | 99% |
| 1D, 2, 3, 4, | | PEG 2k | | | | | | |
| 5 and 6 | 34B | DSG- | 0.15 | 50 | 35 ± 2 | 0.087 | +18 | 99% |
| | | PEG 2k | | | | | | |
| | 35 | DSG- | 0.15 | 50 | 52 ± 1 | 0.142 | +12 | 95% |
| | | PEG 2k | | | | | | |
| | 36B | DSG- | 0.15 | 50 | 35 ± 1 | 0.127 | +13 | 99% |
| | | PEG 2k | | | | | | |
| | 37B | DMG-PEG | 0.15 | 50 | 180 ± 12 | 0.093 | +4 | 93% |
| | 38B | DMG-PEG | 0.16 | 50 | 38 ± 3 | 0.197 | +15 | 95% |
| | 39B | DSG- | 0.15 | 50 | 40 ± 1 | 0.018 | +12 | 86% |
| | | PEG 2k | | | | | | |
| | 40 | DSG- | 0.15 | 50 | 35 ± 2 | 0.1788 | +12 | 95% |
| | | PEG 2k | | | | | | |
| | 41 | DSG- | 0.15 | 50 | ND | ND | ND | ND |
| | | PEG 2k | | | | | | |
| | 42 | DSG- | 0.3 | 50 | 21 ± 1 | 0.2528 | +20 | 95% |
| | | PEG 2k | | | | | | |
| FIG. 7 | 43B | DSG- | 0.15 | 100 | 82 ± 4 | 0.075 | +9 | 92% |
| and 8 GFP | | PEG₂ₖ | | | | | | |
| | 44 | DSG- | 0.3 | 100 | 41 ± 1 | 0.135 | +12 | 91% |
| | | PEG₂ₖ | | | | | | |
| FIG. 9 DNA | 45 | DSG- | 0.15 | 50 | 52 ± 3 | 0.140 | +15 | 100% |
| | | PEG₂ₖ | | | | | | |
| FIG. 10 siRNA | 46 | DSG- | 0.15 | Equivalent | 65 ± 2 | 0.055 | +12 | ND |
| Fluc | | PEG 2k | | 6.8 ng/μL | | | | |
| | | | | siRNA | | | | |
| FIG. 10 siRNA | 47 | DSG- | 0.15 | Equivalent | 59 ± 2 | 0.088 | +12 | ND |
| mismatched | | PEG 2k | | 6.8 ng/μL | | | | |
| | | | | siRNA | | | | |
| FIGS. 11, 12, 13 | 48B, | DSG- | 0.15 | 200 | 97 ± 1 | 0.0894 | +12 | 98% |
| and 14 | and D | PEG₂ₖ | | | | | | |
| In vivo | | | | | | | | |
| FIG. 11 | 49 | DSG- | 0.08 | 200 | 55 ± 2 | 0.1014 | +3 | 95% |
| In vivo | | PEG 2k | | | | | | |
| FIG. 14 | 50B | DSG- | 0.15 | 100 | 59 ± 3 | 0.1038 | +13 | 100% |
| In vivo | | PEG 2k | | | | | | |
| | 51C | DSG-PEG | 0.15 | 200 | 57 ± 2 | 0.1876 | −5 | 98% |
| | 52 | DSG- | 0.3 | 200 | 34 ± 3 | 0.1612 | +13 | 100% |
| | | PEG 2k | | | | | | |
| | 53 | DSG- | 0.5 | 200 | 29 ± 2 | 0.1626 | +8 | 100% |
| | | PEG 2k | | | | | | |
| | 54 | DSG- | 0.15 | 200 | 69 ± 3 | 0.1048 | +15 | 100% |
| | | PEG 2k | | | | | | |
| | 55 | DSG- | 0.15 | 200 | 36 ± 3 | 0.2004 | +26 | 100% |
| | | PEG 2k | | | | | | |

ND: not determined;
EE % = Encapsulation efficiency

### Part B: Cell Lines

Transfection efficiency were evaluated in vitro on several cell type listed below: Caco-2/HepG2/HeLa/A549/Jurkat/T Cells

a) Caco2 Cells were Transfected with in Vivo-jetRNA®/Fluc mRNA Complexes or LNP 1, LNP 2, LNP 3, LNP 4, LNP 5, LNP 6, LNP 7, LNP 8, LNP 9, LNP 10.

Transfection efficiency was assessed 24 hours post-transfection by luminescence reading. Highest transfection efficiency was reached with in vivo-jetRNA®/mRNA complexes. When cells were transfected with LNPs, the highest efficacy was reached with LNP 10. When the amount of W21.7 was increased (between LNP 1 and LNP 2), there was no impact on the transfection efficiency neither when DSPC was used instead of DPyPE (LNP 3). However, when DPyPE was replaced by DOPE (LNP 4) or cholesterol by stigmasterol or beta-sitosterol (LNP 5 and LNP 6), there was a high decreased of transfection efficiency. When the concentration of mRNA in LNPs was increased (LNP 7, LNP 8, LNP 9), there was no impact on the transfection efficiency except with the LNP 9 with a decreased in transfection efficiency (FIG. 1A).

b) Caco2 Cells were Transfected with in Vivo-jetRNA®/Fluc mRNA Complexes or, LNP 11C, LNP 11L, LNP11 N, LNP16, LNP14, LNP15, LNP12, LNP13,or LNP17.

Transfection efficiency was assessed 24 hours post-transfection by luminescence reading. When cells were transfected with LNP 11C, LNP 11L or LNP 11 N, the transfection efficiency was closed to the highest transfection efficiency reached with in vivo-jetRNA®/mRNA complexes. When DODMA was replaced by DLin-MC3-DMA (LNP 16) the transfection efficiency was decreased. When the Cholesterol was removed from the LNP formulation (LNP 14) there was no impact on the transfection efficiency however the transfection efficiency was decreased when the DPyPE was removed (LNP 15). The increased in Cholesterol amount (LNP 12) didn't impact the transfection efficiency however when it was replaced by DC-Cholesterol (LNP 13) or when DODMA was replaced by DC-Cholesterol (LNP 17), the transfection efficiency was lower (FIG. 1B).

c) Caco2 Cells were Transfected with in Vivo-jetRNA®/Fluc mRNA Complexes or LNP 11L, LNP 11 M, LNP 18, LNP 19, LNP20, LNP 21, LNP 22, LNP 23, LNP 24, LNP 25, LNP 26, LNP 27, LNP 28, LNP 29, LNP 30, LNP 31, LNP 32 (T: Quantity Based on the Theoretical Concentration and M: Quantity Based on the Concentration Measured).

Transfection efficiency was assessed 24 hours post-transfection by luminescence reading. With all the LNP 18, LNP 19, LNP 20, LNP 21, LNP 22, LNP 23, LNP 24, LNP 25, LNP 26 and LNP 29, LNP 30, LNP 31, LNP 32, the transfection efficiency was lower than with the LNP 11L and LNP 11M. A slight decreased was observed when the DODMA (LNP 18) was removed from the formulation. A high decreased was observed when W21.7 or the DSG-PEG was removed from the formulation. The increased amount of DSG-PEG (LNP 21 and LNP 22) decreased size of LNPs but also slightly decreased the transfection efficiency. No impact on the transfection efficiency was observed when the DSG-PEG was replaced by the DMG-PEG (LNP 23). For the LNP 24, LNP 25, LNP 26, and LNP 27, LNP 28, LNP 29, LNP 30, LNP 31, LNP 32, different ratios of W21.7:DODMA were tested to compare with the LNP 11L and LNP 11M, and the best one were the LNP 11L, LNP 11 M and LNP 28. The increase of the amount of W21.7 in LNP 27 and LNP 32 induced some toxicity (FIG. 1C).

d) Caco2 Cells were Transfected with jetMESSENGER®/Fluc mRNA Complexes or LNP 110, LNP 33B, LNP 34B, LNP 36B, LNP 37B, LNP 38B, LNP 39B, LNP 42, LNP 40, LNP 35.

Transfection efficiency was assessed 24 hours post-transfection by luminescence reading. With the other cationic lipid W22.7 (LNP 33B), W12.7 (LNP 34B), W20.7 (LNP 35) and W16.7 (LNP 36B), a good transfection efficiency was observed but slightly lower than with the LNP 110 except for the LNP 36B which gave similar transfection efficiency. Lower transfection efficiency was observed with the LNP 37B (SM-102 instead of W21.7 and DODMA), LNP 38B (ALC-0315 instead of W21.7 and DODMA), LNP 39B, LNP 40 and LNP 42 (DOTAP or DOTMA instead of W21.7) (FIG. 1D).

e) HepG2 Cells were Transfected with jetMESSENGER®/Fluc mRNA Complexes or LNP 110, LNP 33B, LNP 34B, LNP 36B, LNP 37B, LNP 38B, LNP 39B, LNP 42, LNP 40, LNP 35.

Transfection efficiency was assessed 24 hours post-transfection by luminescence reading. With the other cationic lipid W22.7 (LNP 33B), W12.7 (LNP 34B), W20.7 (LNP 35) and W16.7 (LNP 36B), a good transfection efficiency was observed similar or higher than with the LNP 110. Lower transfection efficiency was observed with the LNP 37B (SM-102 instead of W21.7 and DODMA), LNP 38B (ALC-0315 instead of W21.7 and DODMA), LNP 39B, LNP 40 and LNP 42 (DOTAP or DOTMA instead of W21.7) (FIG. 2).

f) HeLa Cells were Transfected with jetMESSENGER®/Fluc mRNA Complexes or LNP 110, LNP 33B, LNP 34B, LNP 36B, LNP 37B, LNP 38, LNP 39B, LNP 42, LNP 40, LNP 35.

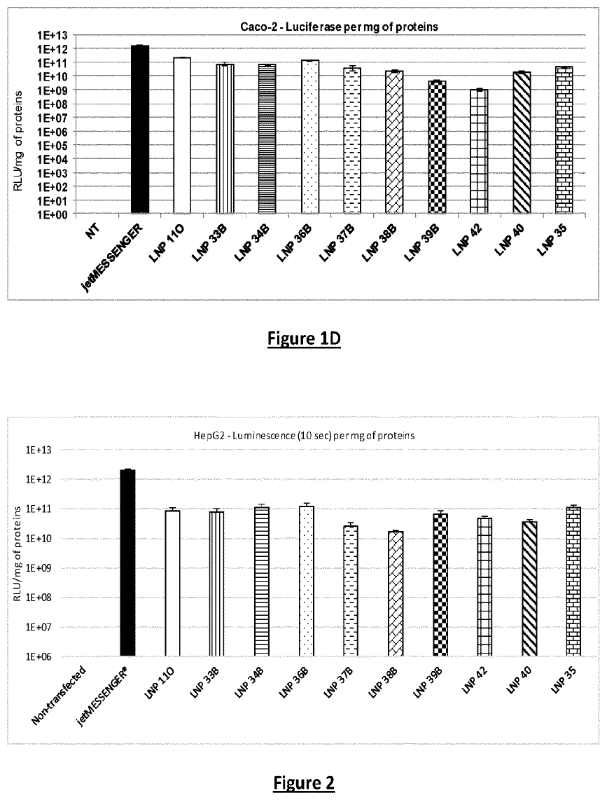

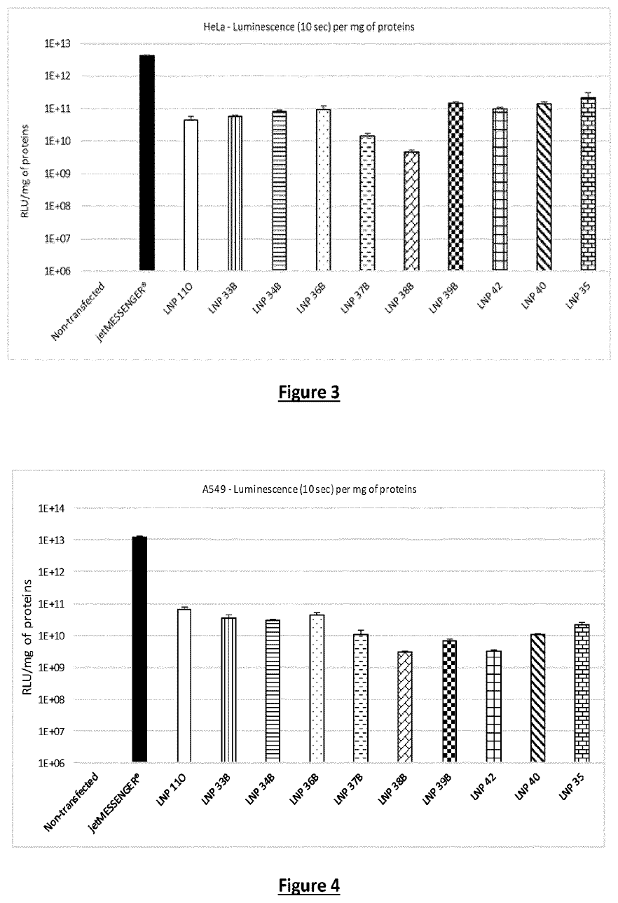

Transfection efficiency was assessed 24 hours post-transfection by luminescence reading. With the other cationic lipid W22.7 (LNP 33B), W12.7 (LNP 34B), W20.7 (LNP 35) and W16.7 (LNP 36B), higher transfection efficiency was observed compared to the LNP 110 except for the LNP 33B which gave similar results. Lower transfection efficiency was observed with the LNP 37B (SM-102 instead of W21.7 and DODMA) and LNP 38B (ALC-0315 instead of W21.7 and DODMA). The LNP 39B, LNP 40 and LNP 42 (DOTAP or DOTMA instead of W21.7) gave higher transfection efficiency compared to LNP 110 but similar to LNP 34B, LNP 36B and LNP 35 (FIG. 3).

g) A459 Cells were Transfected with jetMESSENGER®/Fluc mRNA Complexes or LNP 110, LNP 33B, LNP 34B, LNP 36B, LNP 37B, LNP 38B, LNP 39B, LNP 42, LNP 40, LNP 35.

Transfection efficiency was assessed 24 hours post-transfection by luminescence reading. With the other cationic lipid W22.7 (LNP 33B), W12.7 (LNP 34B), W20.7 (LNP 35) and W16.7 (LNP 36B), a similar transfection efficiency was observed compared to the LNP 110. Lower transfection efficiency was observed with the LNP 37B (SM-102 instead of W21.7 and DODMA), LNP 38B (ALC-0315 instead of W21.7 and DODMA), LNP 39B, LNP 40 and LNP 42 (DOTAP or DOTMA instead of W21.7) (FIG. 4).

h) Jurkat Cells were Transfected with jetMESSENGER®/Fluc mRNA Complexes or LNP 110, LNP 33B, LNP 34B, LNP 36B and LNP 35.

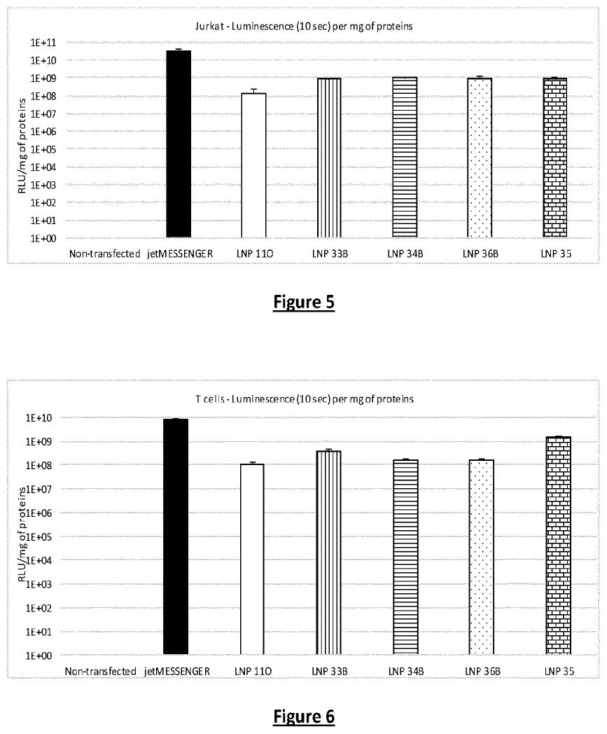

Transfection efficiency was assessed 24 hours post-transfection by luminescence reading. Slightly higher transfection efficiency was observed with the other cationic lipid W22.7 (LNP 33B), W12.7 (LNP 34B), W20.7 (LNP 35) and W16.7 (LNP 36B) compared to W21.7 (LNP 110) (FIG. 5).

i) Human Primary T Cells were Transfected with jetMESSENGER®/Fluc mRNA Complexes or LNP 110, LNP 33B, LNP 34B, LNP 36B and LNP 35.

Transfection efficiency was assessed 48 hours post-transfection by luminescence reading. Higher transfection efficiency was observed with the cationic lipid W20.7 (LNP 35) compared to W21.7 (LNP 110) and similar transfection efficiency was observed with the other cationic lipid W22.7 (LNP 33B), W12.7 (LNP 34B) and W16.7 (LNP 36B) compared to W21.7 (LNP 110) (FIG. 6).

j) Human Primary T Cells were Transfected with jetMESSENGER®/GFP mRNA Complexes or LNP 43B and LNP 44.

Transfection efficiency was assessed 48 hours post-transfection by GFP fluorescence. Good transfection efficiency was observed with LNP 43B and LNP 44 with low amount of mRNA. Transfection efficiency was lower with the LNPs compared to jetMESSENGER®/GFP mRNA complexes but with a higher amount of mRNA. Higher transfection efficiency was observed with LNP 44 (40 nm) compared to LNP 43B (80 nm) (FIG. 7).

k) Human Primary CD34+ Cells were Transfected with jetMESSENGER®/GFP mRNA Complexes or LNP 43B.

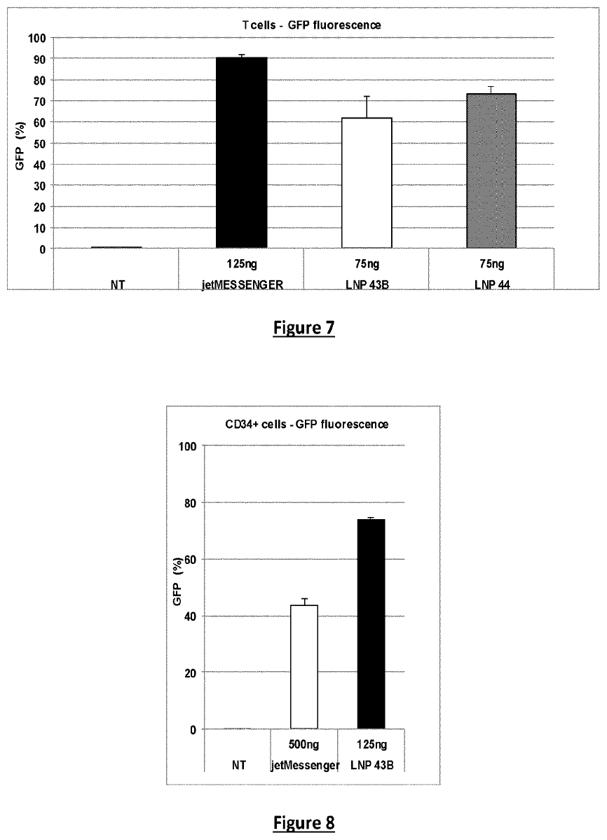

Transfection efficiency was assessed 24 hours post-transfection by GFP fluorescence. Higher transfection efficiency was observed with LNP 43B with lower amount of mRNA compared to jetMESSENGER®/GFP mRNA complexes (FIG. 8).

### Part C: Application to Various Nucleic Acids

i) HeLa Cells were Transfected with jetPRIME®/GFP mRNA Complexes or LNP 45.

Transfection efficiency was assessed 24 hours post-transfection by GFP fluorescence. Good transfection efficiency (GFP percentage) was observed with LNP 45 slightly lower compared to jetPRIME®/GFP mRNA complexes (FIG. 9).

ii) A549 Luc Cells were Transfected with INTERFERin®/siRNA GL3 Luc or GL2 Mm Complexes or LNP 46 or LNP 47.

Transfection efficiency was assessed 48 hours post-transfection by luminescence reading. Slightly higher transfection efficiency (luciferase extinction) was observed with LNP 46 compared to INTERFERin®/siRNA GL3_Luc mRNA complexes (FIG. 10).

### Example 7. In Vivo Activity

mRNA encoding Luciferase was administered into OF1 mice using in vivo-jetRNA® through different administration routes. Complexes were formed with a mRNA/in vivo-jetRNA®+ratio of 1:1 (μgmRNA:pLreagent) in mRNA Buffer. 5 μg or 10 μg of mRNA were injected for respectively intramuscular or intravenous (retro orbital injection) injections. Luciferase expression was assessed 24 h post-injection (FIGS. 11, 12 and 13). Higher transfection efficiency was observed in the lung with LNP 48B compared to LNP 49 and mRNA/in vivo-jetRNA® complexes via intravenous injection. Lower transfection efficiency was observed in the liver with LNP 48B and mRNA/in vivo-jetRNA® complexes compared to LNP 49 via intravenous injection. Similar transfection efficiency in the spleen was observed with LNP 48B and LNP 49 and mRNA/in vivo-jetRNA® complexes via intravenous injection (FIG. 11).

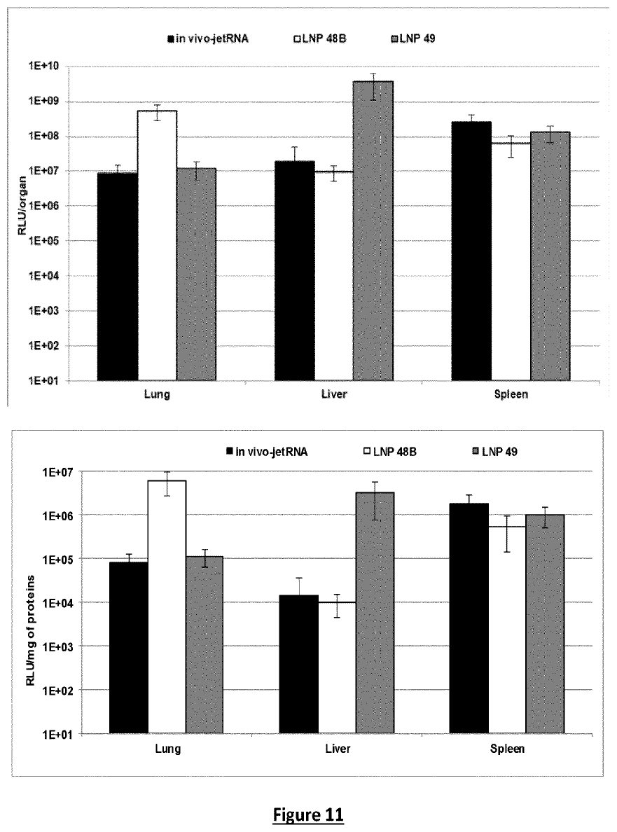

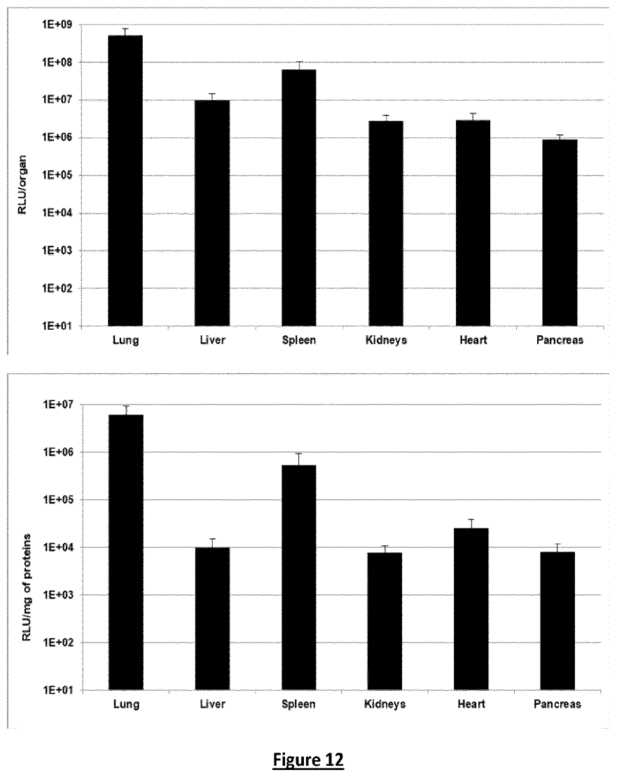

Luciferase expression was also observed in kidneys, heart and pancreas with the LNP 48B (FIG. 12).

Slightly higher transfection efficiency was observed in the muscle with LNP 48B compared to mRNA/in vivo-jetRNA® complexes via intramuscular injection (FIG. 13).

mRNA encoding Luciferase was administered into OF1 mice using in vivo-jetRNA® through retro-orbital injection with LNP 48D, LNP 50B, LNP 51C, LNP 52, LNP 53, LNP 54, LNP 55. 7.5 μg of mRNA were injected. Luciferase expression was assessed 24h post-injection. Higher transfection efficiency in the lung was observed with W21.7 (LNP 99C and LNP 50B) compared to SM-102 (LNP 51C) and LNPs with higher ratio of DSG-PEG (LNP 52 and LNP 53), without DODMA (LNP 54) or with DOTAP instead of W21.7 (LNP 55). Higher transfection efficiency in the liver was observed with SM-102 (LNP 51C) compared to other LNPs. Similar transfection efficiency in the spleen was observed with LNP 48B, LNP 50, LNP 49 and LNP 52 but the luciferase expression was slightly higher for theses LNPs compared to LNP 53, LNP 54, LNP 55 (FIG. 14).

### Example 8. Stability

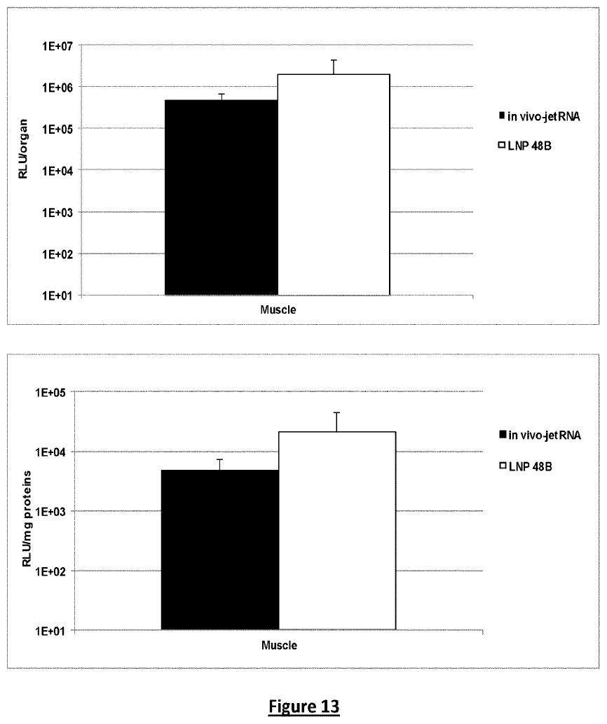

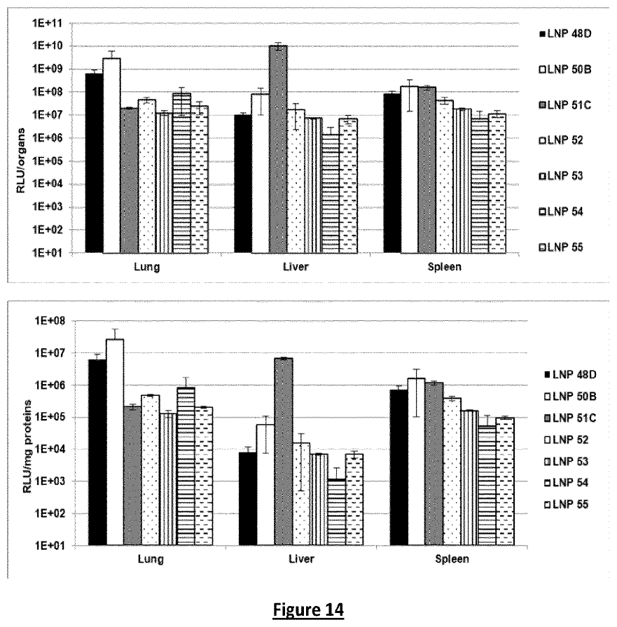

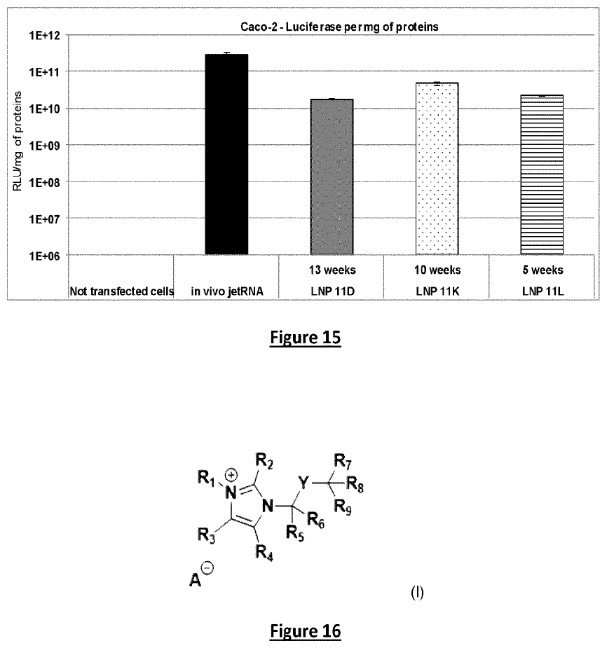

LNPs were stored at 4° C. for several weeks. Caco2 cells were transfected with in vivo-jetRNA®/Fluc mRNA complexes or different batches of LNP 11 (D, K and L). Transfection efficiency was assessed 24 hours post-transfection by luminescence reading. LNP 11 is stable at least 13 weeks at 4° C. as similar transfection efficiency was observed with the LNP 11D stored 13 weeks with the LNP 11K stored 10 weeks and with the LNP 11L stored 5 weeks (FIG. 15).

### Example 9. Synthesis of RNA/DNA-LNPs and Related Physico-Chemical Data

### 9.1 Formulations

Several RNA/DNA-lipid nanoparticles were formulated to define optimized compositions following several parameters (size, charge, encapsulation efficiency (EE), polydispersity (PDI), transfection efficiency, stability). The results are depicted in Table 3.

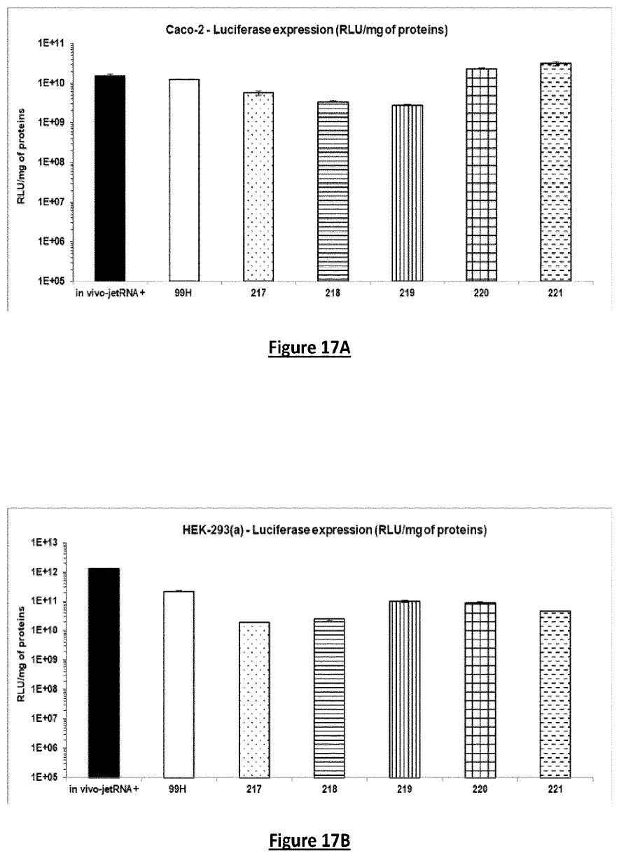

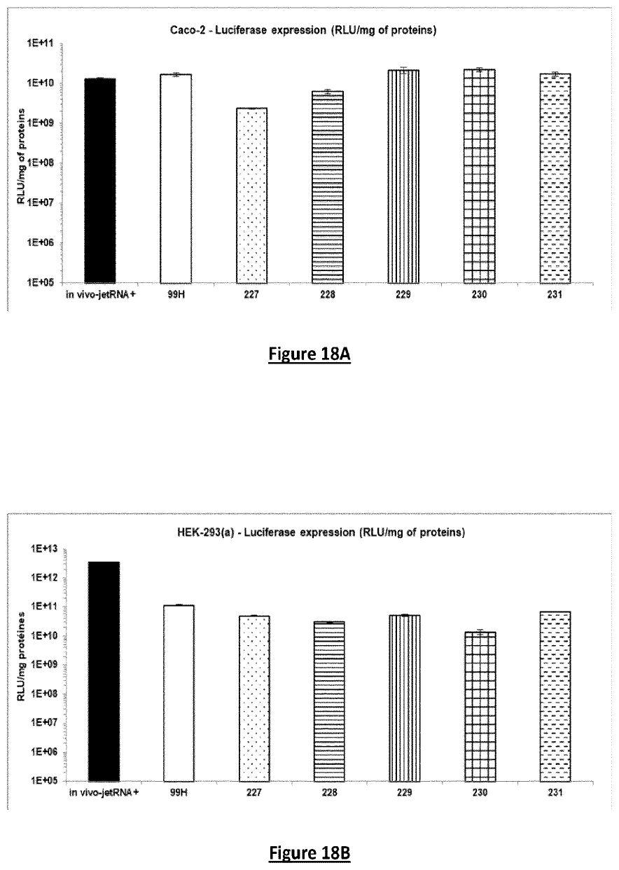

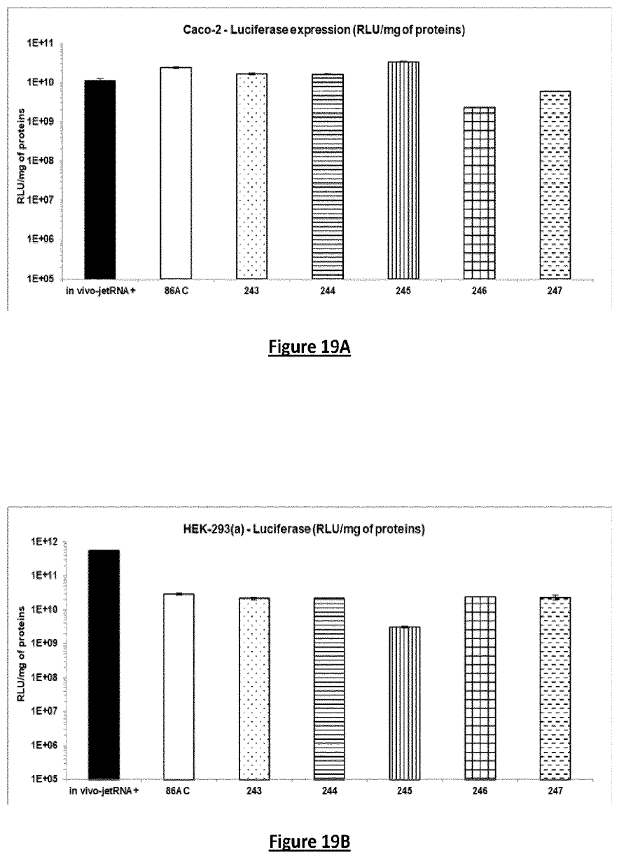

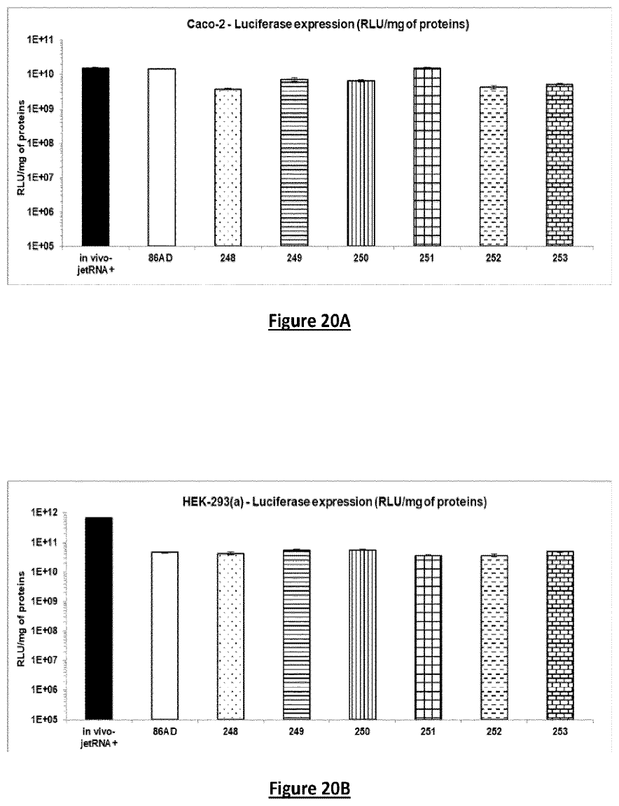

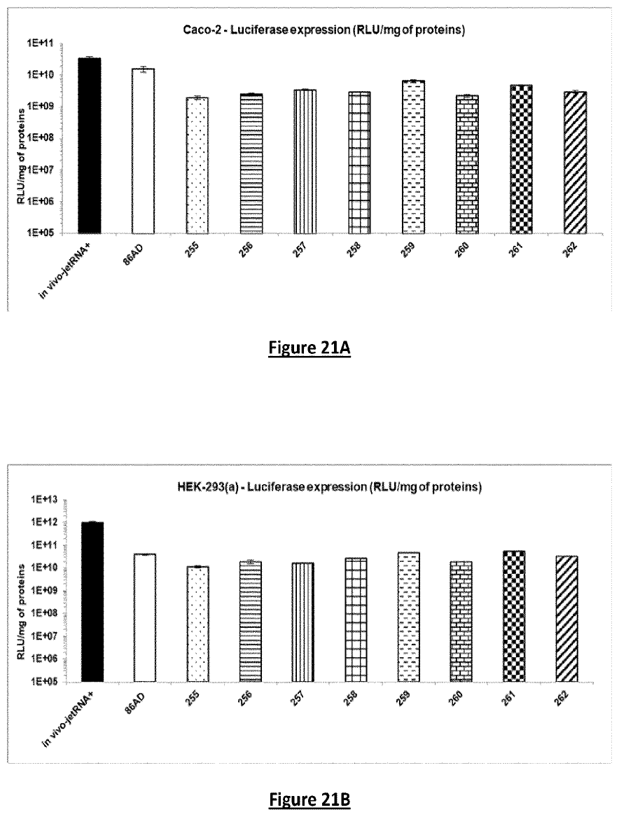

TABLE 3
LNP chemical composition and related physico-chemical data.

| | | Cationic lipid | | Ionizable lipid | | Helper lipid | | Stabilizing | |
|---|---|---|---|---|---|---|---|---|---|
| | LNP No | (mM) | | (mM) | | (mM) | | lipid (mM) | |
| FIG. 17 | 99H | W21.7 | 4.00 | DODMA | 3.00 | DPyPE | 1.00 | Cholesterol | 1.85 |
| | 217 | W3.5 | 4.00 | DODMA | 3.00 | DPyPE | 1.00 | Cholesterol | 1.85 |
| | 218 | W12.7 | 4.00 | DODMA | 3.00 | DPyPE | 1.00 | Cholesterol | 1.85 |
| | 219 | W13.7 | 4.00 | DODMA | 3.00 | DPyPE | 1.00 | Cholesterol | 1.85 |
| | 220 | W15.7 | 4.00 | DODMA | 3.00 | DPyPE | 1.00 | Cholesterol | 1.85 |
| | 221 | W16.7 | 4.00 | DODMA | 3.00 | DPyPE | 1.00 | Cholesterol | 1.85 |
| FIG. 18 | 227 | W18.9 | 4.00 | DODMA | 3.00 | DPyPE | 1.00 | Cholesterol | 1.85 |
| | 228 | W20.7 | 4.00 | DODMA | 3.00 | DPyPE | 1.00 | Cholesterol | 1.85 |
| | 229 | W22.7 | 4.00 | DODMA | 3.00 | DPyPE | 1.00 | Cholesterol | 1.85 |
| | 230 | W23.7 | 4.00 | DODMA | 3.00 | DPyPE | 1.00 | Cholesterol | 1.85 |
| | 231 | W25.7 | 4.00 | DODMA | 3.00 | DPyPE | 1.00 | Cholesterol | 1.85 |
| FIG. 19 | 86AC | W21.7 | 4.00 | DODMA | 3.00 | DPyPE | 1.00 | Cholesterol | 1.85 |
| | 243 | W44 | 4.00 | DODMA | 3.00 | DPyPE | 1.00 | Cholesterol | 1.85 |
| | 244 | W56 | 4.00 | DODMA | 3.00 | DPyPE | 1.00 | Cholesterol | 1.85 |
| | 245 | W57 | 4.00 | DODMA | 3.00 | DPyPE | 1.00 | Cholesterol | 1.85 |
| | 246 | W54 | 4.00 | DODMA | 3.00 | DPyPE | 1.00 | Cholesterol | 1.85 |
| | 247 | W58 | 4.00 | DODMA | 3.00 | DPyPE | 1.00 | Cholesterol | 1.85 |
| FIG. 20 | 86AD | W21.7 | 4.00 | DODMA | 3.00 | DPyPE | 1.00 | Cholesterol | 1.85 |
| | 248 | W55 | 4.00 | DODMA | 3.00 | DPyPE | 1.00 | Cholesterol | 1.85 |
| | 249 | W39 | 4.00 | DODMA | 3.00 | DPyPE | 1.00 | Cholesterol | 1.85 |
| | 250 | W36 | 4.00 | DODMA | 3.00 | DPyPE | 1.00 | Cholesterol | 1.85 |
| | 251 | W59 | 4.00 | DODMA | 3.00 | DPyPE | 1.00 | Cholesterol | 1.85 |
| | 252 | W51 | 4.00 | DODMA | 3.00 | DPyPE | 1.00 | Cholesterol | 1.85 |
| | 253 | W43 | 4.00 | DODMA | 3.00 | DPyPE | 1.00 | Cholesterol | 1.85 |
| FIG. 21 | 86AD | W21.7 | 4.00 | DODMA | 3.00 | DPyPE | 1.00 | Cholesterol | 1.85 |
| | 255 | W31 | 4.00 | DODMA | 3.00 | DPyPE | 1.00 | Cholesterol | 1.85 |
| | 256 | W60 | 4.00 | DODMA | 3.00 | DPyPE | 1.00 | Cholesterol | 1.85 |
| | 257 | W32 | 4.00 | DODMA | 3.00 | DPyPE | 1.00 | Cholesterol | 1.85 |
| | 258 | W61 | 4.00 | DODMA | 3.00 | DPyPE | 1.00 | Cholesterol | 1.85 |
| | 259 | W33 | 4.00 | DODMA | 3.00 | DPyPE | 1.00 | Cholesterol | 1.85 |
| | 260 | W62 | 4.00 | DODMA | 3.00 | DPyPE | 1.00 | Cholesterol | 1.85 |
| | 261 | W34 | 4.00 | DODMA | 3.00 | DPyPE | 1.00 | Cholesterol | 1.85 |
| | 262 | W35 | 4.00 | DODMA | 3.00 | DPyPE | 1.00 | Cholesterol | 1.85 |
| IP | 99F | W21.7 | 4.00 | DODMA | 3.00 | DPyPE | 1.00 | Cholesterol | 1.85 |
| OVA | 161 | W21.7 | 4.00 | DODMA | 3.00 | DPyPE | 1.00 | Cholesterol | 1.85 |
| FIGS. 22A | 99J | W21.7 | 4.00 | DODMA | 3.00 | DPyPE | 1.00 | Cholesterol | 1.85 |
| and 22B | 285 | W63 | 4.00 | DODMA | 3.00 | DPyPE | 1.00 | Cholesterol | 1.85 |
| | 286 | W64 | 4.00 | DODMA | 3.00 | DPyPE | 1.00 | Cholesterol | 1.85 |
| | 287 | W50 | 4.00 | DODMA | 3.00 | DPyPE | 1.00 | Cholesterol | 1.85 |
| | 288 | W40 | 4.00 | DODMA | 3.00 | DPyPE | 1.00 | Cholesterol | 1.85 |
| | 290 | W30 | 4.00 | DODMA | 3.00 | DPyPE | 1.00 | Cholesterol | 1.85 |
| | 291 | W42 | 4.00 | DODMA | 3.00 | DPyPE | 1.00 | Cholesterol | 1.85 |
| | 292 | W45 | 4.00 | DODMA | 3.00 | DPyPE | 1.00 | Cholesterol | 1.85 |
| | 293 | W46 | 4.00 | DODMA | 3.00 | DPyPE | 1.00 | Cholesterol | 1.85 |
| | 294 | W49 | 4.00 | DODMA | 3.00 | DPyPE | 1.00 | Cholesterol | 1.85 |
| | 295 | W65 | 4.00 | DODMA | 3.00 | DPyPE | 1.00 | Cholesterol | 1.85 |
| Cellular | 190B | W21.7 | 4.00 | DODMA | 3.00 | DPyPE | 1.00 | Cholesterol | 1.85 |
| distribution | 222 | — | — | ALC-0315 | 4.630 | DSPC | 0.940 | Cholesterol | 4.270 |
| | 190C | W21.7 | 4.00 | DODMA | 3.00 | DPyPE | 1.00 | Cholesterol | 1.85 |
| | 222B | — | — | ALC-0315 | 4.630 | DSPC | 0.940 | Cholesterol | 4.270 |
| DNA in | 109C | W21.7 | 4.00 | DODMA | 3.00 | DPyPE | 1.00 | Cholesterol | 1.85 |
| vitro | A32 | DOTAP | 3.00 | DODMA | 3.00 | — | — | Cholesterol | 7.92 |
| | 99G | W21.7 | 4.00 | DODMA | 3.00 | DPyPE | 1.00 | Cholesterol | 1.85 |
| DNA in | 191 | W21.7 | 4.00 | DODMA | 3.00 | DPyPE | 1.00 | Cholesterol | 1.85 |
| vivo | 215 | W21.7 | 4.00 | DODMA | 3.00 | DPyPE | 1.00 | Cholesterol | 1.85 |
| saRNA | 216 | — | — | Dlin- | 2.75 | DSPC | 0.55 | Cholesterol | 2.118 |
| | | | | MC3-DMA | | | | | |
| | 284 | W21.7 | 4.00 | DODMA | 3.00 | DPyPE | 1.00 | Cholesterol | 1.85 |
| MoDCs | 123E | W21.7 | 4.000 | DODMA | 3.000 | DPyPE | 1.000 | Cholesterol | 1.850 |
| | 141E | W21.7 | 4.000 | DODMA | 3.000 | DPyPE | 1.000 | Cholesterol | 1.700 |

| | | | | NA | | | | | |
|---|---|---|---|---|---|---|---|---|---|
| | | PEG-lipid | | concentration | | Size | PDI | Zeta | EE |
| | LNP No | (mM) | | (ng/μL) | | (nm) | (—) | (mV) | (%) |
| FIG. 17 | 99H | DSG- | 0.15 | FLuc | 200 | 45 ± 2 | 0.158 | +10 | 100 |
| | | PEG 2k | | mRNA | | | | | |
| | 217 | DSG- | 0.15 | FLuc | 50 | 41 ± 2 | 0.104 | +16 | 100 |
| | | PEG 2k | | mRNA | | | | | |
| | 218 | DSG- | 0.15 | FLuc | 50 | 29 ± 1 | 0.120 | +12 | 100 |
| | | PEG 2k | | mRNA | | | | | |
| | 219 | DSG- | 0.15 | FLuc | 50 | 74 ± 4 | 0.090 | +11 | 100 |
| | | PEG 2k | | mRNA | | | | | |
| | 220 | DSG- | 0.15 | FLuc | 50 | 66 ± 6 | 0.113 | +9 | 100 |
| | | PEG 2k | | mRNA | | | | | |
| | 221 | DSG- | 0.15 | FLuc | 50 | 56 ± 3 | 0.132 | +13 | 100 |
| | | PEG 2k | | mRNA | | | | | |
| FIG. 18 | 227 | DSG- | 0.15 | FLuc | 50 | 83 ± 3 | 0.064 | +15 | 100 |
| | | PEG 2k | | mRNA | | | | | |
| | 228 | DSG- | 0.15 | FLuc | 50 | 46 ± 2 | 0.130 | +12 | 100 |
| | | PEG 2k | | mRNA | | | | | |
| | 229 | DSG- | 0.15 | FLuc | 50 | 40 ± 2 | 0.140 | +14 | 100 |
| | | PEG 2k | | mRNA | | | | | |
| | 230 | DSG- | 0.15 | FLuc | 50 | 58 ± 1 | 0.066 | +15 | 100 |
| | | PEG 2k | | mRNA | | | | | |
| | 231 | DSG- | 0.15 | FLuc | 50 | 86 ± 2 | 0.053 | +10 | 00 |
| | | PEG 2k | | mRNA | | | | | |
| FIG. 19 | 86AD | DSG- | 0.15 | FLuc | 50 | 54 ± 1 | 0.085 | +16 | 100 |
| | | PEG 2k | | mRNA | | | | | |
| | 243 | DSG- | 0.15 | FLuc | 50 | 41 ± 2 | 0.134 | +12 | 100 |
| | | PEG 2k | | mRNA | | | | | |
| | 244 | DSG- | 0.15 | FLuc | 50 | 38 ± 3 | 0.130 | +12 | 100 |
| | | PEG 2k | | mRNA | | | | | |
| | 245 | DSG- | 0.15 | FLuc | 50 | 60 ± 3 | 0.101 | +12 | 100 |
| | | PEG 2k | | mRNA | | | | | |
| | 246 | DSG- | 0.15 | FLuc | 50 | 48 ± 3 | 0.092 | +12 | 100 |
| | | PEG 2k | | mRNA | | | | | |
| | 247 | DSG- | 0.15 | FLuc | 50 | 47 ± 2 | 0.162 | +11 | 100 |
| | | PEG 2k | | mRNA | | | | | |
| FIG. 20 | 86AD | DSG- | 0.15 | FLuc | 50 | 53 ± 3 | 0.112 | +14 | 100 |
| | | PEG 2k | | mRNA | | | | | |
| | 248 | DSG- | 0.15 | FLuc | 50 | 58 ± 3 | 0.126 | +17 | 100 |
| | | PEG 2k | | mRNA | | | | | |
| | 249 | DSG- | 0.15 | FLuc | 50 | 58 ± 3 | 0.134 | +13 | 100 |
| | | PEG 2k | | mRNA | | | | | |
| | 250 | DSG- | 0.15 | FLuc | 50 | 45 ± 4 | 0.136 | +13 | 100 |
| | | PEG 2k | | mRNA | | | | | |
| | 251 | DSG- | 0.15 | FLuc | 50 | 53 ± 4 | 0.142 | +11 | 100 |
| | | PEG 2k | | mRNA | | | | | |
| | 252 | DSG- | 0.15 | FLuc | 50 | 37 ± 3 | 0.172 | +11 | 100 |
| | | PEG 2k | | mRNA | | | | | |
| | 253 | DSG- | 0.15 | FLuc | 50 | 44 ± 2 | 0.185 | +12 | 100 |
| | | PEG 2k | | mRNA | | | | | |
| FIG. 21 | 86AD | DSG- | 0.15 | FLuc | 50 | 53 ± 3 | 0.112 | +14 | 100 |
| | | PEG 2k | | mRNA | | | | | |
| | 255 | DSG- | 0.15 | FLuc | 50 | 65 ± 4 | 0.081 | +16 | 100 |
| | | PEG 2k | | mRNA | | | | | |
| | 256 | DSG- | 0.15 | FLuc | 50 | 43 ± 4 | 0.141 | +15 | 100 |
| | | PEG 2k | | mRNA | | | | | |
| | 257 | DSG- | 0.15 | FLuc | 50 | 44 ± 3 | 0.090 | +11 | 100 |
| | | PEG 2k | | mRNA | | | | | |
| | 258 | DSG- | 0.15 | FLuc | 50 | 43 ± 6 | 0.157 | +13 | 100 |
| | | PEG 2k | | mRNA | | | | | |
| | 259 | DSG- | 0.15 | FLuc | 50 | 44 ± 1 | 0.156 | +12 | 100 |
| | | PEG 2k | | mRNA | | | | | |
| | 260 | DSG- | 0.15 | FLuc | 50 | 40 ± 3 | 0.156 | +13 | 100 |
| | | PEG 2k | | mRNA | | | | | |
| | 261 | DSG- | 0.15 | FLuc | 50 | 38 ± 2 | 0.139 | +13 | 100 |
| | | PEG 2k | | mRNA | | | | | |
| | 262 | DSG- | 0.15 | FLuc | 50 | 35 ± 5 | 0.181 | +13 | 100 |
| | | PEG 2k | | mRNA | | | | | |
| IP | 99F | DSG- | 0.15 | FLuc | 200 | 69 ± 4 | 0.104 | +11 | 98.5 |
| | | PEG 2k | | mRNA | | | | | |
| OVA | 161 | DSG- | 0.15 | OVA | 200 | 40 ± 1 | 0.188 | +11 | 99.6 |
| | | PEG 2k | | mRNA | | | | | |
| FIGS. 22A | 99J | DSG- | 0.15 | FLuc | 200 | 68 ± 5 | 0.097 | +14 | 99 |
| and 22B | | PEG 2k | | mRNA | | | | | |
| | 285 | DSG- | 0.15 | FLuc | 50 | 67 ± 4 | 0.103 | +14 | 86 |
| | | PEG 2k | | mRNA | | | | | |
| | 286 | DSG- | 0.15 | FLuc | 50 | 52 ± 11 | 0.168 | +13 | 94 |
| | | PEG 2k | | mRNA | | | | | |
| | 287 | DSG- | 0.15 | FLuc | 50 | 58 ± 3 | 0.121 | +11 | 96 |
| | | PEG 2k | | mRNA | | | | | |
| | 288 | DSG- | 0.15 | FLuc | 50 | 76 ± 9 | 0.128 | +11 | 96 |
| | | PEG 2k | | mRNA | | | | | |
| | 290 | DSG- | 0.15 | FLuc | 50 | 54 ± 3 | 0.127 | +11 | 96 |
| | | PEG 2k | | mRNA | | | | | |
| | 291 | DSG- | 0.15 | FLuc | 50 | 48 ± 2 | 0.123 | +12 | 96 |
| | | PEG 2k | | mRNA | | | | | |
| | 292 | DSG- | 0.15 | FLuc | 50 | 43 ± 3 | 0.177 | +13 | 96 |
| | | PEG 2k | | mRNA | | | | | |
| | 293 | DSG- | 0.15 | FLuc | 50 | 56 ± 4 | 0.271 | +11 | 96 |
| | | PEG 2k | | mRNA | | | | | |
| | 294 | DSG- | 0.15 | FLuc | 50 | 48 ± 4 | 0.166 | +11 | 96 |
| | | PEG 2k | | mRNA | | | | | |
| | 295 | DSG- | 0.15 | FLuc | 50 | 54 ± 3 | 0.138 | +12 | 96 |
| | | PEG 2k | | mRNA | | | | | |
| Cellular | 190B | DSG- | 0.15 | GFP | 250 | 44 ± 2 | 0.184 | +12 | 100 |
| distribution | | PEG 2k | | mRNA | | | | | |
| | 222 | DMG- | 0.160 | GFP | 250 | 44 ± 1 | 0.179 | −2 | 88.4 |
| | | PEG | | mRNA | | | | | |
| | 190C | DSG- | 0.15 | GFP | 250 | 54 ± 4 | 0.106 | +14 | 99.6 |
| | | PEG 2k | | mRNA | | | | | |
| | 222B | DMG- | 0.160 | GFP | 250 | 50 ± 1 | 0.107 | −3 | 64.4 |
| | | PEG | | mRNA | | | | | |
| DNA in | 109C | DSG- | 0.15 | GFP | 50 | 73 ± 2 | 0.038 | +16 | 100.0 |
| vitro | | PEG 2k | | DNA | | | | | |
| | A32 | DMG- | 0.08 | GFP | 176 | 111 ± 15 | 0.637 | +15 | 99.4 |
| | | PEG | | DNA | | | | | |
| | 99G | DSG- | 0.15 | Fluc | 200 | 44 ± 2 | 0.156 | +12 | 87.8 |
| | | PEG 2k | | mRNA | | | | | |
| DNA in | 191 | DSG- | 0.15 | FLuc | 200 | 55 ± 3 | 0.086 | +12 | 89.0 |
| vivo | | PEG 2k | | DNA | | | | | |
| saRNA | 215 | DSG- | 0.15 | GFP | 50 | 36 ± 3 | 0.170 | +16 | 91.2 |
| | | PEG 2k | | saRNA | | | | | |
| | 216 | DMG- | 0.083 | GFP | 50 | 42 ± 1 | 0.137 | −1 | 89.7 |
| | | PEG 2k | | saRNA | | | | | |
| | 284 | DSG- | 0.15 | nLUC | 50 | 87 ± 2 | 0.097 | +12 | 91 |
| | | PEG 2k | | saRNA | | | | | |
| MoDCs | 123E | DSG- | 0.150 | eGFP | 100 | 64 ± 4 | 0.109 | +15 | 98.4 |
| | | PEG 2k | | mRNA | | | | | |
| | 141E | DSG- | 0.300 | eGFP | 100 | 36 ± 3 | 0.212 | +12 | 98.9 |
| | | PEG 2k | | mRNA | | | | | |

9.2 Evaluation of Cationic Lipids as Active Component within a Lipid Nanoparticle

The inventors evaluated the properties (size, charge, encapsulation efficiency, polydispersity, transfection efficiency, stability) of LNPs 99H, 217-221 (see Table 4 below).

TABLE 4
Chemical compositions of screened LNPs 99H, 217-221.

| | | | | | | | | | | | NA | | | | | |
|---|---|---|---|---|---|---|---|---|---|---|---|---|---|---|---|---|
| LNP | Cationic | | Ionizable | | Helper | | Stabilizing | | PEG- | | concentration | | Size | PDI | Zeta | |
| No | lipid (mM) | | lipid (mM) | | lipid (mM) | | lipid (mM) | | lipid (mM) | | (ng/μL) | | (nm) | (—) | (mV) | EE % |
| 99H | W21.7 | 4.00 | DODMA | 3.00 | DPyPE | 1.00 | Cholesterol | 1.85 | DSG- | 0.15 | FLuc | 200 | 45 ± 2 | 0.158 | +10 | 100 |
| | | | | | | | | | PEG 2k | | mRNA | | | | | |
| 217 | W3.5 | 4.00 | DODMA | 3.00 | DPyPE | 1.00 | Cholesterol | 1.85 | DSG- | 0.15 | FLuc | 50 | 41 ± 2 | 0.104 | +16 | 100 |
| | | | | | | | | | PEG 2k | | mRNA | | | | | |
| 218 | W12.7 | 4.00 | DODMA | 3.00 | DPyPE | 1.00 | Cholesterol | 1.85 | DSG- | 0.15 | FLuc | 50 | 29 ± 1 | 0.120 | +12 | 100 |
| | | | | | | | | | PEG 2k | | mRNA | | | | | |
| 219 | W13.7 | 4.00 | DODMA | 3.00 | DPyPE | 1.00 | Cholesterol | 1.85 | DSG- | 0.15 | FLuc | 50 | 74 ± 4 | 0.090 | +11 | 100 |
| | | | | | | | | | PEG 2k | | mRNA | | | | | |
| 220 | W15.7 | 4.00 | DODMA | 3.00 | DPyPE | 1.00 | Cholesterol | 1.85 | DSG- | 0.15 | FLuc | 50 | 66 ± 6 | 0.113 | +9 | 100 |
| | | | | | | | | | PEG 2k | | mRNA | | | | | |
| 221 | W16.7 | 4.00 | DODMA | 3.00 | DPyPE | 1.00 | Cholesterol | 1.85 | DSG- | 0.15 | FLuc | 50 | 56 ± 3 | 0.132 | +13 | 100 |
| | | | | | | | | | PEG 2k | | mRNA | | | | | |

All the nanoparticles prepared displayed slightly positively charged zeta potential (9-16 mV) and had a size lower than 75 nm. A low PDI (<0.2) and complete mRNA encapsulation efficiency were observed for all LNPs assembled with cationic lipids.

Caco-2 and HEK-293(a) cells were transfected with in vivo-jetRNA©+/Fluc mRNA complexes or LNP 99H, 217-221 (FIGS. 17A and 17B). Transfection efficiency was assessed 24 hours post-transfection by luminescence reading. With the different cationic lipids W21.7 (LNP99H), W3.5 (LNP217), W12.7 (LNP218), W13.7 (LNP219), W15.7 (LNP220) and W16.7 (LNP221), a good luciferase expression was observed (>1·10⁹ or >1·10¹⁰ RLU/mg of proteins for Caco-2 and HEK-293 cells, respectively). For Caco-2 cells, a lower mRNA expression was observed with LNP 217-219 (3-6.109 RLU/mg of proteins) compared to LNP99H (1·10¹⁰ RLU/mg of proteins) whereas a higher luciferase expression was observed with LNP220 and 221 (2-3·10¹⁰ RLU/mg of proteins). For HEK-293(a) cells, lower luciferase expression was observed with LNP 217, 218, 220 and 221 (2-9·10¹⁰ RLU/mg of proteins) compared to LNP99H (2·10¹¹ RLU/mg of proteins) whereas a similar luciferase expression was observed with LNP219 (1·10¹¹ RLU/mg of proteins).

The inventors also evaluated the properties (size, charge, encapsulation efficiency, polydispersity, transfection efficiency, stability) of LNPs 227-231 (see Table 5 below).

TABLE 5
Chemical compositions of screened LNPs 227-231.

| | | | | | | | | | | | NA | | | | | |
|---|---|---|---|---|---|---|---|---|---|---|---|---|---|---|---|---|
| LNP | Cationic | | Ionizable | | Helper | | Stabilizing | | PEG- | | concentration | | Size | PDI | Zeta | |
| No | lipid (mM) | | lipid (mM) | | lipid (mM) | | lipid (mM) | | lipid (mM) | | (ng/μL) | | (nm) | (—) | (mV) | EE % |
| 227 | W18.9 | 4.00 | DODMA | 3.00 | DPyPE | 1.00 | Cholesterol | 1.85 | DSG- | 0.15 | FLuc | 50 | 83 ± 3 | 0.064 | +15 | 100 |
| | | | | | | | | | PEG 2k | | mRNA | | | | | |
| 228 | W20.7 | 4.00 | DODMA | 3.00 | DPyPE | 1.00 | Cholesterol | 1.85 | DSG- | 0.15 | FLuc | 50 | 46 ± 2 | 0.130 | +12 | 100 |
| | | | | | | | | | PEG 2k | | mRNA | | | | | |
| 229 | W22.7 | 4.00 | DODMA | 3.00 | DPyPE | 1.00 | Cholesterol | 1.85 | DSG- | 0.15 | FLuc | 50 | 40 ± 2 | 0.140 | +14 | 100 |
| | | | | | | | | | PEG 2k | | mRNA | | | | | |
| 230 | W23.7 | 4.00 | DODMA | 3.00 | DPyPE | 1.00 | Cholesterol | 1.85 | DSG- | 0.15 | FLuc | 50 | 58 ± 1 | 0.066 | +15 | 100 |
| | | | | | | | | | PEG 2k | | mRNA | | | | | |
| 231 | W25.7 | 4.00 | DODMA | 3.00 | DPyPE | 1.00 | Cholesterol | 1.85 | DSG- | 0.15 | FLuc | 50 | 86 ± 2 | 0.053 | +10 | 100 |
| | | | | | | | | | PEG 2k | | mRNA | | | | | |

All the nanoparticles assembled displayed slightly positively charged zeta potential. LNP with W18.9 (LNP227) or W25.7 (LNP231) as cationic component had slightly bigger size (83-86 nm) than with W21.7 (LNP99H), W20.7 (LNP228), W22.7 (LNP229) or W23.7 (LNP230). A low PDI (<0.2) and good encapsulation efficiency (100%) were observed for all LNPs with cationic lipids.

Caco-2 and HEK-293(a) cells were transfected with in vivo-jetRNA®+/Fluc mRNA complexes or LNP 99H, 227-231 (FIGS. 18A and 18B). Transfection efficiency was assessed 24 hours post-transfection by luminescence reading. With the different cationic lipids W21.7 (LNP99H), W18.9 (LNP227), W20.7 (LNP228), W22.7 (LNP229), W23.7 (LNP230) and W25.7 (LNP231), a good transfection efficiency was observed (>1·10⁹ or >1·10¹⁰ RLU/mg of proteins for Caco-2 and HEK-293 cells, respectively). For Caco-2 cells, a lower transfection efficiency was observed with LNP227 and LNP228 (2 and 6.109 RLU/mg of proteins respectively) compared to LNP99H (2·10¹⁰ RLU/mg of proteins) whereas a similar transfection efficiency was observed with LNP229-231. For HEK-293(a) cells, a lower transfection efficiency was observed with LNP 227-231 (1-7·10¹⁰ RLU/mg of proteins) compared to LNP99H (1·10¹¹ RLU/mg of proteins).

9.3 Evaluation of Cationic Lipids with Alkylated Cationic Heads or Thioether Linker

The inventors evaluated the properties (size, charge, encapsulation efficiency, polydispersity, transfection efficiency, stability) of LNPs 86AC, 243-247 (see Table 6 below).

TABLE 6
Chemical composition of screened LNPs 86AC, 243-247
(alkylated imidazoliums and thioether linker).

| | LNP | Cationic | | Ionizable | | Helper | | Stabilizing | |
|---|---|---|---|---|---|---|---|---|---|
| | No | lipid (mM) | | lipid (mM) | | lipid (mM) | | lipid (mM) | |
| | 86AC | W21.7 | 4.00 | DODMA | 3.00 | DPyPE | 1.00 | Cholesterol | 1.85 |
| Alkylated | 243 | W44 | 4.00 | DODMA | 3.00 | DPyPE | 1.00 | Cholesterol | 1.85 |
| cationic | 244 | W56 | 4.00 | DODMA | 3.00 | DPyPE | 1.00 | Cholesterol | 1.85 |
| heads | 245 | W57 | 4.00 | DODMA | 3.00 | DPyPE | 1.00 | Cholesterol | 1.85 |
| Thioether | 246 | W54 | 4.00 | DODMA | 3.00 | DPyPE | 1.00 | Cholesterol | 1.85 |
| linker | 247 | W58 | 4.00 | DODMA | 3.00 | DPyPE | 1.00 | Cholesterol | 1.85 |

| | | | | NA | | | | | |
|---|---|---|---|---|---|---|---|---|---|
| | LNP | PEG- | | concentration | | Size | PDI | Zeta | |
| | No | lipid (mM) | | (ng/μL) | | (nm) | (—) | (mV) | EE % |
| | 86AC | DSG- | 0.15 | FLuc | 50 | 54 ± 1 | 0.085 | +16 | 100 |
| | | PEG 2k | | mRNA | | | | | |
| Alkylated | 243 | DSG- | 0.15 | FLuc | 50 | 41 ± 2 | 0.134 | +12 | 100 |
| cationic | | PEG 2k | | mRNA | | | | | |
| heads | 244 | DSG- | 0.15 | FLuc | 50 | 38 ± 3 | 0.130 | +12 | 100 |
| | | PEG 2k | | mRNA | | | | | |
| | 245 | DSG- | 0.15 | FLuc | 50 | 60 ± 3 | 0.101 | +12 | 100 |
| | | PEG 2k | | mRNA | | | | | |
| Thioether | 246 | DSG- | 0.15 | FLuc | 50 | 48 ± 3 | 0.092 | +12 | 100 |
| linker | | PEG 2k | | mRNA | | | | | |
| | 247 | DSG- | 0.15 | FLuc | 50 | 47 ± 2 | 0.162 | +11 | 100 |
| | | PEG 2k | | mRNA | | | | | |

All LNP formulations were slightly charged positively (11-16 mV) and had a size lower than 60 nm. A good PDI (<0.2) and good encapsulation efficiency (100%) were observed for all LNPs with cationic lipids. It is noteworthy that, despite the inductive effect of alkyl chains on the imidazolium residue, the zeta potential was not significantly changed.

Caco-2 and HEK-293(a) cells were transfected with in vivo-jetRNA®+/Fluc mRNA complexes or LNP 86AC, 243-247 (FIGS. 19A and 19B). Transfection efficiency was assessed 24 hours post-transfection by luminescence reading. With the different cationic lipids W21.7 (LNP86AC), W44 (LNP243), W56 (LNP244), and W57 (LNP245) with alkylated cationic heads, and W54 (LNP246) and W58 (LNP247) with a thioether linker, a good transfection efficiency was observed (>1·10⁹ or >1·10¹⁰ RLU/mg of proteins for Caco-2 and HEK-293 cells, respectively). For Caco-2 cells, a lower transfection efficiency was observed with LNP 246 and 247 (2 and 6.109 RLU/mg of proteins respectively) compared to LNP86AC (2·10¹⁰ RLU/mg of proteins) whereas a higher or similar transfection efficiency was observed with LNP243-245 (2-3·10¹⁰ RLU/mg of proteins). For HEK-293(a) cells, a lower transfection efficiency was observed with LNP 245 (3.109 RLU/mg of proteins) compared to LNP86AC (3·10¹⁰ RLU/mg of proteins) whereas a similar transfection efficiency was observed with LNP243, 244, 246 and 247 (2·10¹⁰ RLU/mg of proteins).

9.4 Evaluation of Cationic Lipids with Sulfone, Amide or Ether Linker

The inventors evaluated the properties (size, charge, encapsulation efficiency, polydispersity, transfection efficiency, stability) of LNPs 86AD, 248-253 (see Table 7 below).

TABLE 7
Chemical composition of screened LNPs 86AD, 248-253 (sulfone, amide, ether linkers)

| | LNP | Cationic | | Ionizable | | Helper | | Stabilizing | |
|---|---|---|---|---|---|---|---|---|---|
| | No | lipid (mM) | | lipid (mM) | | lipid (mM) | | lipid (mM) | |
| | 86AD | W21.7 | 4.00 | DODMA | 3.00 | DPyPE | 1.00 | Cholesterol | 1.85 |
| Sulfone linker | 248 | W55 | 4.00 | DODMA | 3.00 | DPyPE | 1.00 | Cholesterol | 1.85 |
| Amide linker | 249 | W39 | 4.00 | DODMA | 3.00 | DPyPE | 1.00 | Cholesterol | 1.85 |
| Ether | 250 | W36 | 4.00 | DODMA | 3.00 | DPyPE | 1.00 | Cholesterol | 1.85 |
| linker | 251 | W59 | 4.00 | DODMA | 3.00 | DPyPE | 1.00 | Cholesterol | 1.85 |
| | 252 | W51 | 4.00 | DODMA | 3.00 | DPyPE | 1.00 | Cholesterol | 1.85 |
| | 253 | W43 | 4.00 | DODMA | 3.00 | DPyPE | 1.00 | Cholesterol | 1.85 |

| | | | | NA | | | | | |
|---|---|---|---|---|---|---|---|---|---|
| | LNP | PEG- | | concentration | | Size | PDI | Zeta | EE |
| | No | lipid (mM) | | (ng/μL) | | (nm) | (—) | (mV) | % |
| | 86AD | DSG-PEG 2k | 0.15 | FLuc mRNA | 50 | 53 ± 3 | 0.112 | +14 | 100 |
| Sulfone linker | 248 | DSG-PEG 2k | 0.15 | FLuc mRNA | 50 | 58 ± 3 | 0.126 | +17 | 100 |
| Amide linker | 249 | DSG-PEG 2k | 0.15 | FLuc mRNA | 50 | 58 ± 3 | 0.134 | +13 | 100 |
| Ether | 250 | DSG-PEG 2k | 0.15 | FLuc mRNA | 50 | 45 ± 4 | 0.136 | +13 | 100 |
| linker | 251 | DSG-PEG 2k | 0.15 | FLuc mRNA | 50 | 53 ± 4 | 0.142 | +11 | 100 |
| | 252 | DSG-PEG 2k | 0.15 | FLuc mRNA | 50 | 37 ± 3 | 0.172 | +11 | 100 |
| | 253 | DSG-PEG 2k | 0.15 | FLuc mRNA | 50 | 44 ± 2 | 0.185 | +12 | 100 |

All assembled nanoparticles displayed slightly positively charged zeta potential (11-17 mV) and had a size lower than 60 nm. A low PDI (<0.2) and good encapsulation efficiency (100%) were observed for all LNPs with cationic lipids.

Caco-2 and HEK-293(a) cells were transfected with in vivo-jetRNA©+/Fluc mRNA complexes or LNP86AD, 248-253 (FIGS. 20A and 20B). Transfection efficiency was assessed 24 hours post-transfection by luminescence reading. With the different cationic lipids W21.7 (LNP86AD), W55 with a sulfone linker (LNP248), W39 with an amide linker (LNP249) and W36 (LNP250), W59 (LNP251), W51 (LNP252) and W43 (LNP253) with an ether linker, a good mRNA expression was observed (>4.109 or >4·10¹⁰ RLU/mg of proteins for Caco-2 and HEK-293 cells, respectively). For Caco-2 cells, a lower transfection efficiency was observed with LNP248, LNP249, LNP250, LNP252 and LNP253 (4-7.109 RLU/mg of proteins) compared to LNP86AD (1·10¹⁰ RLU/mg of proteins) whereas a similar mRNA expression was observed with LNP251. For HEK-293(a) cells, a similar luciferase expression was observed for all LNPs.

### 9.5 Evaluation of Biodegradable Lipids

The inventors evaluated the properties (size, charge, encapsulation efficiency, polydispersity, transfection efficiency, stability) of LNPs 86AD, 255-262 (see Table 8 below).

TABLE 8
Chemical composition of screened LNPs 86 AD, 255-262

| | | | | | | | | | | | NA concen- | | | | | |
|---|---|---|---|---|---|---|---|---|---|---|---|---|---|---|---|---|
| LNP | Cationic | | Ionizable | | Helper | | Stabilizing | | PEG | | tration | | Size | PDI | Zeta | EE |
| No | lipid (mM) | | lipid (mM) | | lipid (mM) | | lipid (mM) | | lipid (mM) | | (ng/μL) | | (nm) | (—) | (mV) | (%) |
| 86AD | W21.7 | 4.00 | DODMA | 3.00 | DPyPE | 1.00 | Choles- | 1.85 | DSG- | 0.15 | FLuc | 50 | 53 ± 3 | 0.112 | +14 | 100 |
| | | | | | | | terol | | PEG 2k | | mRNA | | | | | |
| 255 | W31 | 4.00 | DODMA | 3.00 | DPyPE | 1.00 | Choles- | 1.85 | DSG- | 0.15 | FLuc | 50 | 65 ± 4 | 0.081 | +16 | 100 |
| | | | | | | | terol | | PEG 2k | | mRNA | | | | | |
| 256 | W60 | 4.00 | DODMA | 3.00 | DPyPE | 1.00 | Choles- | 1.85 | DSG- | 0.15 | FLuc | 50 | 43 ± 4 | 0.141 | +15 | 100 |
| | | | | | | | terol | | PEG 2k | | mRNA | | | | | |
| 257 | W32 | 4.00 | DODMA | 3.00 | DPyPE | 1.00 | Choles- | 1.85 | DSG- | 0.15 | FLuc | 50 | 44 ± 3 | 0.090 | +11 | 100 |
| | | | | | | | terol | | PEG 2k | | mRNA | | | | | |
| 258 | W61 | 4.00 | DODMA | 3.00 | DPyPE | 1.00 | Choles- | 1.85 | DSG- | 0.15 | FLuc | 50 | 43 ± 6 | 0.157 | +13 | 100 |
| | | | | | | | terol | | PEG 2k | | mRNA | | | | | |
| 259 | W33 | 4.00 | DODMA | 3.00 | DPyPE | 1.00 | Choles- | 1.85 | DSG- | 0.15 | FLuc | 50 | 44 ± 1 | 0.156 | +12 | 100 |
| | | | | | | | terol | | PEG 2k | | mRNA | | | | | |
| 260 | W62 | 4.00 | DODMA | 3.00 | DPyPE | 1.00 | Choles- | 1.85 | DSG- | 0.15 | FLuc | 50 | 40 ± 3 | 0.156 | +13 | 100 |
| | | | | | | | terol | | PEG 2k | | mRNA | | | | | |
| 261 | W34 | 4.00 | DODMA | 3.00 | DPyPE | 1.00 | Choles- | 1.85 | DSG- | 0.15 | FLuc | 50 | 38 ± 2 | 0.139 | +13 | 100 |
| | | | | | | | terol | | PEG 2k | | mRNA | | | | | |
| 262 | W35 | 4.00 | DODMA | 3.00 | DPyPE | 1.00 | Choles- | 1.85 | DSG- | 0.15 | FLuc | 50 | 35 ± 5 | 0.181 | +13 | 100 |
| | | | | | | | terol | | PEG 2k | | mRNA | | | | | |

All assembled nanoparticles displayed slightly positively charged zeta potential (11-17 mV) and had a size lower than 65 nm. A low PDI (<0.2) and good encapsulation efficiency (100%) were observed for all LNPs with cationic lipids.

Caco-2 and HEK-293(a) cells were transfected with in vivo-jetRNA®+/Fluc mRNA complexes or LNP86AD, 255-262 (FIGS. 21A and 21B). Transfection efficiency was assessed 24 hours post-transfection by luminescence reading. With W21.7 (LNP86AD) and different biodegradable lipids W31 (LNP255), W60 (LNP256), W32 (LNP257), W61 (LNP258), W33 (LNP259), W62 (LNP260), W34 (LNP261) and W35 (LNP262), a good transfection efficiency was observed (>2·10⁹ or >1·10¹⁰ RLU/mg of proteins for Caco-2 and HEK-293 cells, respectively). For Caco-2 cells, a lower luciferase expression was observed with all biodegradable lipids (2-6.109 RLU/mg of proteins) compared to LNP86AD (2·10¹⁰ RLU/mg of proteins). However, for HEK-293(a) cells a similar luciferase expression was observed with all LNPs.

### 9.6 Evaluation of Cationic Lipids

The inventors evaluated the properties (size, charge, encapsulation efficiency, polydispersity, transfection efficiency, stability) of LNPs 99J, 285-288, 290-295 (see Table 9 below).

TABLE 9
Chemical composition of screened LNPs 99J, 285-288, 290-295

| | | | | | | | | | | | NA | | | | | |
|---|---|---|---|---|---|---|---|---|---|---|---|---|---|---|---|---|
| LNP | Cationic | | Ionizable | | Helper | | Stabilizing | | PEG | | concentration | | | PDI | Zeta | EE |
| No | lipid (mM) | | lipid (mM) | | lipid (mM) | | lipid (mM) | | lipid (mM) | | (ng/μL) | | Size (nm) | (—) | (mV) | (%) |
| 99J | W21.7 | 4.00 | DODMA | 3.00 | DPyPE | 1.00 | Cholesterol | 1.85 | DSG- | 0.15 | FLuc | 200 | 68 ± 5 | 0.097 | +14 | 99 |
| | | | | | | | | | PEG 2k | | mRNA | | | | | |
| 285 | W63 | 4.00 | DODMA | 3.00 | DPyPE | 1.00 | Cholesterol | 1.85 | DSG- | 0.15 | FLuc | 50 | 67 ± 4 | 0.103 | +14 | 86 |
| | | | | | | | | | PEG 2k | | mRNA | | | | | |
| 286 | W64 | 4.00 | DODMA | 3.00 | DPyPE | 1.00 | Cholesterol | 1.85 | DSG- | 0.15 | FLuc | 50 | 52 ± 11 | 0.168 | +13 | 94 |
| | | | | | | | | | PEG 2k | | mRNA | | | | | |
| 287 | W50 | 4.00 | DODMA | 3.00 | DPyPE | 1.00 | Cholesterol | 1.85 | DSG- | 0.15 | FLuc | 50 | 58 ± 3 | 0.121 | +11 | 96 |
| | | | | | | | | | PEG 2k | | mRNA | | | | | |
| 288 | W40 | 4.00 | DODMA | 3.00 | DPyPE | 1.00 | Cholesterol | 1.85 | DSG- | 0.15 | FLuc | 50 | 76 ± 9 | 0.128 | +11 | 96 |
| | | | | | | | | | PEG 2k | | mRNA | | | | | |
| 290 | W30 | 4.00 | DODMA | 3.00 | DPyPE | 1.00 | Cholesterol | 1.85 | DSG- | 0.15 | FLuc | 50 | 54 ± 3 | 0.127 | +11 | 96 |
| | | | | | | | | | PEG 2k | | mRNA | | | | | |
| 291 | W42 | 4.00 | DODMA | 3.00 | DPyPE | 1.00 | Cholesterol | 1.85 | DSG- | 0.15 | FLuc | 50 | 48 ± 2 | 0.123 | +12 | 96 |
| | | | | | | | | | PEG 2k | | mRNA | | | | | |
| 292 | W45 | 4.00 | DODMA | 3.00 | DPyPE | 1.00 | Cholesterol | 1.85 | DSG- | 0.15 | FLuc | 50 | 43 ± 3 | 0.177 | +13 | 96 |
| | | | | | | | | | PEG 2k | | mRNA | | | | | |
| 293 | W46 | 4.00 | DODMA | 3.00 | DPyPE | 1.00 | Cholesterol | 1.85 | DSG- | 0.15 | FLuc | 50 | 56 ± 4 | 0.271 | +11 | 96 |
| | | | | | | | | | PEG 2k | | mRNA | | | | | |
| 294 | W49 | 4.00 | DODMA | 3.00 | DPyPE | 1.00 | Cholesterol | 1.85 | DSG- | 0.15 | FLuc | 50 | 48 ± 4 | 0.166 | +11 | 96 |
| | | | | | | | | | PEG 2k | | mRNA | | | | | |
| 295 | W65 | 4.00 | DODMA | 3.00 | DPyPE | 1.00 | Cholesterol | 1.85 | DSG- | 0.15 | FLuc | 50 | 54 ± 3 | 0.138 | +12 | 96 |
| | | | | | | | | | PEG 2k | | mRNA | | | | | |

All assembled nanoparticles displayed slightly positively charged zeta potential (11-14 mV) and had a size lower than 76 nm. A low PDI (<0.2) and good encapsulation efficiency (86-99%) were observed for all LNPs with cationic lipids.

Caco-2 and HEK-293(a) cells were transfected with in vivo-jetRNA©+/Fluc mRNA complexes or LNP99J, 285-288, 290-295 (FIGS. 22A and 22B). Transfection efficiency was assessed 24 hours post-transfection by luminescence reading. A lower luciferase expression was observed (0.5-1 log lower than other LNPs) with W63 (LNP285) on both cell lines due to a low mRNA concentration compared to other LNPs. For LNPs with cationic ionizable lipids W64 (LNP286), W50 (LNP287), W40 (LNP288), W30 (LNP290), W42 (LNP291), W45 (LNP292), W46 (LNP293), W49 (LNP294) or W65 (LNP295), a good transfection efficiency was observed (>2.109 or >1·10¹⁰ RLU/mg of proteins for Caco-2 and HEK-293 cells, respectively). For Caco-2 cells, a lower luciferase expression was observed with lipids W64 (LNP286), W50 (LNP287) and W45 (LNP292) (2-5.109 RLU/mg of proteins) compared to LNP99J (2.101 RLU/mg of proteins). However, for HEK-293(a) cells a similar luciferase expression was observed with all LNPs.

### 9.7 Intra-Peritoneal Injection

The inventors evaluated the transfection efficiency of LNP 99F (see Table 10 below) through intra-peritoneal injection.

TABLE 10
Chemical composition of screened LNP 99F

| | | | | | | | | | | | NA | | | | | |
|---|---|---|---|---|---|---|---|---|---|---|---|---|---|---|---|---|
| LNP | Cationic | | Ionizable | | Helper | | Stabilizing | | PEG- | | concentration | | Size | PDI | Zeta | |
| No | lipid (mM) | | lipid (mM) | | lipid (mM) | | lipid (mM) | | lipid (mM) | | (ng/μL) | | (nm) | (—) | (mV) | EE % |
| 99F | W21.7 | 4.00 | DODMA | 3.00 | DPyPE | 1.00 | Cholesterol | 1.85 | DSG- | 0.15 | FLuc | 200 | 69 ± 4 | 0.104 | +11 | 98.5 |
| | | | | | | | | | PEG 2k | | mRNA | | | | | |

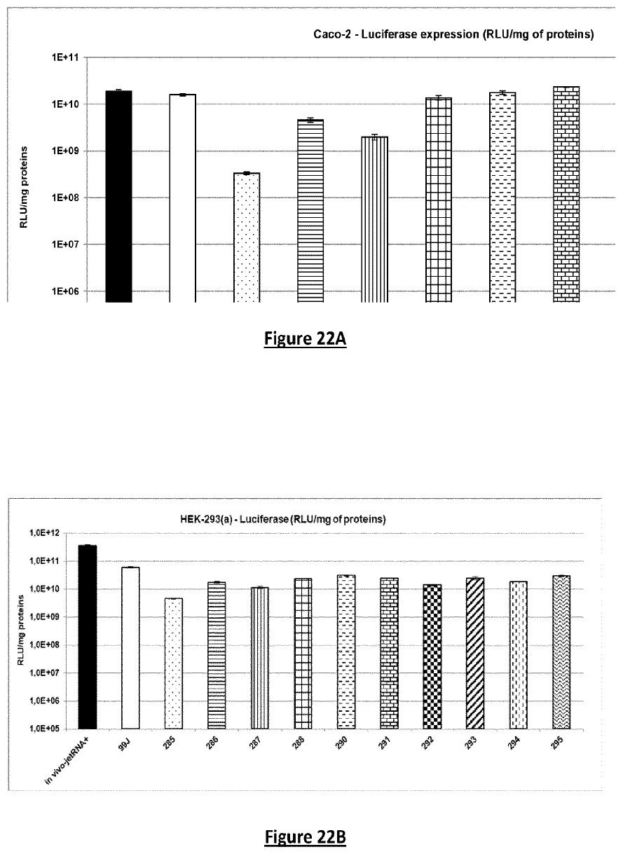

20 μg mRNA encoding Luciferase was administered into OF1 mice using in vivo-jetRNA®+ or LNP99F through intraperitoneal injection. Complexes were formed with a mRNA/in vivo-jetRNA*+ratio of 1:2 (μg(mRNA):μL(reagent)) in mRNA Buffer. Luciferase expression was assessed 24 h post-injection. With LNP 99F, luciferase expression (>1·10⁴ RLU/mg of proteins) was observed in all the organs collected (lungs, liver, spleen, kidneys, uterus, ovaries, mesenteric nodes, intestinal nodes, intestine, stomach and pancreas). Highest expression (>1·106 RLU/mg of proteins) were observed in spleen, uterus, ovaries and pancreas similar with mRNA/in vivo-jetRNA® complexes (FIGS. 23A and 23B).

9.8 Vaccination with OVA mRNA

The inventors evaluated vaccination with OVA mRNA using LNP 161 (see Table 11 below).

TABLE 11
Chemical composition of screened LNP 161

| | | | | | | | | | | | NA con- | | | | | |
|---|---|---|---|---|---|---|---|---|---|---|---|---|---|---|---|---|
| LNP | Cationic | | Ionizable | | Helper | | Stabilizing | | PEG-Lipid | | centration | | Size | PDI | Zeta | |
| No | lipid (mM) | | lipid (mM) | | Lipid (mM) | | lipid (mM) | | (mM) | | (ng/μL) | | (nm) | (—) | (mV) | EE % |
| 161 | W21.7 | 4.00 | DODMA | 3.00 | DPyPE | 1.00 | Choles- | 1.85 | DSG- | 0.15 | OVA | 200 | 40 ± 1 | 0.188 | +11 | 99.6 |
| | | | | | | | terol | | PEG 2k | | mRNA | | | | | |

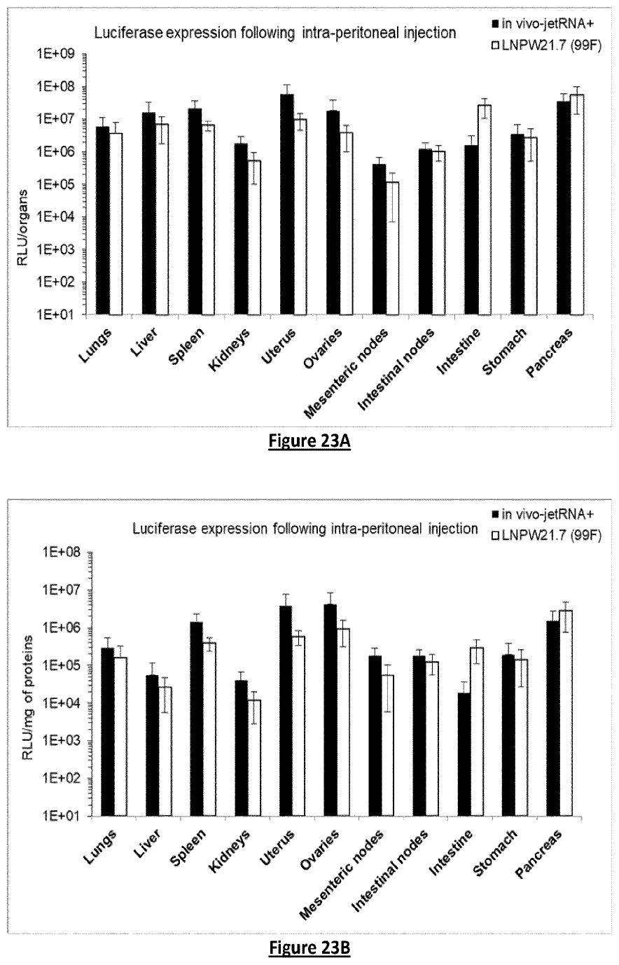

Mice were immunized intramuscularly with 5 μg mRNA coding for OVA using in vivo-jetRNA©+ or LNP161 W21.7 (200 ng/μL of mRNA) or PBS at week 0. At week 2, all mice received a boost of vaccination. Sera were collected from all the mice at week 3 for antibody responses. High humoral immune response following vaccination with LNP161 was obtained (˜60 μg/ml of anti-OVA IgG) similar to mRNA/in vivo-jetRNA®+ complexes (FIG. 24).

### 9.9 Cellular Distribution

The inventors evaluated cellular distribution in lungs and spleen using LNP 161 (see Table 12 below).

TABLE 12
Chemical composition of screened LNPs 190B, 222, 190C, 222B

| | | | | | | | | | | | NA con- | | | | | |
|---|---|---|---|---|---|---|---|---|---|---|---|---|---|---|---|---|
| LNP | Cationic | | Ionizable | | Helper | | Stabilizing | | PEG-Lipid | | centration | | Size | PDI | Zeta | |
| No | lipid (mM) | | lipid (mM) | | Lipid (mM) | | lipid (mM) | | (mM) | | (ng/μL) | | (nm) | (—) | (mV) | EE % |
| 190B | W21.7 | 4.00 | DODMA | 3.00 | DPyPE | 1.00 | Choles- | 1.85 | DSG- | 0.15 | GFP | 250 | 44 ± 2 | 0.184 | +12 | 100 |
| | | | | | | | terol | | PEG 2k | | mRNA | | | | | |
| 222 | — | — | ALC- | 4.630 | DSPC | 0.940 | Choles- | 4.270 | DMG- | 0.160 | GFP | 250 | 44 ± 1 | 0.179 | −2 | 88.4 |
| | | | 0315 | | | | terol | | PEG | | mRNA | | | | | |
| 190C | W21.7 | 4.00 | DODMA | 3.00 | DPyPE | 1.00 | Choles- | 1.85 | DSG- | 0.15 | GFP | 250 | 54 ± 4 | 0.106 | +14 | 99.6 |
| | | | | | | | terol | | PEG 2k | | mRNA | | | | | |
| 222B | — | — | ALC- | 4.630 | DSPC | 0.940 | Choles- | 4.270 | DMG- | 0.160 | GFP | 250 | 50 ± 1 | 0.107 | −3 | 64.4 |
| | | | 0315 | | | | terol | | PEG | | mRNA | | | | | |

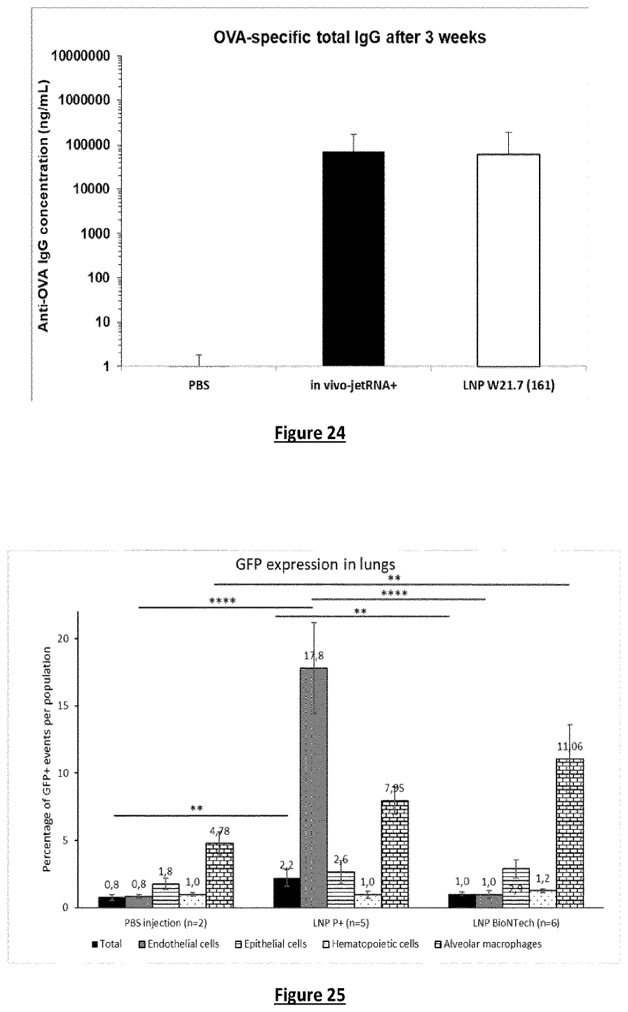

10 μg mRNA encoding eGFP was administered into OF1 mice using LNP190B (W21.7) or comparative LNP222 (Comirnaty-like formulation, BioNTech COVID's vaccine) through retro-orbital injection. GFP expression was assessed by flow cytometry 23 h post-injection for lung cells and 4 h post-injection for spleen cells (FIG. 25). Higher transfection efficiency was observed with LNP190B (2.2%) compared to comparative LNP222 (1%) in lung cells (p<0.001). In the lung, LNP190B mainly targeted endothelial cells (17.8% of endothelial cells express GFP, p<0.0001) and alveolar macrophages (7.9% of alveolar macrophages express GFP) whereas comparative LNP222 only targeted alveolar macrophages (11·1%, p<0.001). In the spleen, no GFP expression was observed in total cells for both LNPs (FIG. 26). When looking at each cell type, LNP190C mainly target dendritic cells like LNP222B.

### 9.10 DNA-LNP (Size, Zeta, Encapsulation, in Vitro and in Vivo)

The inventors evaluated the properties (charge, encapsulation efficiency, in vitro and in vivo transfection) of DNA-LNPs (see Table 13 below).

TABLE 13
Chemical composition of screened LNPs 109C, A32, 99G, 191

| | | | | | | | | | | | | NA con- | | | | | |
|---|---|---|---|---|---|---|---|---|---|---|---|---|---|---|---|---|---|
| | LNP | Cationic | | Ionizable | | Helper | | Stabilizing | | PEG-Lipid | | centration | | Size | PDI | Zeta | |
| | No | lipid (mM) | | lipid (mM) | | Lipid (mM) | | lipid (mM) | | (mM) | | (ng/μL) | | (nm) | (—) | (mV) | EE % |
| DNA | 109C | W21.7 | 4.00 | DODMA | 3.00 | DPyPE | 1.00 | Choles- | 1.85 | DSG- | 0.15 | GFP | 50 | 73 ± 2 | 0.038 | +16 | 100.0 |
| in | | | | | | | | terol | | PEG 2k | | DNA | | | | | |
| vitro | A32 | DOTAP | 3.00 | DODMA | 3.00 | — | — | Choles- | 7.92 | DMG- | 0.08 | GFP | 176 | 111 ± 15 | 0.637 | +15 | 99.4 |
| | | | | | | | | terol | | PEG | | DNA | | | | | |
| DNA | 99G | W21.7 | 4.00 | DODMA | 3.00 | DPyPE | 1.00 | Choles- | 1.85 | DSG- | 0.15 | Fluc | 200 | 44 ± 2 | 0.156 | +12 | 87.8 |
| in | | | | | | | | terol | | PEG 2k | | mRNA | | | | | |
| vivo | 191 | W21.7 | 4.00 | DODMA | 3.00 | DPyPE | 1.00 | Choles- | 1.85 | DSG- | 0.15 | FLuc | 200 | 55 ± 3 | 0.086 | +12 | 89.0 |
| | | | | | | | | terol | | PEG 2k | | DNA | | | | | |

### In Vitro Transfection

HEK-293 cells were transfected with jetPRIME©/eGFP DNA complexes (positive control) or LNP109C (W21.7) or LNP A32 (LNP positive control from the literature; Zhu et al., Nature Comm. 2022, 13, 4282). Transfection efficiency was assessed 24 hours post-transfection by flow cytometry. As expected, high transfection efficiency was observed with the positive control jetPRIME© (>80% of GFP) and a good transfection efficiency was observed with LNP A32 with up to 65% of GFP expression with 100 ng of DNA. Higher transfection efficiency was observed with LNP 109C (>60% with 10 ng and >70% with 25, 50 and 100 ng of DNA) compared to LNP 32A (FIG. 27).

### In Vivo Transfection

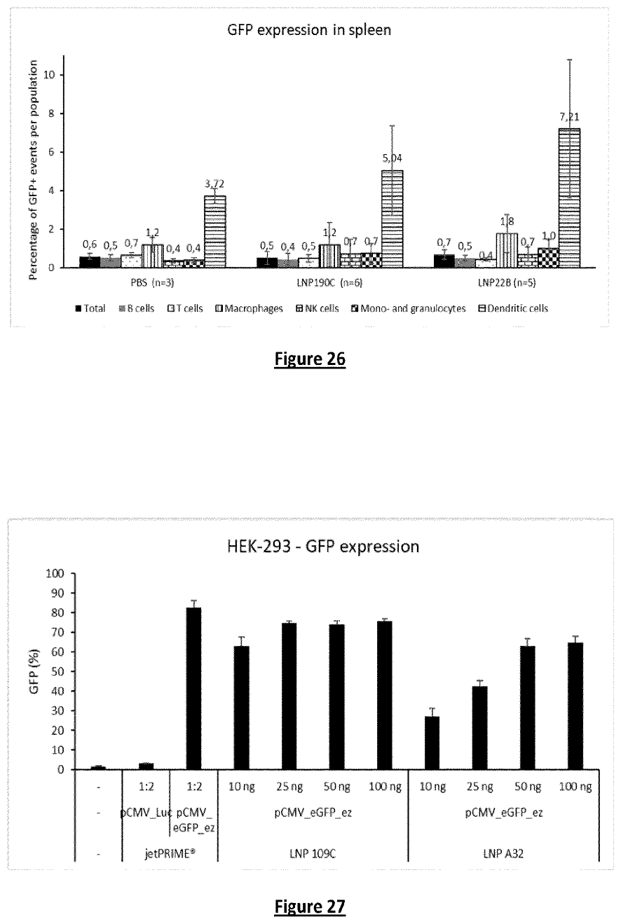

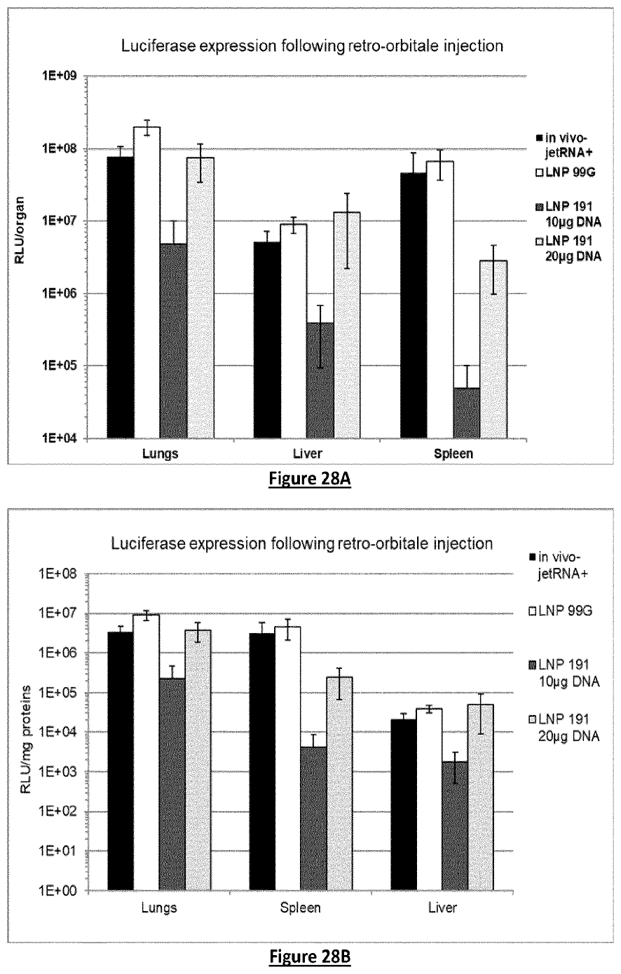

10 or 20 μg mRNA or DNA encoding Luciferase was administered into OF1 mice using in vivo-jetRNA© or LNP99G (mRNA) or LNP1941 (DNA) through intra-veinous injection. Complexes were formed with a mRNA/in vivo-jetRNA®+ ratio of 1:2 (μgmRNA:μLreagent) in mRNA Buffer. Luciferase expression was assessed 24 h post-injection. Lower luciferase expression was observed in all the organs collected (lungs, liver, spleen) with 10 μg of DNA-LNP191 (n=6) compared to 10 μg of mRNA-LNP99G (n=6). Similar luciferase expression was observed in lungs and liver with 20 μg of DNA-LNP191 (n=2) compared to 10 μg of mRNA-LNP99G but with a lower expression in the spleen (FIGS. 28A and 28B).

9.11 saRNA-LNP (Size, Zeta and Encapsulation)

The inventors evaluated the properties (size, charge, encapsulation efficiency) of saRNA-LNPs (see Table 14 below).

TABLE 14
Chemical composition of screened LNPs 215, 216 and 284

| | | | | | | | | | | | NA con- | | | | | |
|---|---|---|---|---|---|---|---|---|---|---|---|---|---|---|---|---|
| | Cationic | | Ionizable | | Helper | | Stabilizing | | PEG-Lipid | | centration | | Size | PDI | Zeta | |
| LNP No | lipid (mM) | | lipid (mM) | | Lipid (mM) | | lipid (mM) | | (mM) | | (ng/μL) | | (nm) | (—) | (mV) | EE % |
| 215 | W21.7 | 4.00 | DODMA | 3.00 | DPyPE | 1.00 | Choles- | 1.85 | DSG- | 0.15 | GFP | 50 | 36 ± 3 | 0.170 | +16 | 91.2 |
| | | | | | | | terol | | PEG 2k | | saRNA | | | | | |
| Comparative | — | — | Dlin-MC3- | 2.75 | DSPC | 0.55 | Choles- | 2.118 | DSG- | 0.083 | GFP | 50 | 42 ± 1 | 0.137 | −1 | 89.7 |
| 216 | | | DMA | | | | terol | | PEG 2k | | saRNA | | | | | |
| 284 | W21.7 | 4.00 | DODMA | 3.00 | DPyPE | 1.00 | Choles- | 1.85 | DSG- | 0.15 | nLUC | 50 | 87 ± 2 | 0.097 | +12 | 91 |
| | | | | | | | terol | | PEG 2k | | saRNA | | | | | |

LNP were formulated with AldGFP saRNA™ (8.6 kb) with W21.7 (LNP215) or comparative Onpattro-like formulation (LNP216). Similar size (40 nm) and encapsulation efficiency (90%) were obtained for both LNP formulation. As expected LNPs with W21.7 and saRNA were slightly charged whereas Onpattro-like formulation with saRNA was neutral.

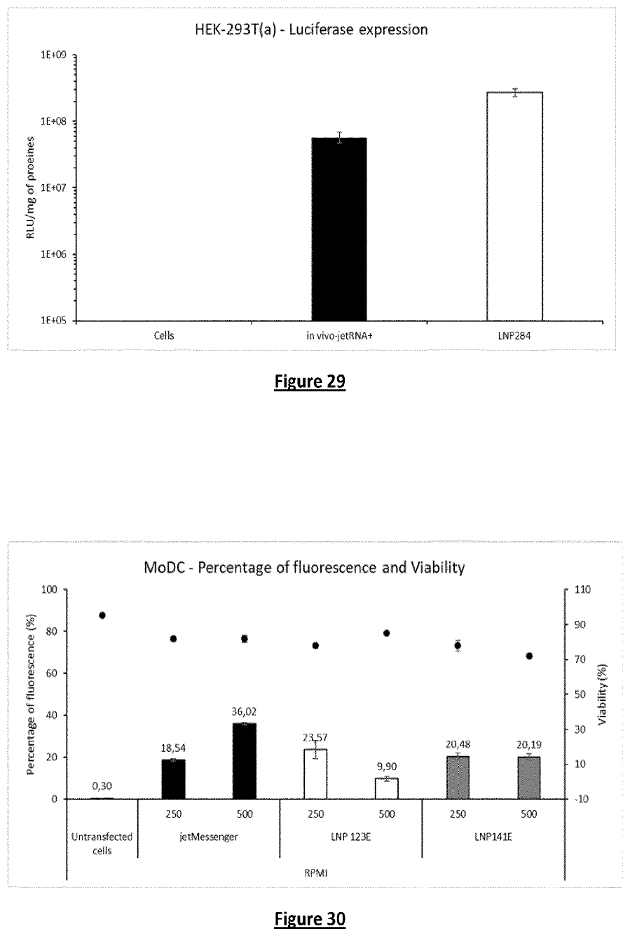

LNP were also formulated with AldnLUC saRNA™ (8.4 kb) with W21.7 (LNP284). Good size (87 nm) and good encapsulation efficiency were obtained. As expected, saRNA-LNP was slightly positively charged (+12 mV) (FIG. 29).

HEK-293 cells were transfected with in vivo-jetRNA8+/nLUC saRNA complexes (positive control) or LNP284. Transfection efficiency was assessed 24 hours post-transfection by luminescence. As expected, good luciferase expression was observed with the positive control in vivo-jetRNA®+(6.107 RLU/mg of proteins) and higher luciferase expression was observed with LNP284 (3.108 RLU/mg of proteins).

### 9.12 Monocyte-Derived Dendritic Cells (MoDCs) Transfection

The inventors evaluated MoDCs transfection using LNPs 123E and 141E (see Table 15 below).

TABLE 15
Chemical composition of screened LNPs 123E and 141E

| | | | | | | | | | | | NA con- | | | | | |
|---|---|---|---|---|---|---|---|---|---|---|---|---|---|---|---|---|
| LNP | Cationic | | Ionizable | | Helper | | Stabilizing | | PEG-Lipid | | centration | | Size | PDI | Zeta | |
| No | lipid (mM) | | lipid (mM) | | Lipid (mM) | | lipid (mM) | | (mM) | | (ng/μL) | | (nm) | (—) | (mV) | EE % |
| 123E | W21.7 | 4.000 | DODMA | 3.000 | DPγPE | 1.000 | Choles- | 1.850 | DSG- | 0.150 | eGFP | 100 | 64 ± 4 | 0.109 | +15 | 98.4 |
| | | | | | | | terol | | PEG 2k | | mRNA | | | | | |
| 141E | W21.7 | 4.000 | DODMA | 3.000 | DPγPE | 1.000 | Choles- | 1.700 | DSG- | 0.300 | eGFP | 100 | 36 ± 3 | 0.212 | +12 | 98.9 |
| | | | | | | | terol | | PEG 2k | | mRNA | | | | | |

MoDCs cells were transfected with jetMESSENGER®/eGFP mRNA complexes (positive control) or LNP123E (0.15 mM PEG) or 141E (0.3 mM PEG). Transfection efficiency was assessed 24 hours post-transfection by flow cytometry. GFP expression was observed with both LNPs (123E and 141E) with a similar expression compared to jetMessenger. Smaller LPNs (LNP141E, 36 nm) seemed to give more consistent GFP expression with different amount of mRNA compared to bigger LNPs (LNP123E, 64 nm) (FIG. 30).

### 9.13 Experimental Part

### Cell Lines

### Caco-2 Cells

Human epithelial cells from colorectal adenocarcinoma (Caco-2) were grown on cell flask coated with fibronectin (0.05 mg/ml) and cultured in DMEM glucose 4.5 g/L supplemented with Fetal bovine serum (FBS, 20%), Na Pyruvate (1%), L-Glutamine (1%), non-essential amino acids (AANE, 1%) and Penicillin-Streptomycin (1%).

### HEK-293(a) Cells

Human embryonic kidney cells (HEK-293(a)) were grown on cell flask coated with fibronectin (0.05 mg/ml) and cultured in MEM Eagle supplemented with Fetal bovine serum (FBS, 10%), L-Glutamine (1%), non-essential amino acids (AANE, 1%) and Penicillin-Streptomycin (1%).

### Monocyte Isolation and Differentiation in Dendritic Cells

Human primary monocytes from healthy donors were isolated from peripheral blood by magnetic activated cell sorting (positive selection via CD14 Microbeads, Miltenyi Biotec, 130-050-201) and used directly. Monocytes were cultivated in X-VIVO 15 with gentamicin (Lonza, BE02-060F) at 2 to 4×10⁶ cells/mL. To induce the differentiation in dendritic cells, 500 IU/mL IL-4 and 1,000 IU/mL GM-CSF were added. The cells were incubated 3 to 4 days at 37° C. and 5% CO₂ then the medium was changed, and cells were re-incubated for 2 to 3 days at 37° C. and 5% CO₂. After 5 to 6 days of incubation, the differentiation was checked by flow cytometry with CD11c/CD14 antibodies.

### In Vitro Transfection

Fluc mRNA Transfection of Adherent Cell Lines

For transfection experiments, 4×10⁴ Caco-2 cells or 5×10⁴ HEK-293(a) cells were seeded per well of 24-well plates in complete medium 1 day before transfection. On the day of transfection, in vivo-jetRNA®+/Fluc mRNA complexes were prepared according to the manufacturers' recommendations. Briefly, transfection with in vivo-jetRNA®+was performed as described: 500 ng of Fluc-encoding mRNA (per well of 24-well plate) were first diluted in the provided mRNA Buffer, followed by the mixing-in of 1 μl in vivo-jetRNA®+. Following an incubation of 15 minutes at room temperature, in vivo-jetRNA®+complexes or 500 ng of LNP X¹ (a list of the different LNP may be found in Table 3) were simply added dropwise to cells in their complete growth medium. Transfection efficiency was assessed 24 hours post-transfection by luminescence reading.

### GFP DNA Transfection of HEK-293(a) Cells

For transfection experiments, HEK-293(a) cells were seeded at 12 500 cells per well of 96-well plates in complete medium 1 day before transfection. On the day of transfection, jetPRIME®/GFP DNA complexes were prepared according to the manufacturers' recommendations. Briefly, transfection with jetPRIME® was performed as described: 150 ng of GFP-encoding DNA (per well of 96-well plate) were first diluted in the provided jetPRIME® Buffer, followed by the mixing-in of 0.3 μl jetPRIME®. Following an incubation of 10 minutes at room temperature, jetPRIME® complexes or 10 to 100 ng of LNP X were simply added dropwise to cells in their complete growth medium. Transfection efficiency was assessed 24 hours post-transfection by flow cytometry.

Nluc saRNA Transfection of HEK-293T(a) Cells

For transfection experiments, 5×10⁴ HEK-293(a) cells were seeded per well of 24-well plates in complete medium 1 day before transfection. On the day of transfection, in vivo-jetRNA®+/nLUC saRNA complexes were prepared according to the manufacturers' recommendations. Briefly, transfection with in vivo-jetRNA®+was performed as described: 125 ng of nLUC saRNA (per well of 24-well plate) were first diluted in the provided mRNA Buffer, followed by the mixing-in of 1 μl in vivo-jetRNA®+. Following an incubation of 15 minutes at room temperature, in vivo-jetRNA®+ complexes or 125 ng of LNP X² (a list of the different LNP may be found in Table 3) were simply added dropwise to cells in their complete growth medium. Transfection efficiency was assessed 24 hours post-transfection by luminescence reading.

Gfp mRNA Transfection of MoDCs

On the day of transfection, jetMESSENGER®/GFP mRNA complexes were prepared according to the manufacturers' recommendations. Briefly, transfection with jetMESSENGER® was performed as described: 250 or 500 ng of eGFP-encoding mRNA (per well of 96-well plate) were first diluted in the provided mRNA Buffer, followed by the mixing-in of 0.25 or 0.5 μl jetMESSENGER®, respectively. Following an incubation of 15 minutes at room temperature, jetMESSENGER® complexes or 250 or 500 ng of LNPX were simply added to the plate and 187 500 cells of MoDCs in RPMI 1640 media (Sigma-Aldrich, R0883-500 mL)+10% FBS+1% 200 mM L-glutamine+2% penicillin/streptomycin were added per well of 96-well plates on top of the complexes in their growth medium. 4 hrs after 175 ul of complete growth medium were added. Transfection efficiency was assessed 24-hours post-transfection by flow cytometry.

### In Vivo Transfection: IP Injection

40 μg of mRNA encoding Luciferase was administered into OF1 mice using LNPs via intra-peritoneal (IP) injection. Complexes were formed with a mRNA/in vivo-jetRNA®+ratio of 1:2 (μgmRNA:μLreagent) in mRNA Buffer. Luciferase expression was assessed 24 h post-injection. The organs of interest were dissected, rinsed in PBS (×1) and mixed with an ULTRA-TURRAX™ homogenized. Each organ mix was frozen at −80° C., thawed and an aliquot of 0.5 mL was taken for luciferase analysis. The aliquot was centrifuged for 5 min at 12 000 rpm at 4° C. Luciferase enzyme activity was assessed on 5 μL of organ lysate supernatant using 100 μl of luciferin solution. The luminescence (expressed as RLU) was measured by using a luminometer and normalized per mg of organ protein with Pierce BCA Assay Protein Kit.

### Ova Vaccination

Mice were immunized intramuscularly with 5 μg mRNA coding for ovalbumine (OVA) or PBS at week 0. At week 2, all mice received a boost of vaccination. Sera were collected from all the mice on days 7, 14 and 21 for antibody responses. Blood was collected by retro-orbital puncture. Samples were centrifuged 15,000 g for 5 min and the supernatants were collected and stored at −80° C. and used for ELISA. OVA-specific antibody responses in immunized sera were determined by enzyme-linked immunosorbent assay (ELISA assay). 96-well plates were coated with 100 μL of coating buffer containing 1 μg/well Albumin from chicken egg white (Sigma) at 37° C. for 90 minutes and at 4° C. overnight. Plates were blocked with 4% bovine serum albumin solution in PBST at room temperature for 2 hours. Plates were incubated with diluted immunized mice sera at room temperature for 2 hours. For determination of OVA-specific antibody response, plates were incubated with goat anti-mouse IgG HRP (Southern Biotech) at 37° C. for 1 hour and the substrate tetramethylbenzidine (TMB) solution (Thermo-Fisher) was used to develop. The color reaction was quenched with stop solution (Thermo-Fisher) and the optical density was measured at a length 450 nm by FLUOstar® Omega microplate reader (BMG Labtech).

### Cellular Distribution

10 μg of mRNA encoding eGFP was administered into OF1 mice using LNPs via intravenous (retro orbital) injection.

Lungs were collected 23 hours after injection. Digest media was added containing 3 mg/mL of collagenase I in DMEM high glucose and lung were disrupted using scissors. After 45 minutes of incubation at 37° C., digested lungs were then passed through a 70 μm stainer, incubated with M-lyse buffer to remove red blood cells and resuspended in FACS buffer (PBS+2% FBS+2 mM EDTA). Cells were stained with Live/dead eFluor 506 (Thermofisher #15560607), anti-CD326 (EpCAM) VioBLue (Miltenyi Biotec #130-117-870), anti-CD31 PE-V10770 (Miltenyi Biotec #130-111-542), anti-CD45 APC-Vio770 (Miltenyi Biotec #130-110-800), anti-CD11b APC (Miltenyi Biotec #130-113-802), anti-F4/80 PerCP-V10700 (Miltenyi Biotec #130-118-466), anti-CD3 PE (Miltenyi Biotec #130-121-133). Samples were analyzed on the MACSQuant X flow cytometer (Miltenyi Biotec).

Spleens were collected 4 hours after injection. Spleens were manually dissociated by forcing spleens through a 70 μm strainer. The strainer was then washed cold PBS. Cells were centrifuged at 1500 rpm for 5 min at 4° C. then red blood cells were lysed by adding 2 mL M-lyse buffer to pellets for 10 min at room temperature. Lysis was stopped by adding 4 mL wash buffer, and then cells were centrifuged again. Cells were resuspended in FACS buffer (PBS+2% FBS+2 mM EDTA) and then stained with Live/dead eFluor 506 (Thermofisher #15560607), anti-CD19 VioBLue (Miltenyi Biotec #130-111-889), anti-CD11b PE-V10770 (Miltenyi Biotec #130-113-808), anti-CD11c APC-Vio770 (Miltenyi Biotec #130-110-841), anti-NKp46 APC (Miltenyi Biotec #130-112-359), anti-F4/80 PerCP-Vio700 (Miltenyi Biotec #130-118-466), anti-CD3 PE (Miltenyi Biotec #130-121-133). Samples were analyzed on the MACSQuant X flow cytometer (Miltenyi Biotec).

While the invention has been described in terms of various preferred embodiments, the skilled person will appreciate that various modifications, substitutions, omissions and changes may be made without departing from the scope thereof. Accordingly, it is intended that the scope of the present invention be limited by the scope of the following claims, including equivalents thereof.

### 9.14 Synthesis of Compounds

### Synthesis of W31 1-(2-((2,6-dimethyl-14-octadecyldotriacontan-9-yl)oxy)-2-oxoethyl)-3-methyl-1H-imidazol-3-ium bromide

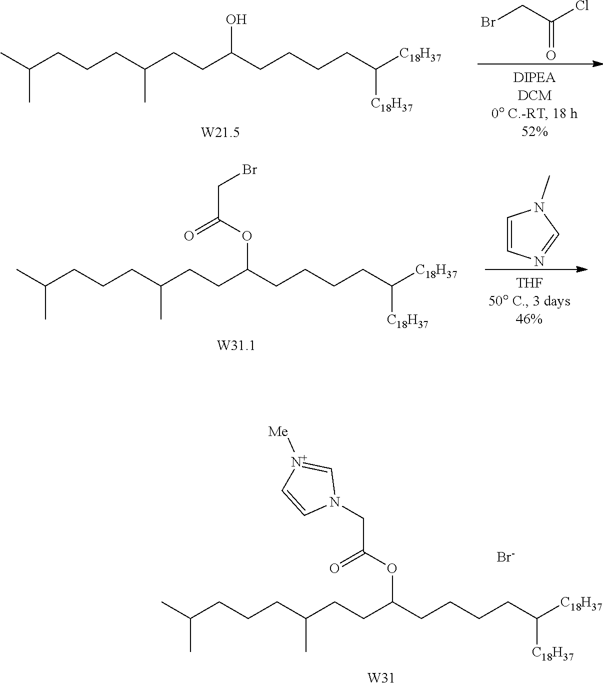

### Synthesis of Compound W31.1

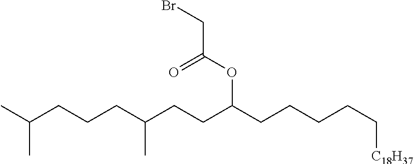

To a stirred solution of compound W21.5 (2.00 g, 2.68 mmol) in anhydrous DCM (20.0 mL) were sequentially added N,N-diisopropylethylamine (932 μL, 5.35 mmol) and bromoacetyl chloride (524 μL, 5.35 mmol) under argon at room temperature. The reaction mixture is stirred at room temperature overnight (18 h). The mixture was diluted with DCM and with water. The layers were separated, and the aqueous layer was extracted with DCM (50 mL). The combined organic layers were washed with brine, dried over Na₂SO₄, filtered, and concentrated to dryness. The residue was purified by column chromatography on silica gel (neat Heptane to heptane/DCM 9/1) to afford compound W31.1 (1.23 g, 52%) as pale-yellow oil. ¹H-NMR (CDCl₃, 298 K, 400 MHz) δ (ppm): 4.95-4.86 (m, 1H), 4.04 (s, 2H), 1.65-1.46 (m, 4H), 1.26 (s, 85H), 0.95-0.79 (m, 15H).

### Synthesis of Compound W31

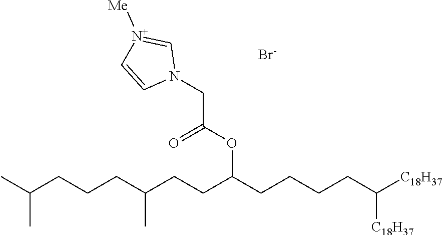

To a stirred solution of 1-methylimidazole (181 μL, 2.27 mmol) in THE (1.0 mL) was added a solution of compound W31.1 (500 mg, 567 μmol) in THE (4.0 mL) under argon. The pale orange solution was stirred at 50° C. for 3 days. The reaction mixture was diluted with DCM (50 mL) and the organic layer is washed with saturated aqueous NH₄Cl solution (3*50 mL). The aqueous layer was back extracted with DCM. The combined organic layers were washed with brine, dried over Na₂SO₄, filtered, and concentrated to dryness. Final purification is achieved by column chromatography (neat DCM to DCM/MeOH 9/1) to afford compound W31 as a white solid (251 mg, 46%). ¹H-NMR (CDCl₃, 298 K, 400 MHz) δ (ppm): 10.66 (s, 1H), 7.39 (s, 1H), 7.34 (s, 1H), 5.36 (s, 2H), 4.89 (t, J=6.1 Hz, 1H), 4.08 (s, 3H), 1.62-1.45 (m, 5H), 1.41-0.98 (m, 84H), 0.90-0.81 (m, 15H). ¹³C-NMR (CDCl₃, 298 K, 101 MHz) δ (ppm): 165.95, 139.74, 123.34, 122.76, 78.97, 77.48, 77.16, 76.84, 50.35, 39.37, 37.58, 37.18, 37.09, 36.92, 33.73, 32.87, 32.77, 32.50, 32.03, 30.30, 29.87, 29.83, 29.82, 29.77, 29.47, 28.05, 26.83, 26.73, 24.87, 22.82, 22.79, 22.71, 19.64, 19.60, 14.22.

### Synthesis of W32 3-butyl-1-(3-((2,6-dimethyl-14-octadecyldotriacontan-9-yl)oxy)-3-oxopropyl)-1H-imidazol-3-ium bromide

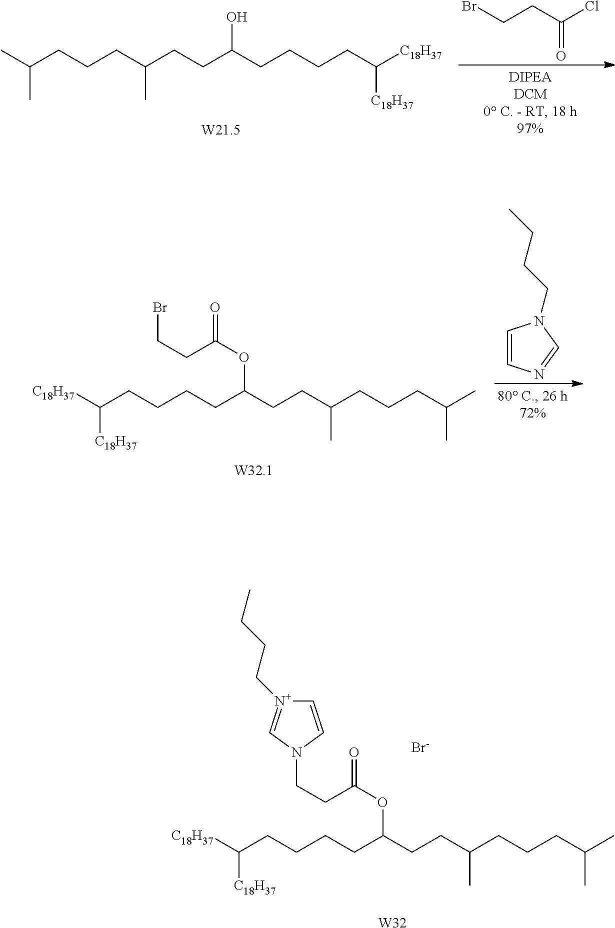

### Synthesis of Compound W32.1

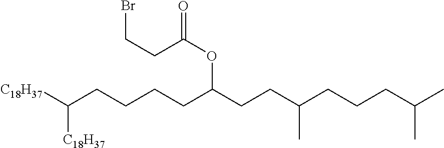

To an ice-chilled solution of compound W21.5 (1.00 g, 1.34 mmol) in anhydrous DCM (10.0 mL) are successively added dropwise N,N-Diisopropylethylamine (1.17 mL, 6.69 mmol) and 3-bromopropionyl chloride (966 mg, 5.35 mmol). The reaction mixture is allowed to warm up to reach room temperature overnight (18 h). At room temperature, methanol (5 mL) is added, and the reaction mixture is stirred at room temperature half an hour. The reaction mixture is diluted with DCM (50 mL) and water (20 mL) are added. The aqueous layer is extracted twice with DCM (2*30 mL). The combined organic layer is washed with brine, dried over Na₂SO₄, filtered off and concentrated to dryness. The residue is purified by column chromatography (heptane/DCM eluting system) to afford the pure expected W32.1 compound as a colorless oil (1.14 g, 97%). ¹H-NMR (CDCl₃, 298 K, 400 MHz) δ (ppm): 4.95-4.85 (m, 1H), 3.59 (t, J=6.8 Hz, 2H), 2.90 (t, J=6.8 Hz, 2H), 1.63-1.45 (m, 6H), 1.41-1.03 (m, 83H), 0.92-0.80 (m, 15H).

### Synthesis of Compound W32

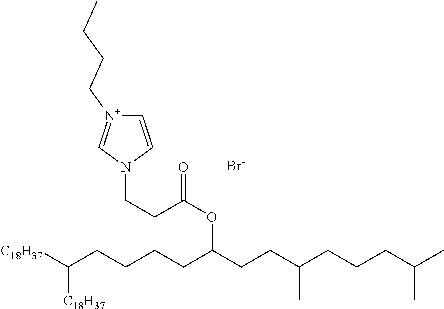

Compound W32.1 (400 mg, 0.467 mmol) is solubilized in 1-Butylimidazole (1·41 mL, 11·7 mmol). The reaction mixture is stirred at 80° C. overnight (26h). Once cooled down to RT, the reaction mixture is purified by column chromatography (neat DCM to DCM/MeOH 9/1) to afford the pure compound W32 as a white solid (303 mg, 72%). ¹H-NMR (CDCl₃, 298 K, 400 MHz) δ (ppm): δ 11.18 (s, 1H), 7.50 (s, 1H), 7.06 (s, 1H), 4.83 (m, 1H), 4.73 (t, J=5.6 Hz, 2H), 4.27 (t, J=7.5 Hz, 2H), 3.05 (t, J=5.6 Hz, 2H), 1.96-1.84 (m, 2H), 1.40 (h, J=7.5 Hz, 8H), 1.25 (s, 83H), 0.97 (d, J=7.4 Hz, 3H), 0.92-0.79 (m, 15H). ¹³C-NMR (CDCl₃, 298 K, 101 MHz) δ (ppm): 170.73, 138.17, 123.21, 121.28, 77.48, 77.37, 77.16, 76.84, 76.44, 49.93, 45.48, 39.28, 37.45, 37.11, 37.01, 35.19, 33.94, 33.80, 33.64, 32.71, 32.66, 32.45, 32.09, 31.92, 31.36, 31.27, 30.18, 29.75, 29.71, 29.66, 29.36, 27.93, 26.71, 26.64, 25.81, 25.78, 24.73, 22.71, 22.68, 22.60, 19.55, 19.50, 14.10, 13.43.

### Synthesis of W33 3-butyl-1-(4-((2,6-dimethyl-14-octadecyldotriacontan-9-yl)oxy)-4-oxobutyl)-1H-imidazol-3-ium bromide

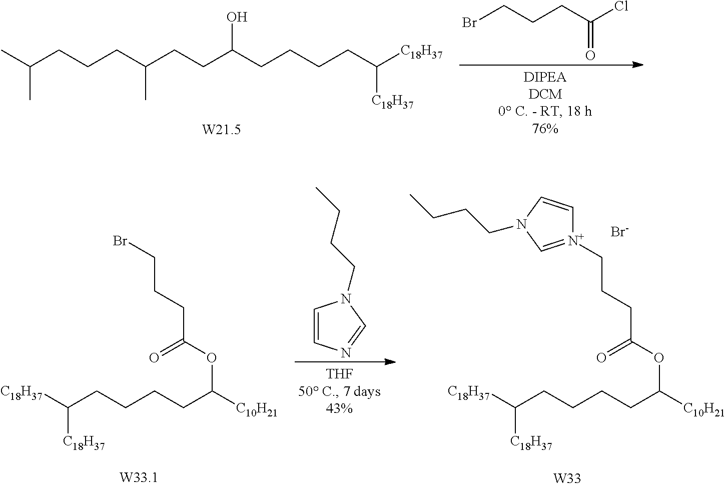

### Synthesis of Compound W33.1

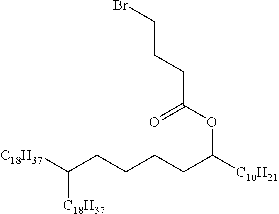

To an ice-chilled solution of compound W21.5 (1.50 g, 2.01 mmol) in anhydrous DCM (15.0 mL) were successively added N,N-Diisopropylethylamine (699 μL, 4.01 mmol) and 4-Bromobutyryl chloride (785 mg, 4.23 mmol). The reaction mixture was allowed to warm up to room temperature and is further stirred at this temperature overnight (21 h). The solution was diluted with DCM (50 mL) and extracted with water (50 mL). The organic layer was washed 3 times with water (3*50 mL) and dried over Na₂SO₄, filtered and concentrated to dryness. The residue is purified by column chromatography (heptane/DCM 8/2 to neat DCM) to afford the expected compound W33.1 as colorless oil (1.36 g, 76%). ¹H-NMR (CDCl₃, 298 K, 400 MHz) δ (ppm) 4.90-4.80 (m, 1H), 3.47 (t, J=6.5 Hz, 2H), 2.49 (t, J=7.2 Hz, 2H), 2.17 (p, J=6.8 Hz, 2H), 1.26 (s, 89H), 0.92-0.80 (m, 15H).

### Synthesis of Compound W33

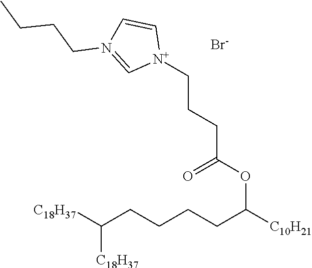

Compound W33.1 (300 mg, 335 μmol) was solubilized in THE (2.52 mL) and 1-Butylimidazole (166 mg, 1.34 mmol) was added to the mixture. The reaction was stirred at 50° C. under argon for 7 days. The reaction mixture was diluted with DCM (30 mL) and the organic layer was washed with saturated NH₄Cl (2*50 mL) and water (50 mL). The organic layer was dried over Na₂SO₄, filtered off and concentrated. The residue was purified by column chromatography (neat DCM to DCM/MeOH 9/1) to afford compound W33 as a white solid (140 mg, 43%). ¹H-NMR (CDCl₃, 298 K, 400 MHz) δ (ppm): 11.10 (s, 1H), 7.31 (d, J=1.7 Hz, 1H), 7.20 (t, J=1.7 Hz, 1H), 4.81 (m, 1H), 4.50 (t, J=7.5 Hz, 2H), 4.32 (t, J=7.5 Hz, 2H), 2.43 (t, J=6.9 Hz, 2H), 2.24 (p, J=7.0 Hz, 2H), 1.95-1.86 (m, 2H), 1.58-1.45 (m, 5H), 1.24 (s, 86H), 0.97 (t, J=7.4 Hz, 3H), 0.91-0.80 (m, 15H). ¹³C-NMR (CDCl₃, 298 K, 101 MHz) δ (ppm): 172.32, 138.85, 121.86, 121.33, 77.48, 77.16, 76.84, 50.12, 49.06, 39.39, 37.55, 37.25, 37.15, 34.08, 33.75, 32.82, 32.61, 32.24, 32.04, 31.54, 30.65, 30.30, 29.87, 29.84, 29.83, 29.78, 29.48, 28.06, 26.82, 25.98, 24.86, 22.83, 22.81, 22.72, 19.71, 19.65, 14.24, 13.55.

### Synthesis of W34 3-butyl-1-(5-((2,6-dimethyl-14-octadecyldotriacontan-9-yl)oxy)-5-oxopentyl)-1H-imidazol-3-ium bromide

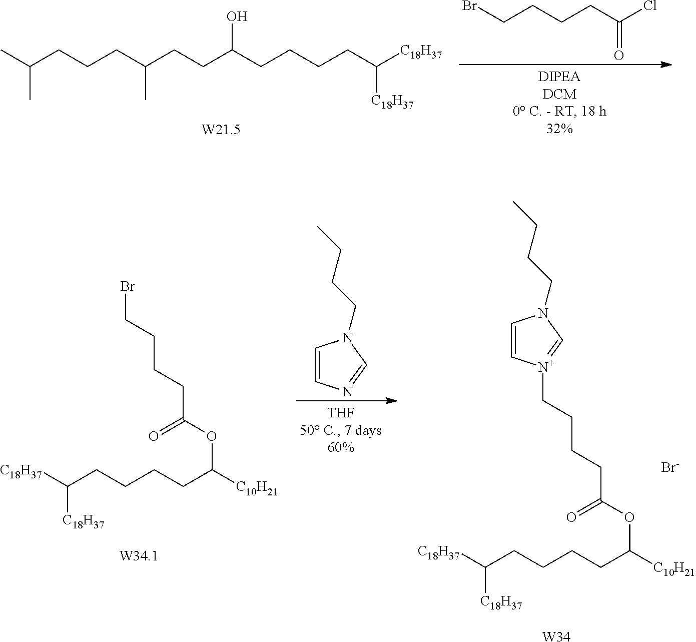

### Synthesis of Compound W34.1

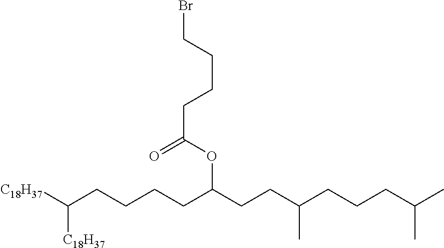

To an ice-chilled solution of compound W21.5 (1.00 g, 1.34 mmol) in anhydrous DCM (10.0 mL) were successively added N,N-Diisopropylethylamine (466 μL, 2.68 mmol) and 5-bromovaleryl chloride (378 μL, 2.82 mmol). The reaction mixture was allowed to warm up to room temperature overnight (18 h). The reaction was monitored by TLC (Heptane/DCM 7/3, vanillin). The reaction mixture is diluted with water (50 mL) and DCM (50 mL). The organic layer was dried over Na₂SO₄, filtered, and concentrated to dryness. The residue was purified by column chromatography (neat heptane to heptane/DCM 1/1) to afford compound W34.1 as a colorless oil (391 mg, 32%). ¹H-NMR (CDCl₃, 298 K, 400 MHz) δ (ppm): 4.90-4.80 (m, 1H), 3.41 (t, J=6.6 Hz, 2H), 2.33 (t, J=7.2 Hz, 2H), 1.96-1.87 (m, 2H), 1.83-1.73 (m, 2H), 1.63-1.00 (m, 89H), 0.97-0.80 (m, 15H).

### Synthesis of Compound W34

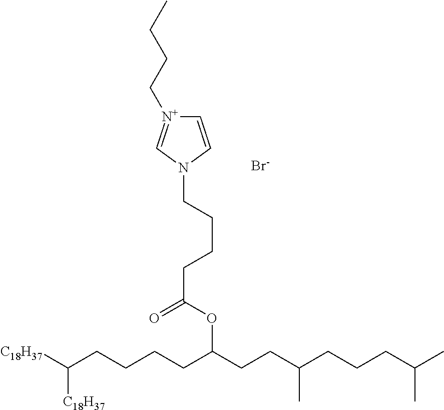

Compound W34.1 (391 mg, 429 μmol) was solubilized in THE (3.80 mL) and 1-Butylimidazole (311 μL, 2.58 mmol) was added. The reaction mixture was stirred at 50° C. under argon for 7 days. The reaction is diluted with EtOAc (50 mL) and washed with saturated aqueous NH₄Cl solution (3*50 mL). The organic layer was dried with Na₂SO₄ filtered and concentrated to dryness. The residue was purified by column chromatography (neat DCM to DCM/MeOH 9/1) to afford the pure compound W34 as a white solid (254 mg, 60%). ¹H-NMR (CDCl₃, 298 K, 400 MHz) δ (ppm): 11.03 (s, 1H), 7.27 (bs, 1H), 7.20 (bs, 1H), 4.84-4.76 (m, 1H), 4.43 (t, J=7.3 Hz, 2H), 4.33 (t, J=7.3 Hz, 2H), 2.37 (t, J=7.0 Hz, 2H), 2.08-1.86 (m, 7H), 1.66 (m, 2H), 1.57-1.01 (m, 88H), 0.97 (t, J=7.3 Hz, 3H), 0.93-0.80 (m, 15H). ¹³C-NMR (CDCl₃, 298 K, 101 MHz) δ (ppm): 172.96, 138.74, 121.59, 121.38, 50.08, 49.91, 39.40, 37.55, 37.25, 37.17, 34.11, 33.75, 33.44, 32.82, 32.60, 32.25, 32.04, 31.60, 30.30, 29.87, 29.84, 29.83, 29.78, 29.58, 29.48, 28.07, 26.82, 25.98, 24.86, 22.83, 22.81, 22.73, 21.52, 19.74, 19.68, 19.64, 14.24, 13.56.

### Synthesis of W35 3-butyl-1-(6-((2,6-dimethyl-14-octadecyldotriacontan-9-yl)oxy)-6-oxohexyl)-1H-imidazol-3-ium bromide

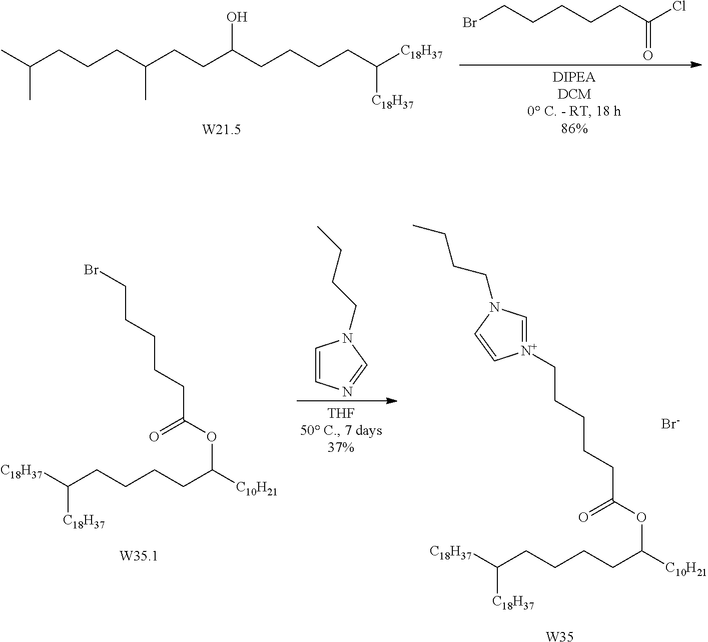

### Synthesis of Compound W35.1

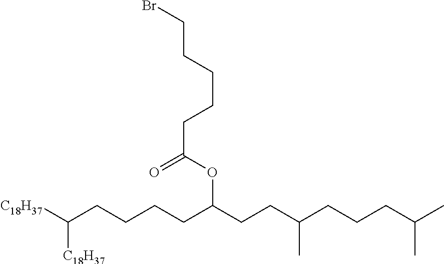

To an ice-chilled solution of compound W21.5 (500 mg, 669 μmol) in anhydrous DCM (5.0 mL) were successively added N,N-Diisopropylethylamine (233 μL, 1.34 mmol) and 6-bromohexanoyl chloride (216 μL, 1.41 mmol). The reaction mixture was stirred under argon overnight (18 h). The mixture was diluted with DCM (50 mL) and washed with water (3*50 mL), brine, dried over Na₂SO₃ and concentrated to dryness. The product was purified by column chromatography (Heptane/DCM 9/1 to 1/1) to afford compound W35.1 as a colorless oil (534 mg, 86%). ¹H-NMR (CDCl₃, 298 K, 400 MHz) δ (ppm): 4.88-4.00 (m, 1H), 3.40 (t, J=6.8 Hz, 2H), 2.31 (t, J=7.4 Hz, 2H), 1.94-1.86 (m, 2H), 1.66 (p, J=7.4 Hz, 2H), 1.60-1.43 (m, 5H), 1.41-1.00 (m, 86H), 0.95-0.81 (m, 15H).

### Synthesis of Compound W35

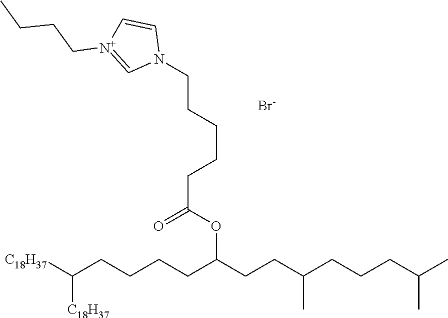

Compound W35.1 (500 mg, 541 μmol) was solubilized in anhydrous THE (4.8 mL) and 1-Butylimidazole (403 mg, 3.25 mmol) was added to the solution. The solution was stirred at 50° C. under argon for 7 days. The mixture was diluted with DCM (20 mL) and the organic layer was washed with saturated aqueous NH₄Cl solution (2*50 mL). The organic layer was dried over Na₂SO₄, filtered and concentrated. The product was purified by column chromatography (DCM to DCM/MeOH 9/1) to afford compound W35 as a white solid (207 mg, 37%). ¹H-NMR (CDCl₃, 298 K, 400 MHz) δ (ppm): 11.02 (s, 1H), 7.24 (s, 1H), 7.20 (t, J=1.6 Hz, 1H), 4.80 (t, J=6.5 Hz, 1H), 4.38 (t, J=7.4 Hz, 2H), 4.34 (t, J=7.4 Hz, 2H), 2.30 (t, J=7.3 Hz, 2H), 2.03-1.86 (m, 7H), 1.66 (m, 2H), 1.61-1.04 (m, 90H), 0.97 (t, J=7.3 Hz, 3H), 0.92-0.79 (m, 15H). ¹³C-NMR (CDCl₃, 298 K, 101 MHz) δ (ppm): 173.31, 138.94, 121.49, 121.30, 75.03, 74.96, 50.11, 49.95, 39.43, 37.57, 37.29, 37.20, 34.29, 34.14, 33.79, 32.84, 32.60, 32.56, 32.29, 32.06, 31.61, 30.31, 30.18, 29.88, 29.85, 29.84, 29.79, 29.49, 28.09, 26.84, 26.01, 25.79, 24.87, 24.26, 22.84, 22.82, 22.74, 19.78, 19.71, 19.67, 14.24, 13.57.

### Synthesis of W36 l-(2-((2,6-dimethyl-14-octadecyldotriacontan-9-yl)oxy)ethyl)-3-methyl-1H-imidazol-3-ium bromide

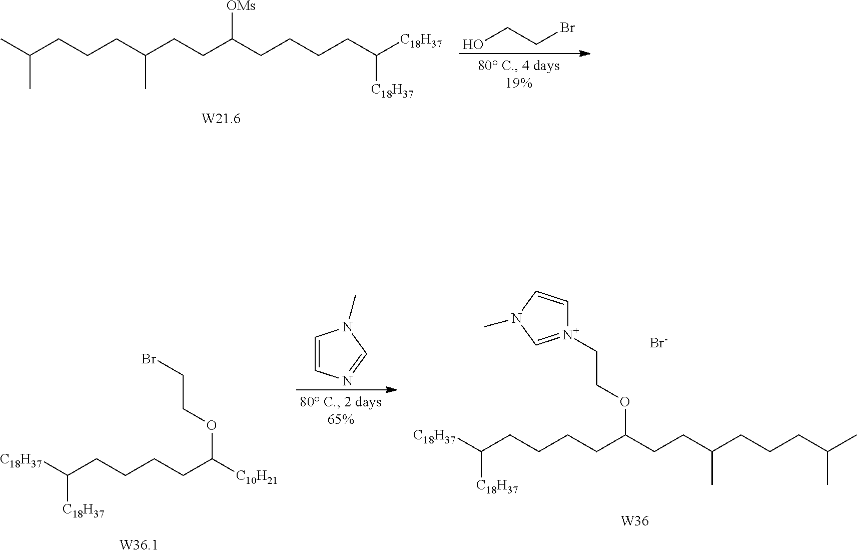

### Synthesis of Compound W36.1

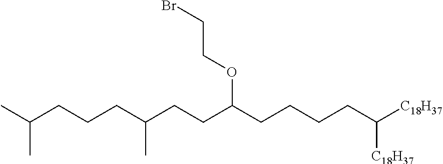

C₁₈H₃₇ Compound W21.6 (3.00 g, 3.63 mmol) was stirred at 80° C. in 2-bromoethanol (6.44 mL, 90.9 mmol) for 4 days. TLC monitoring (Heptane/DCM 7/3—Vaniline) indicates completion of the reaction. The reaction mixture is concentrated to dryness, and the residue is column chromatography (neat heptane to heptane/DCM 9/1) to afford compound W36.1 as a pale yellow oil (603 mg, 19%). ¹H-NMR (CDCl₃, 298 K, 400 MHz) δ (ppm): 3.74 (t, J=6.2 Hz, 2H), 3.44 (t, J=6.4 Hz, 2H), 3.30-3.21 (m, 1H), 1.50-1.02 (m, 90H), 0.97-0.75 (m, 15H).

### Synthesis of Compound W36

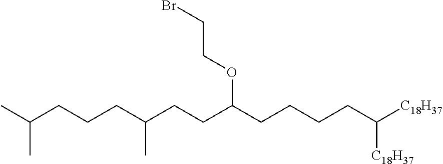

Compound W36.1 was stirred in 1-methylimidazole (700 μL, 8.78 mmol) at 80° C. for 2 days. The yellow solution was diluted with DCM (50 mL), and washed with 1M HCl (50 mL), brine, dried over Na₂SO₄, filtered, and concentrated to dryness. The residue was purified by column chromatography (DCM to DCM/MeOH 9/1) to afford compound W36 as a white solid (213 mg, 65%). ¹H-NMR (CDCl₃, 298 K, 400 MHz) δ (ppm): 10.95 (s, 1H), 7.43 (bs, 1H), 7.08 (bs, 1H), 4.60 (t, J=4.5 Hz, 2H), 4.06 (s, 3H), 3.78 (t, J=4.5 Hz, 2H), 3.27-3.19 (m, 1H), 1.51 (m, 1H), 1.40 (m, 4H), 1.34-0.95 (m, 84H), 0.92-0.78 (m, 15H). ¹³C-NMR (CDCl₃, 298 K, 101 MHz) δ (ppm): 139.01, 123.50, 121.93, 80.99, 77.48, 77.36, 77.16, 76.84, 67.03, 50.65, 39.43, 37.58, 37.35, 37.28, 36.70, 33.92, 33.78, 33.66, 33.59, 33.06, 33.02, 32.54, 32.05, 31.00, 30.91, 30.31, 29.89, 29.87, 29.85, 29.83, 29.78, 29.48, 28.09, 27.03, 26.87, 26.85, 25.92, 25.87, 24.90, 24.88, 22.83, 22.81, 22.73, 19.81, 19.76, 14.23, 1.13.

### Synthesis of W39 l-(2-((2,6-dimethyl-14-octadecyldotriacontan-9-yl)amino)-2-oxoethyl)-3-methyl-1H-imidazol-3-ium bromide

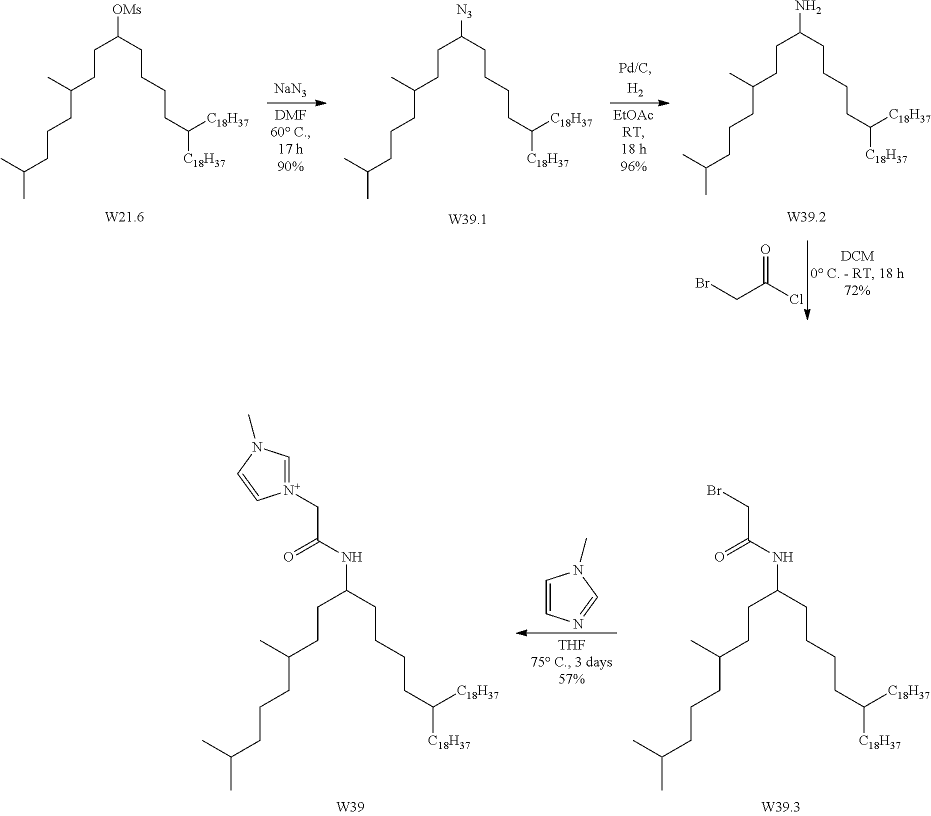

### Synthesis of Compound W39.1

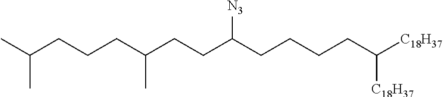

Sodium azide (74.2 mg, 1.14 mmol) was added to a solution of compound W21.6 (314 mg, 0.380 mmol) in anhydrous DMF (3.00 mL). The reaction mixture was stirred at 60° C. overnight (17 h). TLC monitoring (heptane/DCM 7/3, KMnO₄) indicated completion of the reaction. The reaction mixture was poured into water (20 mL) and the resulting suspension was diluted with EtOAc (50 mL). The organic layer is thoroughly washed with water (3*20 mL), dried over Na₂SO₄, filtered off and concentrated to dryness. The resulting colorless oil is purified by column chromatography (neat heptane to heptane/DCM 7/3) to afford compound W39.1 a colorless oil (265 mg, 90%). ¹H-NMR (CDCl₃, 298 K, 400 MHz) δ (ppm): 3.25-3.15 (m, 1H), 1.60-1.00 (m, 89H), 0.93-0.79 (m, 15H).

### Synthesis of Compound W39.2

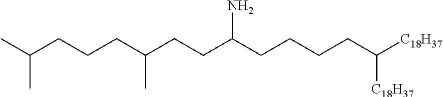

A solution of compound W39.1 (265 mg, 0.343 mmol) in EtOAc (3.18 mL) was degassed by bubbling argon for half an hour. Pd on activated charcoal (36.5 mg, 0.0343 mmol in Pd) was then added. The reaction was saturated with hydrogen by bubbling it straight into the solution for half an hour. The reaction is then stirred at room temperature under hydrogen atmosphere for 4 hours. The reaction mixture was filtered through a pad of celite (the cake was rinsed with 100 mL of EtOAc). Evaporation affords compound W39.2 as colorless oil (245 mg, 96%). ¹H-NMR (CDCl₃, 298 K, 400 MHz) δ (ppm): 2.73-2.61 (m, 1H), 1.26 (s, 91H), 0.94-0.78 (m, 15H).

### Synthesis of Compound W39.3

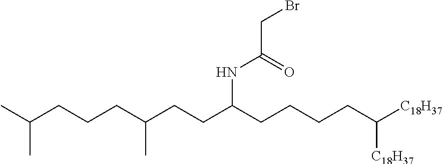

To a stirred solution of compound W39.3 (245 mg, 328 μmol) in anhydrous DCM (2.45 mL) was added triethylamine (54.9 μL, 394 μmol) at RT under argon. The resulting solution was cooled at 0° C. before bromoacetyl chloride (32.8 μL, 394 μmol) was added. The reaction mixture was allowed to warm up to room temperature overnight (18 h). TLC monitoring (DCM/MeOH 9/1, Vanillin or Ninhydrin) indicates completion of the reaction. The reaction mixture was diluted with DCM and was washed twice with water (2*50 mL), brine (50 mL), dried over Na₂SO₄, filtered off and concentrated to dryness. The residue was purified by column chromatography (Heptane/DCM 9/1 to neat DCM) to afford compound W39.3 as white solid (204 mg, 72%). (The two amide isomers are in equilibrium, and some NMR signals are consequently split). ¹H-NMR (CDCl₃, 298 K, 400 MHz) δ (ppm) 6.24 (dd, J=9.2, 3.8 Hz, 0.6H), 6.13 (dd, J=9.2, 3.8 Hz, 0.4H), 4.05 (s, 1.2H), 3.94-3.80 (m, 1.8H), 1.60-1.44 (m, 5H), 1.26 (s, 84H), 0.93-0.78 (m, 15H).

### Synthesis of Compound W39

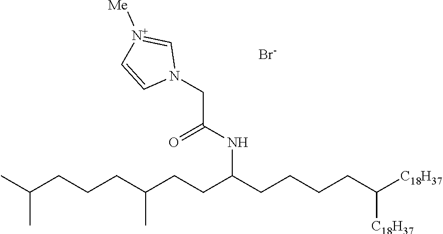

Compound W39.3 (200 mg, 231 μmol) is solubilized into anhydrous THE (2.0 mL). 1-Methylimidazole (1.39 mL, 17.4 mmol) was added, and the reaction mixture was stirred at 75° C. for 3 days. TLC monitoring (Heptane/DCM 1/1, KMnO₄) indicates completion of the reaction. The reaction mixture was diluted with DCM (50 mL) and poured into 0.1 M HCl solution (50 mL). The organic layer was washed with water, brine, dried over Na₂SO₄, filtered off and concentrated to dryness. The residue was purified by column chromatography (neat DCM to DCM/MeOH 9/1) to afford compound W39 as white solid (125 mg, 57%). ¹H-NMR (CDCl₃, 298 K, 400 MHz) δ (ppm): δ 10.97 (bs, 1H), 8.67 (d, J=8.7 Hz, 1H), 7.68 (s, 1H), 7.14 (s, 1H), 5.31 (s, 2H), 4.00 (s, 3H), 3.79-3.66 (m, 1H), 2.33 (bs, 4H), 1.58-0.95 (s, 85H), 0.92-0.74 (m, 15H). ¹³C-NMR (CDCl₃, 298 K, 101 MHz) δ (ppm): 164.07, 138.07, 123.98, 122.12, 77.16, 52.08, 51.03, 39.44, 37.62, 37.38, 37.27, 36.80, 34.84, 34.53, 33.90, 33.77, 33.74, 33.59, 33.52, 32.88, 32.80, 32.20, 32.05, 30.34, 29.90, 29.86, 29.84, 29.83, 29.78, 29.48, 28.08, 26.84, 26.81, 26.80, 26.77, 24.95, 24.90, 22.86, 22.81, 22.74, 19.73, 14.24.

### Synthesis of W43 3-butyl-1-(5-((2,6-dimethyl-14-octadecyldotriacontan-9-yl)oxy)pentyl)-1H-imidazol-3-ium bromide

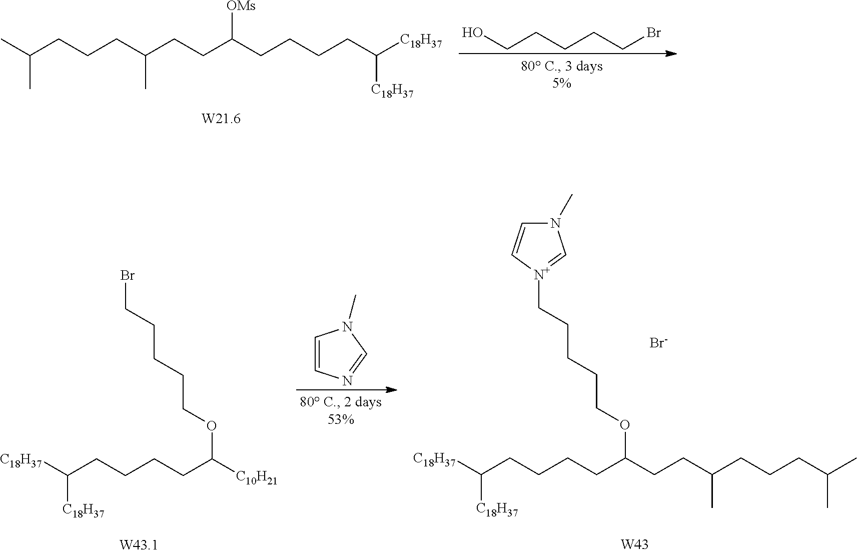

### Synthesis of Compound W43.1

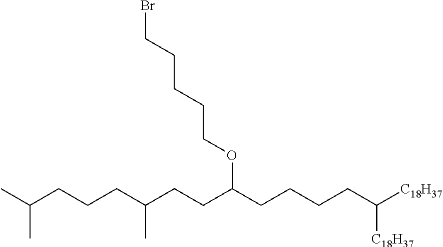

Compound W21.6 (2.10 g, 2.54 mmol) is stirred at 80° C. in 5-bromo-1-pentanol (10.6 g, 63.6 mmol) for 3 days. The brown solution was concentrated to dryness under high vacuum. The residue was purified by column chromatography (neat heptane to heptane/DCM 4/1) to afford compound W43.1 as yellow oil (107 mg, 5%). ¹H-NMR (CDCl₃, 298 K, 400 MHz) δ (ppm): 3.44-3.36 (t, J=6.8 Hz, 4H), 3.22-3.10 (m, 1H), 1.89 (p, J=6.9 Hz, 2H), 1.25 (s, 96H), 0.93-0.81 (m, 15H).

### Synthesis of Compound W43

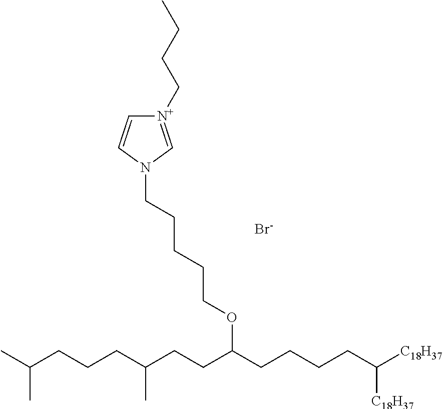

Compound W43.1 (107 mg, 119 μmol) was heated at 80° C. in 1-Butylimidazole (392 μL, 2.98 mmol). TLC monitoring (Heptane/DCM 9/1, Vanilline) indicated completion of the reaction. The yellow solution was diluted with DCM (50 mL). The organic layer was washed with brine (50 mL), dried over Na₂SO₄, filtered off and concentrated to dryness. The residue is purified by column chromatography (DCM to DCM/MeOH 9/1) to afford compound W43 (65.0 mg, 53%) as an off-white solid. ¹H-NMR (CDCl₃, 298 K, 400 MHz) δ (ppm): δ 11.21 (s, 1H), 7.15 (bs, 1H), 7.14 (bs, 1H), 4.41-4.31 (m, 4H), 3.40 (t, J=6.2 Hz, 2H), 3.19-3.11 (m, 1H), 2.01-1.85 (m, 4H), 1.64-1.01 (m, 95H), 0.98 (t, J=7.4 Hz, 3H), 0.91-0.80 (m, 15H). ¹³C-NMR (CDCl₃, 298 K, 101 MHz) δ (ppm): 138.53, 121.57, 80.13, 80.10, 77.48, 77.36, 77.16, 76.84, 68.26, 50.10, 49.98, 39.43, 39.42, 37.56, 37.33, 37.31, 34.11, 34.03, 33.88, 33.77, 33.08, 33.05, 32.64, 32.28, 32.01, 31.37, 31.28, 30.32, 30.27, 29.83, 29.80, 29.79, 29.74, 29.63, 29.44, 28.05, 27.08, 26.82, 26.81, 26.07, 26.03, 24.88, 24.87, 23.28, 22.80, 22.77, 22.71, 19.83, 19.80, 19.60, 14.19, 13.54.

### Synthesis of W44 1-(2,6-dimethyl-14-octadecyldotriacontan-9-yl)-2,3-dimethyl-1H-imidazol-3-ium chloride

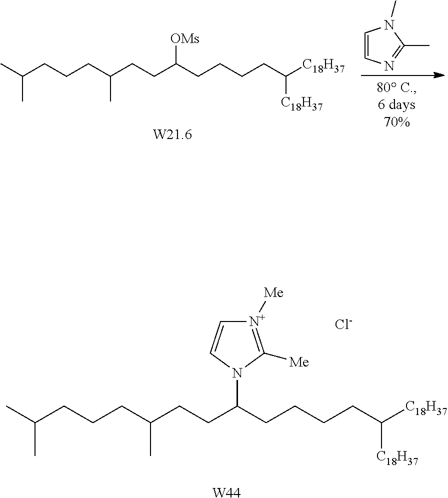

Compound W21.6 (250 mg, 303 μmol) was heated in 1,2-Dimethylimidazole (728 mg, 7.57 mmol) at 80° C. for 6 days. TLC monitoring (Heptane/DCM 1/1—Vanillin) indicates completion of the reaction. After cooling down to room temperature, the mixture was transferred, diluted with MeOH (5 mL) and placed in an ice bath. The mixture was slowly acidified with 3M HCl solution (3 mL) and evaporated to dryness. The residue was taken up in water (10 mL) and triturated at 0° C. The resulting precipitate was collected by filtration, rinsed with water and dried under vacuum. The residue is purified by column chromatography (neat DCM to DCM/MeOH 9/1) to afford compound W44 as a white solid (183 mg, 70%). ¹H-NMR (CDCl₃, 298 K, 400 MHz) δ (ppm): 8.16 (dd, J=6.0, 1.9 Hz, 1H), 7.18 (t, J=1.9 Hz, 1H), 4.19 (s, 3H), 4.12-4.05 (m, 1H), 2.78 (s, 3H), 1.91-1.66 (m, 4H), 1.54-1.43 (m, 1H), 1.42-0.97 (m, 84H), 0.91-0.78 (m, 15H). ¹³C-NMR (CDCl₃, 298 K, 101 MHz) δ (ppm): 143.41, 125.21, 117.18, 60.84, 39.34, 37.52, 37.04, 36.89, 36.61, 35.51, 35.34, 33.65, 33.38, 32.85, 32.66, 32.08, 30.36, 29.69, 29.35, 28.03, 26.79, 26.67, 24.82, 22.88, 19.53, 14.31, 10.89.

### Synthesis of W51 1-(2-((2,6-dimethyl-14-octadecyldotriacontan-9-yl)oxy)ethyl)-2,3-dimethyl-1H-imidazol-3-ium bromide

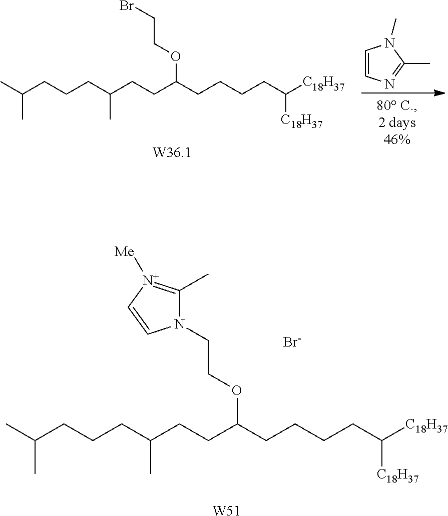

Compound W36.1 (300 mg, 351 μmol) is heated at 80° C. in 1,2-Dimethylimidazole (844 mg, 8.78 mmol) for 2 days. TLC monitoring (Heptane/DCM 9/1, Vanilline) indicates completion of the reaction. The pale-yellow suspension is diluted with DCM, and the organic layer was washed twice with 1M HCl aqueous solution (50 mL), brine (50 mL), dried over Na₂SO₄, filtered, and concentrated to dryness. The residue was purified by column chromatography (neat DCM to DCM/MeOH 9/1) to afford compound W51 as a white solid (152 mg, 46%). ¹H-NMR (CDCl₃, 298 K, 400 MHz) δ (ppm): 7.78 (d, J=2.0 Hz, 1H), 7.50 (t, J=2.4 Hz, 1H), 4.51 (t, J=4.8 Hz, 2H), 3.95 (s, 3H), 3.77 (t, J=4.8 Hz, 2H), 3.21-3.14 (m, 1H), 2.78 (s, 3H), 1.56-1.46 (m, 1H), 1.44-1.00 (m, 88H), 0.91-0.76 (m, 15H). ¹³C-NMR (CDCl₃, 298 K, 101 MHz) δ (ppm): 144.91, 122.60, 122.16, 80.88, 80.84, 77.48, 77.16, 76.84, 67.10, 49.65, 39.42, 37.58, 37.37, 37.27, 35.89, 33.94, 33.77, 33.60, 33.52, 33.04, 32.98, 32.56, 32.48, 32.05, 30.99, 30.82, 30.31, 29.89, 29.88, 29.85, 29.83, 29.78, 29.48, 28.09, 27.02, 26.87, 26.85, 25.83, 25.79, 24.90, 22.84, 22.81, 22.73, 19.83, 19.77, 14.23, 10.94.

### Synthesis of W54 1-(3-((2,6-dimethyl-14-octadecyldotriacontan-9-yl)thio)propyl)-3-methyl-1H-imidazol-3-ium chloride

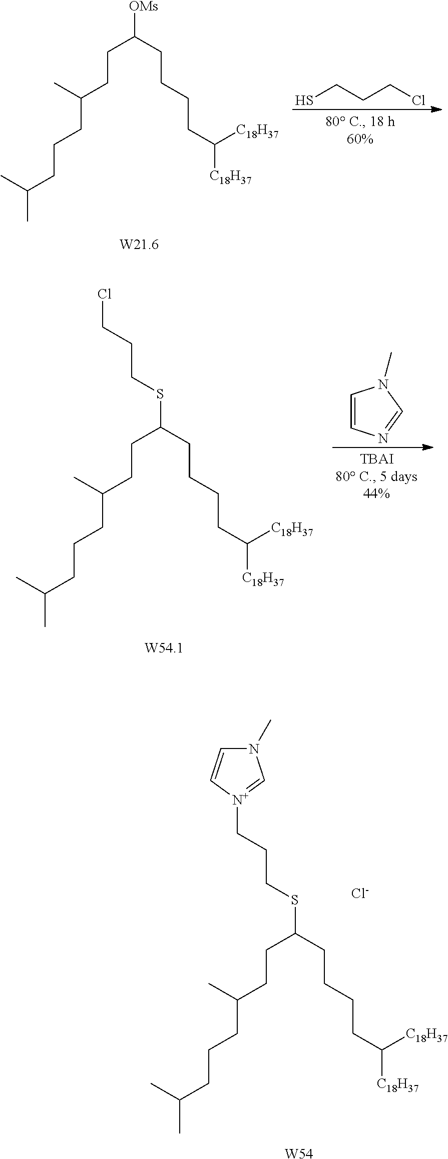

### Synthesis of Compound W54.1

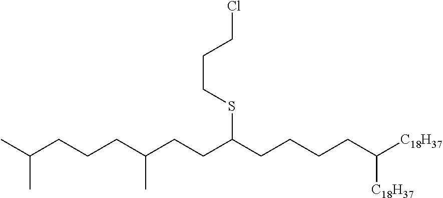

Compound W21.6 (1.50 g, 1.82 mmol) was dissolved in 3-Chloro-1-propanethiol (2.65 mL, 27.3 mmol) and the reaction mixture was heated at 80° overnight (18 h). TLC monitoring (Heptane/DCM 7/3—Vanilline) indicated completion of the reaction. The reaction mixture was concentrated to dryness. The residue was purified by column chromatography (neat heptane) to afford compound W54.1 as a colorless oil (910 mg, 60%). ¹H-NMR (CDCl₃, 298 K, 400 MHz) δ (ppm): 3.66 (t, J=6.3 Hz, 2H), 2.67-2.47 (m, 3H), 2.02 (p, J=6.7 Hz, 2H), 1.63-1.02 (m, 89H), 0.97-0.79 (m, 15H).

### Synthesis of Compound W54

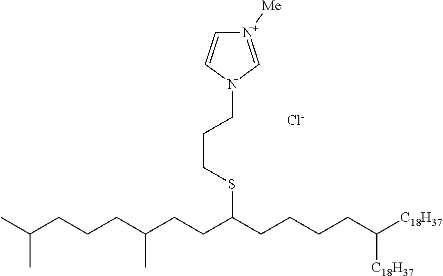

Compound W54.1 (250 mg, 298 μmol) and tetrabutylammonium iodide (22.0 mg, 59.5 μmol) were dissolved in 1-Methylimidazole (712 μL, 8.93 mmol) and the reaction mixture was heated at 80° C. for 5 days. TLC monitoring (Heptane 100%, Vanilline) indicates completion of the reaction. The yellow solution was diluted with DCM (50 mL), was washed twice with NH₄Cl saturated aqueous solution (2*50 mL), dried over Na₂SO₄, filtered off and concentrated to dryness. The residue was purified by column chromatography (neat DCM to DCM/MeOH 9/1) to afford compound W54.2 as a white and sticky solid (122 mg, 44%). ¹H-NMR (CDCl₃, 298 K, 400 MHz) δ (ppm): 10.90 (s, 1H), 7.28 (s, 2H), 4.47 (t, J=7.0 Hz, 2H), 4.11 (s, 3H), 2.65-2.46 (m, 3H), 2.32-2.13 (m, 4H), 1.58-1.00 (m, 87H), 0.91-0.77 (m, 15H). ¹³C-NMR (CDCl₃, 298 K, 101 MHz) δ (ppm): 139.16, 122.83, 122.06, 77.48, 77.16, 76.84, 48.73, 39.42, 37.57, 37.27, 36.90, 33.85, 33.76, 32.90, 32.04, 30.31, 30.17, 29.87, 29.83, 29.78, 29.48, 28.09, 27.40, 26.90, 26.84, 24.89, 22.84, 22.81, 22.74, 19.79, 14.24.

### Synthesis of W55 1-(3-((2,6-dimethyl-14-octadecyldotriacontan-9-yl)sulfonyl)propyl)-3-methyl-1H-imidazol-3-ium bromide

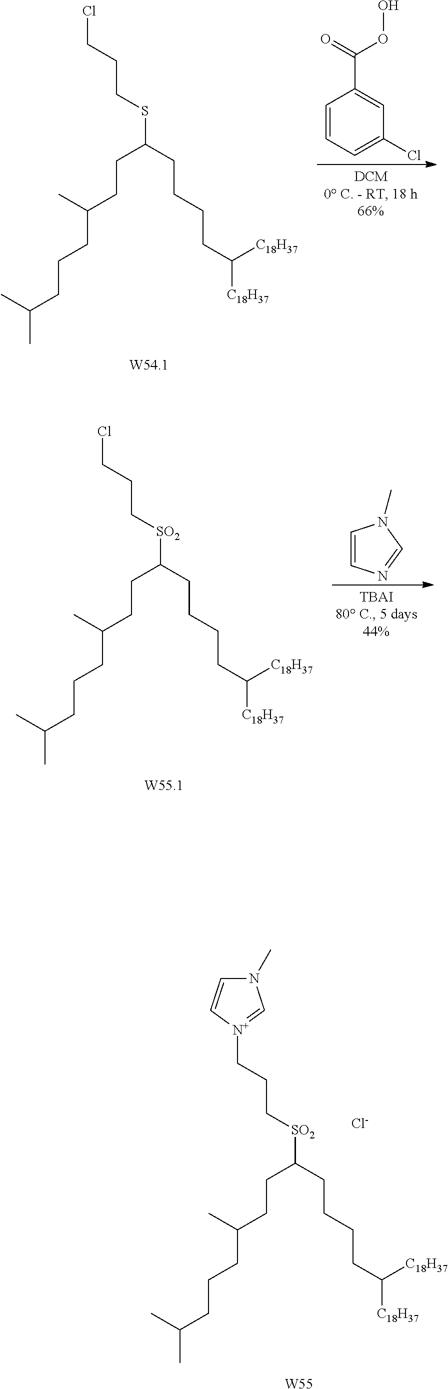

### Synthesis of Compound W55.1

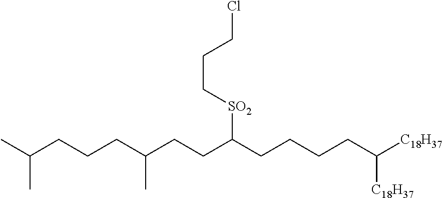

Compound 54.1 (400 mg, 476 μmol) was dissolved in DCM (2.4 mL). At 0° C., a solution 3-chloroperbenzoic acid (77%, 235 mg, 1.05 mmol) in DCM (2.4 mL) was added dropwise. The reaction mixture was stirred at room temperature under argon overnight (18 h). TLC monitoring (Heptane 100%, Vanilline) indicated completion of the reaction. The reaction mixture was diluted with DCM (20 mL), was washed with Na₂S₂O₃ saturated aqueous solution (50 mL). The aqueous layer was extracted twice with DCM (2*50 mL). The combined organic layers were washed with brine, dried over Na₂SO₄, filtered off and concentrated to dryness. The residue was purified by column chromatography (Heptane/EtOAc 97/3 to 92/8) to afford compound W55.1 as a colorless oil (272 mg, 66%). ¹H-NMR (CDCl₃, 298 K, 400 MHz) δ (ppm): 3.72 (t, J=6.0 Hz, 2H), 3.10 (t, J=7.4 Hz, 2H), 2.90-2.73 (m, 1H), 2.40-2.29 (m, 2H), 1.99-1.83 (m, 2H), 1.76-1.60 (m, 2H), 1.26 (m, 87H), 0.93-0.81 (m, 15H).

### Synthesis of Compound W55

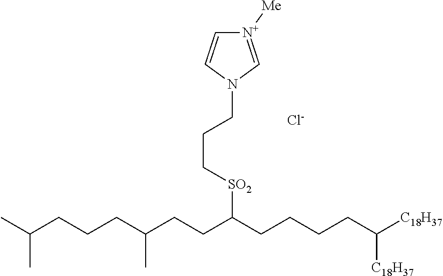

Compound W55.1 (270 mg, 310 μmol) and tetrabutylammonium iodide (22.9 mg, 61.9 μmol) were solubilized into 1-methylimidazole (740 μL, 9.29 mmol) and the reaction mixture was heated up at 80° C. for 3 days. TLC monitoring (Heptane/DCM 1/1, Vanillin) indicated completion of the reaction. The yellow solution was diluted with EtOAc (50 mL), washed with a saturated aqueous solution of NH₄Cl (2*50 mL). The layers were separated, and the aqueous layer was back extracted with EtOAc (50 mL). The combined organic layer was washed with brine (50 mL), dried over Na₂SO₄, filtered off and concentrated to dryness. The residue was purified by column chromatography (neat DCM to DCM/MeOH 9/1) to afford compound W55 as a white and sticky solid (119 mg, 40%). ¹H-NMR (CDCl₃, 298 K, 400 MHz) δ (ppm): 10.99 (s, 1H), 7.40 (s, 1H), 7.15 (s, 1H), 4.70 (t, J=7.1 Hz, 2H), 4.04 (s, 3H), 3.42-3.34 (m, 1H), 3.17-3.10 (m, 2H), 2.87-2.78 (m, 1H), 2.65 (t, J=6.5 Hz, 2H), 1.96-1.78 (m, 2H), 1.70-1.59 (m, 4H), 1.55-0.95 (m, 86H), 0.91-0.80 (m, 15H). ¹³C-NMR (CDCl₃, 298 K, 101 MHz) δ (ppm): 139.13, 122.74, 77.48, 77.36, 77.16, 76.84, 64.03, 63.91, 63.67, 59.33, 48.38, 46.28, 39.45, 39.38, 37.57, 37.46, 37.38, 37.33, 37.24, 37.13, 36.93, 36.88, 34.29, 34.10, 33.85, 33.75, 33.68, 33.14, 33.00, 32.70, 32.05, 30.32, 29.89, 29.86, 29.84, 29.79, 29.49, 28.09, 28.03, 27.89, 27.53, 26.95, 26.86, 26.79, 26.68, 25.54, 25.40, 24.90, 24.78, 24.39, 22.93, 22.84, 22.81, 22.74, 19.98, 19.72, 19.65, 19.51, 14.24, 13.85.

### Synthesis of compound W56: 3-(2,6-dimethyl-14-octadecyldotriacontan-9-yl)-1,2,4,5-tetramethyl-1H-imidazol-3-ium chloride

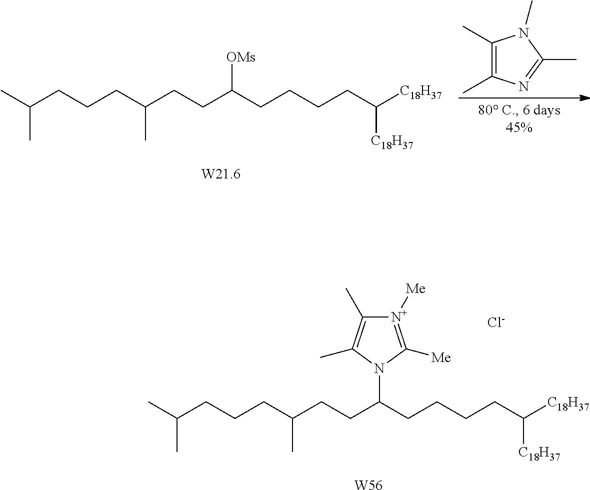

Compound W21.6 (250 mg, 303 μmol) is stirred at 80° C. in 1,2,4,5-Tetramethyl-1H-imidazole (940 mg, 7.57 mmol) for 6 days. After cooling down to room temperature, the mixture was diluted with MeOH (5 mL), 3M HCl aqueous solution (3 mL) was added at 0° C., and the mixture is stirred 5 minutes at 0° C. before being concentrated to dryness. The residue was triturated in water (10 mL) at 0° C. The resulting precipitate was collected by filtration and rinsed with water (30 mL). The product was purified by column chromatography (neat DCM to DCM/MeOH 9/1) to afford compound W56 as a white solid (122 mg, 45%). (The product was obtained as a mixture of atropoisomers, and some of the NMR signals was consequently split). ¹H-NMR (CDCl₃, 298 K, 400 MHz) δ (ppm): 4.24-4.12 (m, 0.6H), 4.10-3.91 (m, 3.4H), 2.92 (s, 1.2H), 2.86 (s, 1.8H), 2.28 (s, 4.4H), 2.17 (s, 1.6H), 1.92-1.75 (m, 4H), 1.56-1.43 (m, 1H), 1.43-0.97 (m, 84H), 0.92-0.78 (m, 15H). ¹³C-NMR (CDCl₃, 298 K, 101 MHz) δ (ppm): 39.14, 37.43, 36.92, 33.55, 31.92, 30.15, 29.74, 29.71, 29.66, 29.36, 27.92, 26.67, 24.69, 22.69, 22.56, 19.42, 14.12.

### Synthesis of Compound W57: 1-butyl-3-(2,6-dimethyl-14-octadecyldotriacontan-9-yl)-2-methyl-1H-imidazol-3-ium chloride

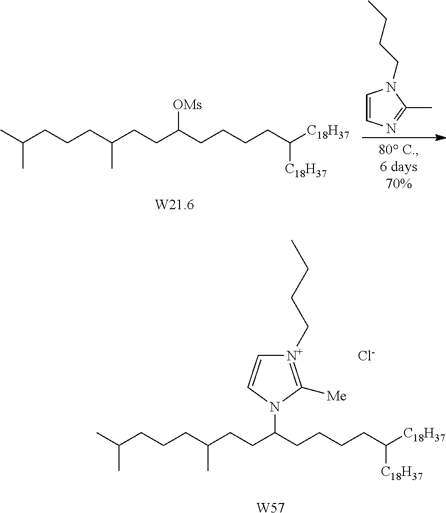

Compound W21.6 (250 mg, 303 μmol) is stirred at 80° C. in 1-butyl-2-methylimidazole (1.05 g, 7.57 mmol) for 6 days. TLC monitoring (Heptane/DCM 1/1—Vanillin) indicated completion of the reaction. After cooling down to room temperature, the mixture was diluted with MeOH (5 mL), cooled down to 0° C., acidified with 3M HCl solution (3 mL), and evaporated to dryness. The residue was taken up in water (10 mL) and triturated at 0° C. The resulting precipitate was collected by filtration, rinsed with cold water and dried under vacuum. The crude product was purified by column chromatography to afford compound W57 as a white solid (164 mg, 60%). ¹H-NMR (CDCl₃, 298 K, 400 MHz) δ (ppm): 8.09 (dd, J=5.8, 2.1 Hz, 1H), 7.29 (d, J=2.1 Hz, 1H), 4.50 (t, J=7.3 Hz, 2H), 4.18-4.07 (m, 1H), 2.78 (s, 3H), 1.90-1.65 (m, 6H), 1.55-1.00 (m, 87H), 0.96 (t, J=7.3 Hz, 3H), 0.90-0.74 (m, 15H). ¹³C-NMR (CDCl₃, 298 K, 101 MHz) δ (ppm): 142.67, 117.65, 77.37, 77.05, 76.73, 60.60, 48.97, 39.14, 37.39, 36.94, 36.86, 33.54, 33.48, 32.83, 32.51, 32.48, 32.06, 31.92, 30.15, 29.75, 29.72, 29.71, 29.66, 29.36, 27.89, 26.68, 26.56, 26.32, 24.68, 22.69, 22.56, 19.59, 19.44, 19.38, 14.12, 13.62, 10.79.

### Synthesis of compound W58: 1-butyl-3-(3-((2,6-dimethyl-14-octadecyldotriacontan-9-yl)thio)propyl)-1H-imidazol-3-ium chloride

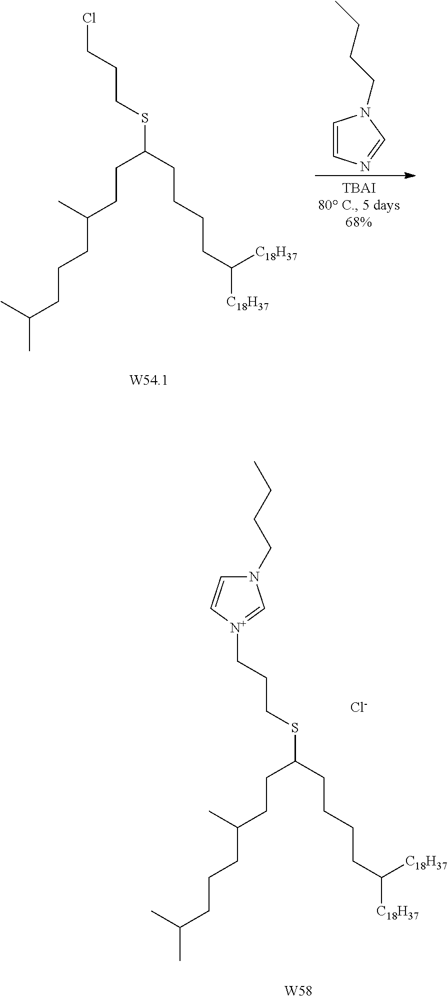

Compound W54.1 (200 mg, 238 μmol) and tetrabutylammonium iodide (17.6 mg, 47.6 μmol) were dissolved in 1-Butylimidazole (939 μL, 7.14 mmol) and the reaction mixture was stirred up at 80° C. for 5 days. TLC monitoring (Heptane 100%, Vanillin) indicated complete reaction. The yellow solution was diluted with EtOAc, transferred into a separating funnel, washed twice with a saturated aqueous solution of NH₄Cl, brine, dried over Na₂SO₄, filtered, and concentrated to dryness. The crude product was purified by column chromatography (neat DCM to DCM/MeOH 9/1) to afford compound W58 as a white and sticky solid (156 mg, 68%). ¹H-NMR (CDCl₃, 298 K, 400 MHz) δ (ppm): 11.15 (s, 1H), 7.25 (s, 1H), 7.16 (s, 1H), 4.52 (t, J=7.0 Hz, 2H), 4.34 (t, J=7.4 Hz, 2H), 2.63-2.47 (m, 3H), 2.26 (p, J=6.9 Hz, 2H), 1.95-1.87 (m, 2H), 1.59-1.02 (m, 87H), 0.97 (t, J=7.4 Hz, 3H), 0.87 (m, 15H). ¹³C-NMR (CDCl₃, 298 K, 101 MHz) δ (ppm): 138.8, 122.03, 121.17, 50.14, 48.74, 47.00, 46.95, 46.91, 46.50, 39.43, 37.57, 37.49, 37.45, 37.42, 37.36, 37.28, 37.22, 37.04, 35.23, 34.92, 34.73, 34.67, 34.44, 34.11, 34.09, 33.87, 33.85, 33.83, 33.77, 33.76, 33.73, 32.27, 32.04, 30.31, 29.87, 29.85, 29.83, 29.78, 29.48, 28.09, 26.84, 26.82, 24.89, 22.81, 19.85, 19.79, 19.77, 19.65, 19.64, 14.24.

### Synthesis of compound W59: 3-butyl-1-(2-((2,6-dimethyl-14-octadecyldotriacontan-9-yl)oxy)ethyl)-1H-imidazol-3-ium bromide

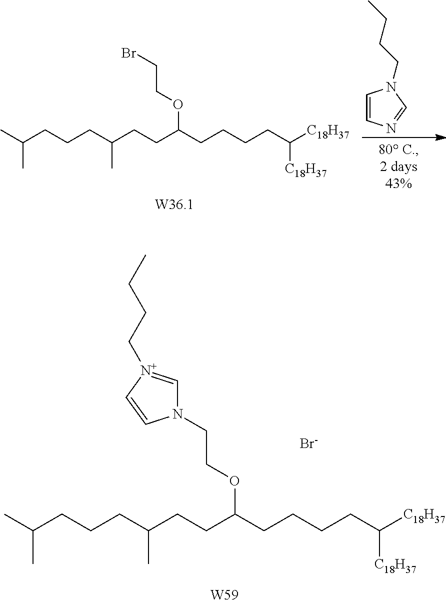

Compound W36.1 (360 mg, 421 μmol) was dissolved in 1-butylimidazole (1.38 mL, 10.5 mmol) and the reaction mixture was heated up at 80° C. overnight (21 h). TLC monitoring (Heptane/DCM 9/1, Vanillin) indicated completion of the reaction. The yellow solution was diluted with EtOAc, transferred into a separating funnel, washed twice with a saturated aqueous solution of NH₄Cl, brine, dried over Na₂SO₄, filtered, and concentrated to dryness. The crude product was purified by column chromatography (neat DCM to DCM/MeOH 9/1) to afford compound W59 as a white and sticky solid (179 mg, 43%). ¹H-NMR (CDCl₃, 298 K, 400 MHz) δ (ppm): 10.97 (s, 1H), 7.44 (bs, 1H), 7.09 (bs, 1H), 4.64 (t, J=4.5 Hz, 2H), 4.28 (t, J=7.5 Hz, 2H), 3.79 (t, J=4.5 Hz, 2H), 3.30-3.16 (m, 1H), 1.95-1.85 (m, 2H), 1.25 (s, 91H), 0.98 (t, J=7.4 Hz, 3H), 0.90-0.78 (m, 15H). ¹³C-NMR (CDCl₃, 298 K, 101 MHz) δ (ppm): 138.45, 123.54, 120.36, 80.88, 67.08, 50.61, 50.00, 39.47, 37.56, 37.35, 37.27, 33.89, 33.76, 33.75, 33.67, 33.59, 33.06, 33.01, 32.55, 32.53, 32.27, 32.04, 31.00, 30.91, 30.31, 29.87, 29.84, 29.83, 29.78, 29.48, 28.08, 27.02, 26.86, 26.84, 25.91, 25.86, 24.90, 24.88, 22.83, 22.81, 22.73, 19.80, 19.74, 19.65, 14.23, 13.55.

### Synthesis of Compound W60: 1-(3-((2,6-dimethyl-14-octadecyldotriacontan-9-yl)oxy)-3-oxopropyl)-3-methyl-1H-imidazol-3-ium bromide

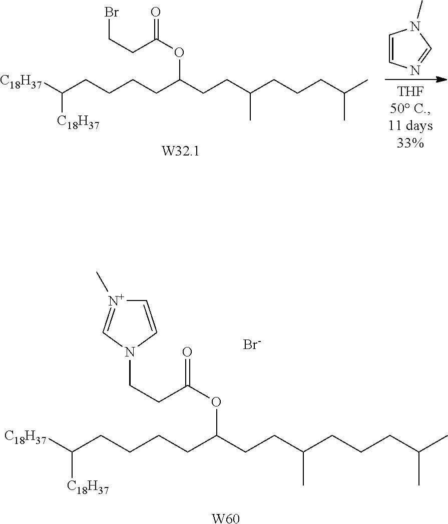

Compound W32.1 (852 mg, 647 μmol) was solubilized in THE (5.7 mL) and 1-Methylimidazole (309 μL, 3.88 mmol) was then added. The reaction mixture was stirred at 50° C. for 11 days. The reaction mixture was diluted with DCM (50 mL) and the organic layer was washed with water (3*50 mL). The organic layer was dried over Na₂SO₄, filtered off, and concentrated to dryness. The product was purified by column chromatography (neat DCM to DCM/MeOH 9/1) to afford compound W60 as a colorless oil (207 mg, 33%). ¹H-NMR (CDCl₃, 298 K, 400 MHz) δ (ppm): 10.87 (s, 1H), 7.51 (bs, 1H), 7.17 (bs, 1H), 4.84-4.77 (m, 1H), 4.68 (t, J=5.7 Hz, 2H), 4.06 (s, 3H), 3.02 (t, J=5.7 Hz, 2H), 1.60-1.43 (m, 4H), 1.40-0.94 (m, 80H), 0.98-0.79 (m, 15H). ¹³C-NMR (CDCl₃, 298 K, 101 MHz) δ (ppm): 170.92, 139.22, 123.38, 122.24, 45.72, 39.41, 37.58, 37.24, 37.15, 36.82, 35.36, 33.78, 32.84, 32.80, 32.59, 32.06, 31.51, 30.32, 29.88, 29.85, 29.84, 29.79, 29.49, 28.07, 26.86, 26.84, 26.76, 25.93, 24.85, 22.84, 22.82, 22.74, 22.73, 19.68, 19.63, 14.24.

### Synthesis of compound W61: 1-methyl-3-(4-((16-octadecyltetratriacontan-11-yl)oxy)-4-oxobutyl)-1H-imidazol-3-ium bromide

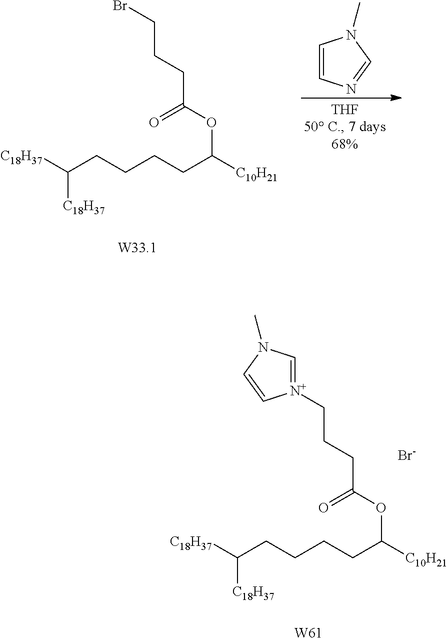

Compound 33.1 (600 mg, 669 μmol) was solubilized in THF (5.9 mL) and 1-Methylimidazole (213 μL, 2.68 mmol) was added. The reaction mixture was heated at 50° C. under argon for 8 days. The reaction mixture was diluted with DCM, washed with saturated aqueous NH4Cl solution (2*50 mL), water (50 mL), dried over Na2SO4, filtered off, and concentrated to dryness. The crude product was purified by column chromatography (DCM to DCM/MeOH 9/1) to afford compound W61 as a white powder (425 mg, 68%). ¹H-NMR (CDCl₃, 298 K, 400 MHz) δ (ppm): 10.97 (s, 1H), 7.31 (bs, 2H), 4.81 (bs, 1H), 4.45 (t, J=7.2 Hz, 2H), 4.10 (bs, 3H), 2.46-2.36 (m, 2H), 2.27-2.15 (m, 2H), 1.58-0.97 (m, 89H), 0.92-0.76 (m, 15H). 13C-NMR (CDCl₃, 298 K, 101 MHz) δ (ppm) 172.20, 139.12, 122.99, 121.93, 49.09, 39.38, 37.55, 37.25, 37.15, 36.82, 34.21, 33.74, 32.82, 32.62, 32.04, 31.52, 30.62, 30.30, 29.87, 29.84, 29.82, 29.77, 29.48, 28.06, 26.81, 25.97, 24.86, 22.80, 22.72, 19.71, 19.66, 14.23.

### Synthesis of Compound W62: 1-methyl-3-(5-((16-octadecyltetratriacontan-11-yl)oxy)-5-oxopentyl)-1H-imidazol-3-ium bromide

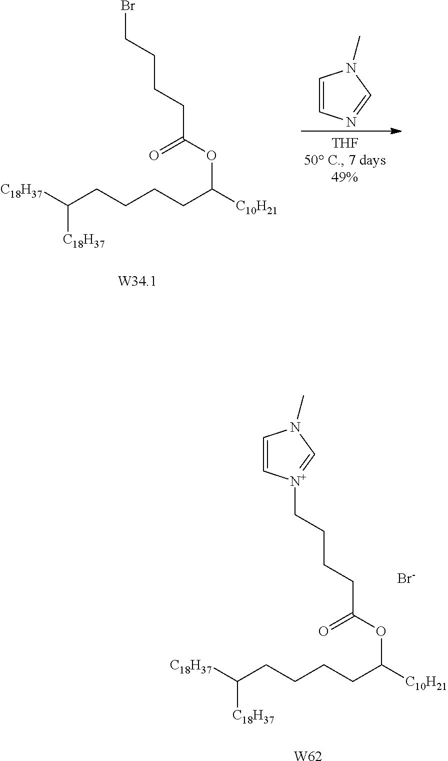

Compound W34.1 (354 mg, 389 μmol) was solubilized in anhydrous THE (3.4 mL) and 1-Methylimidazole (186 μL, 2.33 mmol) was added. The reaction mixture was stirred at 50° C. under argon for 7 days. The reaction mixture was diluted with DCM (30 mL) and the organic layer was washed with saturated NH₄Cl aqueous solution. The organic layer was dried with Na₂SO₄, filtered off, and concentrated to dryness. The crude product was purified by column chromatography (neat DCM to DCM/MeOH 9/1) to afford compound W62 as a white solid (191 mg, 49%). ¹H-NMR (CDCl₃, 298 K, 400 MHz) δ (ppm): 10.83 (s, 1H), 7.25 (bs, 1H), 7.20 (bs, 1H), 4.85-4.76 (m, 1H), 4.39 (t, J=7.2 Hz, 2H), 4.10 (s, 3H), 2.38 (t, J=7.0 Hz, 2H), 2.04-1.93 (m, 2H), 1.71-1.62 (m, 4H), 1.57-0.98 (m, 87H), 0.97-0.72 (m, 15H). ¹³C-NMR (CDCl₃, 298 K, 101 MHz) δ (ppm): 172.92, 138.38, 123.26, 121.82, 49.97, 39.39, 37.54, 37.23, 37.15, 36.83, 34.22, 33.78, 33.73, 33.44, 32.83, 32.80, 32.57, 32.03, 31.57, 30.29, 29.86, 29.84, 29.82, 29.77, 29.56, 29.47, 28.06, 26.81, 25.98, 24.85, 22.83, 22.80, 22.72, 21.51, 19.73, 19.67, 14.23.

### Synthesis of Compound W40 3-(2-(butyl(2,6-dimethyl-14-octadecyldotriacontan-9-yl)amino)ethyl)-1-methyl-1H-imidazol-3-ium chloride

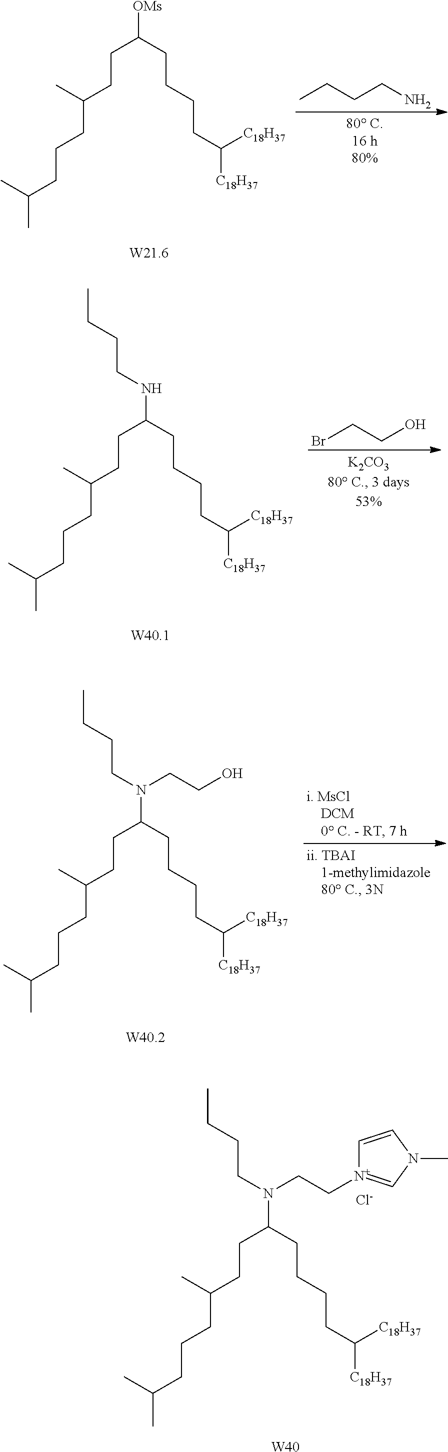

### Synthesis of Compound W40.1

Compound W21.6 (1.50 g, 1.82 mmol) and butylamine 99.5% (4.49 mL, 45.4 mmol) was stirred under argon atmosphere at 80° C. for 16 h. The reaction was monitored by TLC (Heptane/DCM 7/3; vanillin), indicating completion of the reaction. The reaction was concentrated to dryness and was purified by column chromatography (neat DCM to DCM/MeOH 9/2) to afford pure compound W40.1 as a colorless oil (1.17 g, 80%). ¹H-NMR (CDCl₃, 298 K, 400 MHz) δ (ppm): 2.70-2.62 (m, 2H), 2.62-2.50 (m, 1H), 1.65-1 (m, 93H), 0.93 (t, J=7.3 Hz, 3H), 0.90-0.80 (m, 15H).

### Synthesis of Compound W40.2

Compound W40.1 (550 mg, 685 μmol) and potassium carbonate (189 mg, 1.37 mmol) were suspended in 2-bromoethanol (1.28 mL, 17.1 mmol) under argon atmosphere. The suspension was heated up to 80° C. for 3 days. The reaction was monitored by TLC (DCM/MeOH 9/1; ninhydrin), indicating completion of the reaction. The reaction was diluted with DCM (50 mL) then was washed with water (50 mL), with an aqueous and saturated solution of NaHCO₃ (50 mL) then with brine (50 mL). The organic layer was dried over Na₂SO₄, filtered, and concentrated to dryness. The residue was purified by column chromatography (neat DCM to DCM/MeOH 95/05) to afford pure compound W40.2 as a pale orange oil (305 mg, 53%). ¹H-NMR (CDCl₃, 298 K, 400 MHz) δ (ppm): 3.48 (t, J=5.4 Hz, 2H), 2.58 (t, J=5.4 Hz, 2H), 2.48-2.27 (m, 3H), 1.6-1 (m, 93H), 0.95-0.78 (m, 18H).

### Synthesis of Compound W40

To a solution of compound W40.2 (290 mg, 343 μmol) in DCM (6.6 mL) under argon atmosphere was added at 0° C. methanesulfonyl chloride (29.2 μL, 377 μmol). The solution was stirred at 0-5° C. for 3h. TLC monitoring (DCM/MeOH 96/4; KMnO₄ or Ninhydrin) indicated residual starting material. Methanesulfonyl chloride (133 μL, 1.71 mmol) was added at 0° C. to the reaction mixture, that was allowed to warm up to RT for 4h. TLC monitoring (DCM/MeOH 96/4; KMnO₄ or Ninhydrin) showed incomplete. MeOH (5 mL) was added, and the mixture was concentrated to dryness. MS (ESI-TOF): calculated for C₅₈1H₁₁ClN [M+H]⁺=864.90, found 864.9035.

Intermediate (296 mg, 343 μmol), tetrabutylammonium iodide (25.3 mg, 68.4 μmol) and 1-Methylimidazole (818 μL, 10.3 mmol) were stirred at 80° C. for 3 days. TLC monitoring (9/1; I₂ and KMnO₄) indicated completion of the reaction. The reaction mixture was diluted with MeOH (10 mL), cooled down to 0° C., and 3M HCl (3.6 mL) was introduced. The mixture was concentrated to dryness, and the resulting residue was triturated in water (10 mL) at 0° C. for 1 h. The precipitate was collected by filtration, rinsed with cold water (25 mL), and purified by column chromatography (neat DCM to DCM/MeOH 9/1). The fractions that contained the product were concentrated. The residue was dissolved in DCM (50 mL), washed with 3M HCl (2*20 mL), dried over Na₂SO₄, filtered off and concentrated to dryness to afford pure compound W40 as a white and sticky solid (52 mg, 16%). ¹H-NMR (MeOD, 298 K, 400 MHz) δ (ppm): 9.20 (s, 1H), 7.87 (s, 1H), 7.64 (s, 1H), 4.93-4.78 (m, 2H), 3.97 (s, 3H), 3.86-3.64 (m, 2H), 3.37-3.2 (m, 3H), 2-1.14 (m, 93H), 1.02 (t, J=7.3 Hz, 3H), 0.99-0.86 (m, 15H). ¹³C-NMR (MeOD, 298 K, 101 MHz) δ (ppm): 139.16, 125.34, 123.86, 66.50, 66.40, 53.27, 50.93, 45.51, 40.55, 38.53, 38.42, 38.09, 36.78, 35.47, 35.32, 34.96, 34.76, 34.37, 33.22, 31.30, 30.98, 30.95, 30.91, 30.65, 29.24, 29.22, 28.71, 28.34, 27.98, 27.83, 26.03, 25.89, 23.88, 23.37, 23.35, 23.27, 23.25, 21.15, 20.17, 19.94, 14.75, 14.06.

### Synthesis of Compound W65 3-(2-(benzyl(2,6-dimethyl-14-octadecyldotriacontan-9-yl)amino)ethyl)-1-methyl-1H-imidazol-3-ium chloride

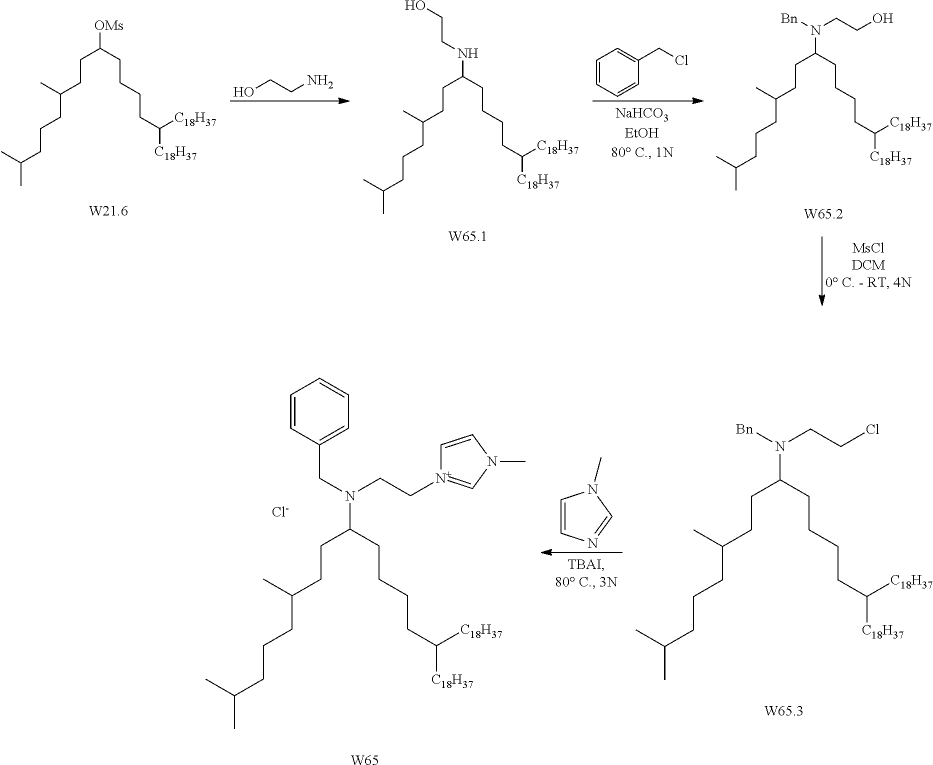

### Synthesis of Compound W65.1

Compound W21.6 (600 mg, 727 μmol) and ethanolamine (1.10 mL, 18.2 mmol) were stirred under argon atmosphere at 80° C. for 22 hours, then 110° C. for 18 hours more. The reaction was monitored by TLC (Heptane/DCM 7/3; vanillin), indicating completion of the reaction. The reaction was cooled down to RT, leading to biphasic mixture. The upper layer was collected and was purified by column chromatography (neat DCM to DCM/MeOH 8/2) to afford W65.1 as a colorless oil (366 mg, 62%). ¹H-NMR (CDCl₃, 298 K, 400 MHz) δ (ppm): 3.61 (t, J=5.2 Hz, 2H), 2.77 (t, J=5.2 Hz, 2H), 2.46 (q, J=5.9 Hz, 1H), 1.26 (m, 91H), 0.88 (dd, J=8.6, 6.5 Hz, 15H).

### Synthesis of Compound W65.2

Compound W65.1 (1.89 g, 2.39 mmol) was suspended in ethanol (10.0 mL). Sodium hydrogenocarbonate (402 mg, 4.78 mmol) and benzylchloride (550 μL, 4.78 mmol) were successively added, and the reaction mixture was stirred at 80° C. under argon atmosphere for 18 hours. The reaction was monitored by TLC (DCM/MeOH 95/5) indicated completion of the reaction. The solvent was evaporated. The residue was taken up in DCM (100 mL), washed with water (100 mL), brine, dried over Na₂SO₄, filtered and concentrated to dryness. The residue was purified by column chromatography (neat heptane to heptane/EtOAc 9/1) to afford compound W65.2 as a colorless oil (1.84 g, 87%). ¹H-NMR (CDCl₃, 298 K, 400 MHz) δ (ppm): 7.35-7.20 (m, 5H), 3.59 (s, 2H), 3.46 (t, J=5.3 Hz, 2H), 2.64 (t, 2H), 2.46-2.36 (m, 1H), 1.61-1.44 (m, 4H), 1.56-1.20 (m, 8H), 1.40-1.00 (m, 79H), 0.92-0.82 (m, 15H).

### Synthesis of Compound W65.3

To a solution of compound W65.2 (1·84 g, 2.09 mmol) in DCM (40.0 mL) under argon atmosphere was added at 0° C. methanesulfonyl chloride (809 μL, 10.4 mmol). The reaction mixture was allowed to warm up to RT overnight (17 h). TLC monitoring (Heptane/EtOAc 95/5, KMnO4) indicated residual starting material. Methanesulfonyl chloride (809 μL, 10.4 mmol) was added at 0° C. to the reaction mixture, that was further stirred at RT for 2 days more. MeOH (5 mL) was added, and the mixture was diluted with DCM (100 mL). The organic layer was washed with water (3*100 mL), dried over Na₂SO₄, filtered off and concentrated to dryness to afford pure compound 65.3 as a colorless oil (1.78 g, 95%). ¹H-NMR (CDCl₃, 298 K, 400 MHz) δ (ppm): 7.66-7.23 (m, 5H), 3.65 (s, 2H), 3.30 (t, J=7.6 Hz, 2H), 2.85-2.73 (m, 2H), 2.39 (s, 1H), 1.56-1.20 (m, 89H), 0.91-0.83 (m, 15H).

### Synthesis of Compound W65

Compound W65.3 (500 mg, 556 μmol), tetrabutylammonium iodide (41.1 mg, 111 μmol) and 1-methylimidazole (266 μL, 3.34 mmol) was stirred at 80° C. overnight, and at 110° C. for 4 hours more. TLC monitoring (9/1, KMnO4) indicated completion of the reaction. The reaction mixture is diluted with DCM (20 mL), washed with 3M HCl (2*20 mL), brine (20 mL), dried over Na₂SO₄, filtered off and concentrated to dryness. The product was purified by column chromatography (neat DCM to DCM/MeOH 9/1) to afford compound W65 (317 mg, 58%). ¹H-NMR (CDCl₃, 298 K, 400 MHz) δ (ppm): 8.59 (s, 1H), 7.54-7.10 (m, 7H), 4.21-4.06 (m, 2H), 3.88 (s, 3H), 3.72-3.60 (m, 2H), 3.04-2.93 (m, 2H), 2.48-2.40 (m, 1H), 1.31 (s, 89H), 0.97-0.85 (m, 15H). ¹³C-NMR (MeOD, 298 K, 101 MHz) δ (ppm): 129.00, 128.09, 122.89, 118.73, 56.93, 53.39, 49.81, 48.25, 48.04, 47.82, 47.61, 47.40, 47.18, 46.97, 39.25, 39.18, 37.13, 36.95, 33.61, 33.40, 32.84, 32.54, 31·77, 29.79, 29.51, 29.50, 29.47, 29.41, 29.18, 27.84, 27.82, 27.42, 26.56, 26.41, 26.39, 24.62, 24.58, 22.42, 21.91, 21.81, 18.91, 16.97, 13.23.

### Synthesis of Compound W30 3-(2-((2,6-dimethyl-14-octadecyldotriacontan-9-yl)amino)ethyl)-1-methyl-1H-imidazol-3-ium chloride

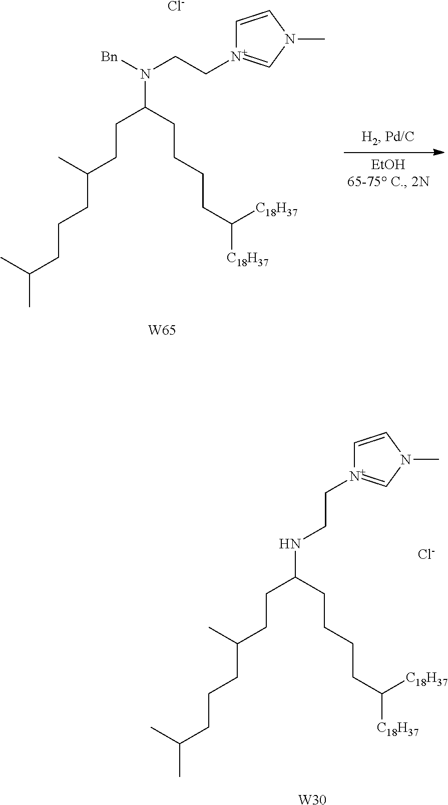

Compound W65 (240 mg, 51.0 μmol) was solubilized in EtOH (1.22 mL). The atmosphere was purged with Argon through 3 vacuum/argon cycle. Palladium on activated charcoal (10%, 15.6 mg, 14.7 μmol) was added and the reaction mixture was stirred at 75° C. overnight (18 h). The reaction mixture was filtered through Celite and the cake as rinsed with ethanol. The filtrate was evaporated to afford pure compound W65 as a white solid (27.6 mg, 61%). ¹H-NMR (MeOD, 298 K, 400 MHz) δ (ppm): δ 7.57 (d, J=2.0 Hz, 1H), 7.48 (d, J=2.0 Hz, 1H), 4.27-4.13 (m, 2H), 3.84 (s, 3H), 3.07-2.97 (m, 2H), 2.55-2.46 (m, 1H), 1.51-0.94 (m, 89H), 0.84-0.73 (m, 15H). ¹³C-NMR (CDCl₃, 298 K, 101 MHz) δ (ppm): 137.21, 123.36, 122.55, 58.13, 57.98, 48.84, 48.32, 48.24, 48.03, 47.82, 47.61, 47.39, 47.18, 46.97, 45.39, 45.36, 39.22, 39.21, 37.17, 37.01, 35.11, 33.65, 33.42, 32.98, 32.80, 32.67, 32.50, 32.27, 31.81, 30.04, 29.85, 29.78, 29.57, 29.55, 29.52, 29.47, 29.23, 27.82, 26.76, 26.42, 25.66, 25.63, 24.60, 24.56, 22.46, 21.92, 21.84, 21.83, 18.91, 13.30, 12.50.

### Synthesis of Compound W45 3-(2-((2,6-dimethyl-14-octadecyldotriacontan-9-yl)amino)ethyl)-1,2-dimethyl-1H-imidazol-3-ium chloride

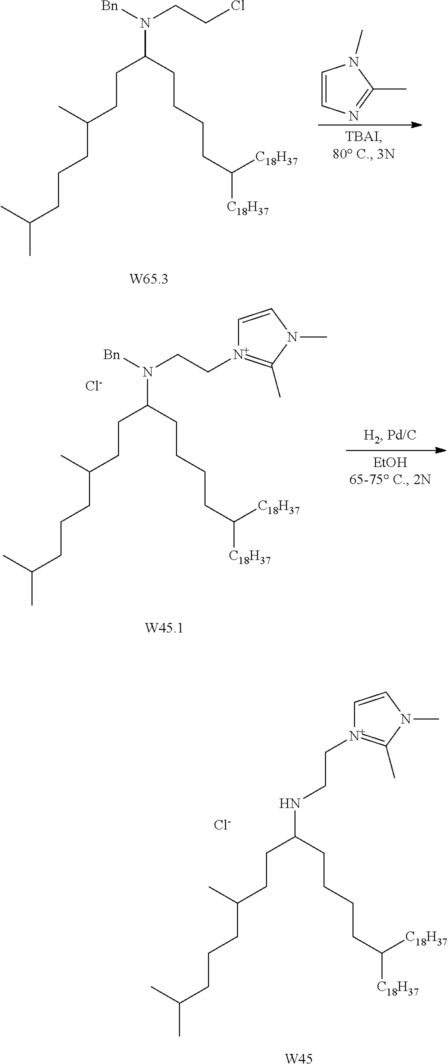

### Synthesis of Compound W45.1

Compound W65.3 (390 mg, 434 μmol) and tetrabutylammonium iodide (32.0 mg, 86.8 μmol) were heated up at 80° C. in 1,2-Dimethylimidazole (1·25 g, 13.0 mmol) for 2 days. TLC monitoring (Heptane/AE 95/5, KMnO4) indicated completion of the reaction. The reaction mixture was diluted with MeOH (10 mL), cooled down to 0° C., and 3M HCl (30 eq, 4.3 mL) was introduced. The mixture was concentrated to dryness, and the resulting residue was triturated in water (20 mL) at 0° C. for 2 h. The precipitate was collected by filtration, rinced with water (50 mL), and purified by column chromatography (neat DCM to DCM/MeOH 9/1). The fractions that contained the product were concentrated. The residue was dissolved in DCM (50 mL), washed with 3M HCl (2*20 mL), dried over Na₂SO₄, filtered off and concentrated to dyrness to afford pure compound W45 as a light yellow and sticky solid (191 mg, 45%). ¹H-NMR (MeOD, 298 K, 400 MHz) δ (ppm): 7.72-7.25 (m, 7H), 4.60-4.33 (m, 3H), 3.72 (s, 3H), 3.66-3.45 (s, 2H), 3.11-2.97 (m, 1H), 2.55 (s, 3H), 2.01-0.91 (m, 90H), 0.90-0.74 (m, 15H).

### Synthesis of Compound W45

Compound 45.1 (170 mg, 171 μmol) was solubilized in EtOH (854 μL). The atmosphere was purged 3 times with argon and Palladium on charcoal 10% (10.9 mg, 10.2 μmol) was added. The atmosphere was purged 3 times with H2 and the reaction mixture was heated up at 65° C. under hydrogene atmosphere overnight. TLC monitoring (DCM/MeOH 9/1, 12+Ninhydrin) indicated completion of the reaction. The reaction mixture was filtered on a pad of celite and the cake was rinced with ethanol. The filtrate is concentrated to dryness to afford pure compound W45 as off-white solid (129 mg, 83%). ¹H-NMR (MeOD, 298K, 400 MHz) δ (ppm): 7.67 (d, J=2.0 Hz, 1H), 7.57 (d, J=2.0 Hz, 1H), 4.59 (t, J=6.9 Hz, 2H), 3.86 (s, 3H), 3.54 (t, J=6.9 Hz, 2H), 3.30-3.20 (m, 1H), 2.73 (s, 3H), 1.88-1.65 (m, 4H), 1.61-1.08 (m, 85H), 1.02-0.83 (m, 15H). ¹³C-NMR (MeOD, 298 K, 101 MHz) δ (ppm): 145.89, 123.03, 120.95, 60.08, 59.91, 43.95, 43.76, 39.17, 39.15, 37.12, 36.92, 36.74, 34.49, 33.50, 33.34, 32.84, 32.65, 31.77, 31.58, 29.83, 29.52, 29.50, 29.48, 29.43, 29.18, 27.82, 27.15, 26.93, 26.50, 26.39, 24.99, 24.58, 24.51, 22.43, 21.90, 21.80, 18.70, 18.65, 13.23, 9.15.

### Synthesis of Compound W46 1-butyl-3-(2-((2,6-dimethyl-14-octadecyldotriacontan-9-yl)amino)ethyl)-2-methyl-1H-imidazol-3-ium chloride

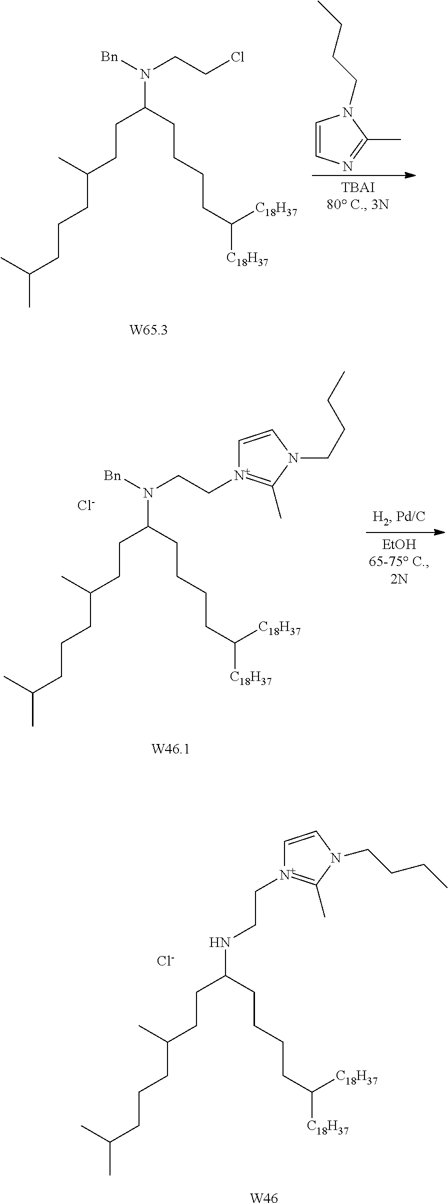

### Synthesis of Compound W46.1

Compound W65.3 (390 mg, 434 μmol) and tetrabutylammonium iodide (32.0 mg, 86.8 μmol) were heated up to 80° C. in 1-butyl-2-methyl-1H-imidazole (1.80 g, 13.0 mmol) overnight (18 h). TLC (Heptane/EtOAc 95/5, KMnO4) indicated completion of the reaction. The reaction mixture was diluted with MeOH (10 mL), cooled down to 0° C., and 3M HCl (4.3 mL) was added. After 5 minutes stirring at 0° C., the mixture is evaporated under reduced pressure. Water (20 mL) was added to the residue. The resulting precipitate is collected by filtration, washed with water, and dried. The product was purified by column chromatography (neat DCM to DCM/MeOH 9/1) to afford pure compound W46.1 as a white and sticky solid (199 mg, 44%). ¹H-NMR (MeOD, 298 K, 400 MHz) δ (ppm): 7.49 (s, 1H), 7.39 (s, 1H), 7.34-7.14 (m, 5H), 4.13-4.04 (m, 4H), 3.70-3.65 (m, 2H), 2.97-2.89 (m, 2H), 2.54-2.45 (m, 1H), 2.36 (s, 3H), 1.84-1.73 (m, 2H), 1.62-1.05 (m, 91H), 1.00 (t, J=7.3 Hz, 3H), 0.96-0.84 (m, 15H).

### Synthesis of Compound W46

Compound W65.3 (199 mg, 192 μmol) was solubilized in EtOH (959 μL). The atmosphere was purged with argon before Palladium on activated charcoal (10%, 12.3 mg, 11.5 μmol) was introduced. The reaction mixture was stirred at 65° C. under hydrogene atmosphere for 3 days. TLC monitoring (DCM/MeOH 9/1, ninhydrin) indicated completion of the reaction. The reaction mixture was filtered through celite, and the cake was rinsed with ethanol (50 mL). Concentration of the filtrate affords pure compound W46 as a white solid (130 mg, 71%). ¹H-NMR (MeOD, 298 K, 400 MHz) δ (ppm): 7.57 (d, J=2.1 Hz, 1H), 7.55 (d, J=2.1 Hz, 1H), 4.23-4.11 (m, 4H), 2.98 (t, J=5.9 Hz, 2H), 2.67 (s, 3H), 2.47-2.38 (m, 1H), 1.87-1.76 (m, 2H), 1.60-1.04 (m, 91H), 1.00 (t, J=7.4 Hz, 3H), 0.95-0.81 (m, 15H). ¹³C-NMR (MeOD, 298 K, 101 MHz) δ (ppm): 144.49, 121.44, 120.95, 57.70, 57.54, 45.83, 45.78, 39.24, 39.22, 37.17, 37.07, 37.05, 33.68, 33.49, 33.43, 33.35, 33.02, 32.85, 32.62, 32.39, 31.81, 31.51, 30.79, 30.55, 29.85, 29.57, 29.56, 29.53, 29.51, 29.47, 29.23, 27.82, 26.82, 26.43, 25.75, 24.62, 24.59, 22.47, 21.93, 21.85, 21.83, 19.21, 19.01, 18.99, 13.30, 12.58, 8.76.

### Synthesis of Compound W49 3-(2-((2,6-dimethyl-14-octadecyldotriacontan-9-yl)amino)ethyl)-1,2,4,5-tetramethyl-1H-imidazol-3-ium chloride

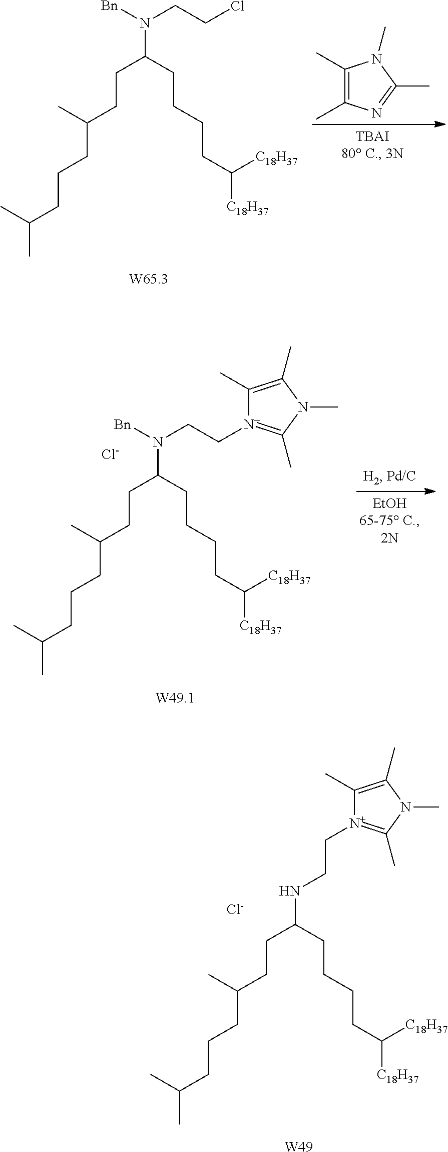

### Synthesis of Compound W49.1

Compound W65.3 (313 mg, 348 μmol) and tetrabutylammonium iodide (25.7 mg, 69.6 μmol were heated up to 80° C. in 1,2,4,5-Tetramethyl-1H-imidazole (1.30 g, 10.4 mmol) overnight (18 h). TLC (Heptane/EtOAc 95/5, KMnO4) indicated completion of the reaction. The reaction mixture was diluted with MeOH (10 mL), cooled down to 0° C., and 3M HCl (4.3 mL) was added. After 5 minutes stirring at 0° C., the mixture is evaporated under reduced pressure. Water (20 mL) was added to the residue. The resulting precipitate is collected by filtration, washed with water, and dried. The product was purified by column chromatography (neat DCM to DCM/MeOH 9/1) to afford pure compound W49.1 as a white and sticky solid (159.4 mg, 45%). ¹H-NMR (MeOD, 298 K, 400 MHz) δ (ppm): 7.35-7.23 (m, 5H), 3.93 (t, J=6.7 Hz, 2H), 3.67-3.64 (m, 2H), 3.59 (s, 3H), 2.80 (t, J=6.9 Hz, 2H), 2.60-2.53 (m, 1H), 2.41 (s, 3H), 2.19 (s, 3H), 2.11 (s, 3H), 1.64-1.10 (m, 89H), 1.02-0.84 (m, 15H).

### Synthesis of Compounds W49

Compound W49.1 (158 mg, 154 μmol) was solubilized in EtOH (772 μL). The atmosphere was purged with argon before Palladium on activated charcoal (9.86 mg, 9.26 μmol). The reaction mixture was stirred at 65° C. under hydrogene atmosphere for 3 days. TLC monitoring (DCM/MeOH 9/1, ninhydrin) indicated completion of the reaction. The reaction mixture was filtered through celite, and the cake was rinsed with DCM (65 mL). Concentration of the filtrate affords pure compound W49 as a white solid (87.2 mg, 60%). ¹H-NMR (MeOD, 298 K, 400 MHz) δ (ppm): 4.13 (t, J=6.5 Hz, 2H), 3.66 (s, 3H), 2.86 (t, J=6.5 Hz, 2H), 2.64 (s, 3H), 2.49-2.40 (m, 1H), 2.30 (s, 3H), 2.26 (s, 3H), 1.60-0.99 (m, 89H), 0.95-0.83 (m, 15H). ¹³C-NMR (MeOD, 298 K, 101 MHz) δ (ppm): 143.21, 125.89, 125.12, 57.79, 57.63, 45.53, 45.42, 45.36, 39.27, 37.28, 37.15, 37.10, 33.81, 33.59, 33.51, 33.44, 33.05, 32.88, 32.59, 32.35, 31.91, 31.89, 31.88, 30.99, 30.72, 30.48, 30.01, 29.99, 29.96, 29.70, 29.68, 29.66, 29.62, 29.35, 29.33, 29.31, 27.85, 26.94, 26.53, 25.88, 24.64, 22.56, 22.55, 22.53, 22.06, 22.04, 21.98, 21.96, 21.94, 19.11, 19.09, 19.07, 13.51, 13.48, 13.44, 9.38, 7.42, 7.40, 7.21.

### Synthesis of Compound W42 1-butyl-3-(3-((2,6-dimethyl-14-octadecyldotriacontan-9-yl)amino)propyl)-1H-imidazol-3-ium chloride

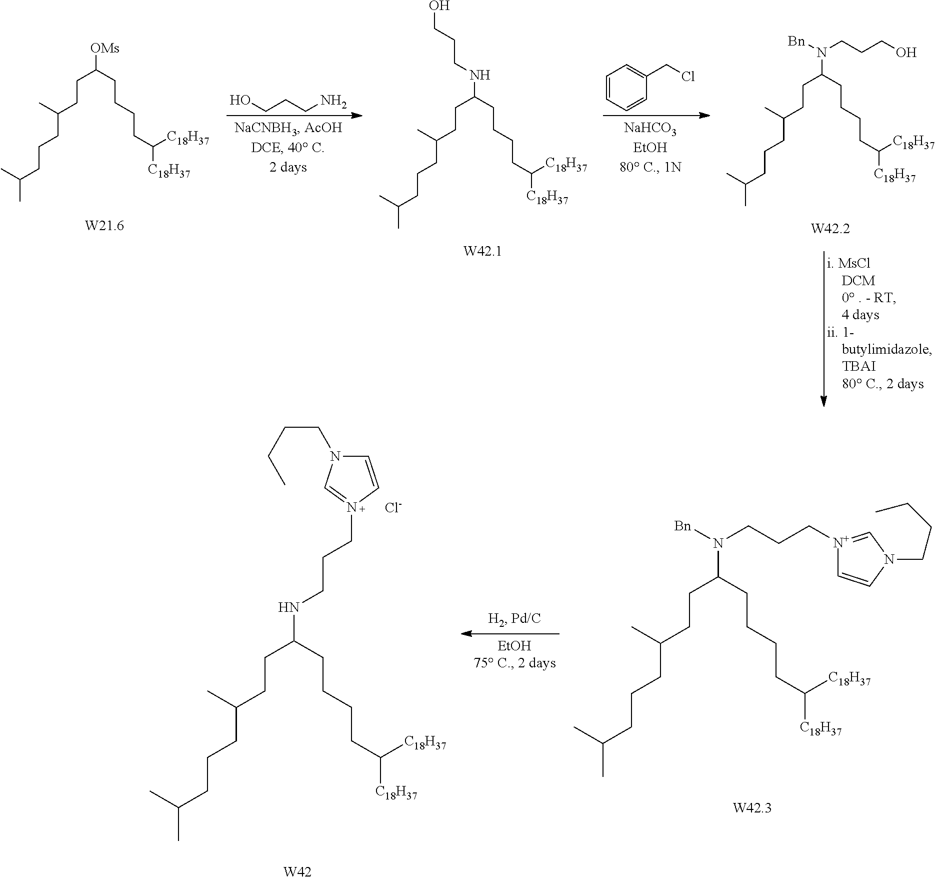

### Synthesis of Compound W42.1

To a stirred solution of compound W21.6 (1.20 g, 1.61 mmol) and 3-Amino-1-Propanol (242 mg, 3.22 mmol) in DCE (9.2 mL) was added acetic acid (95.2 μL, 5.32 mmol). The reaction mixture is stirred at RT overnight (21 h) before sodium cyanoborohydride (118 mg, 1.93 mmol) was added to the mixture at 0° C. The reaction mixture was then stirred at 40° C. overnight. TLC monitoring (9/1 DCM/MeOH—Vanillin) indicated completion of the reaction. Water (20 mL) and DCM (50 mL) were added. The organic was washed with water (20 mL), brine, dried over Na₂SO₄, filtered and concentrated to dryness. The resulting residue was purified by column chromatography (neat DCM to DCM/MeOH 8/2) to afford compound W42.1 as a colorless oil (749 mg, 66%). (Some of the NMR signals are split due to atropoisomerism). ¹H-NMR (MeOD, 298 K, 400 MHz) δ (ppm): 3.80-3.70 (m, 1H), 2.80-2.74 (m, 1H), 2.53-2.26 (m, 3H), 1.58-0.94 (m, 91H), 0.94-0.80 (m, 15H).

### Synthesis of Compound W42.2

To a stirred solution of compound W42.1 (725 mg, 901 μmol) in EtOH (5.0 mL) were successively added sodium hydrogenocarbonate (151 mg, 1.80 mmol) and benzylchloride (207 μL, 1.80 mmol) at RT under argon. The suspension was heated at reflux overnight (18 h). TLC monitoring (DCM/MeOH 95/5, KMnO4) indicated completion of the reaction. The reaction mixture was concentrated to dryness. The resulting residue was diluted with DCM (30 mL), washed with water (50 mL), brine (50 mL), dried over Na₂SO₄, filtered off and concentrated to dryness. The crude product was purified by column chromatography (neat heptane to heptane/EtOAc 95/5) to afford compound W42.2 as a colorless oil (630 mg, 78%). ¹H-NMR (CDCl₃, 298 K, 400 MHz) δ (ppm): 3.74-3.59 (m, 2H), 3.51-3.35 (m, 2H), 2.51-2.36 (m, 2H), 2.31-2.22 (m, 1H), 1.70-1.47 (m, 5H), 1.45-0.97 (m, 90H), 0.93-0.83 (m, 15H), 0.82-0.77 (m, 2H).

### Synthesis of Compound W42.3

To an ice-chilled solution of W42.2 (507 mg, 567 μmol) in anhydrous DCM (10.8 mL) was added methanesulfonyl chloride (219 μL, 2.83 mmol) under argon at 0° C. The reaction mixture was allowed to warm up to RT overnight (18 h). TLC monitoring (neat DCM, KMnO4) indicated residual presence of starting material. Methanesulfonyl chloride (219 μL, 2.83 mmol) was added at 0° C. then the solution was stirred at RT for 4 days more. The solution was concentrated to dryness and the residue taken up in DCM (50 mL). The organic layer was washed with water (2*50 mL), brine (50 mL), dried over Na₂SO₄, filtered off and concentrated to afford W42.3, that was used without any further purification. MS (ESI-TOF): calculated for C₆₂H₁₁₈ClN [M+H]⁺=912.90, found 912.9061.

The previous intermediate (430 mg, 471 μmol) and tetrabutylammonium iodide (34.8 mg, 94.2 μmol) heated in 1-Butylimidazole (1.86 mL, 14.1 mmol) at 80° C. for 2 days. TLC monitoring (Heptane/EtOAc 95/5, KMnO4) indicated completion of the reaction. The reaction mixture was diluted with MeOH (10 mL), cooled down to 0° C., and 3M HCl (1.8 mL) was added. After 5 minutes stirring at 0° C., the mixture is evaporated under reduced pressure. Water (20 mL) was added to the residue. The resulting precipitate is collected by filtration, washed with water, and dried. The crude was purified by column chromatography (neat DCM to DCM/MeOH 9/1) to afford compound W42.3 (294 mg, 60%) as pale-yellow sticky solid. MS (ESI-TOF): calculated for C₆₂H₁₁₈ClN [M]⁺=1001.03, found 1001.0261.

### Synthesis of Compound W42

Compound W42.3 (280 mg, 270 μmol) was solubilized in EtOH (1.35 mL). The atmosphere was purged 3 times with argon and palladium on charcoal (10%, 17.2 mg, 16.2 μmol) was added. The mixture was purged 3 times with H₂ and heated at 65° C. under hydrogene atmosphere overnight (18 h). TLC monitoring (DCM/MeOH 9/1, ninhydrin) indicated completion of the reaction. The reaction mixture was filtered through celite, and the cake was washed with ethanol (50 mL). The filtrate was concentrated to dryness, and the resulting residue was purified by column chromatography (neat DCM to DCM/MeOH 9/1) to afford compound W42 as a white solid (155 mg, 61%). ¹H-NMR (MeOD, 298 K, 400 MHz) δ (ppm): 9.22-9.02 (m, 1H), 7.86-7.66 (m, 2H), 4.36-4.19 (m, 3H), 1.97-1.87 (m, 2H), 1.69-1.11 (m, 97H), 1.03 (t, J=7.4, 3H), 0.96-0.85 (m, 15H). ¹³C-NMR (MeOD, 298 K, 101 MHz) δ (ppm):_124.85, 124.35, 123.81, 122.20, 52.26, 52.19, 51.09, 51.01, 40.89, 38.82, 38.79, 38.67, 38.62, 38.56, 35.31, 35.24, 35.06, 34.62, 34.53, 34.50, 34.43, 33.85, 33.47, 33.43, 33.32, 31.50, 31.21, 31.18, 31.16, 31.12, 30.89, 29.51, 28.42, 28.32, 28.08, 27.17, 26.89, 26.29, 26.23, 24.13, 23.59, 23.49, 20.88, 20.82, 20.56, 20.53, 19.64, 14.93, 14.16, 14.14.

### Synthesis of Compound W63 1-(4-((2,6-dimethyl-14-octadecyldotriacontan-9-yl)amino)butyl)-3-methyl-1H-imidazol-3-ium chloride

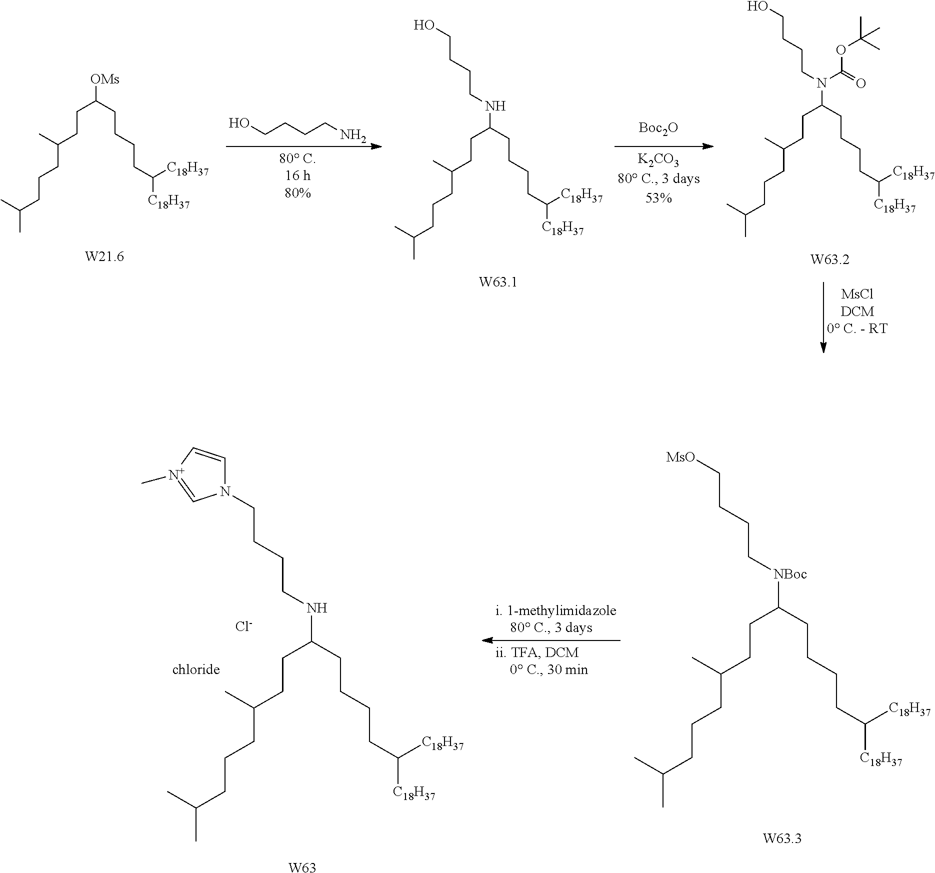

### Synthesis of Compound W63.1

Compound W21.6 (1.16 g, 1.41 mmol) and 4-Amino-1-Butanol (3.21 mL, 35.1 mmol) was stirred under argon atmosphere at 110° C. overnight (18 h). TLC monitoring (DCM/MeOH 9/1—KMnO4) indicated completion of the reaction. Once cooled down to RT, the reaction mixture was diluted with DCM (50 mL). The organic layer was washed with water (3*30 mL), dried over with Na₂SO₄, filtered off and concentrated to dryness. The residue was purified by column chromatography (DCM/MeOH 99/1 to 8/2) to afford pure compound W63.1 as a colorless oil (610 mg, 53%). ¹H-NMR (CDCl₃, 298 K, 400 MHz) δ (ppm): 3.58 (t, J=5.1 Hz, 2H), 2.63 (t, J=5.1 Hz, 2H), 2.54-2.45 (m, 1H), 1.75-1.60 (m, 4H), 1.58-0.98 (m, 91H), 0.93-0.80 (m, 15H).

### Synthesis of Compound W63.2

To a solution of compound W63.1 (610 mg, 745 μmol) in anhydrous DCM (26 mL) were successively added at RT triethylamine (628 mg, 6.21 mmol) and a solution of di-tert-butyl dicarbonate (257 mg, 1.18 mmol) in DCM (9.9 mL). The reaction mixture was stirred at RT overnight (18 h). TLC monitoring (neat DCM-KMnO4) indicated completion of the reaction. The reaction mixture was diluted with DCM (30 mL) and water (70 mL). The layers were separated and the aqueous layer was extracted with DCM (30 mL). The combined organic layer was washed with brine, dried over Na₂SO₄, filtered off and concentrated to dryness. The residue was purified by column chromatography (neat DCM to DCM/MeOH 8/2) to afford pure compound W63.2 as a colorless oil (458 mg, 63%). ¹H-NMR (CDCl₃, 298 K, 400 MHz) δ (ppm): 4.01-3.62 (m, 3H), 3.08-2.88 (m, 2H), 1.77-1.49 (m, 8H), 1.45 (s, 9H), 1.25 (s, 86H), 0.93-0.80 (m, 15H).

### Synthesis of Compound W63.3

To an ice-chilled solution of compound 63.2 (458 mg, 499 μmol) in anhydrous DCM (mL) were successfully added triethylamine (60 mg, 59 μmol) and methanesulfonyl chloride (63 mg, 55 μmol). The reaction mixture was stirred 45 minutes at 0° C. TLC monitoring (DCM/MeOH 9/1) indicated completion of the reaction. MeOH (5 mL) was added, and the reaction mixture was diluted with DCM (20 mL). The organic layer was washed with water (3*20 mL), dried over Na₂SO₄, filtered off and concentrated to dryness. The residue was tritured at 0° C. in MeOH (5 mL), and compound W63.3 was collected by filtration and rinsed with MeOH (10 mL) (483 mg, 73% yield, 75% purity). The product was used in the next steps without further any purification. ¹H-NMR (CDCl₃, 298 K, 400 MHz) δ (ppm): 4.25 (t, J=6.1 Hz, 2H), 3.98-3.66 (m, 1H), 3.07-2.88 (m, 5H), 1.79-1.58 (m, 4H), 1.45 (s, 9H), 1.41-1.00 (m, 89H), 0.96-0.79 (m, 15H).

### Synthesis of Compound W63

Compound W63.3 (200 mg, 161 μmol) was stirred at 80° C. in 1-Methylimidazole (320 μL, 4.01 mmol) overnight (18 h). TLC monitoring (DCM/MeOH 9/1, KMnO4) indicated completion of the reaction. The reaction mixture was diluted with MeOH (10 mL), cooled down to 0° C., and 3M HCl (1.8 mL) was added. After 5 minutes stirring at 0° C., the mixture is evaporated under reduced pressure. Water (20 mL) was added to the residue. The resulting precipitate is collected by filtration, washed with water, and dried. To a solution of the crude product in anhydrous DCM (2.0 mL) was added dropwise at 0° C. TFA (1.0 mL), and the reaction mixture was stirred 15 minutes at 0° C. The reaction mixture was concentrated under vacuum. The product was purified by column chromatography (neat DCM to DCM/MeOH 9/1) to afford pure compound W63 as a white solid (127 mg, 77%). ¹H-NMR (MeOD, 298 K, 400 MHz) δ (ppm): 9.02 (s, 1H), 7.68 (bs, 1H), 7.60 (bs, 1H), 4.30 (t, J=7.3 Hz, 2H), 3.95 (s, 3H), 3.22-3.15 (m, 1H), 3.12-3.03 (m, 2H), 2.06-1.95 (m, 2H), 1.87-1.62 (m, 6H), 160-1.08 (s, 85H), 1.01-0.83 (m, 15H). ¹³C-NMR (CDCl₃, 298 K, 101 MHz) δ (ppm): 136.70, 123.73, 122.29, 44.22, 39.11, 39.09, 36.97, 36.82, 36.68, 35.18, 33.34, 33.24, 32.78, 32.60, 31.67, 31.53, 29.64, 29.38, 29.35, 29.32, 29.31, 29.26, 29.07, 27.78, 27.14, 26.84, 26.35, 26.26, 24.77, 24.48, 24.42, 22.77, 22.33, 21.75, 21.65, 18.57, 18.51, 13.04. ¹⁹F-NMR (CDCl₃, 298 K, 376 MHz) δ (ppm): −75.97.

### Synthesis of Compound W64 3-butyl-1-(4-((2,6-dimethyl-14-octadecyldotriacontan-9-yl)ammonio)butyl)-1H-imidazol-3-ium chloride trifluoroacetate

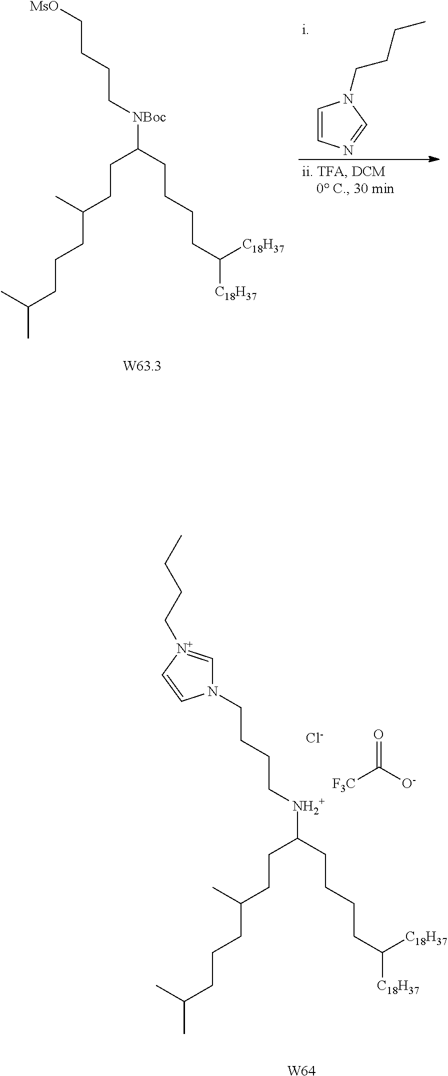

Compound W63.3 (200 mg, 161 μmol) was stirred at 80° C. in 1-Butylimidazole (527 μL, 4.01 mmol) overnight (18 h). TLC monitoring (DCM/MeOH 9/1, KMnO4) indicated completion of the reaction. The reaction mixture was diluted with MeOH (10 mL), cooled down to 0° C., and 3M HCl (1·8 mL) was added. After 5 minutes stirring at 0° C., the mixture is evaporated under reduced pressure. Water (20 mL) was added to the residue. The resulting precipitate is collected by filtration, washed with water, and dried. To a solution of the crude product in anhydrous DCM (2.0 mL) was added dropwise at 0° C. TFA (1·0 mL), and the reaction mixture was stirred 40 minutes at 0° C. The reaction mixture was concentrated under vacuum. The product was purified by column chromatography (neat DCM to DCM/MeOH 9/1) to afford pure compound W64 as a white solid (110 mg, 64%). ¹H-NMR (MeOD, 298 K, 400 MHz) δ (ppm): 9.11 (d, J=1·8 Hz, 1H), 7.69 (s, 2H), 4.30 (t, J=7.4 Hz, 4H), 4.24 (t, J=7.4 Hz, 2H), 3.19-3.12 (m, 1H), 3.08 (t, J=7.5 Hz, 2H), 2.05 1. (p, J=7.5 Hz, 2H), 1.95-1.84 (m, 2H), 1.82-1.63 (m, 6H), 1.59-1.00 (m, 85H), 1.00 (t, J=7.4 Hz, 3H), 0.97-0.83 (m, 15H). ¹³C-NMR (CDCl₃, 298 K, 101 MHz) δ (ppm): 159.74, 159.35, 137.01, 122.72, 121.61, 60.03, 59.84, 50.09, 49.29, 44.53, 39.25, 37.53, 37.12, 36.90, 33.69, 33.63, 32.89, 32.75, 32.68, 32.54, 31.92, 31.89, 30.21, 30.03, 29.77, 29.74, 29.72, 29.70, 29.65, 29.35, 27.96, 27.59, 27.51, 27.11, 26.75, 26.06, 25.97, 24.78, 24.76, 22.7z1, 22.70, 22.68, 22.58, 19.51, 19.43, 19.36, 14.09, 13.34. ¹⁹F-NMR (CDCl₃, 298 K, 376 MHz) δ (ppm): −75.93.

### Example 10. Comparative Data

The inventors carried out comparative data between formulations comprising LNPs according to the invention and SpikeVax (Moderna's COVID vaccine) formulation as disclosed in Schoenmaker et al., Int. J. Pharm., 2021, 601, 120586.

In particular the inventors designed 5 sets of experiments, each of them being specifically dedicated to evaluate the following points:
1. Proof of technical effect of compound W21.7
   SpikeVax (Moderna's COVID vaccine) formulation+addition of compound W21.7
   SpikeVax (Moderna's COVID vaccine) formulation+substitution of SM-102 by compound W21.7
2. Versatility of the LNP transfection system with respect to the ionizable lipid implied (Table 20)
   DODMA (1,2-Dioleyloxy-3-dimethylaminopropane)
   SM-102 (heptadecan-9-yl 8-((2-hydroxyethyl)(6-oxo-6-(undecyloxy)hexyl)amino)octanoate
   Dlin-MC3-DMA (((6Z,9Z,28Z,31Z)-Heptatriaconta-6,9,28,31-tetraen-19-yl 4-(dimethylamino)butanoate)
   ALC-0315 (((4-Hydroxybutyl)azanediyl)bis(hexane-6,1-diyl)bis(2-hexyldecanoate))
3. Versatility of the LNP transfection system with respect to the phospholipid implied (Table 21)
   PLPE ([1-[2-aminoethoxy(hydroxy)phosphoryl]oxy-3-hexadecanoyloxypropan-2-yl](9Z,12Z)-octadeca-9,12-dienoate)
   DOPC (1,2-Dioleoyl-sn-Glycero-3-Phosphocholine)
   DOPE (1,2-Dioleoyl-sn-glycero-3-phosphoethanolamine)
   DPPC (1,2-Dipalmitoyl-sn-glycero-3-phosphocholine)
   DSPC (1,2-Distearoyl-sn-glycero-3-phosphocholine)
   DPyPE (1,2-Diphytanoyl-sn-glycero-3-phosphoethanolamine)
4. Versatility of the LNP transfection system with respect to the sterol implied (Table 22)
   Cholesterol
   Sitosterol
   Stigmasterol
5. Versatility of the LNP transfection system with respect to the lipid-PEG implied (Table 23)
   DSG-PEG 2k
   DMG-PEG 2k
   DSPE-PEG 5k
   DSPE-PEG 2k
   ALC-0159 (Azane; 2-(2-methoxyethoxy)-N,N-di(tetradecyl)acetamide).

TABLE 16
Chemical composition of LNPs according to the invention and comparative LNPs, and related physical data.

| | LNP | Cationic | | Ionizable | | Phospho- | | Sterol | | PEG-lipid | | Size | PDI | Zeta | |
|---|---|---|---|---|---|---|---|---|---|---|---|---|---|---|---|
| | No | lipid (mM) | | lipid (mM) | | lipid (mM) | | (mM) | | (mM) | | (nm) | (—) | (mV) | EE % |
| FIGS. | 86AA | W21.7 | 4.000 | DODMA | 3.000 | DPyPE | 1.000 | Cholesterol | 1.850 | DSG-PEG 2k | 0.150 | 48 ± 3 | 0.112 | +11 | 100 |
| 31A | 199 | N/A | N/A | SM-102 | 7.009 | DSPC | 1.402 | Cholesterol | 5.397 | DMG-PEG 2k | 0.210 | 45 ± 2 | 0.291 | 0 | 84 |
| and | 200 | W21.7 | 1.402 | SM-102 | 5.607 | DSPC | 1.402 | Cholesterol | 5.397 | DMG-PEG 2k | 0.210 | 37 ± 3 | 0.147 | +9 | 100 |
| 31B | 201 | W21.7 | 2.804 | SM-102 | 4.205 | DSPC | 1.402 | Cholesterol | 5.397 | DMG-PEG 2k | 0.210 | 37 ± 1 | 0.146 | +13 | 100 |
| | 202 | W21.7 | 4.205 | SM-102 | 2.804 | DSPC | 1.402 | Cholesterol | 5.397 | DMG-PEG 2k | 0.210 | 37 ± 2 | 0.121 | +16 | 100 |
| | 203 | W21.7 | 5.607 | SM-102 | 1.402 | DSPC | 1.402 | Cholesterol | 5.397 | DMG-PEG 2k | 0.210 | 39 ± 1 | 0.203 | +19 | 100 |
| FIGS. | 86Y | W21.7 | 4.000 | DODMA | 3.000 | DPyPE | 1.000 | Cholesterol | 1.850 | DSG-PEG 2k | 0.150 | 63 ± 2 | 0.067 | +12 | 100 |
| 32A | 199B | N/A | N/A | SM-102 | 7.000 | DSPC | 1.400 | Cholesterol | 5.390 | DMG-PEG 2k | 0.210 | 36 ± 1 | 0.120 | +1 | 95 |
| and | 204 | W21.7 | 1.400 | SM-102 | 7.000 | DSPC | 1.400 | Cholesterol | 5.390 | DMG-PEG 2k | 0.210 | 39 ± 2 | 0.198 | +12 | 99 |
| 32B | 205 | W21.7 | 2.800 | SM-102 | 7.000 | DSPC | 1.400 | Cholesterol | 5.390 | DMG-PEG 2k | 0.210 | 41 ± 1 | 0.159 | +15 | 100 |
| | 206 | W21.7 | 4.200 | SM-102 | 7.000 | DSPC | 1.400 | Cholesterol | 5.390 | DMG-PEG 2k | 0.210 | 41 ± 2 | 0.152 | +15 | 100 |
| | 207 | W21.7 | 5.600 | SM-102 | 7.000 | DSPC | 1.400 | Cholesterol | 5.390 | DMG-PEG 2k | 0.210 | 42 ± 3 | 0.161 | +16 | 100 |
| | 208 | W21.7 | 7.000 | SM-102 | 7.000 | DSPC | 1.400 | Cholesterol | 5.390 | DMG-PEG 2k | 0.210 | 33 ± 1 | 0.297 | +21 | 100 |
| FIGS. | 99I | W21.7 | 4.0 | DODMA | 3.0 | DPyPE | 1.0 | Cholesterol | 1.850 | DSG-PEG 2k | 0.15 | 57 ± 4 | 0.113 | +13 | 99 |
| 33A | 281 | N/A | N/A | SM-102 | 7.0 | DSPC | 1.4 | Cholesterol | 5.390 | DMG-PEG 2k | 0.21 | 39 ± 2 | 0.167 | +1 | 96 |
| and | 282 | W21.7 | 1.4 | SM-102 | 5.6 | DSPC | 1.4 | Cholesterol | 5.397 | DMG-PEG 2k | 0.21 | 37 ± 3 | 0.189 | +11 | 99 |
| 33B | 283 | W21.7 | 1.4 | SM-102 | 7.0 | DSPC | 1.4 | Cholesterol | 5.390 | DMG-PEG 2k | 0.21 | 37 ± 1 | 0.180 | +11 | 99 |
| FIGS. | 86Y | W21.7 | 4.000 | DODMA | 3.000 | DPyPE | 1.000 | Cholesterol | 1.850 | DSG-PEG 2k | 0.150 | 63 ± 2 | 0.067 | +12 | 100 |
| 34A | 199B | N/A | N/A | SM-102 | 7.000 | DSPC | 1.400 | Cholesterol | 5.390 | DMG-PEG 2k | 0.210 | 36 ± 1 | 0.120 | +1 | 95 |
| and | 209 | N/A | N/A | SM-102 | 7.009 | DPyPE | 1.402 | Cholesterol | 5.397 | DSG-PEG 2k | 0.210 | 60 ± 4 | 0.057 | +1 | 95 |
| 34B | 210 | W21.7 | 1.402 | SM-102 | 5.607 | DPyPE | 1.402 | Cholesterol | 5.397 | DSG-PEG 2k | 0.210 | 40 ± 1 | 0.137 | +7 | 97 |
| | 211 | W21.7 | 2.804 | SM-102 | 4.205 | DPyPE | 1.402 | Cholesterol | 5.397 | DSG-PEG 2k | 0.210 | 36 ± 2 | 0.111 | +8 | 98 |
| | 212 | W21.7 | 4.205 | SM-102 | 2.804 | DPyPE | 1.402 | Cholesterol | 5.397 | DSG-PEG 2k | 0.210 | 41 ± 2 | 0.057 | +11 | 98 |
| | 213 | W21.7 | 5.607 | SM-102 | 1.402 | DPyPE | 1.402 | Cholesterol | 5.397 | DSG-PEG 2k | 0.210 | 37 ± 1 | 0.131 | +13 | 98 |
| | 214 | W21.7 | 7.009 | N/A | N/A | DPyPE | 1.402 | Cholesterol | 5.397 | DSG-PEG 2k | 0.210 | 41 ± 2 | 0.155 | +12 | 98 |
| FIG. | 86AB | W21.7 | 4.000 | DODMA | 3.000 | DPyPE | 1.000 | Cholesterol | 1.850 | DSG-PEG 2k | 0.150 | 55 ± 3 | 0.152 | +14 | 100 |
| 35 | 232 | W21.7 | 4.000 | SM-102 | 3.000 | DSPC | 1.000 | Cholesterol | 1.850 | DSG-PEG 2k | 0.150 | 70 ± 8 | 0.208 | +1 | 100 |
| | 233 | W21.7 | 4.000 | DLin- | 3.000 | DSPC | 1.000 | Cholesterol | 1.850 | DSG-PEG 2k | 0.150 | 55 ± 3 | 0.075 | +14 | 100 |
| | | | | MC3- | | | | | | | | | | | |
| | | | | DMA | | | | | | | | | | | |
| | 234 | W21.7 | 4.000 | ALC-0315 | 3.000 | DSPC | 1.000 | Cholesterol | 1.850 | DSG-PEG 2k | 0.150 | 58 ± 2 | 0.072 | +13 | 100 |
| FIG. | 86AC | W21.7 | 4.000 | DODMA | 3.000 | DPyPE | 1.000 | Cholesterol | 1.850 | DSG-PEG 2k | 0.150 | 54 ± 1 | 0.085 | +16 | 100 |
| 36 | 237 | W21.7 | 4.000 | DODMA | 3.000 | PLPE | 1.000 | Cholesterol | 1.850 | DSG-PEG 2k | 0.150 | 52 ± 9 | 0.197 | +14 | 100 |
| | 238 | W21.7 | 4.000 | DODMA | 3.000 | DOPC | 1.000 | Cholesterol | 1.850 | DSG-PEG 2k | 0.150 | 50 ± 10 | 0.163 | +17 | 100 |
| | 239 | W21.7 | 4.000 | DODMA | 3.000 | DOPE | 1.000 | Cholesterol | 1.850 | DSG-PEG 2k | 0.150 | 60 ± 5 | 0.155 | +13 | 100 |
| | 240 | W21.7 | 4.000 | DODMA | 3.000 | DPPC | 1.000 | Cholesterol | 1.850 | DSG-PEG 2k | 0.150 | 59 ± 8 | 0.134 | +17 | 100 |
| | 241 | W21.7 | 4.000 | DODMA | 3.000 | DSPC | 1.000 | Cholesterol | 1.850 | DSG-PEG 2k | 0.150 | 43 ± 3 | 0.162 | +15 | 100 |
| FIG. | 86AB | W21.7 | 4.000 | DODMA | 3.000 | DPyPE | 1.000 | Cholesterol | 1.850 | DSG-PEG 2k | 0.150 | 55 ± 3 | 0.152 | +14 | 100 |
| 37 | 235 | W21.7 | 4.000 | DODMA | 3.000 | DPyPE | 1.000 | Sitosterol | 1.850 | DSG-PEG 2k | 0.150 | 77 ± 9 | 0.163 | +14 | 100 |
| | 236 | W21.7 | 4.000 | DODMA | 3.000 | DPyPE | 1.000 | Stigmasterol | 1.850 | DSG-PEG 2k | 0.150 | 127 ± 11 | 0.177 | +7 | 100 |
| FIG. | 86AD | W21.7 | 4.000 | DODMA | 3.000 | DPyPE | 1.000 | Cholesterol | 1.850 | DSG-PEG 2k | 0.150 | 75 ± 5 | 0.050 | +11 | 100 |
| 38 | 263 | W21.7 | 4.000 | DODMA | 3.000 | DPyPE | 1.000 | Cholesterol | 1.850 | DMG-PEG 2k | 0.150 | 40 ± 3 | 0.198 | +18 | 100 |
| | 264 | W21.7 | 4.000 | DODMA | 3.000 | DPyPE | 1.000 | Cholesterol | 1.850 | DSPE-PEG 5k | 0.150 | 61 ± 4 | 0.082 | +2 | 100 |
| | 265 | W21.7 | 4.000 | DODMA | 3.000 | DPyPE | 1.000 | Cholesterol | 1.850 | DSPE-PEG 2k | 0.150 | 85 ± 3 | 0.056 | +24 | 100 |
| | 266 | W21.7 | 4.000 | DODMA | 3.000 | DPyPE | 1.000 | Cholesterol | 1.850 | ALC-0159 2k | 0.150 | 36 ± 6 | 0.216 | +18 | 100 |

### Experimental Part

### Cell Lines

### Caco-2 Cells

Human epithelial cells from colorectal adenocarcinoma (Caco-2) were grown on cell flask coated with fibronectin (0.05 mg/ml) and cultured in DMEM glucose 4.5 g/L supplemented with Fetal bovine serum (FBS, 20%), Na Pyruvate (1%), L-Glutamine (1%), non-essential amino acids (AANE, 1%) and Penicillin-Streptomycin (1%).

### HEK-293(a) Cells

Human embryonic kidney cells (HEK293(a)) were grown on cell flask coated with fibronectin (0.05 mg/ml) and cultured in MEM Eagle supplemented with Fetal bovine serum (FBS, 10%), L-Glutamine (1%), non-essential amino acids (AANE, 1%) and Penicillin-Streptomycin (1%).

### In Vitro Transfection

Fluc mRNA Transfection of Adherent Cell Lines

For transfection experiments, 4×10⁴ Caco-2 cells or 5×10⁴ HEK-293(a) cells were seeded per well of 24-well plates in complete medium 1 day before transfection. On the day of transfection, in vivo-jetRNA®+/Fluc mRNA complexes were prepared according to the manufacturers' recommendations. Briefly, transfection with in vivo-jetRNA®+was performed as described: 500 ng of Fluc-encoding mRNA (per well of 24-well plate) were first diluted in the provided mRNA Buffer, followed by the mixing-in of 1 μl in vivo-jetRNA®+. Following an incubation of 15 minutes at room temperature, in vivo-jetRNA®+complexes or 500 ng of LNP X³ (a list of the different LNP may be found in Table 16) were simply added dropwise to cells in their complete growth medium. Transfection efficiency was assessed 24 hours post-transfection by luminescence reading.

### In Vivo Transfection

10 μg of mRNA encoding Luciferase was administered into OF1 mice using LNPs via intravenous injection. Luciferase expression was assessed 24 h post-injection. The organs of interest were dissected, rinsed in PBS (×1) and mixed with the tissue homogenizer Precellys Evolution touch. Each organ mix was frozen at −80° C., thawed and an aliquot of 0.5 mL was taken for luciferase analysis. The aliquot was centrifuged for 5 min at 12 000 rpm at 4° C. Luciferase enzyme activity was assessed on 5 μL of organ lysate supernatant using 100 μl of luciferin solution. The luminescence (expressed as RLU) was measured by using a luminometer and normalized per mg of organ protein with Pierce BCA Assay Protein Kit.

### 10.1 Beneficial Technical Effect of W21.7

### 1. Substitution of SM-102 by W21.7 in SpikeVax Formulation

TABLE 17
Chemical composition of screened LNPs 86AA, 199-203

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86AA | W21.7 | 4.000 | DODMA | 3.000 | DPyPE | 1.000 | Cholesterol | 1.850 | DSG-PEG 2k | 0.150 | 48 ± 3 | 0.112 | +11 | 100 |
| 199 | N/A | N/A | SM-102 | 7.009 | DSPC | 1.402 | Cholesterol | 5.397 | DMG-PEG 2k | 0.210 | 45 ± 2 | 0.291 | 0 | 84 |
| 200 | W21.7 | 1.402 | SM-102 | 5.607 | DSPC | 1.402 | Cholesterol | 5.397 | DMG-PEG 2k | 0.210 | 37 ± 3 | 0.147 | +9 | 100 |
| 201 | W21.7 | 2.804 | SM-102 | 4.205 | DSPC | 1.402 | Cholesterol | 5.397 | DMG-PEG 2k | 0.210 | 37 ± 1 | 0.146 | +13 | 100 |
| 202 | W21.7 | 4.205 | SM-102 | 2.804 | DSPC | 1.402 | Cholesterol | 5.397 | DMG-PEG 2k | 0.210 | 37 ± 2 | 0.121 | +16 | 100 |
| 203 | W21.7 | 5.607 | SM-102 | 1.402 | DSPC | 1.402 | Cholesterol | 5.397 | DMG-PEG 2k | 0.210 | 39 ± 1 | 0.203 | +19 | 100 |

The substitution of the ionizable lipid SM-102 by the cationic lipid W21.7 in SpikeVax formulation increased the mRNA encapsulation efficiency (from 84% to 100%). The same substitution slightly reduced LNP size from 45 to 37 nm and increased the LNP zeta potential from 0 to +19 mV with ratio W21.7/SM-102 8/2 (LNP203). This increase of zeta potential could be related with the permanently cationic character of W21.7.

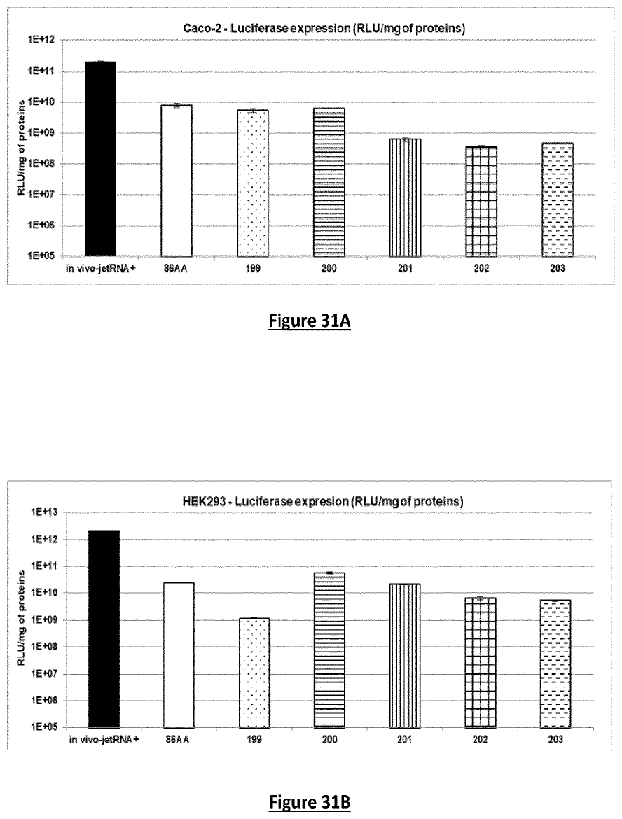

On the hard-to-transfect Caco-2 cells, roughly same luciferase expression was obtained with LNP86AA and LNP199 (SpikeVax-like LNP formulation 5-8.109 RLU/mg of proteins). When 20% of SM-102 was substituted by W21.7 (LNP 200) there was no impact on the luciferase expression. However, when SM-102 was substituted with higher amount of W21.7 (LNP201 ratio W21.7/SM-102 4/6, LNP202 ratio W21.7/SM-102 6/4 and LNP203 ratio W21.7/SM-102 8/2) there was a decrease in mRNA expression (4-6.109 RLU/mg of proteins) (see FIG. 31A).

On HEK-293(a) cells, LNP86AA (2·10¹⁰ RLU/mg of proteins) gave 10-fold higher luciferase expression than LNP199 (SpikeVax-like, 1.109 RLU/mg of proteins). When SM-102 was substituted by W21.7, LNPs formulation (LNP200-203) gave higher luciferase expression (5.109 to 6·10¹⁰ RLU/mg of proteins) than LNP199 (see FIG. 31B).

### 2. Effect of W21.7 Addition in SpikeVax Formulation

TABLE 18
Chemical composition of screened LNPs 86Y, 199B, 204-208.

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86Y | W21.7 | 4.000 | DODMA | 3.000 | DPyPE | 1.000 | Cholesterol | 1.850 | DSG-PEG 2k | 0.150 | 63 ± 2 | 0.067 | +12 | 100 |
| 199B | N/A | N/A | SM-102 | 7.000 | DSPC | 1.400 | Cholesterol | 5.390 | DMG-PEG 2k | 0.210 | 36 ± 1 | 0.120 | +1 | 95 |
| 204 | W21.7 | 1.400 | SM-102 | 7.000 | DSPC | 1.400 | Cholesterol | 5.390 | DMG-PEG 2k | 0.210 | 39 ± 2 | 0.198 | +12 | 99 |
| 205 | W21.7 | 2.800 | SM-102 | 7.000 | DSPC | 1.400 | Cholesterol | 5.390 | DMG-PEG 2k | 0.210 | 41 ± 1 | 0.159 | +15 | 100 |
| 206 | W21.7 | 4.200 | SM-102 | 7.000 | DSPC | 1.400 | Cholesterol | 5.390 | DMG-PEG 2k | 0.210 | 41 ± 2 | 0.152 | +15 | 100 |
| 207 | W21.7 | 5.600 | SM-102 | 7.000 | DSPC | 1.400 | Cholesterol | 5.390 | DMG-PEG 2k | 0.210 | 42 ± 3 | 0.161 | +16 | 100 |
| 208 | W21.7 | 7.000 | SM-102 | 7.000 | DSPC | 1.400 | Cholesterol | 5.390 | DMG-PEG 2k | 0.210 | 33 ± 1 | 0.297 | +21 | 100 |

The addition of W21.7 in SpikeVax formulation had almost no impact on LNP size and encapsulation efficiency. However, addition of W21.7 in SpikeVax formulation increased the LNP zeta potential from +1 to +21 mV for LNP208 with an equimolar ratio of W21.7 and SM-102 (7.0 mM). Here again, this phenomenon could be related to the cationic character of W21.7.

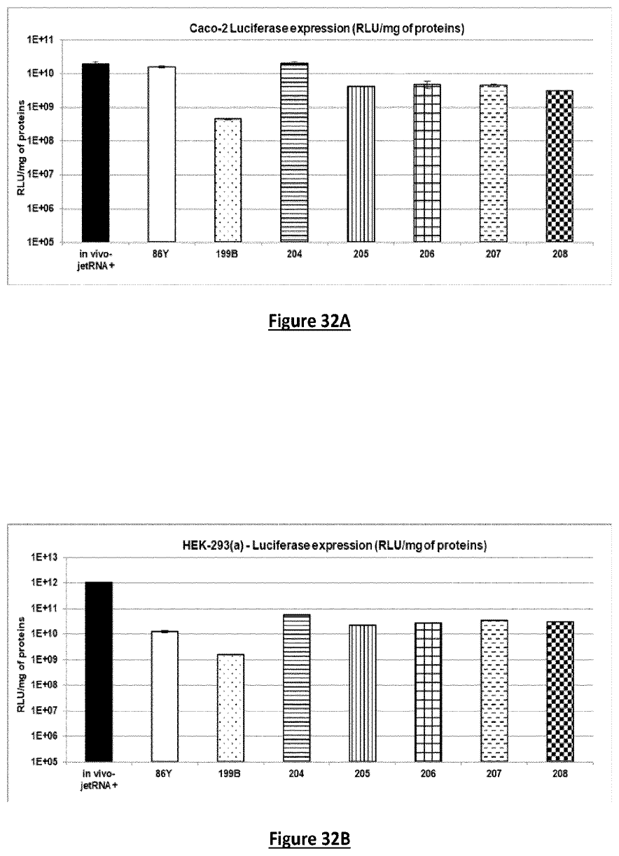

On the hard-to-transfect Caco-2 cells, the addition of W21.7 in SpikeVax formulation increased mRNA expression compared to SpikeVax formulation (5·10⁸ RLU/mg of proteins). LNP204 with a ratio of W21.7/SM-102 of 1·4 mM/7 mM gave same transfection efficiency (2·10¹⁰ RLU/mg of proteins) than LNP86Y (W21.7/DODMA 4 mM/3 mM). In contrast, LNPs 205-208 gave lower luciferase expression compared to LNP86Y (3-5.109 RLU/mg of proteins) but higher than SpikeVax formulation (see FIG. 32A).

On HEK-293(a) cells, the addition of W21.7 in SpikeVax formulation increased luciferase expression compared to the initial formulation LNP199B but also compared to LNP86Y (W21.7/DODMA 4 mM/3 mM) (see FIG. 32B).

TABLE 19
Chemical composition of screened LNPs 99I, 281-283

| LNP | Cationic | | Ionizable | | Phospho- | | | | PEG-lipid | | size | PDI | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | (—) | (mV) | EE % |
| 99I | W21.7 | 4.0 | DODMA | 3.0 | DPyPE | 1.0 | Cholesterol | 1.850 | DSG-PEG 2k | 0.15 | 57 ± 4 | 0.113 | +13 | 99 |
| 281 | N/A | N/A | SM-102 | 7.0 | DSPC | 1.4 | Cholesterol | 5.390 | DMG-PEG 2k | 0.21 | 39 ± 2 | 0.167 | +1 | 96 |
| 282 | W21.7 | 1.4 | SM-102 | 5.6 | DSPC | 1.4 | Cholesterol | 5.397 | DMG-PEG 2k | 0.21 | 37 ± 3 | 0.189 | +11 | 99 |
| 283 | W21.7 | 1.4 | SM-102 | 7.0 | DSPC | 1.4 | Cholesterol | 5.390 | DMG-PEG 2k | 0.21 | 37 ± 1 | 0.180 | +11 | 99 |

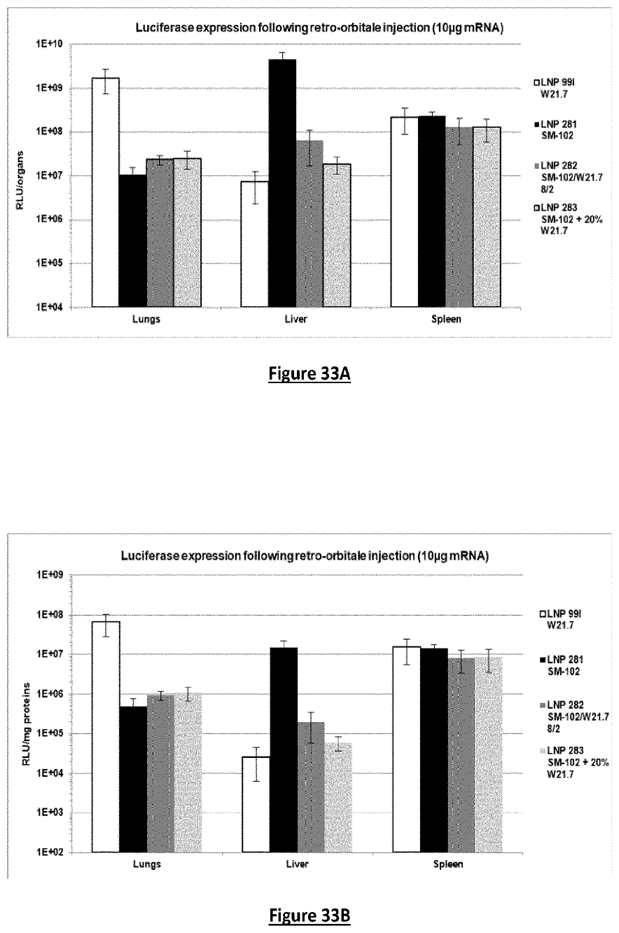

mRNA encoding Luciferase was administered into OF1 mice using 10 μg of mRNA injected via intravenous (retro orbital) route. Luciferase expression was assessed 24 h post-injection. As expected, due to their positively zeta potential, lower luciferase expression was observed in the liver with the 3 different LNP formulations containing W21.7 (LNP991, LNP282 and LNP283 with 3.104-2.105 RLU/mg of proteins) compared to SpikeVax formulation (LNP281, 1·107 RLU/mg of proteins). Higher luciferase expression was observed in the lung with LNP containing the highest amount of the lipid W21.7 (LNP 991, 7.107 RLU/mg of proteins) compared to SpikeVax formulation with W21.7 (LNP282 and LNP283, 1·10⁶ RLU/mg of proteins) or without W21.7 (LNP281, 5.105 RLU/mg of proteins). Similar luciferase expression in the spleen was observed with all LNPs (8·10⁶-1.107 RLU/mg of proteins, FIGS. 33 A and B).

### 3. Substitution of SM-102 by W21.7 in SpikeVax Formulation in More Efficient System

TABLE 19
Chemical composition of screened LNPs 86Y, 199B, 209-214

| LNP | Cationic | | Ionizable | | Phospho- | | | | PEG-lipid | | size | PDI | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | (—) | (mV) | EE % |
| 86Y | W21.7 | 4.000 | DODMA | 3.000 | DPyPE | 1.000 | Cholesterol | 1.850 | DSG-PEG 2k | 0.150 | 63 ± 2 | 0.067 | +12 | 100 |
| 199B | N/A | N/A | SM-102 | 7.000 | DSPC | 1.400 | Cholesterol | 5.390 | DMG-PEG 2k | 0.210 | 36 ± 1 | 0.120 | +1 | 95 |
| 209 | N/A | N/A | SM-102 | 7.009 | DPyPE | 1.402 | Cholesterol | 5.397 | DSG-PEG 2k | 0.210 | 60 ± 4 | 0.057 | +1 | 95 |
| 210 | W21.7 | 1.402 | SM-102 | 5.607 | DPyPE | 1.402 | Cholesterol | 5.397 | DSG-PEG 2k | 0.210 | 40 ± 1 | 0.137 | +7 | 97 |
| 211 | W21.7 | 2.804 | SM-102 | 4.205 | DPyPE | 1.402 | Cholesterol | 5.397 | DSG-PEG 2k | 0.210 | 36 ± 2 | 0.111 | +8 | 98 |
| 212 | W21.7 | 4.205 | SM-102 | 2.804 | DPyPE | 1.402 | Cholesterol | 5.397 | DSG-PEG 2k | 0.210 | 41 ± 2 | 0.057 | +11 | 98 |
| 213 | W21.7 | 5.607 | SM-102 | 1.402 | DPyPE | 1.402 | Cholesterol | 5.397 | DSG-PEG 2k | 0.210 | 37 ± 1 | 0.131 | +13 | 98 |
| 214 | W21.7 | 7.009 | N/A | N/A | DPyPE | 1.402 | Cholesterol | 5.397 | DSG-PEG 2k | 0.210 | 41 ± 2 | 0.155 | +12 | 98 |

LNP formulation (LNP209) with ionizable lipid SM-102, lipids ratio from SpikeVax formulation but with phospholipid, sterol and PEG-lipid adapted for W21.7 gave same zeta potential, and mRNA encapsulation efficiency but higher particle size (from 36 nm to 60 nm). The substitution of the ionizable lipid SM-102 by the cationic lipid W21.7 in this adapted SpikeVax formulation had no impact on the mRNA encapsulation efficiency. The same substitution slightly reduced LNP size from 60 to 36 nm and increased the LNP zeta potential from +1 to +13 mV with ratio W21.7/SM-102 8/2 (LNP213). This increase of zeta potential could be related with the permanently cationic character of W21.7.

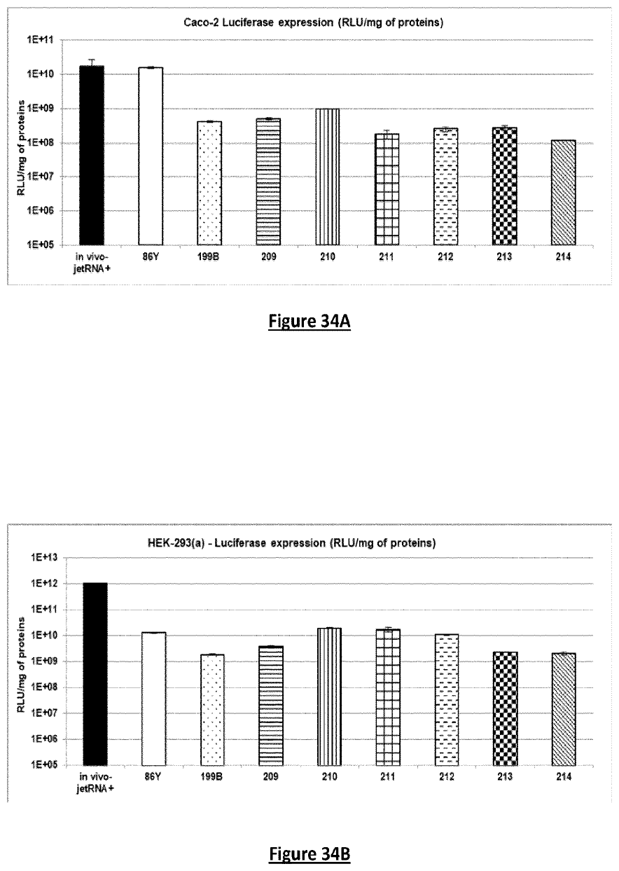

On the hard-to-transfect Caco-2 cells, as previously shown lower luciferase expression was observed for LNP199B (SpikeVax-like LNP formulation, 4·10⁴ RLU/mg of proteins) compared to LNP86Y (2·10¹⁰ RLU/mg of proteins). LNP formulation (LNP209) with ionizable lipid SM-102, lipids ratio from SpikeVax formulation but with phospholipid, sterol and PEG-lipid adapted for W21.7 gave similar luciferase expression than LNP199B. When 20% of SM-102 was substituted by W21.7 (LNP210) there was an increase in luciferase expression. However, when SM-102 was substituted with higher amount of W21.7 (LNP211 ratio W21.7/SM-102 4/6, LNP212 ratio W21.7/SM-102 6/4, LNP213 ratio W21.7/SM-102 8/2 and LNP214 ratio W21.7/SM-102 10/0) there was a decrease in transfection efficiency (4-6.109 RLU/mg of proteins) (FIG. 34A).

On HEK-293(a) cells, LNP86AY gave higher luciferase expression than LNP199B (SpikeVax-like). LNP formulation (LNP209) with ionizable lipid SM-102, lipids ratio from SpikeVax formulation but with phospholipid, sterol lipid and PEG-lipid adapted for W21.7 gave higher luciferase expression than LNP199B. When SM-102 was substituted by W21.7 in this formulation, LNPs LNP210, 211 and 212 (LNP210 ratio W21.7/SM-102 8/2, LNP211 ratio W21.7/SM-102 4/6 and LNP212 ratio W21.7/SM-102 6/4) gave higher luciferase expression than LNP199B (1-2·10¹⁰ compared to 2.109 RLU/mg of proteins). When 80 or 100% of SM-102 was substituted by W21.7 (LNP213 and 214) there was no impact on luciferase expression (FIG. 34B).

4. Versatility of the Delivery Platform with Reference to Ionizable Lipid

TABLE 20
Chemical composition of screened LNPs 86AB, 232-234

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86AB | W21.7 | 4.000 | DODMA | 3.000 | DPyPE | 1.000 | Cholesterol | 1.850 | DSG-PEG 2k | 0.150 | 55 ± 3 | 0.152 | +14 | 100 |
| 232 | W21.7 | 4.000 | SM-102 | 3.000 | DSPC | 1.000 | Cholesterol | 1.850 | DSG-PEG 2k | 0.150 | 70 ± 8 | 0.208 | +1 | 100 |
| 233 | W21.7 | 4.000 | DLin-MC3-DMA | 3.000 | DSPC | 1.000 | Cholesterol | 1.850 | DSG-PEG 2k | 0.150 | 55 ± 3 | 0.075 | +14 | 100 |
| 234 | W21.7 | 4.000 | ALC-0315 | 3.000 | DSPC | 1.000 | Cholesterol | 1.850 | DSG-PEG 2k | 0.150 | 58 ± 2 | 0.072 | +13 | 100 |

When swapping the ionizable lipid from DODMA to DLin-MC3-DMA or ALC-0315, similar particle size (55 nm) and zeta potential (+14 mV) were observed. However, when considering SM-102 as ionizable lipid, LNP size was bigger (70 nm) and displayed a lower zeta potential (+1 mV). This suggested a high impact of ionizable lipid to particle nanomorphology.

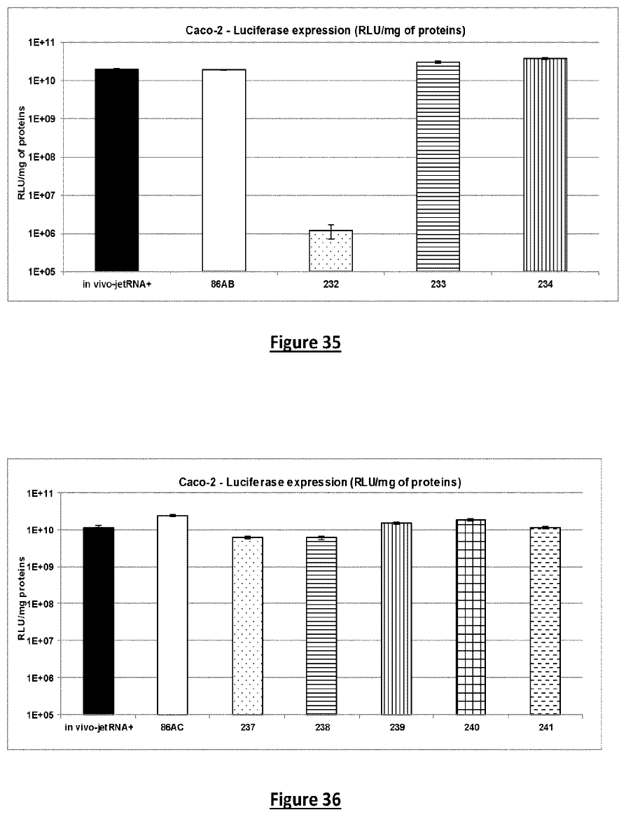

On the hard-to-transfect Caco-2 cells, LNP formulations with DLin-MC3-DMA (LNP233) or ALC-0315 (LNP234) as ionizable lipids gave slightly higher luciferase expression (3-4·10¹⁰ RLU/mg of proteins) compared to LNP86AB with DODMA as ionizable lipid (2·10¹⁰ RLU/mg of proteins). However, LNP232 with SM-102 as ionizable lipid gave lower mRNA expression compared to LNP86AB (2·10⁶ RLU/mg of proteins) (FIG. 35).

5. Versatility of Delivery Platform with Reference to Phospholipid

TABLE 21
Chemical composition of screened LNPs 86AC, 237-241

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86AC | W21.7 | 4.000 | DODMA | 3.000 | DPyPE | 1.000 | Cholesterol | 1.850 | DSG-PEG 2k | 0.150 | 54 ± 1 | 0.085 | +16 | 100 |
| 237 | W21.7 | 4.000 | DODMA | 3.000 | PLPE | 1.000 | Cholesterol | 1.850 | DSG-PEG 2k | 0.150 | 52 ± 9 | 0.197 | +14 | 100 |
| 238 | W21.7 | 4.000 | DODMA | 3.000 | DOPC | 1.000 | Cholesterol | 1.850 | DSG-PEG 2k | 0.150 | 50 ± 10 | 0.163 | +17 | 100 |
| 239 | W21.7 | 4.000 | DODMA | 3.000 | DOPE | 1.000 | Cholesterol | 1.850 | DSG-PEG 2k | 0.150 | 60 ± 5 | 0.155 | +13 | 100 |
| 240 | W21.7 | 4.000 | DODMA | 3.000 | DPPC | 1.000 | Cholesterol | 1.850 | DSG-PEG 2k | 0.150 | 59 ± 8 | 0.134 | +17 | 100 |
| 241 | W21.7 | 4.000 | DODMA | 3.000 | DSPC | 1.000 | Cholesterol | 1.850 | DSG-PEG 2k | 0.150 | 43 ± 3 | 0.162 | +15 | 100 |

On the hard-to-transfect Caco-2 cells, LNP239, 240 and 214 with phospholipid DOPE, DPPC and DSPC, respectively, gave similar luciferase expression than LNP86AC with DPyPE (1-2·10¹⁰ RLU/mg of proteins). LNP 237 and 238 with PLPE and DOPC gave slightly lower mRNA expression (6.109 RLU/mg of proteins) than LNP86AC (FIG. 36).

6. Versatility of Delivery Platform with Regards to Sterol Type

TABLE 22
Chemical composition of screened LNPs 86AB, 235, 236

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86AB | W21.7 | 4.000 | DODMA | 3.000 | DPyPE | 1.000 | Cholesterol | 1.850 | DSG-PEG 2k | 0.150 | 55 ± 3 | 0.152 | +14 | 100 |
| 235 | W21.7 | 4.000 | DODMA | 3.000 | DPyPE | 1.000 | Sitosterol | 1.850 | DSG-PEG 2k | 0.150 | 77 ± 9 | 0.163 | +14 | 100 |
| 236 | W21.7 | 4.000 | DODMA | 3.000 | DPyPE | 1.000 | Stigmasterol | 1.850 | DSG-PEG 2k | 0.150 | 127 ± 11 | 0.177 | +7 | 100 |

LNP formulation with Sitosterol (LNP235) gave higher particle size (77 nm) compared to LNP with cholesterol (LNP86AB, 55 nm) but both formulations had similar zeta potential (+14 mV). LNP formulation with stigmasterol gave higher particle size than LNP 86AB and 235 (127 nm) and lower zeta potential (+7 mV).

On the hard-to-transfect Caco-2 cells, similar luciferase expression (2-3·10¹⁰ RLU/mg of proteins) was observed with both LNP formulations with similar zeta potential (LNP235 with Sitosterol and LNP86AB with Cholesterol). Lower mRNA expression (1·107 RLU/mg of proteins) was observed with LNP formulation with stigmasterol (LNP 236, presenting a lower zeta potential) compared to LNP 86AB (FIG. 37).

7. Versatility of the Delivery Platform with Reference to PEG-Lipid

TABLE 23
Chemical composition of screened LNPs 86AD, 263-266

| LNP | Cationic | | Ionizable | | Phospho- | | | | PEG-lipid | | size | | Zeta | |
|---|---|---|---|---|---|---|---|---|---|---|---|---|---|---|
| No | lipid (mM) | | lipid (mM) | | lipid (mM) | | Sterol (mM) | | (mM) | | (nm) | PDI | (mV) | EE % |
| 86AD | W21.7 | 4.000 | DODMA | 3.000 | DPyPE | 1.000 | Cholesterol | 1.850 | DSG-PEG 2k | 0.150 | 75 ± 5 | 0.050 | +11 | 100 |
| 263 | W21.7 | 4.000 | DODMA | 3.000 | DPyPE | 1.000 | Cholesterol | 1.850 | DMG-PEG 2k | 0.150 | 40 ± 3 | 0.198 | +18 | 100 |
| 264 | W21.7 | 4.000 | DODMA | 3.000 | DPyPE | 1.000 | Cholesterol | 1.850 | DSPE-PEG 5k | 0.150 | 61 ± 4 | 0.082 | +2 | 100 |
| 265 | W21.7 | 4.000 | DODMA | 3.000 | DPyPE | 1.000 | Cholesterol | 1.850 | DSPE-PEG 2k | 0.150 | 85 ± 3 | 0.056 | +24 | 100 |
| 266 | W21.7 | 4.000 | DODMA | 3.000 | DPyPE | 1.000 | Cholesterol | 1.850 | ALC-0159 2k | 0.150 | 36 ± 6 | 0.216 | +18 | 100 |

The size of the assembled nanoparticles highly depended on the lipid-PEG nature (from 40 nm for LNP263 to 85 nm for LNP265). The zeta potential depended more on the length of the PEG corona rather than its lipid anchor: roughly +20 mV for 2 kDa PEGs (LNPs 86AD, 263, 265, 266), which fell to +2 mV when increasing the length of the hydrophilic corona (LNP 265). This observation could be rationalized considering the additional charge shielding offered by a longer PEG chain.

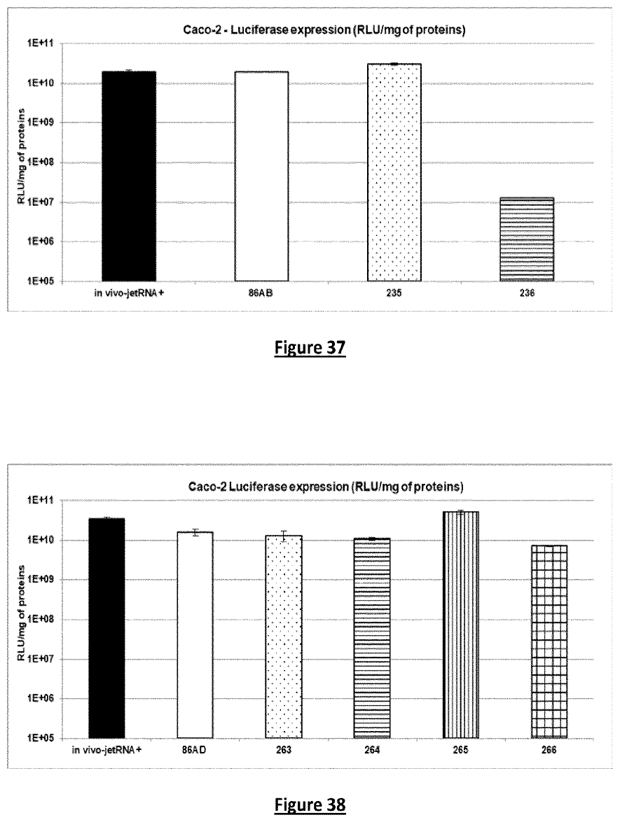

On the hard-to-transfect Caco-2 cells, similar luciferase expression (1-2·10¹⁰ RLU/mg of proteins) was observed with LNP formulations with DSG-PEG₂ₖ (LNP86AD), DMG-PEG₂ₖ (LNP263) and DSPE-PEG₅ₖ (LNP264) whereas a higher luciferase (5·10¹⁰ RLU/mg of proteins) was observed with DSPE-PEG₂ₖ (LNP265) presenting a higher zeta potential and a lower luciferase expression (7.109 RLU/mg of proteins) was observed with ALC-0159₂ₖ (LNP266) (FIG. 38).

### CONCLUSIONS

Compound W21.7 has been identified as potent cationic lipid for nucleic acid delivery.
Used as an additive within competitor's nanoparticle formulations, two main advantages have been identified:
   It can increase the nucleic acid expression in vitro.
   It can modulate the biodistribution of the LNPs to potentially reach more efficiently their therapeutic target.
Compound W21.7 displays a high versatility, both in terms of biology (cell lines; nucleic acid types) and chemistry (compatibility with many ionizable lipids, phospholipids, sterols, PEG-lipids).

1-24. (canceled)

25. A composition comprising:
(A) at least one nucleic acid; and
(B) at least one lipid nanoparticle (LNP) comprising:
(i) at least one ionizable lipid;
(ii) at least one phospholipid;
(iii) at least one sterol;
(iv) at least one poly(ethyleneglycol)-lipid (PEG-lipid); and
(v) an imidazolium-based cationic lipid of formula (I): 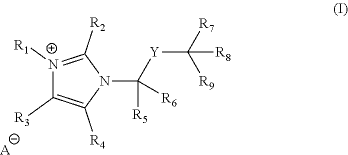
wherein
R₁ represents a C₁-C₁₀ linear or branched hydrocarbon chain or a C₁-C₁₀ hydroxylated chain;
R₂, R₃ and R₄, which may be identical or different, represent H; a C₁-C₁₀ linear or branched hydrocarbon chain or a C₁-C₁₀ hydroxylated chain;
R₅, R₆, R₇, R₈ and R₉, which may be identical or different, represent H; a saturated or unsaturated, linear or branched hydrocarbon chain selected among a C₆-C₃₃ chain, a NH—C₆-C₃₃, a —COO—C₆-C₃₃, a —O—C₆-C₃₃, a —S—C₆-C₃₃; or a saturated or unsaturated C₆ cycle;
Y represents —(CR₁₀R₁₁)ₘ—SO₂—; —(CR₁₀R₁₁)ₘ—COO—; —(CR₁₀R₁₁)ₘ—(CH₂)ₙ—; —(CR₁₀R₁₁)ₘ—CO—NH₂; —(CR₁₀R₁₁)ₘ—CH₂—[O—(CH₂)₂O]ₚ—; —(CR₁₀R₁₁)ₘ—(CH₂)ₙ—S—S—; —(CR₁₀R₁₁)ₘ (CH₂)ₙ—S—; —(CR₁₀R₁₁)ₘ—(CH₂)ₙ—O—; —(CR₁₀R₁₁)ₘ—(CH₂)ₙ—NH—; —CH₂—; —COO—; or —CO—NH— with:
m representing an integer between 1 and 4 inclusive;
n representing an integer between 1 and 10 inclusive; and
p representing an integer between 1 and 4 inclusive;
R₁₀ and R₁₁ representing H; a C₂-C₃₃ saturated or unsaturated, linear or branched hydrocarbon chain; or a saturated or unsaturated C₆ cycle;
A⁻ represents a biocompatible anion;
wherein the at least one nucleic acid is encapsulated in the at least one LNP.

26. The composition according to claim 25, wherein the at least one nucleic acid is either single-, or double-stranded, or combined single and double-stranded on distinct regions of the nucleic acid strand; and is selected from the group consisting of deoxyribonucleic acid (DNA), ribonucleic acid (RNA), small interfering RNA (siRNA), asymmetrical interfering RNA (aiRNA), dicer-substrate short interfering RNA (dsiRNA), small hairpin RNA (shRNA), RNA transcripts, microRNA (miRNA), messenger RNA (mRNA), circular RNA (circRNA), guide RNA (gRNA), small activating RNA (saRNA), small regulatory RNA (srRNA), long non-coding (lncRNA) and antisense oligonucleotide.

27. The composition according to claim 25, wherein the at least one ionizable lipid is selected from the group consisting of 2-dioleyloxy-N,N-dimethyl-3-aminopropane (DODMA), dilinoleylmethyl-4-dimethylaminobutyrate (DLin-MC3-DMA), Heptadecan-9-yl 8-{(2-hydroxyethyl) [6-oxo-6-(undecyloxy)hexyl]amino}octanoate (SM-102) and [(4-Hydroxybutyl)azanediyl]di(hexane-6,1-diyl) bis(2-hexyldecanoate) (ALC-0315) and 30-[N—(N′,N′-dimethylaminoethane)-carbamoyl]cholesterol hydrochloride (DC-cholesterol).

28. The composition according to claim 25, which comprises from 1 mole % to 50 mole % of the at least one ionizable lipid.

29. The composition according to claim 25, wherein the at least one phospholipid is selected from the group consisting of phosphatidylserine (PS), phosphatidylcholine (PC), phosphatidylinositol (PI), 1,2-Dioleoyl-sn-glycero-3-phosphoethanolamine (DOPE), 1,2-diphenytanoyl-sn-glycero-3-phosphoethanolamine (DPyPE), 1,2-distearoyl-sn-glycero-3-phosphocholine (DSPC), palmitoyl linoleoyl phosphatidylethanolamine (PaLiPE), dilinoleoyl phosphatidylethanolamine (DiLiPE) and phosphatidylethanolamine (PE).

30. The composition according to claim 25, which comprises from 1 mole % to 50 mole % of the at least one phospholipid.

31. The composition according to claim 25, wherein the at least one sterol is selected from the group consisting of cholesterol, stigmasterol, beta-sitosterol, 1, ergosterol, campesterol, oxysterol, antrosterol, desmosterol and nicasterol.

32. The composition according to claim 25, which comprises from 1 mole % to 50 mole % of the at least one sterol.

33. The composition according to claim 25, wherein the at least one PEG-lipid is selected from the group consisting of 1,2-Dimyristoyl-sn-glycero-3-methoxypolyethylene glycol (DMG-PEG), 1,2-Distearoyl-rac-glycero-3-methylpolyoxyethylene (DSG-PEG), diacylglycerol-polyethylene glycol (DAG-PEG), 1,2-dipalmitoyl-sn-glycero-3-phosphoethanolamine-N-[azido(polyethylene glycol) (DPPE-PEG) and 3-N-[(ω-methoxypoly(ethylene glycol)2000)carbamoyl]-1,2-dimyristyloxy-propylamine (PEG-c-DMA).

34. The composition according to claim 25, which comprises from 0.1 mole % to 10 mole % of the at least one PEG-lipid.

35. The composition according to claim 25, wherein the imidazolium-based cationic lipid of formula (I) is selected from the group consisting of the following compounds:

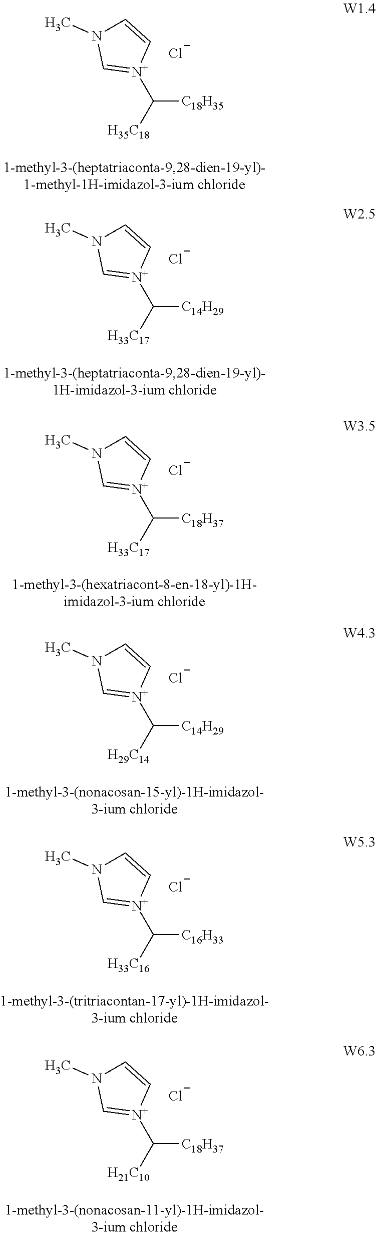

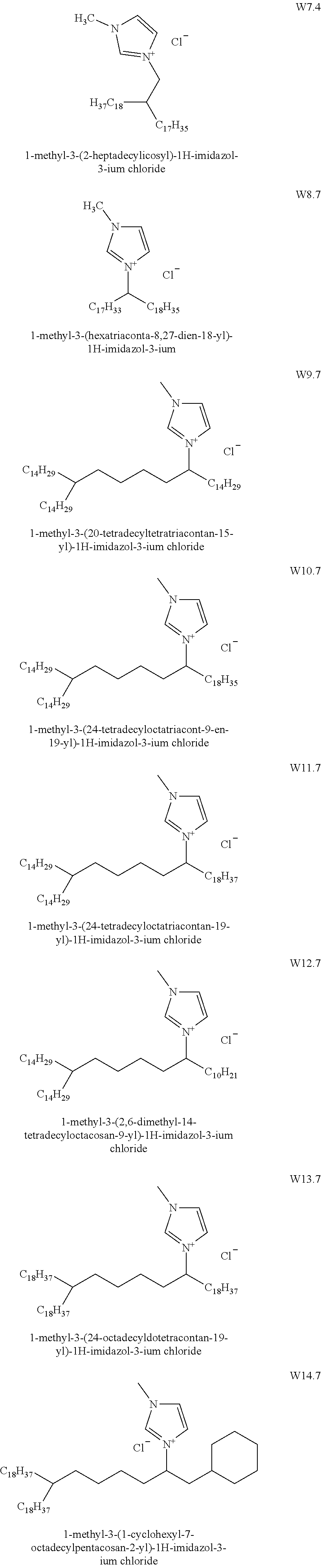

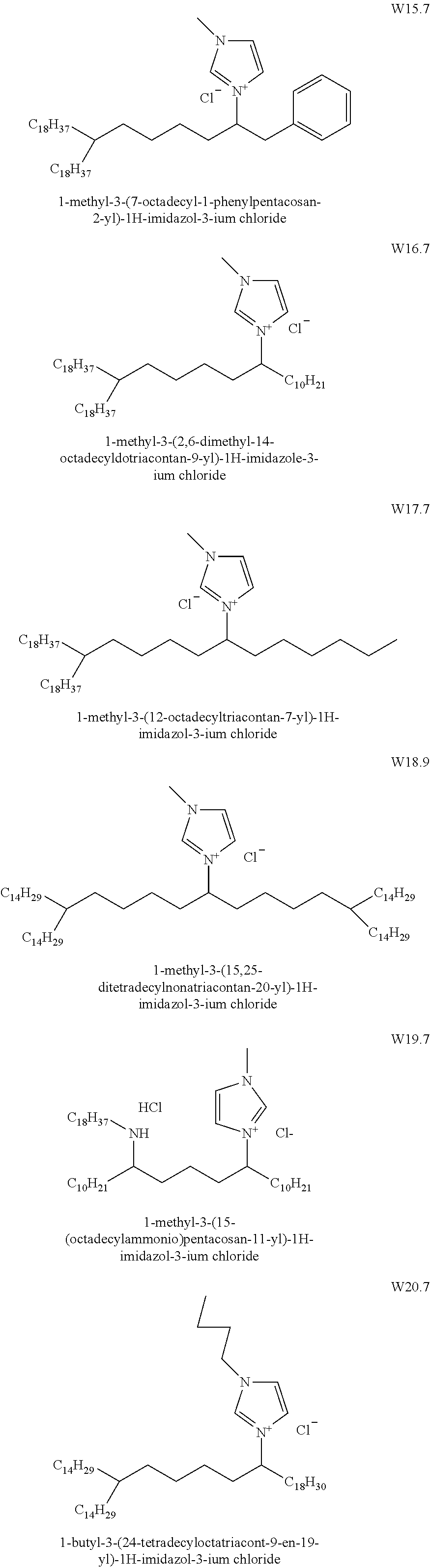

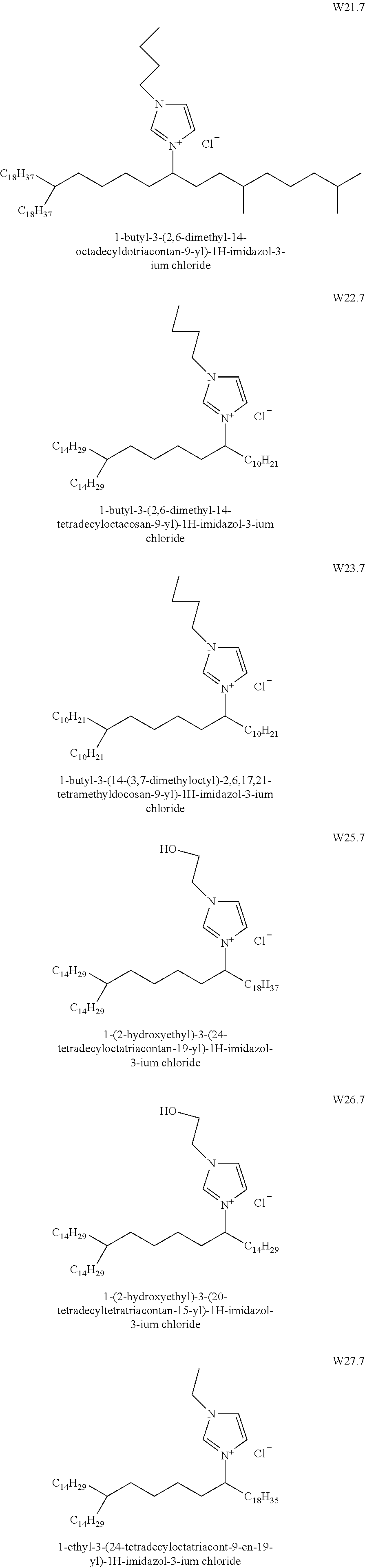

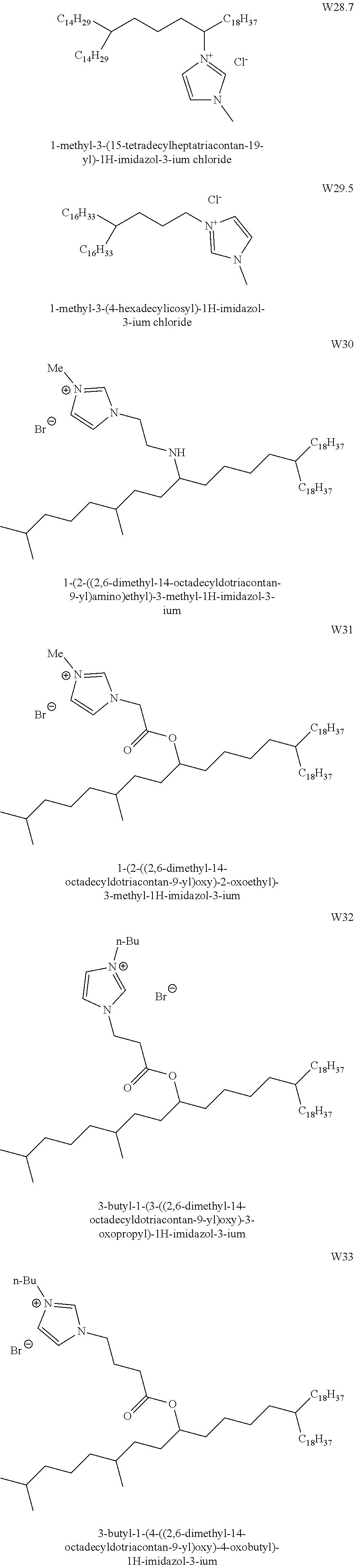

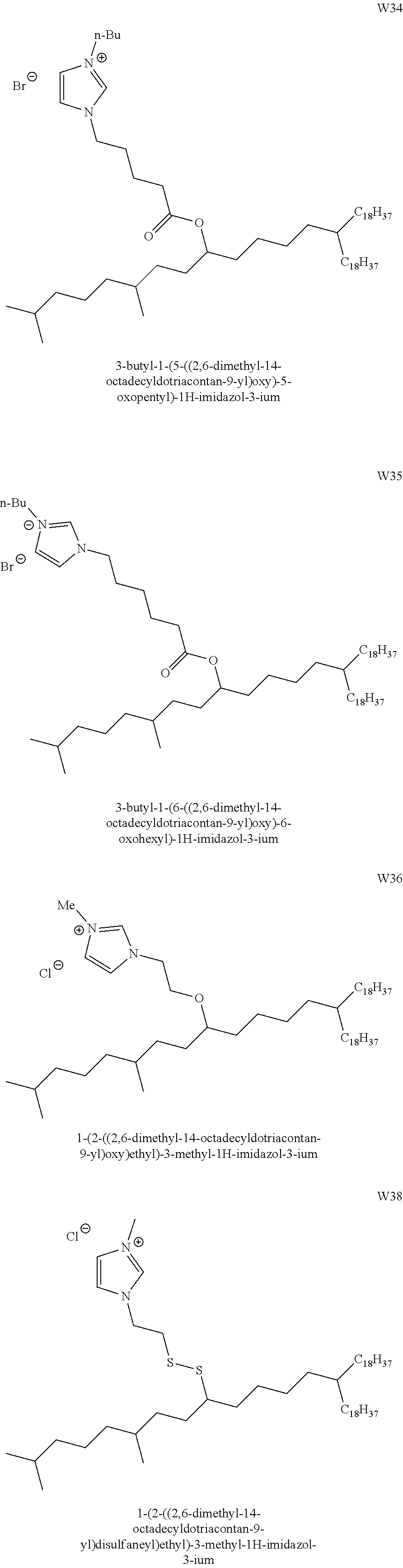

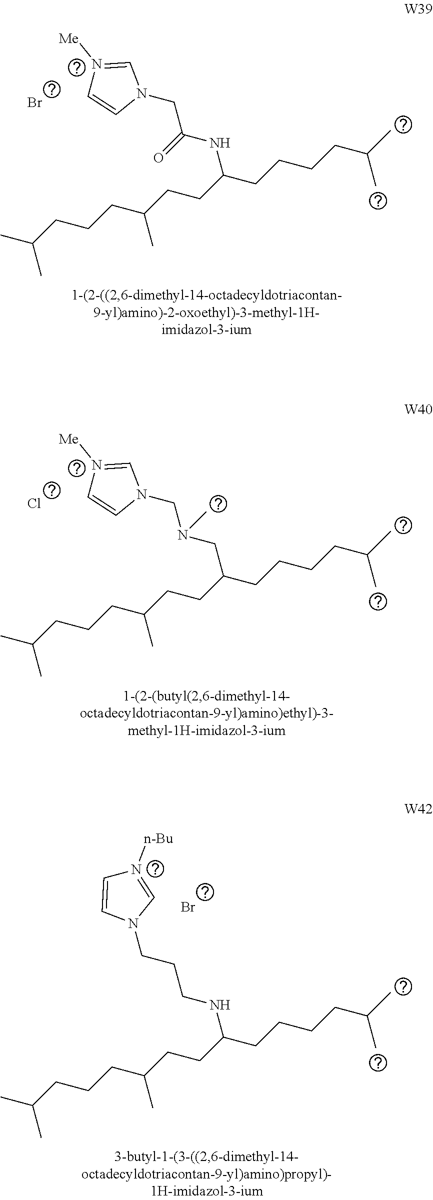

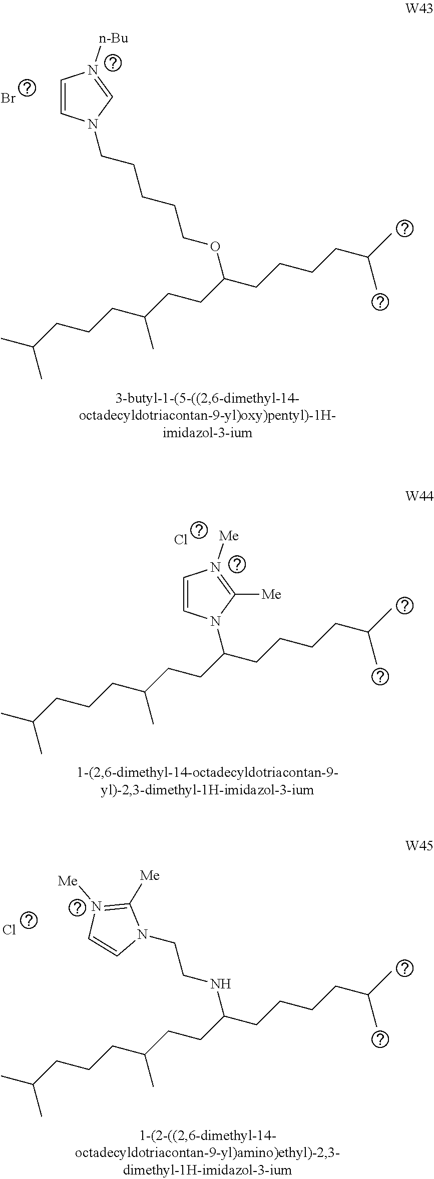

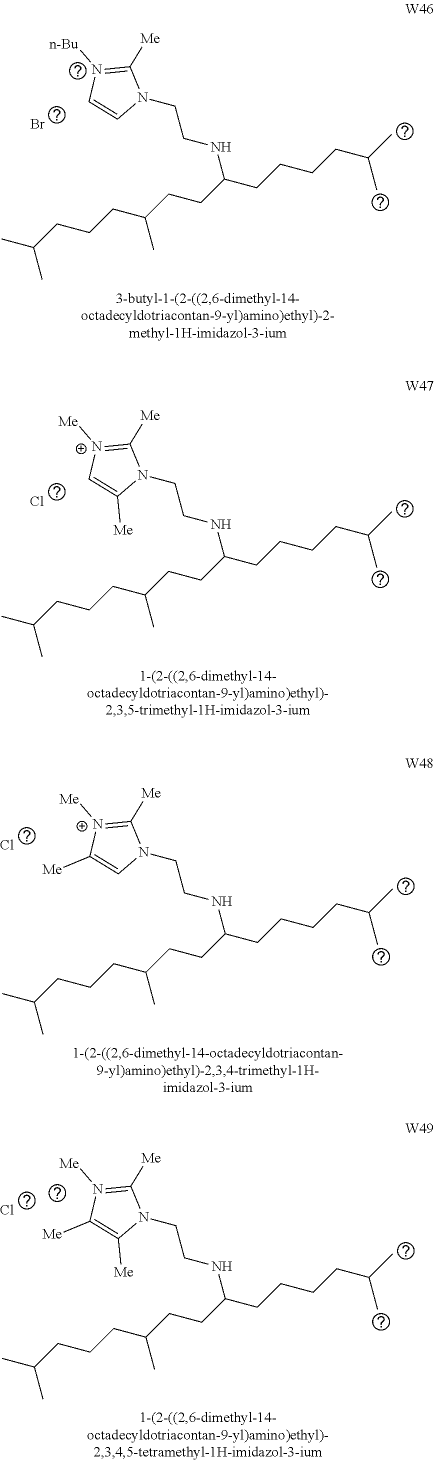

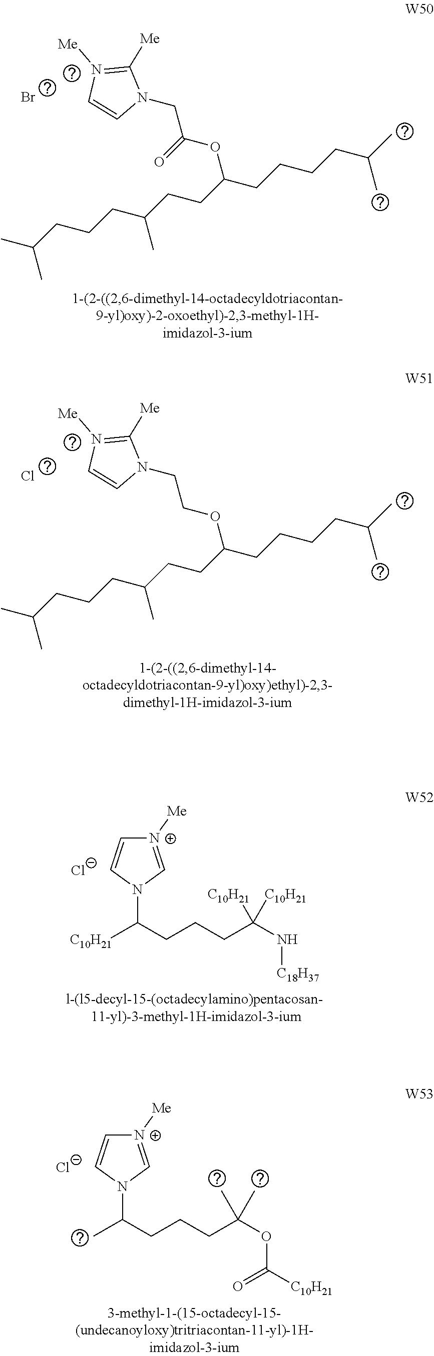

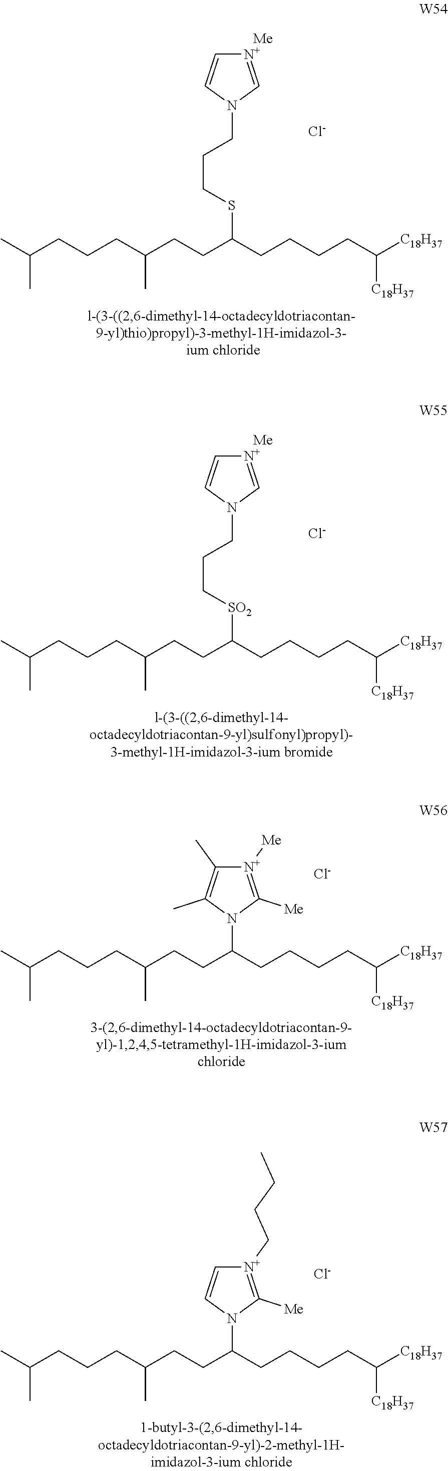

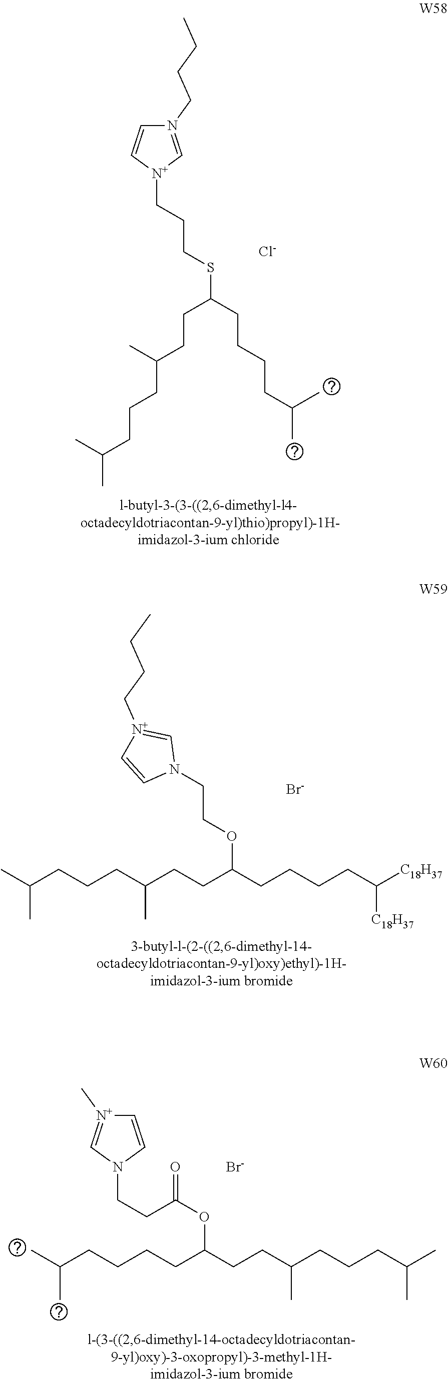

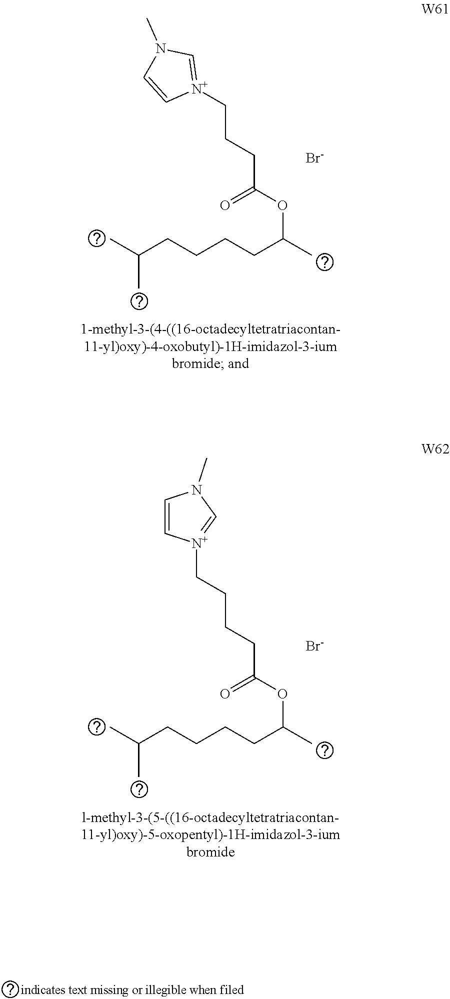

36. The composition according to claim 35, wherein the imidazolium-based cationic lipid of formula (I) is selected from the group consisting of compounds W12.7, W16.7, W19.7, W20.7, W21.7 and W22.7.

37. The composition according to claim 25, which comprises from 5 mole % to 40 mole % of the imidazolium-based cationic lipid.

38. The composition according to claim 25, wherein a percentage of encapsulation of the at least one nucleic acid in the at least one LNP is at least 80%.

39. The composition according to claim 25, wherein the at least one nucleic acid encodes a protein.

40. The composition according to claim 25, wherein the at least one nucleic acid is a therapeutic ingredient for use as a therapeutic agent or a prophylactic vaccine against viral infections, or a therapeutic vaccine against cancers.

41. The composition according to claim 26, wherein the at least one nucleic acid is mRNA.

42. The composition according to claim 36, wherein the imidazolium-based cationic lipid of formula (I) is W21.7.

43. A method for in vivo transfection of live cells comprising delivering the composition according to claim 25 in vivo to a target cell.

44. The method according to claim 43, wherein the target cell is a cell of a target organ selected from the group consisting of lungs, heart, brain, spleen, the nodes, bone marrow, bones, skeletal muscles, stomach, small intestine, large intestine, kidneys, bladder, breast, liver, testes, ovaries, uterus, spleen, thymus, brainstem, cerebellum, spinal cord, eye, ear, tongue and skin.

45. The method according to claim 44, wherein the target cell is a cell of lungs or a cell of spleen.

46. A method for in vitro transfection of live cells comprising introducing into live cells in vitro the composition according to claim 25.

47. The method according to claim 46, wherein the live cells are selected from the group consisting of mammalian cells, insect cells, cell lines, primary cells, adherent cells, cell suspensions cell, cancer cells and tumor cells.

48. A method of performing in vivo applications for nucleic acid-based therapy, comprising administering the composition according to claim 25, wherein the at least one nucleic acid is a therapeutic ingredient for use as a therapeutic agent or a prophylactic vaccine against viral infections, or a therapeutic vaccine against cancers.

49. A method for cell reprogramming, for differentiating cells, for gene-editing or for genome engineering, comprising applying to the cells, gene or genome the composition according to claim 25.

50. A method for the production of (i) biologics encoding a recombinant protein or antibody or (ii) recombinant virus, the method comprising applying the composition of claim 25.
